SEQUENCE LISTING (261 sequences)

SEQ ID NO: 1 - protein, 266 aa, Artificial Sequence
  DG01 SCFV
MEWTWVFLFLLSVTAGVHSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSAGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 2 - protein, 266 aa, Artificial Sequence
  DG02 SCFV
MEWTWVFLFLLSVTAGVHSEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSAGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIK

SEQ ID NO: 3 - protein, 259 aa, Artificial Sequence
  DG03 SCFV
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIK

SEQ ID NO: 4 - protein, 269 aa, Artificial Sequence
  DG04 SCFV
MEWTWVFLFLLSVTAGVHSQVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSAGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIK

SEQ ID NO: 5 - protein, 261 aa, Artificial Sequence
  DG05 SCFV
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 6 - protein, 259 aa, Artificial Sequence
  DG06 SCFV
MEWTWVFLFLLSVTAGVHSQVQLVESGGGVVQPGRSLRLSCAASGFTFSNYGIHWVRQAPGKGLEWVAIIWHDGSNSYYVDSVKGRFTISRDNSKNTLYLQMNSLRAEDTAVYFCARIIGGAFDIWGQGTMVTVSSAGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPITFGQGTRLEIK

SEQ ID NO: 7 - protein, 256 aa, Artificial Sequence
  DG07 SCFV
MEWTWVFLFLLSVTAGVHSEVQLVESGGGLVQPGGSLRLSCAASGFSISGYWMSWVRQAPGKGLEWVANIKQDGGEKYYGDSVKGRFTISRDNAKNSLYLQMNSLRAEDTAVYYCVMAGGLDYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWYTFGQGTKLEIK

SEQ ID NO: 8 - protein, 264 aa, Artificial Sequence
  DG08 SCFV
MEWTWVFLFLLSVTAGVHSQVQLVQSGAEVKKPGASVRLSCRASGYNFIDFHIHWVRQAPGEGLEWMGWSNPQSGNSSSAQRFQGRVTMTTDTSMSAAYMDLNWLTLDDTAVYYCTRPHDGAGNYRFDTWGQGTLVTVSSAGGGGSGGGGSGGGGSSYELTQPPSVSVAPGQTARITCSGDALPKHYAHWYQQKPGQVPIVVIYKDTERPSGIPERFSGSTSGTTVTLTISGVQAEDEAHYYCQSADVSSTYVVFGGGTKLTVL

SEQ ID NO: 9 - protein, 256 aa, Artificial Sequence
  DG09 SCFV
MEWTWVFLFLLSVTAGVHSEVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSAGGGGSGGGGSGGGGSSYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVL

SEQ ID NO: 10 - protein, 272 aa, Artificial Sequence
  DG10 SCFV
MEWTWVFLFLLSVTAGVHSQVQLVQSGAEVKKPGASVKVSCKASGYTFTNYAMHWVRQAPGQRLEWMGWINAGNGKRKYSQKFQDRVTINRDTSASTIYMELSSLGSEDTAVYYCAREEDHAGSGSYLSMDVWGQGSTVTVSSAGGGGSGGGGSGGGGSDIVMTQSPDSLAVSLGERATINCKSSQNVLYSSNNKNYLAWYQQKPGHPPKLLIYWASTRESGVPDRFSGSGSGTDFTLTITSLQTEDVAVYYCQQYYSSPLTFGGGTKVEIK

SEQ ID NO: 11 - protein, 268 aa, Artificial Sequence
  DG11 SCFV
MEWTWVFLFLLSVTAGVHSEVQLVETGGGLVQPKGSLKLSCATSGFTFNTYAMNWVRQAPGKGLEWVARIRTKSNDYATYYADSVKGRITISRDDSQSMLYLQMNNLKTEDTAMYYCVRVGYRPYAMDYWGQGTSVTVSSAGGGGSGGGGSGGGGSDVLMTQTPLSLPVSLGDQASISCRSSQNIVHSNGNTYLEWYLQKPGQSPTLLIYKVSNRFSGVPDRFSGSGSGTDFTLKISRVEAEDLGVYYCFQGSHVPLTFGAGTKLELK

SEQ ID NO: 12 - protein, 42 aa, Artificial Sequence
  4-1BB CO-STIMULATORY DOMAIN
KRGRKKLLYIFKQPFMRPVQTTQEEDGCSCRFPEEEEGGCEL

SEQ ID NO: 13 - protein, 15 aa, Artificial Sequence
  (G4S)3 LINKER
GGGGSGGGGSGGGGS

SEQ ID NO: 14 - protein, 26 aa, Artificial Sequence
  T2A
RAKRSGSGEGRGSLITCGDVEENPGP

SEQ ID NO: 15 - protein, 27 aa, Artificial Sequence
  P2A
RAKRSGSGATNFSLLKQAGDVEENPGP

SEQ ID NO: 16 - protein, 112 aa, Artificial Sequence
  CD3Z
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 770 aa, Artificial Sequence
  HUMAN AMYLOID BETA,  ISOFORM APP770 (IDENTIFIER: P05067-1)
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 18 - protein, 154 aa, Artificial Sequence [sequence omitted]

SEQ ID NO: 19 - protein, 140 aa, Artificial Sequence
  H HUMAN ALPHA-SYNUCLEIN, ISOFORM 1 (IDENTIFIER: P37840-1)
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 20 - protein, 467 aa, Artificial Sequence
  DG01.28.Z CAR
MEWTWVFLFLLSVTAGVHSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSAGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 21 - protein, 467 aa, Artificial Sequence
  DG02.28.Z CAR
MEWTWVFLFLLSVTAGVHSEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSAGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 22 - protein, 460 aa, Artificial Sequence
  DG03.28.Z CAR
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 23 - protein, 470 aa, Artificial Sequence
  DG04.28.Z CAR
MEWTWVFLFLLSVTAGVHSQVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSAGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 24 - protein, 462 aa, Artificial Sequence
  DG05.28.Z CAR
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 25 - protein, 460 aa, Artificial Sequence
  DG06.28.Z CAR
MEWTWVFLFLLSVTAGVHSQVQLVESGGGVVQPGRSLRLSCAASGFTFSNYGIHWVRQAPGKGLEWVAIIWHDGSNSYYVDSVKGRFTISRDNSKNTLYLQMNSLRAEDTAVYFCARIIGGAFDIWGQGTMVTVSSAGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPITFGQGTRLEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 26 - protein, 457 aa, Artificial Sequence
  DG07.28.Z CAR
MEWTWVFLFLLSVTAGVHSEVQLVESGGGLVQPGGSLRLSCAASGFSISGYWMSWVRQAPGKGLEWVANIKQDGGEKYYGDSVKGRFTISRDNAKNSLYLQMNSLRAEDTAVYYCVMAGGLDYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWYTFGQGTKLEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 27 - protein, 465 aa, Artificial Sequence
  DG08.28.Z CAR
MEWTWVFLFLLSVTAGVHSQVQLVQSGAEVKKPGASVRLSCRASGYNFIDFHIHWVRQAPGEGLEWMGWSNPQSGNSSSAQRFQGRVTMTTDTSMSAAYMDLNWLTLDDTAVYYCTRPHDGAGNYRFDTWGQGTLVTVSSAGGGGSGGGGSGGGGSSYELTQPPSVSVAPGQTARITCSGDALPKHYAHWYQQKPGQVPIVVIYKDTERPSGIPERFSGSTSGTTVTLTISGVQAEDEAHYYCQSADVSSTYVVFGGGTKLTVLASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 28 - protein, 457 aa, Artificial Sequence
  DG09.28.Z CAR
MEWTWVFLFLLSVTAGVHSEVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSAGGGGSGGGGSGGGGSSYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 29 - protein, 473 aa, Artificial Sequence
  DG10.28.Z CAR
MEWTWVFLFLLSVTAGVHSQVQLVQSGAEVKKPGASVKVSCKASGYTFTNYAMHWVRQAPGQRLEWMGWINAGNGKRKYSQKFQDRVTINRDTSASTIYMELSSLGSEDTAVYYCAREEDHAGSGSYLSMDVWGQGSTVTVSSAGGGGSGGGGSGGGGSDIVMTQSPDSLAVSLGERATINCKSSQNVLYSSNNKNYLAWYQQKPGHPPKLLIYWASTRESGVPDRFSGSGSGTDFTLTITSLQTEDVAVYYCQQYYSSPLTFGGGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 30 - protein, 469 aa, Artificial Sequence
  DG11.28.Z CAR
MEWTWVFLFLLSVTAGVHSEVQLVETGGGLVQPKGSLKLSCATSGFTFNTYAMNWVRQAPGKGLEWVARIRTKSNDYATYYADSVKGRITISRDDSQSMLYLQMNNLKTEDTAMYYCVRVGYRPYAMDYWGQGTSVTVSSAGGGGSGGGGSGGGGSDVLMTQTPLSLPVSLGDQASISCRSSQNIVHSNGNTYLEWYLQKPGQSPTLLIYKVSNRFSGVPDRFSGSGSGTDFTLKISRVEAEDLGVYYCFQGSHVPLTFGAGTKLELKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 31 - protein, 19 aa, Artificial Sequence
  SIGNAL SEQUENCE
MEWTWVFLFLLSVTAGVHS

SEQ ID NO: 32 - protein, 18 aa, Artificial Sequence
  HUMAN CD28 HINGE
VKGKHLCPSPLFPGPSKP

SEQ ID NO: 33 - protein, 15 aa, Artificial Sequence
  MOUSE CD28 HINGE
IKEKHLCHTQSSPKL

SEQ ID NO: 34 - protein, 45 aa, Artificial Sequence
  HUMAN CD8A HINGE
TTTPAPRPPTPAPTIASQPLSLRPEACRPAAGGAVHTRGLDFACD

SEQ ID NO: 35 - protein, 30 aa, Artificial Sequence
  HUMAN DAP10 HINGE
QTTPGERSSLPAFYPGTSGSCSGCGSLSLP

SEQ ID NO: 36 - protein, 27 aa, Artificial Sequence
  HUMAN CD28 TM
FWVLVVVGGVLACYSLLVTVAFIIFWV

SEQ ID NO: 37 - protein, 27 aa, Artificial Sequence
  MOUSE CD28 TM
FWALVVVAGVLFCYGLLVTVALCVIWT

SEQ ID NO: 38 - protein, 24 aa, Artificial Sequence
  HUMAN CD8A TM
IYIWAPLAGTCGVLLLSLVITLYC

SEQ ID NO: 39 - protein, 21 aa, Artificial Sequence
  HUMAN DAP10 TM
LLAGLVAADAVASLLIVGAVF

SEQ ID NO: 40 - protein, 451 aa, Artificial Sequence
  DG05-CD28tm-DAP10-CD3Z
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASVKGKHLCPSPLFPGPSKPFWVLVVVGGVLACYSLLVTVAFIIFWVRSKRSLCARPRRSPAQEDGKVYINMPGRGKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 491 aa, Artificial Sequence
  DG05-CD28tm-CD44-CD3Z
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASVKGKHLCPSPLFPGPSKPFWVLVVVGGVLACYSLLVTVAFIIFWVSRRRCGQKKKLVINSGNGAVEDRKPSGLNGEASKSQEMVHLVNKESSETPDQFMTADETRNLQNVDMKIGVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 426 aa, Artificial Sequence
  DG05-CD28tm-CD3Z
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASVKGKHLCPSPLFPGPSKPFWVLVVVGGVLACYSLLVTVAFIIFWVRSKRSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 349 aa, Artificial Sequence
  DG05-CD28
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 44 - protein, 319 aa, Artificial Sequence
  DG05-CD28tm
MEWTWVFLFLLSVTAGVHSEVQLVQSGGGLVKPGGSLRLSCAGSGFTFSSYSMHWLRQAPGKGLEWVSAIGTAGGTYYADSVKGRFTISRDNAKNSLYLQMNSLRAEDTAVYYCAREYFFGSGNYGYWGQGTLVTVSSAGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASVKGKHLCPSPLFPGPSKPFWVLVVVGGVLACYSLLVTVAFIIFWVRSKRSRLLHSD

SEQ ID NO: 45 - protein, 449 aa, Artificial Sequence
  DG03-CD28tm-DAP10-CD3Z
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWVRSKRSLCARPRRSPAQEDGKVYINMPGRGKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 489 aa, Artificial Sequence
  DG03-CD28tm-CD44-CD3Z
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWVSRRRCGQKKKLVINSGNGAVEDRKPSGLNGEASKSQEMVHLVNKESSETPDQFMTADETRNLQNVDMKIGVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 469 aa, Artificial Sequence
  DG03-CD28tm-4-1-BB-CD3Z
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWVRSKRSLEKRGRKKLLYIFKQPFMRPVQTTQEEDGCSCRFPEEEEGGCELKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 424 aa, Artificial Sequence
  DG03-CD28tm-CD3Z
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWVRSKRSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 347 aa, Artificial Sequence
  DG03-CD28
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 50 - protein, 306 aa, Artificial Sequence
  DG03-CD28tm
MEWTWVFLFLLSVTAGVHSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSAGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKASVKGKHLCPSPLFPGPSKPFWVLVVVGGVLACYSLLVTVAFIIFWV

SEQ ID NO: 51 - protein, 90 aa, Artificial Sequence
  Construct for expression of the NDMM Nrf2 (Keap1 inhibitor peptide)
MMDLELPPPGLPSQQDMDLIDILWRQDIDLGVSREVFDFSQRRKEYELEKQKKLEKERQEQLQKEQEKAFFAQLQLDEETGEFLPIQPAQ

SEQ ID NO: 52 - protein, 527 aa, Artificial Sequence
  Construct for expression of the NDMM human catalase
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 53 - protein, 247 aa, Artificial Sequence
  Construct for expression of the NDMM BDNF
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 54 - protein, 195 aa, Artificial Sequence
  Construct for expression of the NDMM IGF-1
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 55-200 - skipped (146 entries)

SEQ ID NO: 201 - DNA, 798 bp, Artificial Sequence
  DG01 SCFV
atggaatggacctgggtgttcctgtttctgctgtccgtgaccgctggcgtgcacagccaggtgcagctggtggaaagcggcggaggagtcgtgcagcctggcagaagcctgaggctgagctgtgccgccagcggcttcgccttcagctcctacggcatgcactgggtgagacaggcccctggcaagggactggagtgggtggctgtgatctggttcgacggcaccaagaagtactacaccgacagcgtcaagggcaggttcaccatctccagggacaatagcaagaataccctgtacctccaaatgaacaccctgagggccgaggacaccgccgtgtattactgcgccagggataggggaatcggcgccaggagaggcccctactacatggacgtgtggggcaagggcacaacagtgaccgtttcttctgctggaggaggaggttctggaggaggaggaagcggaggaggaggctccgacatccagatgacacagtcccccagctccctgtccgccagcgtgggcgatagagtgaccatcacctgcagggccagccagagcatctccagctacctgaactggtatcaacagaagcccggcaaagcccccaaactgctgatctacgctgccagcagcctgcagagcggcgtgccttccagattcagcggctccggcagcggcaccgatttcacactgaccatctccagcctgcagcccgaggacttcgccacctactactgccagcagagctacagcacccccctgacctttggcggaggcaccaaggtggagatcaaa

SEQ ID NO: 202 - DNA, 798 bp, Artificial Sequence
  DG02 SCFV
atggaatggacctgggtgtttctgttcctgctgagcgtgacagccggcgtgcacagcgaggtgcagctgctggagagcggaggaggactggtgcaacccggcggaagcctcagactgagctgtgccgccagcggcttcaccttcagcaactatggcatgagctgggtgaggcaggcccctggcaaaggcctcgaatgggtggcttccattaggagcggcggcggcaggacctattacagcgacaacgtgaagggcaggttcaccatctccagggacaatagcaagaacaccctgtacctgcagatgaactccctgagggccgaggataccgccgtgtactactgcgtgaggtacgaccactacagcggctccagcgactattggggacagggcaccctggtgacagtgtccagcgccggaggaggcggcagcggcggcggcggcagcggcggcggcggaagcgatgtggtgatgacccagtcccccctgagcctgcctgtgacacctggagagcccgccagcatcagctgtaagagcagccagagcctcctggacagcgacggcaaaacctacctgaactggctcctgcagaagcccggacaaagcccccagaggctgatctacctggtgagcaaactggacagcggcgtgcctgacagattctccggctccggcagcggcaccgacttcacactgaagatcagcagagtggaggctgaggacgtgggcgtctactactgctggcagggcacccacttccccaggaccttcggccagggaaccaaggtggagatcaag

SEQ ID NO: 203 - DNA, 777 bp, Artificial Sequence
  DG03 SCFV
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaa

SEQ ID NO: 204 - DNA, 807 bp, Artificial Sequence
  DG04 SCFV
atggaatggacctgggtgtttctgttcctcctgagcgtgaccgccggagtgcacagccaagtggagctggtggagagcggaggaggactggtgcagcctggaggctccctgaggctgagctgtgctgccagcggcttcaccttcagctcctatgctatgagctgggtgagacaggcccctggcaaaggcctggagtgggtgagcgccatcaacgcctccggcaccaggacctactatgccgactccgtgaagggcaggttcaccatctccagggacaacagcaagaacaccctgtacctgcagatgaacagcctgagggctgaagacaccgccgtgtactactgtgccaggggcaagggcaacacacacaagccctatggctacgtgagatacttcgacgtgtggggacagggcaccctggtgacagtgagcagcgccggaggaggaggttctggaggaggaggaagcggcggaggaggaagcgacatcgtgctgacacaatcccccgccacactgtccctgtcccctggcgagagggccacactgagctgcagggccagccagagcgtgtcctcctcctacctggcctggtaccagcagaaacctggccaggcccccaggctgctgatctatggcgccagcagcagagccacaggagtgcctgccagatttagcggcagcggcagcggcaccgactttaccctgaccatttccagcctggagcccgaggacttcgccacctactactgcctgcagatctacaacatgcctatcaccttcggccagggcacaaaagtggaaatcaag

SEQ ID NO: 205 - DNA, 783 bp, Artificial Sequence
  DG05 SCFV
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaag

SEQ ID NO: 206 - DNA, 777 bp, Artificial Sequence
  DG06 SCFV
atggaatggacctgggtgttcctgttcctcctgagcgtgaccgctggcgtccacagccaggtgcagctggtggaaagcggaggcggagtggtgcagcctggaaggtccctcagactgagctgcgctgccagcggcttcaccttcagcaactacggcatccactgggtgagacaagcccccggcaaaggcctggagtgggtggccatcatctggcacgacggcagcaactcctactacgtggactccgtgaagggcaggttcacaatcagcagagacaacagcaagaataccctgtacctgcagatgaacagcctcagggccgaagataccgccgtgtacttctgcgccaggatcatcggcggcgcctttgacatttggggccaaggcactatggttaccgtgagcagcgctggcggaggcggcagcggcggcggcggcagcggcggcggcggaagcgacatccagatgacccagagcccttccagcctcagcgcctccgtgggagacagagtgaccatcacctgcagggccagccagggcatctccagctggctggcctggtaccagcagaagcctgagaaagcccccaagagcctgatctacgctgcctccagcctgcagtccggcgtgccttccagattctccggcagcggcagcggcaccgactttaccctgaccatttccagcctgcaacccgaggacttcgccacctactactgccagcagtacaacagctaccccatcacctttggccagggcaccagactggagatcaag

SEQ ID NO: 207 - DNA, 768 bp, Artificial Sequence
  DG07 SCFV
atggaatggacctgggtgttcctgttcctgctgtccgtgaccgccggagtccacagcgaggtgcagctggtggaaagcggcggaggactggtgcagcctggaggcagcctgaggctgagctgtgctgccagcggcttctccatcagcggctactggatgtcctgggtgaggcaggcccctggaaagggcttagaatgggtggccaacatcaaacaggacggcggcgagaagtactacggagacagcgtcaagggcagattcaccatcagcagggacaacgccaagaacagcctgtacctgcagatgaacagcctgagggccgaggacaccgctgtgtactactgcgtgatggccggaggcctggattattggggccagggcacactggtgacagtgagcagcgccggcggcggcggcagcggcggcggcggcagcggcggcggcggcagcgagatcgtgctgacccagagccctgccacactgagcctgagccccggcgaaagagccaccctcagctgcagggccagccagagcgtgagcagctacctggcctggtaccagcagaagcccggacaggcccctaggctgctgatctacgatgccagcaacagagccaccggcatccctgctaggttcagcggttctggcagcggcaccgacttcaccctgaccatcagcagcctggagcctgaggacttcgctgtctactactgccagcagaggagcaactggtataccttcggccagggaaccaagctggagatcaag

SEQ ID NO: 208 - DNA, 792 bp, Artificial Sequence
  DG08 SCFV
atggaatggacctgggtgttcctgttcctgctcagcgtgaccgccggagtgcactcccaggtgcagctggtgcagagcggcgccgaagtgaaaaagcccggcgccagcgtgagactctcctgtagagccagcggctacaacttcatcgacttccacatccactgggtgagacaggcccctggagagggcctggagtggatgggctggagcaacccccagagcggcaatagcagcagcgcccagaggttccagggcagagtgaccatgaccaccgatacctccatgagcgccgcctacatggacctgaactggctgaccctggacgacaccgccgtgtactactgcaccaggcctcacgacggcgctggcaactacaggttcgacacctggggacagggaaccctggtgacagtcagcagcgcgggaggtggtggtagcggtggtgggggttccggtggaggtggcagttcatatgagcttacacaacccccaagtgtgagcgtggctcctggccagacagccaggatcacctgcagcggagacgccctgcccaaacactacgcccactggtatcagcagaaacccggccaggtgcccatcgtggtgatctacaaggacaccgagagacctagcggcatccccgagagattcagcggcagcaccagcggcaccacagtcaccctgaccatctccggcgtgcaggccgaggatgaagcccactactactgccaaagcgccgacgtgtcctccacctatgtggtgttcggcggcggcaccaagctgaccgtcctc

SEQ ID NO: 209 - DNA, 768 bp, Artificial Sequence
  DG09 SCFV
atggagtggacttgggtattcctgtttttgttgtccgtaactgctggggtacactcagaggtgcagctggtagagtctggaggggggctcgtcgaaccaggcggctctctcaggctttcctgtgccgtaagcgggtttgattttgagaaagcatggatgtcctgggtaaggcaagctccagggcagggactccagtgggtagcgcggataaagtcaacagctgatggcggaaccacctcttatgcagcaccggttgagggaaggttcatcatctcacgagacgattcccgcaacatgttgtatctgcagatgaacagtttgaaaactgaagacacagctgtttactactgtacttcagcgcattggggacagggtactcttgtgacggtctctagcgccggggggggaggctctggaggggggggttcagggggtggtggcagctcctatgagctgactcaaccgccttcagtaagcgtaagccctggtcagaccgctagaataacctgtagtggagaggccctgccgatgcaattcgcccactggtatcagcagaggcctggaaaagccccagtgattgtcgtttacaaagattccgaacgccctagcggggttcccgaacgctttagcggtagttcaagcgggacaacagcaacccttacgataaccggtgtacaagcggaagacgaagcggattactattgccaatcacctgatagtacaaatacttatgaggtatttggcgggggaacgaagttgactgtactg

SEQ ID NO: 210 - DNA, 816 bp, Artificial Sequence
  DG10 SCFV
atggaatggacctgggtgttcctcttcctcctgtccgtcaccgctggcgtgcacagccaggtgcagctggtccagagcggagccgaggtgaaaaagcccggcgcctccgtgaaggtcagctgcaaggcctccggctacaccttcaccaactacgccatgcactgggtgagacaggcccctggccagagactggagtggatgggctggatcaacgccggcaacggcaagagaaagtacagccagaagtttcaggacagggtgaccatcaacagggacaccagcgcctccaccatctacatggagctgtccagcctgggcagcgaggataccgccgtgtactactgtgccagagaggaggatcacgctggcagcggcagctacctgagcatggacgtctggggacagggcagcaccgtgacagtgagcagcgctggaggcggcggctccggcggcggaggaagcggaggcggaggctccgacatcgtgatgacccagtcccccgatagcctggctgtgagcctgggcgagagggccacaatcaactgtaagagcagccagaacgtgctgtactcctccaacaacaagaactacctggcctggtaccagcagaaacctggccatccccccaagctgctgatctactgggccagcaccagggagagcggagtgcctgacaggtttagcggcagcggcagcggcacagactttaccctgaccatcacctccctgcagaccgaggacgtggccgtgtactattgccagcagtactacagctcccctctgaccttcggcggcggcaccaaagtggagatcaaa

SEQ ID NO: 211 - DNA, 804 bp, Artificial Sequence
  DG11 SCFV
atggagtggacttgggtattccttttcctcctctccgtgacagcgggtgtgcactctgaagtacaacttgtagagaccggtgggggattggtgcaacccaagggttccctgaaactctcatgtgctacctctggttttactttcaacacctatgcaatgaattgggttaggcaagcacccggtaaaggacttgagtgggtggcacggatacgcactaagagtaatgactatgctacgtactacgcagactccgtaaaaggccggatcaccatatctcgagacgatagccagtctatgctgtatcttcaaatgaacaacctcaaaacggaagatacggcgatgtattactgcgtgcgagttggttataggccttatgctatggattactggggacagggcacgtctgtcacggtaagttctgccggaggggggggcagcggaggaggaggatctggcggagggggctccgatgtccttatgacacagactcccctcagtttgcccgtgtccttgggggaccaggcttctatatcatgccgcagttcccaaaatatcgtccattcaaatggcaatacttaccttgagtggtatttgcagaagcctggacagagcccgacgcttctgatctataaggtaagcaacaggttcagtggtgtacccgacagatttagtggaagtgggtccggaactgatttcactcttaagattagtcgggtagaggctgaagaccttggggtgtattattgctttcaagggagtcacgtccctcttacatttggtgctgggactaagttggagctgaag

SEQ ID NO: 212 - DNA, 126 bp, Artificial Sequence
  4-1BB CO-STIMULATORY DOMAIN
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 213 - DNA, 45 bp, Artificial Sequence
  (G4S)3 LINKER
ggcggaggcggatcaggaggaggaggatcaggcggaggaggatca

SEQ ID NO: 214 - DNA, 78 bp, Artificial Sequence
  T2A
agagccaaaaggtctggctccggtgagggcagaggaagtcttataacatgcggtgacgtggaggagaatcccggccct

SEQ ID NO: 215 - DNA, 81 bp, Artificial Sequence
  P2A
agagccaaaaggtccggaagcggcgccaccaacttcagcctgctgaagcaggccggcgacgtggaagagaatcctggcccc

SEQ ID NO: 216 - DNA, 336 bp, Artificial Sequence
  CD3Z
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 217-219 - skipped (3 entries)

SEQ ID NO: 220 - DNA, 1401 bp, Artificial Sequence
  DG01.28.Z CAR
atggaatggacctgggtgttcctgtttctgctgtccgtgaccgctggcgtgcacagccaggtgcagctggtggaaagcggcggaggagtcgtgcagcctggcagaagcctgaggctgagctgtgccgccagcggcttcgccttcagctcctacggcatgcactgggtgagacaggcccctggcaagggactggagtgggtggctgtgatctggttcgacggcaccaagaagtactacaccgacagcgtcaagggcaggttcaccatctccagggacaatagcaagaataccctgtacctccaaatgaacaccctgagggccgaggacaccgccgtgtattactgcgccagggataggggaatcggcgccaggagaggcccctactacatggacgtgtggggcaagggcacaacagtgaccgtttcttctgctggaggaggaggttctggaggaggaggaagcggaggaggaggctccgacatccagatgacacagtcccccagctccctgtccgccagcgtgggcgatagagtgaccatcacctgcagggccagccagagcatctccagctacctgaactggtatcaacagaagcccggcaaagcccccaaactgctgatctacgctgccagcagcctgcagagcggcgtgccttccagattcagcggctccggcagcggcaccgatttcacactgaccatctccagcctgcagcccgaggacttcgccacctactactgccagcagagctacagcacccccctgacctttggcggaggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 221 - DNA, 1401 bp, Artificial Sequence
  DG02.28.Z CAR
atggaatggacctgggtgtttctgttcctgctgagcgtgacagccggcgtgcacagcgaggtgcagctgctggagagcggaggaggactggtgcaacccggcggaagcctcagactgagctgtgccgccagcggcttcaccttcagcaactatggcatgagctgggtgaggcaggcccctggcaaaggcctcgaatgggtggcttccattaggagcggcggcggcaggacctattacagcgacaacgtgaagggcaggttcaccatctccagggacaatagcaagaacaccctgtacctgcagatgaactccctgagggccgaggataccgccgtgtactactgcgtgaggtacgaccactacagcggctccagcgactattggggacagggcaccctggtgacagtgtccagcgccggaggaggcggcagcggcggcggcggcagcggcggcggcggaagcgatgtggtgatgacccagtcccccctgagcctgcctgtgacacctggagagcccgccagcatcagctgtaagagcagccagagcctcctggacagcgacggcaaaacctacctgaactggctcctgcagaagcccggacaaagcccccagaggctgatctacctggtgagcaaactggacagcggcgtgcctgacagattctccggctccggcagcggcaccgacttcacactgaagatcagcagagtggaggctgaggacgtgggcgtctactactgctggcagggcacccacttccccaggaccttcggccagggaaccaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 222 - DNA, 1380 bp, Artificial Sequence
  DG03.28.Z CAR
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 223 - DNA, 1410 bp, Artificial Sequence
  DG04.28.Z CAR
atggaatggacctgggtgtttctgttcctcctgagcgtgaccgccggagtgcacagccaagtggagctggtggagagcggaggaggactggtgcagcctggaggctccctgaggctgagctgtgctgccagcggcttcaccttcagctcctatgctatgagctgggtgagacaggcccctggcaaaggcctggagtgggtgagcgccatcaacgcctccggcaccaggacctactatgccgactccgtgaagggcaggttcaccatctccagggacaacagcaagaacaccctgtacctgcagatgaacagcctgagggctgaagacaccgccgtgtactactgtgccaggggcaagggcaacacacacaagccctatggctacgtgagatacttcgacgtgtggggacagggcaccctggtgacagtgagcagcgccggaggaggaggttctggaggaggaggaagcggcggaggaggaagcgacatcgtgctgacacaatcccccgccacactgtccctgtcccctggcgagagggccacactgagctgcagggccagccagagcgtgtcctcctcctacctggcctggtaccagcagaaacctggccaggcccccaggctgctgatctatggcgccagcagcagagccacaggagtgcctgccagatttagcggcagcggcagcggcaccgactttaccctgaccatttccagcctggagcccgaggacttcgccacctactactgcctgcagatctacaacatgcctatcaccttcggccagggcacaaaagtggaaatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 224 - DNA, 1386 bp, Artificial Sequence
  DG05.28.Z CAR
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 225 - DNA, 1380 bp, Artificial Sequence
  DG06.28.Z CAR
atggaatggacctgggtgttcctgttcctcctgagcgtgaccgctggcgtccacagccaggtgcagctggtggaaagcggaggcggagtggtgcagcctggaaggtccctcagactgagctgcgctgccagcggcttcaccttcagcaactacggcatccactgggtgagacaagcccccggcaaaggcctggagtgggtggccatcatctggcacgacggcagcaactcctactacgtggactccgtgaagggcaggttcacaatcagcagagacaacagcaagaataccctgtacctgcagatgaacagcctcagggccgaagataccgccgtgtacttctgcgccaggatcatcggcggcgcctttgacatttggggccaaggcactatggttaccgtgagcagcgctggcggaggcggcagcggcggcggcggcagcggcggcggcggaagcgacatccagatgacccagagcccttccagcctcagcgcctccgtgggagacagagtgaccatcacctgcagggccagccagggcatctccagctggctggcctggtaccagcagaagcctgagaaagcccccaagagcctgatctacgctgcctccagcctgcagtccggcgtgccttccagattctccggcagcggcagcggcaccgactttaccctgaccatttccagcctgcaacccgaggacttcgccacctactactgccagcagtacaacagctaccccatcacctttggccagggcaccagactggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 226 - DNA, 1371 bp, Artificial Sequence
  DG07.28.Z CAR
atggaatggacctgggtgttcctgttcctgctgtccgtgaccgccggagtccacagcgaggtgcagctggtggaaagcggcggaggactggtgcagcctggaggcagcctgaggctgagctgtgctgccagcggcttctccatcagcggctactggatgtcctgggtgaggcaggcccctggaaagggcttagaatgggtggccaacatcaaacaggacggcggcgagaagtactacggagacagcgtcaagggcagattcaccatcagcagggacaacgccaagaacagcctgtacctgcagatgaacagcctgagggccgaggacaccgctgtgtactactgcgtgatggccggaggcctggattattggggccagggcacactggtgacagtgagcagcgccggcggcggcggcagcggcggcggcggcagcggcggcggcggcagcgagatcgtgctgacccagagccctgccacactgagcctgagccccggcgaaagagccaccctcagctgcagggccagccagagcgtgagcagctacctggcctggtaccagcagaagcccggacaggcccctaggctgctgatctacgatgccagcaacagagccaccggcatccctgctaggttcagcggttctggcagcggcaccgacttcaccctgaccatcagcagcctggagcctgaggacttcgctgtctactactgccagcagaggagcaactggtataccttcggccagggaaccaagctggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 227 - DNA, 1395 bp, Artificial Sequence
  DG08.28.Z CAR
atggaatggacctgggtgttcctgttcctgctcagcgtgaccgccggagtgcactcccaggtgcagctggtgcagagcggcgccgaagtgaaaaagcccggcgccagcgtgagactctcctgtagagccagcggctacaacttcatcgacttccacatccactgggtgagacaggcccctggagagggcctggagtggatgggctggagcaacccccagagcggcaatagcagcagcgcccagaggttccagggcagagtgaccatgaccaccgatacctccatgagcgccgcctacatggacctgaactggctgaccctggacgacaccgccgtgtactactgcaccaggcctcacgacggcgctggcaactacaggttcgacacctggggacagggaaccctggtgacagtcagcagcgcgggaggtggtggtagcggtggtgggggttccggtggaggtggcagttcatatgagcttacacaacccccaagtgtgagcgtggctcctggccagacagccaggatcacctgcagcggagacgccctgcccaaacactacgcccactggtatcagcagaaacccggccaggtgcccatcgtggtgatctacaaggacaccgagagacctagcggcatccccgagagattcagcggcagcaccagcggcaccacagtcaccctgaccatctccggcgtgcaggccgaggatgaagcccactactactgccaaagcgccgacgtgtcctccacctatgtggtgttcggcggcggcaccaagctgaccgtcctcgctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 228 - DNA, 1371 bp, Artificial Sequence
  DG09.28.Z CAR
atggagtggacttgggtattcctgtttttgttgtccgtaactgctggggtacactcagaggtgcagctggtagagtctggaggggggctcgtcgaaccaggcggctctctcaggctttcctgtgccgtaagcgggtttgattttgagaaagcatggatgtcctgggtaaggcaagctccagggcagggactccagtgggtagcgcggataaagtcaacagctgatggcggaaccacctcttatgcagcaccggttgagggaaggttcatcatctcacgagacgattcccgcaacatgttgtatctgcagatgaacagtttgaaaactgaagacacagctgtttactactgtacttcagcgcattggggacagggtactcttgtgacggtctctagcgccggggggggaggctctggaggggggggttcagggggtggtggcagctcctatgagctgactcaaccgccttcagtaagcgtaagccctggtcagaccgctagaataacctgtagtggagaggccctgccgatgcaattcgcccactggtatcagcagaggcctggaaaagccccagtgattgtcgtttacaaagattccgaacgccctagcggggttcccgaacgctttagcggtagttcaagcgggacaacagcaacccttacgataaccggtgtacaagcggaagacgaagcggattactattgccaatcacctgatagtacaaatacttatgaggtatttggcgggggaacgaagttgactgtactggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 229 - DNA, 1419 bp, Artificial Sequence
  DG10.28.Z CAR
atggaatggacctgggtgttcctcttcctcctgtccgtcaccgctggcgtgcacagccaggtgcagctggtccagagcggagccgaggtgaaaaagcccggcgcctccgtgaaggtcagctgcaaggcctccggctacaccttcaccaactacgccatgcactgggtgagacaggcccctggccagagactggagtggatgggctggatcaacgccggcaacggcaagagaaagtacagccagaagtttcaggacagggtgaccatcaacagggacaccagcgcctccaccatctacatggagctgtccagcctgggcagcgaggataccgccgtgtactactgtgccagagaggaggatcacgctggcagcggcagctacctgagcatggacgtctggggacagggcagcaccgtgacagtgagcagcgctggaggcggcggctccggcggcggaggaagcggaggcggaggctccgacatcgtgatgacccagtcccccgatagcctggctgtgagcctgggcgagagggccacaatcaactgtaagagcagccagaacgtgctgtactcctccaacaacaagaactacctggcctggtaccagcagaaacctggccatccccccaagctgctgatctactgggccagcaccagggagagcggagtgcctgacaggtttagcggcagcggcagcggcacagactttaccctgaccatcacctccctgcagaccgaggacgtggccgtgtactattgccagcagtactacagctcccctctgaccttcggcggcggcaccaaagtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 230 - DNA, 1407 bp, Artificial Sequence
  DG11.28.Z CAR
atggagtggacttgggtattccttttcctcctctccgtgacagcgggtgtgcactctgaagtacaacttgtagagaccggtgggggattggtgcaacccaagggttccctgaaactctcatgtgctacctctggttttactttcaacacctatgcaatgaattgggttaggcaagcacccggtaaaggacttgagtgggtggcacggatacgcactaagagtaatgactatgctacgtactacgcagactccgtaaaaggccggatcaccatatctcgagacgatagccagtctatgctgtatcttcaaatgaacaacctcaaaacggaagatacggcgatgtattactgcgtgcgagttggttataggccttatgctatggattactggggacagggcacgtctgtcacggtaagttctgccggaggggggggcagcggaggaggaggatctggcggagggggctccgatgtccttatgacacagactcccctcagtttgcccgtgtccttgggggaccaggcttctatatcatgccgcagttcccaaaatatcgtccattcaaatggcaatacttaccttgagtggtatttgcagaagcctggacagagcccgacgcttctgatctataaggtaagcaacaggttcagtggtgtacccgacagatttagtggaagtgggtccggaactgatttcactcttaagattagtcgggtagaggctgaagaccttggggtgtattattgctttcaagggagtcacgtccctcttacatttggtgctgggactaagttggagctgaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccct

SEQ ID NO: 231 - DNA, 54 bp, Artificial Sequence
  SIGNAL SEQUENCE
atggaatggacctgggtctttctcttcctcctgtcagtaactgcaggtgtccac

SEQ ID NO: 232 - DNA, 54 bp, Artificial Sequence
  HUMAN CD28 HINGE
gtgaaagggaaacacctttgtccaagtcccctatttcccggaccttctaagccc

SEQ ID NO: 233 - DNA, 45 bp, Artificial Sequence
  MOUSE CD28 HINGE
ataaaagagaaacatctttgtcatactcagtcatctcctaagctg

SEQ ID NO: 234 - DNA, 135 bp, Artificial Sequence
  HUMAN CD8A HINGE
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 235 - DNA, 90 bp, Artificial Sequence
  HUMAN DAP10 HINGE
cagacgaccccaggagagagatcatcactccctgccttttaccctggcacttcaggctcctgttccggatgtgggtccctctctctgccg

SEQ ID NO: 236 - DNA, 81 bp, Artificial Sequence
  HUMAN CD28 TM
ttttgggtgctggtggtggttggtggagtcctggcttgctatagcttgctagtaacagtggcctttattattttctgggtg

SEQ ID NO: 237 - DNA, 81 bp, Artificial Sequence
  MOUSE CD28 TM
ttttgggcactggtcgtggttgctggagtcctgttttgttatggcttgctagtgacagtggctctttgtgttatctggaca

SEQ ID NO: 238 - DNA, 72 bp, Artificial Sequence
  HUMAN CD8A TM
atctacatctgggcgcccttggccgggacttgtggggtccttctcctgtcactggttatcaccctttactgc

SEQ ID NO: 239 - DNA, 63 bp, Artificial Sequence
  HUMAN DAP10 TM
ctcctggcaggcctcgtggctgctgatgcggtggcatcgctgctcatcgtgggggcggtgttc

SEQ ID NO: 240 - DNA, 1353 bp, Artificial Sequence
  DG05-CD28tm-DAP10-CD3Z
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcctgtgcgcacgcccacgccgcagccccgcccaagaagatggcaaagtctacatcaacatgccaggcaggggcaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 241 - DNA, 1473 bp, Artificial Sequence
  DG05-CD28tm-CD44-CD3Z
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 242 - DNA, 1278 bp, Artificial Sequence
  DG05-CD28tm-CD3Z
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 243 - DNA, 1047 bp, Artificial Sequence
  DG05-CD28
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 244 - DNA, 957 bp, Artificial Sequence
  DG05-CD28tm
atggaatggacctgggtgtttctgttcctcctgtccgtgaccgccggagtgcactccgaagtgcagctggtgcagtccggcggaggactggtgaaacccggaggaagcctcagactgagctgcgccggcagcggctttaccttctccagctactccatgcactggctgagacaggcccctggcaagggcctggaatgggtcagcgccatcggcaccgccggaggcacatactatgccgacagcgtgaagggcaggttcaccatcagcagggacaacgccaagaacagcctgtacctgcagatgaactctctgagggccgaggataccgctgtgtactactgcgccagggagtacttctttggcagcggcaactacggatactggggccagggcaccctggtgacagtgagctccgccggaggaggaggaagcggaggaggcggaagcggaggaggcggcagcgaaatcgtgctgacccagagccctgccaccctgagcctgagccctggcgaaagggccaccctgagctgcagagccagccagagcgtgagcagctacctggcctggtaccagcagaagcccggacaggcccccagactgctgatctacgacgccagcaacagagccaccggcattcccgccagattctccggcagcggcagcggaaccgacttcacactgaccatcagctccttagaacccgaggacttcgccgtgtactactgtcagcagagaagcaactggcctcccaccttcggccagggcacaaaggtggagatcaaggctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgac

SEQ ID NO: 245 - DNA, 1347 bp, Artificial Sequence
  DG03-CD28tm-DAP10-CD3Z
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcctgtgcgcacgcccacgccgcagccccgcccaagaagatggcaaagtctacatcaacatgccaggcaggggcaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 246 - DNA, 1467 bp, Artificial Sequence
  DG03-CD28tm-CD44-CD3Z
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 247 - DNA, 1407 bp, Artificial Sequence
  DG03-CD28tm-4-1-BB-CD3Z
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcctcgagaaacggggcagaaagaaactcctgtatatattcaaacaaccatttatgagaccagtacaaactactcaagaggaagatggctgtagctgccgatttccagaagaagaagaaggaggatgtgaactgaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 248 - DNA, 1272 bp, Artificial Sequence
  DG03-CD28tm-CD3Z
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 249 - DNA, 1041 bp, Artificial Sequence
  DG03-CD28
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 250 - DNA, 918 bp, Artificial Sequence
  DG03-CD28tm
atggaatggacctgggtgttcctgtttctgctctccgtgaccgccggagtgcacagcgaggtgcagctggtcgaaagcggcggaggactggtgcagcctggcggcagcctgagactgagctgtgccgcctccggcttcacctttagcagctacggaatgtcctgggtgagacaggctcctggcaagggcctggaactggtggccagcatcaatagcaacggcggcagcacctactaccctgatagcgtgaagggcaggttcaccatctccagggacaacgccaagaacagcctgtacctgcagatgaacagcctcagggccgaggacacagccgtgtactactgcgccagcggcgactattggggacagggaacaaccgtgaccgtcagcagcgccggcggcggcggcagcggcggcggcggcagcggcggcggcggctccgatatcgtgatgacccagagccccctgtccctgcctgtcacacctggcgaacccgccagcattagctgcaggtccagccagagcctggtgtacagcaatggcgacacctacctgcactggtacctgcagaagcctggccagagcccccagctgctgatctacaaggtgagcaacaggttctccggagtgcctgacaggttcagcggctccggcagcggaaccgatttcaccctcaagatcagcagagtggaggccgaggacgtgggcgtctactattgtagccagagcacccacgtgccctggacctttggccagggcaccaaggtggagatcaaagctagcgtgaaagggaaacacctttgtccaagtcccctatttcccggaccttctaagcccttttgggtgctggtggtggttggtggagtcctggcttgctatagcttgctagtaacagtggcctttattattttctgggtg

SEQ ID NO: 251 - DNA, 270 bp, Artificial Sequence
  Construct for expression of the NDMM Nrf2 (Keap1 inhibitor peptide)
atgatggatttggaacttccccccccagggctcccatcccaacaagacatggatctcatagacatactgtggagacaggacatcgatctgggggtcagccgcgaagttttcgacttttcacaaaggcggaaagaatatgaattggaaaagcagaaaaaattggaaaaagaacgccaggaacagcttcagaaggagcaggaaaaagccttttttgcccagcttcagctggacgaggaaacaggggaatttctccccatccaaccagcccag

SEQ ID NO: 252 - DNA, 1581 bp, Artificial Sequence
  Construct for expression of the NDMM human catalase
atggcagacagtcgagaccctgctagcgaccaaatgcaacattggaaagagcaacgggcggcccagaaagccgacgttttgaccactggggcaggtaatcctgttggagataagctgaacgtcatcacggttggaccccggggaccgctgctcgttcaagacgtggtttttacggatgagatggcccattttgatcgagagaggataccagaaagggttgtgcacgctaagggcgcaggtgccttcggatatttcgaggtaactcacgacattactaagtatagcaaggccaaggtatttgaacacattggcaagaagacgccgatagcggtccgattcagtacagtggcgggcgagtcaggttcagccgataccgtgagagatccgagaggatttgccgtgaaattttatacagaggacggcaactgggacttggtaggaaacaataccccaatatttttcataagggacccaatcctttttcccagctttattcattcacagaagcggaacccacaaacgcacttgaaagatcctgacatggtgtgggatttttggagcttgaggccagagagcctgcaccaagtgagcttcttgttcagcgacagaggcataccggacggtcatagacacatgaacggttacggtagtcacaccttcaaactggtgaacgccaacggagaggctgtctattgtaagttccactataaaaccgatcaaggcatcaaaaacctgagcgtagaggacgcagcccgcctttctcaagaagatccagactatgggatccgggatctctttaacgccatagctacgggtaaatatccctcttggacgttctatatccaggtaatgacattcaatcaagcagagactttcccctttaacccgtttgaccttactaaagtatggccgcataaggactaccctctgattcccgtcggcaaactcgtgcttaacaggaatccagtcaactatttcgcagaagtcgagcaaatcgcctttgacccttctaacatgccgccgggaatcgaagcgtcaccggacaagatgcttcaaggtcggcttttcgcataccccgacactcaccgacacagactgggtccgaattatcttcacatacctgtcaactgcccatatagagcacgcgttgcgaactaccagcgcgatggtccgatgtgcatgcaggacaaccaggggggggcacccaactattatccaaattcatttggggcgccggaacaacaaccgtcagcccttgaacactccatccagtattctggcgaagtaagacgcttcaacacggctaatgatgacaacgttacacaggttagagcgttttatgtgaacgtcttgaacgaggaacaacggaaacgactttgcgaaaacatagcgggtcatttgaaagatgctcagatttttatccaaaaaaaagccgtcaaaaattttaccgaagtccaccccgattacggttcacatattcaggccctgttggataagtacaacgcggaaaagcccaagaatgcaatacacacgtttgttcagagcgggagccacctcgctgctcgagagaaagcaaatctg

SEQ ID NO: 253 - DNA, 741 bp, Artificial Sequence
  Construct for expression of the NDMM BDNF
atgacgatcctgtttctgacaatggtgattagctatttcggatgtatgaaagccgccccgatgaaggaggccaatatcaggggacaaggtgggctggcttatccgggcgtaaggacacacggcacactggagagtgtgaacggcccgaaggccggatcacgaggattgacgagcctcgcagatacgtttgagcatgtaatcgaagagctcttggatgaagaccaaaaggtccgccccaatgaggagaacaacaaagacgcagacctgtacacatcacgagttatgctgtcaagtcaagtgccgctcgaaccaccactcctctttctgctggaggagtacaaaaactatttggacgctgctaacatgtctatgcgagtgcgcagacatagtgaccctgccagacgcggtgagctttcagtctgtgattctataagtgagtgggtaaccgcagcagataagaagactgccgtagacatgtcagggggaactgtgactgtacttgaaaaggttcccgtttctaaagggcagctcaaacagtatttctatgaaacaaagtgtaatccaatggggtacaccaaggaaggttgcaggggaatcgacaagcgacattggaacagtcaatgtcggaccactcagagctacgtccgcgctctcacgatggatagtaagaaacgcatcgggtggagattcatcagaatcgacacctcttgcgtctgtactcttacaattaagcgagggcga

SEQ ID NO: 254 - DNA, 585 bp, Artificial Sequence
  Construct for expression of the NDMM IGF-1
atggggaaaatctcctctctccctacccagttgttcaagtgttgcttttgtgacttcttgaaggtaaaaatgcacactatgtcatccagtcaccttttttatttggctctgtgcctcctcacattcaccagttcagctactgccgggcctgaaacactctgcggcgccgaactcgttgatgcgcttcaattcgtgtgtggagatagggggttttactttaacaagccgacgggttatggtagctcaagtagacgagcgccacagactggaatagtagatgaatgctgtttccgctcatgcgaccttcgcagattggaaatgtactgcgctcctcttaaaccagcaaagagtgcgcggtccgtgcgagcccaacgacataccgatatgccaaaaacccagaaatatcagccgccgtctaccaacaagaacaccaagagtcagaggagaaagggttggcccaagacgcacccgggtggcgaacaaaaagaaggtactgaggcaagtttgcaaattcgaggaaagaagaaagaacaacgaagagagataggttctcgcaatgcggaatgtcgaggcaaaaaaggtaag

SEQ ID NO: 255 - DNA, 19 bp, Artificial Sequence
  Forward primer human B-actin
ggccgaggactttgattgc

SEQ ID NO: 256 - DNA, 20 bp, Artificial Sequence
  Reverse primer human B-actin
tggggtggcttttaggatgg

SEQ ID NO: 257 - DNA, 20 bp, Artificial Sequence
  Forward primer human IL-4
gcttccccctctgttcttcc

SEQ ID NO: 258 - DNA, 22 bp, Artificial Sequence
  Reverse primer human IL-4
gatgtctgttacggtcaactcg

SEQ ID NO: 259 - DNA, 20 bp, Artificial Sequence
  Forward primer human IL-10
tcaaggcgcatgtgaactcc

SEQ ID NO: 260 - DNA, 22 bp, Artificial Sequence
  Reverse primer human IL-10
cagggaagaaatcgatgacagc

SEQ ID NO: 261 - protein, 137 aa, Artificial Sequence
  Amino acid sequence of Mature BDNF and Signal Peptide
MTILFLTMVISYFGCMKAHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

(19) United States
(12) Patent Application Publication
(10) Pub. No.: US 2021/0015861 A1
(43) Pub. Date: Jan. 21, 2021

(54) CHIMERIC ANTIGEN RECEPTORS FOR TREATMENT OF NEURODEGENERATIVE DISEASES AND DISORDERS

(71) Applicant: THE TRUSTEES OF DARTMOUTH COLLEGE, Hanover, NH (US)

(72) Inventors: Charles SENTMAN, Grantham, NH (US); David GRABER, Hanover, NH (US); W. James COOK, Hanover, NH (US)

(21) Appl. No.: 16/985,432

(22) Filed: Aug. 5, 2020

Related U.S. Application Data
(63) Continuation-in-part of application No. PCT/US19/17489, filed on Feb. 11, 2019.
(60) Provisional application No. 62/882,710, filed on Aug. 5, 2019.
(60) Provisional application No. 62/628,632, filed on Feb. 9, 2018.

(51) Int. Cl. A61K 35/17; A61P 25/28; C07K 14/54; C07K 14/705; C07K 14/725; C07K 16/18; C07K 16/40; C12N 5/0783; A61P 25/16; C07K 14/47; C12N 9/08; C07K 14/48; C07K 14/65

(52) U.S. Cl. CPC A61K 35/17; A61K 2039/505; C07K 14/5428; C07K 14/70521; C07K 14/7051; C07K 16/18; C07K 16/40; C12N 5/0637; A61P 25/16; C07K 14/4702; C12N 9/0065; C12Y 111/01006; C07K 14/48; C07K 14/65; C07K 2317/622; C07K 2319/30; C07K 2319/33; C07K 2317/76; C12N 2510/00; C07K 2319/03; A61P 25/28

(57) ABSTRACT

The present disclosure generally relates to novel chimeric antigen receptors (“CARs”), modified regulatory T cells (“Tregs”) expressing such CARs and/or Tregs which are engineered to express neurodegenerative disease modifying molecules, e.g., which express molecules which prevent oxidative/inflammatory activity, or which promote neuronal growth/survival such as nerve growth factors or non-classical neurotrophic factors. The present disclosure also generally relates to compositions containing such modified Tregs, and methods of use thereof as therapeutics, in particular for treating and preventing neurodegenerative diseases and symptoms associated with therewith, and/or for slowing the onset of such neurodegenerative diseases, particularly in persons at risk because of genetic factors or in persons exhibiting early signs of developing such a neurodegenerative disease.

### RELATED APPLICATIONS

The instant application claims priority to 62/882,710 filed on Aug. 5, 2019 and further claims priority to, and is a continuation-in-part of PCT/US2019/017489, filed Aug. 9, 2019, which PCT application claims priority to 62/628,632, filed on Feb. 9, 2018. The contents of all of the afore-identified applications are incorporated by reference in their entirety.

### GRANTS

The instant application was made with government support under grant no. R21 NS102556 awarded by the National Institutes of Health (“NIH”). The government has certain rights in the invention.

### SEQUENCE DISCLOSURE

The instant application contains a Sequence Listing which has been submitted in ASCII format via EFS-Web and is hereby incorporated by reference in its entirety. Said ASCII copy, created on Aug. 4, 2020, is named 11432520002604.txt and is 204,929 bytes in size.

### FIELD OF THE ART

The present disclosure generally relates to novel chimeric antigen receptors (“CARs”), nucleic acids encoding such CARs and constructs containing same, modified regulatory T cells (“Tregs”) expressing such CARs and/or Tregs which are engineered to express neurodegenerative disease modifying molecules, e.g., which prevent oxidative/inflammatory activity, or which promote neuronal growth/survival such as nerve growth factors or non-classical neurotrophic factors. The present disclosure also generally relates to compositions containing such modified Tregs, and methods of use thereof as therapeutics, in particular for treating and preventing neurodegenerative diseases and symptoms associated therewith, and/or for slowing the onset of such neurodegenerative diseases, particularly in persons at risk because of genetic factors or in persons exhibiting early signs of developing such neurodegenerative diseases.

### BACKGROUND

Neurodegenerative diseases can generally be characterized by a slow progressive loss of neurons in the central nervous system (CNS), which often leads to deficits in specific brain functions (e.g. memory, movement, cognition) performed by the affected CNS region. These neurodegenerative diseases include, for example, Alzheimer's disease (AD), Parkinson's disease (PD), amyotrophic lateral sclerosis (ALS), multiple sclerosis, Huntington's disease, and multiple system atrophy. Neurodegenerative diseases usually extend over a decade, and the actual onset of neurodegeneration may precede clinical manifestations by many years.

Alzheimer's disease (AD) is a progressive neurodegenerative disease that is one of the primary reasons for memory dysfunction and dementia after 60 years of age. Neuronal dysfunction and death in the frontal cortex and hippocampus, along with microglia-mediated neuroinflammation and formation of aberrant protein aggregates and fibrils are hallmarks of AD. Advancing age increases its prevalence, with an estimated 4.5 million individuals over the age of 65 living with clinical AD in the United States. This number is projected to rise to over 13 million, and to over 130 million worldwide, by 2050 (Hebert et al., Alzheimer disease in the United States (2010-2050) estimated using the 2010 census. Neurology 2013, 80:1778-83)

Sporadic and familial forms of AD have an overproduction and/or decreased clearance of extracellular amyloid-beta (Aβ) peptides and intraneuronal tangles of twisted tau protein fibers. The genetic basis for inheritable autosomal dominant, early-onset AD involves mutations in genes that alter AP production, aggregation, or clearance: these genes include amyloid precursor protein (APP), presenilin-1 (PS1), and presenilin-2 (PS2). Aβ peptides self-oligomerize into small aggregates that can develop into diffuse plaques. Multiple antibodies that bind Aβ in its monomeric, oligomeric, and plaque forms have been created (Montoliu-Gaya L, Villegas S: Aβ-Immunotherapeutic strategies: a wide range of approaches for Alzheimer's disease treatment. Expert reviews in molecular medicine 2016, 18:e13). Neuroinflammation is known to occur in AD, and when associated near Aβ plaques there is a greater neurodegeneration (Heneka M T, et al.: Neuroinflammation in Alzheimer's disease. The Lancet Neurology 2015, 14:388-405; Kreisl W C, et al.: Distinct patterns of increased translocator protein in posterior cortical atrophy and amnestic Alzheimer's disease. Neurobiol. Aging 2017, 51:132-40). Data suggest that inflammatory microglia—the resident macrophages of the central nervous system—have a role in neurodegeneration and cognitive decline (Kreisl W C et al.: Distinct patterns of increased translocator protein in posterior cortical atrophy and amnestic Alzheimer's disease. Neurobiol Aging 2017, 51:132-40; Paolicelli R C, et al.: TDP-43 Depletion in Microglia Promotes Amyloid Clearance but Also Induces Synapse Loss. Neuron 2017, 95:297,308. e6).

There are approximately 7.5 million people with Parkinson's disease worldwide and the disease prevalence increases progressively as age increases (Hebert L E et al.: Alzheimer disease in the United States (2010-2050) estimated using the 2010 census. Neurology 2013, 80:1778-83). It is expected that there will be 9 million people living with PD by 2030. Clinical signs of this neurodegenerative disease are resting tremor, muscular rigidity, slowness of movements, and postural instability. These disabilities are caused by the chronic death of dopamine-producing neurons in the substantia nigra pars compacta of the midbrain. Standard treatment generally involves enhancement of the amount of the neurotransmitter dopamine produced by remaining neurons with medicines that are chemical precursors (levodopa) or that block its inherent breakdown (monoamine oxidase B inhibitors). Several dopamine agonists have also been approved for use in treating PD. While these three types of drugs provide transient improvement in symptom relief, there is little impact on the long-term outcome of PD (Montoliu-Gaya L, Villegas S: Aβ-Immunotherapeutic strategies: a wide range of approaches for Alzheimer's disease treatment. Expert reviews in molecular medicine 2016, 18:e13). While a possible therapeutic strategy for PD would be to prevent the continual death of dopamine neurons, no such treatment is currently available.

The precise etiology of dopamine neuron loss in PD is not well understood. Aberrantly processed proteins and inflammation mediated by microglia, the resident macrophage of the central nervous system, coincide with the loss of neurons. Lewy bodies that contain the protein α-synuclein are inclusions found in dopamine neurons in sporadic and familial PD. In addition to being found in intracellular inclusion bodies, abnormal α-synuclein is also detected extracellularly (El-Agnaf O M, Salem S A, Paleologou K E, Curran M D, Gibson M J, Court J A, Schlossmacher M G, Allsop D: Detection of oligomeric forms of alpha-synuclein protein in human plasma as a potential biomarker for Parkinson's disease. FASEB J 2006, 20:419-25; Alvarez-Erviti L, Couch Y, Richardson J, Cooper J M, Wood M J: Alpha-synuclein release by neurons activates the inflammatory response in a microglial cell line. Neurosci Res 2011, 69:337-42; Tokuda T, Qureshi M M, Ardah M T, Varghese S, Shehab S A, Kasai T, Ishigami N, Tamaoka A, Nakagawa M, El-Agnaf O M: Detection of elevated levels of alpha-synuclein oligomers in CSF from patients with Parkinson disease. Neurology 2010, 75:1766-72). The physiological role of monomeric α-synuclein is not well understood. The formation of abnormal α-synuclein oligomers, though, is thought to be important in the etiology of PD. This is further supported by a mutation in the α-synuclein gene that results in protein self-aggregation and causes a hereditary form of PD. One hypothesis by which aberrant α-synuclein results in dopamine neuron death is by causing or enhancing toxic neuroinflammation. Oligomeric α-synuclein fibrils activate microglia to produce free radicals and pro-inflammatory cytokines, and leads to neurodegeneration (Reynolds A D, Stone D K, Mosley R L, Gendelman H E: Nitrated {alpha}-synuclein-induced alterations in microglial immunity are regulated by CD4+ T cell subsets. J Immunol 2009, 182:4137-49; Alvarez-Erviti L, Couch Y, Richardson J, Cooper J M, Wood M J: Alpha-synuclein release by neurons activates the inflammatory response in a microglial cell line. Neurosci Res 2011, 69:337-42; Theodore S, Cao S, McLean P J, Standaert D G: Targeted overexpression of human alpha-synuclein triggers microglial activation and an adaptive immune response in a mouse model of Parkinson disease. J Neuropathol Exp Neurol 2008, 67:1149-58; Zhang W, Wang T, Pei Z, Miller D S, Wu X, Block M L, Wilson B, Zhang W, Zhou Y, Hong J S, Zhang J: Aggregated alpha-synuclein activates microglia: a process leading to disease progression in Parkinson's disease. FASEB J 2005, 19:533-42).

Amyotrophic lateral sclerosis (ALS) patients develop fatal paralysis as a result of progressive motor neuron loss in the brain and spinal cord. There are approximately 6,000 new cases of ALS per year in the United States, and the typical age of onset is between 40 and 70 years of age, although onset can occur to people in their twenties. ALS can be either idiopathic or hereditary (˜10%), and only about 20% of individuals live more than five years after diagnosis. The absence of an effective therapeutic intervention is especially problematic because the incidence is rising (See e.g., Caller T A et al.: Spatial analysis of amyotrophic lateral sclerosis in Northern New England, USA, 1997-2009. Muscle Nerve 2013, 48:235-41). Inhibiting the persistent neuroinflammation, which is driven primarily by the resident macrophages of the brain and spinal cord (the microglia), is considered a promising therapeutic strategy. However, these processes are difficult to regulate with conventional anti-inflammatory drugs.

Cell therapies with genetically engineered T cells have been demonstrated to provide for the effective treatment of various diseases and disease conditions. For example, effector T cells (Teff) are emerging as a “living drug” treatment for cancer (June C H et al.: Adoptive cellular therapy: A race to the finish line. Sci Transl Med 2015, 7:280ps7). In such treatment methods T cells are typically isolated from patients, modified to express chimeric antigen receptors (CARs) against a tumor ligand, and then transferred back into patients for targeted killing of tumors and activation of host immunity.

T regulatory cells (Tregs) are a subset of T cells that have inherent immunosuppressive and anti-inflammatory properties. Tregs are found in the CNS under steady state conditions and have been observed to migrate or traffic in increased numbers to regions of CNS inflammation (Xie L et al.: Cerebral regulatory T cells restrain microglia/macrophage-mediated inflammatory responses via IL-10. Eur J Immunol 2015, 45:180-91; Gong N et al.: Brain ingress of regulatory T cells in a murine model of HIV-1 encephalitis. J Neuroimmunol 2011, 230:33-41).

### BRIEF SUMMARY

The present disclosure generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed at site(s) of neurodegeneration associated with a specific neurodegenerative disease and is associated with the pathology of said specific neurodegenerative disease or condition and/or (ii) a protein which is aberrantly expressed (e.g. overexpressed) at site(s) of neurodegeneration associated with a specific neurodegenerative disease and/or is associated with the pathology of said specific neurodegenerative disease or condition, wherein said CAR cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is associated with the expression of said aberrant or aberrantly expressed protein. In exemplary embodiments, said site(s) of neurodegeneration may be present in the central nervous system, and/or said site(s) of neurodegeneration are present in the peripheral nervous system. In exemplary embodiments, said CAR-expressing cells may comprise immune cells, optionally wherein said CAR-expressing immune cells comprise T cells or T cell progenitors, preferably T regulatory cells (Tregs) such as FOXP3⁺ Tregs. In exemplary embodiments, the administered cells may comprise a CAR which recognizes at least one aberrant protein expressed at a site of neurodegeneration.

In some embodiments, the CAR comprised on said administered cells may comprise DG01 (SEQ ID NO: 1), DG02 (SEQ ID NO: 2), DG03 (SEQ ID NO: 3), DG04 (SEQ ID NO: 4), DG05 (SEQ ID NO: 5), DG06 (SEQ ID NO: 6), DG07 (SEQ ID NO: 7), DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs. In some embodiments, said administered cells may be engineered to express one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44) and/or a construct comprising at least 90%, at least 95%, at least 98%, or at least 99% sequence identity to any one or more of the aforementioned constructs, optionally wherein each of said one or more constructs targets mutSOD1. In some embodiments, said administered cells may be engineered to express one or more of the following: DG03-CD28-CD3ζ (also referred to as DG03-28-3ζ) (SEQ ID NO: 22); DG03-CD28tm-DAP10-CD3 (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (also referred to as DG03-28tm) (SEQ ID NO: 50), and/or a construct comprising at least 90%, at least 95%, at least 98%, or at least 99% sequence identity to any one or more of the aforementioned constructs, optionally wherein each of said one or more constructs targets amyloid beta.

In further exemplary embodiments, the CAR may comprise an scFv or ligand which recognizes at least one aberrant protein expressed at a site of neurodegeneration. In exemplary embodiments, said cells may be further engineered to express at least one pro-neuronal factor or nerve growth factor, optionally wherein said CAR and said NDMM are on the same or are on different cells. In some embodiments, said administered cells may be engineered to express NDMM Nrf2 (Keap1 inhibitor peptide) (SEQ ID NO: 51) and/or a construct comprising at least 90%, at least 95%, at least 98%, or at least 99% sequence identity to the aforementioned construct. In some embodiments, said administered cells may be engineered to express human catalase (SEQ ID NO: 52), and/or a construct comprising at least 90%, at least 95%, at least 98%, or at least 99% sequence identity to the aforementioned construct. In some embodiments, said administered cells may be engineered to express BDNF (SEQ ID NO: 53), and/or a construct comprising at least 90%, at least 95%, at least 98%, or at least 99% sequence identity to the aforementioned construct. In some embodiments, said administered cells are engineered to express IGF-1 (SEQ ID NO: 54), and/or a construct comprising at least 90%, at least 95%, at least 98%, or at least 99% sequence identity to the aforementioned construct. In exemplary embodiments, said cells may be further engineered to express at least one anti-oxidative protein which inhibits or protects neurons from anti-oxidative stress and/or inhibits or prevents the death of neurons at the site of neurodegeneration optionally wherein said CAR and said anti-oxidative protein are on the same or are on different cells, further optionally wherein the anti-oxidant also promotes T cell function or lifespan.

In some embodiments, the administered cells may express increased levels of IL-10 in response to mSOD1 antigen in the treated subject. In some embodiments, the administered cells may express increased levels of cell surface markers including one or more of GITR, PD-1 and/or CTLA-4 in response to mSOD1 antigen in the treated subject. In some embodiments, the administered cells may inhibit superoxide generation in response to mSOD1 antigen and/or anti-CD3 in the treated subject. In some embodiments, the administered cells may inhibit TNF-α production in response to mSOD1 antigen in the treated subject. In some embodiments, the administered cells may be engineered to express a construct comprising DG05 (SEQ ID NO: 5) and/or a construct comprising at least 90%, at least 95%, at least 98%, or at least 99% sequence identity to the aforementioned construct. In some embodiments, the administered cells may be engineered to express one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs. In some embodiments, the administered cells may express increased levels of IL-10 and/or IL-4 in response to amyloid beta antigen in the treated subject. In some embodiments, the administered cells inhibit superoxide generation in response to amyloid beta antigen and/or anti-CD3 in the treated subject. In some embodiments, the administered cells may inhibit IL-6 production in response to amyloid beta antigen and/or anti-CD3 in the treated subject. In some embodiments, the administered cells may protect cells of the treated subject from hydrogen peroxide toxicity. In some embodiments, the administered cells may be engineered to express a construct comprising DG03 (SEQ ID NO: 3), and/or a construct comprising at least 90%, at least 95%, at least 98%, or at least 99% sequence identity to the aforementioned construct. In some embodiments, the administered cells may be engineered to express one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs. In some embodiments, the administered cells may protect cells of the treated subject from hydrogen peroxide toxicity, optionally wherein said administered cells are engineered to express one or more of the following constructs: NDMM human catalase construct (SEQ ID NO: 52), NDMM Nrf2 (Keap1 inhibitor peptide) construct (SEQ ID NO: 51), NDMM BDNF construct (SEQ ID NO: 53), and/or NDMM IGF-1 construct (SEQ ID NO: 54), and/or a construct comprising at least 90%, at least 95%, at least 98%, or at least 99% sequence identity to any one or more of the aforementioned constructs.

In further exemplary embodiments, the neurodegenerative disease or condition may comprise at least one of Parkinson's disease, Alzheimer's disease, Prion disease, a Motor neurone disease (MND) such as amyotrophic lateral sclerosis (ALS), Huntington's disease (HD), Spinocerebellar ataxia (SCA), Spinal muscular atrophy (SMA), Friedreich's ataxia, Lewy body disease, epilepsy, encephalitis, hydrocephalus, stroke, chronic traumatic encephalopathy (CTE); a synucleinopathy; a tauopathy, a spongiform encephalopathy; familial amyloidotic polyneuropathy; Dutch hereditary cerebral hemorrhage with amyloidosis; congophilic angiopathy; corticobasal degeneration; Pick's disease; progressive supranuclear palsy; Creutzfeldt-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; and Icelandic hereditary cerebral hemorrhage with amyloidosis. In exemplary embodiments, the neurodegenerative disease may comprise Parkinson's disease. In exemplary embodiments, the neurodegenerative disease may comprise Alzheimer's disease. In exemplary embodiments, the neurodegenerative disease may comprise amyotrophic lateral sclerosis (ALS). IN exemplary embodiments, the CAR may bind to one or more of human amyloid beta, amyloid-beta 1-42, alpha-synuclein, superoxide dismutase-1 (SOD-1), hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; and Leucine-rich repeat serine/threonine-protein kinase 2 (LRRK2), Huntingtin with tandem glutamine repeats; prion proteins; transthyretin; dentatorubral pallidoluysian atrophy (DRPLA) protein; androgen receptor; an ataxin; P/Q-type calcium channel α1A subunit; TATA-box-binding protein; glial fibrillary acidic protein; DNA excision repair protein ERCC-6; survival motor neuron protein; and cystatin C.

In exemplary embodiments, the administered cells may express at least one pro-neuronal factor, neurotrophic factor, or nerve growth factor selected from brain-derived neurotrophic factor (BDNF), ciliary neurotrophic factor (CNTF), glial cell-line derived neurotrophic factor (GDNF), interleukin-1 receptor antagonist (IL-1ra); interleukin-6 (IL-6); activated protein C (APC); thrombomodulin; tissue plasminogen activator (tPA); Protein deglycase DJ-1; a tissue inhibitor of metalloproteinases (TIMP), insulin-like growth factor-1 (IGF-1), vascular endothelial growth factor (VEGF), fibroblast growth factor (FGF), a bone morphogenetic protein (BMP), erythropoietin (EPO), thrombopoietin (TPO), and granulocyte-colony stimulating factor (G-CSF), optionally wherein said at least one pro-neuronal factor, neurotrophic factor, or nerve growth factor are on the same cell as said CAR or on a different cell as said CAR. In exemplary embodiments, the anti-oxidative protein is a protein selected from superoxide dismutases such as human superoxide dismutase, Cu/Zn superoxide dismutase, HO-1, ferritin, glutathione reductase, glutathione peroxidase, ferritin (H), metallothionein I, thioredoxin, thioredoxin reductase, peroxiredoxins (Prxs) such as pereoxiredoxin MSP₂₃; activity-dependent neuroprotector homeobox (ADNP); phycocyanin; neuroglobin, catalase, and NRF2, optionally wherein said at least one anti-oxidative protein is on the same cell as said CAR or on a different cell as said CAR. In exemplary embodiments, the administered cells may reduce or stabilize the amount of inflammation present at said site(s) of neurodegeneration. In exemplary embodiments, the administered cells may inhibit or prevent at least one of: (i) microglia cell over-activation wherein over-activation includes microglia which possess at least one activity or increase in an activity characteristic of activated microglia such as (1) a change in morphology, (2) migration to inflammatory sites, (3) production of neurotoxic or inflammatory cytokines such as IL-1, (4) interaction with neural plaques or (3 amyloid deposits, (5) synthesis of neurotoxic proteins, (6) secretion of proteases and/or reactive oxygen species, (7) induction of amyloid production by neighboring cells, (8) destruction of myelin; (ii) increased numbers of microglia; (iii) the production of inflammatory proteins or inflammatory activities at sites of neurodegeneration; (iv) macrophage activity (v) the expression of inflammatory or neurotoxic moieties by cells within such sites such as cytokines, oxidants, proteases e.g., by macrophages and microglia and/or (vi) neuronal death or impaired neuronal function.

Moreover, the present disclosure generally encompasses a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory domain, the expression of which are optionally controlled by the same or different inducible or constitutive promoters. In exemplary embodiments, the protein associated with a neurodegenerative disease or condition is selected from human amyloid beta, amyloid-beta 1-42, alpha-synuclein, superoxide dismutase-1 (SOD-1), hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK′; and Leucine-rich repeat serine/threonine-protein kinase 2 (LRRK2), Huntingtin with tandem glutamine repeats; prion proteins; transthyretin; dentatorubral pallidoluysian atrophy (DRPLA) protein; androgen receptor; an ataxin; P/Q-type calcium channel α1A subunit; TATA-box-binding protein; glial fibrillary acidic protein; DNA excision repair protein ERCC-6; survival motor neuron protein; and cystatin C. Additionally, in exemplary embodiments, the present disclosure generally relates to recombinant or engineered cell which comprises at least one nucleic acid encoding a CAR as described herein.

Furthermore, the present disclosure generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (e.g. overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease. The present disclosure also generally encompasses a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease, and a CAR of said CAR-expressing cells includes DG01 (SEQ ID NO: 1), DG02 (SEQ ID NO: 2), DG03 (SEQ ID NO: 3), and/or DG04 (SEQ ID NO: 4), and/or a construct comprising at least 90%, at least 95%, at least 98%, or at least 99% sequence identity to any one or more of the aforementioned constructs. The present disclosure additionally generally encompasses a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease, and a CAR of said CAR-expressing cells includes DG01 (SEQ ID NO: 1), DG02 (SEQ ID NO: 2), DG03 (SEQ ID NO: 3), and/or DG04 (SEQ ID NO: 4), and/or a construct comprising at least 90%, at least 95%, at least 98%, or at least 99% sequence identity to any one or more of the aforementioned constructs, and further wherein said cells are engineered to express one or more neurodegenerative disease modifying molecules (NDMMs).

The present disclosure also generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS. The present disclosure additionally generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS, and a CAR of said CAR-expressing cells includes DG05 (SEQ ID NO: 5), DG06 (SEQ ID NO: 6), and/or DG07 (SEQ ID NO: 7), and/or a construct comprising at least 90%, at least 95%, at least 98%, or at least 99% sequence identity to any one or more of the aforementioned constructs. The present disclosure also generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS, and a CAR of said CAR-expressing cells includes DG05 (SEQ ID NO: 5), DG06 (SEQ ID NO: 6), and/or DG07 (SEQ ID NO: 7), and/or a construct comprising at least 90%, at least 95%, at least 98%, or at least 99% sequence identity to any one or more of the aforementioned constructs, and further wherein said cells are engineered to express one or more neurodegenerative disease modifying molecules (NDMMs).

Furthermore, the present disclosure generally encompasses a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Parkinson's disease. The present disclosure additionally generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Parkinson's disease, and a CAR of said CAR-expressing cells includes DG08 (SEQ ID NO: 8), DG09 (SEQ ID NO: 9), DG10 (SEQ ID NO: 10), and/or DG11 (SEQ ID NO: 11), and/or a construct comprising at least 90%, at least 95%, at least 98%, or at least 99% sequence identity to any one or more of the aforementioned constructs. The present disclosure also generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Parkinson's disease, and a CAR of said CAR-expressing cells includes DG08 (SEQ ID NO: 8), DG09 (SEQ ID NO: 9), DG10 (SEQ ID NO: 10), and/or DG11 (SEQ ID NO: 11), and/or a construct comprising at least 90%, at least 95%, at least 98%, or at least 99% sequence identity to any one or more of the aforementioned constructs, and further wherein said cells are engineered to express one or more neurodegenerative disease modifying molecules (NDMMs).

The present disclosure also generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said protein is a protein associated with Alzheimer's disease. Additionally, the present disclosure generally encompasses a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said protein is a form of amyloid beta associated with Alzheimer's disease. The present disclosure also generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said protein is a protein associated with ALS disease. The instant disclosure additionally generally encompasses a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said protein is mutated or aberrantly expressed SOD1. Also, the present disclosure generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said protein is a protein associated with Parkinson's disease.

Furthermore, the present disclosure generally encompasses a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said protein is a form of alpha-synuclein associated with Parkinson's disease. The present disclosure also generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said nucleic acid encodes DG01 (SEQ ID NO: 1), DG02 (SEQ ID NO: 2), DG03 (SEQ ID NO: 3), and/or DG04 (SEQ ID NO: 4), and/or a construct comprising at least 90%, at least 95%, at least 98%, or at least 99% sequence identity to any one or more of the aforementioned constructs. The present disclosure additionally generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said nucleic acid encodes DG05 (SEQ ID NO: 5), DG06 (SEQ ID NO: 6), and/or DG07 (SEQ ID NO: 7), and/or a construct comprising at least 90%, at least 95%, at least 98%, or at least 99% sequence identity to any one or more of the aforementioned constructs. Additionally, the present disclosure generally encompasses a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, the expression of which are optionally controlled by the same or different inducible or constitutive promoters, wherein said nucleic acid encodes DG08 (SEQ ID NO: 8), DG09 (SEQ ID NO: 9), DG10 (SEQ ID NO: 10), and/or DG11 (SEQ ID NO: 11), and/or a construct comprising at least 90%, at least 95%, at least 98%, or at least 99% sequence identity to any one or more of the aforementioned constructs.

Furthermore, the present disclosure generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease, and said cells are engineered to express any one or more of the following: DG03 (SEQ ID NO: 3); DG03-CD28-CD3ζ (also referred to as DG03-28-(SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs. Moreover, the present disclosure generally relates to a method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS, and said cells are engineered to express any one or more of the following: DG05 (SEQ ID NO: 5); DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs. Furthermore, the present disclosure generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes any one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3 (also referred to as DG03-28tm-3ζ) (SEQ ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs. Moreover, the present disclosure generally relates to a nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes any one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); and/or DG05-CD28tm (also referred to as DG05-28tm) (SEQ ID NO: 44), and/or a construct comprising at least 90%, at least 95%, at least 98%, or at least 99% sequence identity to any one or more of the aforementioned constructs.

### BRIEF DESCRIPTION OF THE SEVERAL VIEWS OF THE DRAWING

FIG. 1 presents a schematic design of anti-Aβ CARs consisting of an extracellular scFv fused to a CD28 transmembrane and CD3 intracellular signaling domains, and with a co-expressed non-functional truncated CD19 (tCD19) for enriching and tracking transduced cells, in accordance with Example 1.

FIG. 2A-FIG. 2D present data related to in vitro expansion and phenotype validation of Tregs isolated from human PBMCs in accordance with Example 1. FIG. 2A presents data that demonstrated that CD4+CD25hi Tregs (R2 box) represented a small percentage of total T cells in human PBMCs prior to CD4 and CD25 enrichment isolation. FIG. 2B presents data that demonstrated that that CD4+CD25hi isolated Tregs expanded 1760-fold after 17 days in culture using the present Treg expansion protocol. FIG. 2C presents data which demonstrate that Day 17 Tregs expressed intracellular FoxP3. FIG. 2D presents data that demonstrated that FoxP3, truncated CD19 (tCD19), and the CAR scFv were detected on most day 17 Tregs transduced on days 10 and 11.

FIG. 3A-FIG. 3B presents data related to functional validation of the exemplary modified CARs in accordance with Example 1. FIG. 3A presents data related to oligomerization of Aβ₁₋₄₂ peptides, and FIG. 3B presents data related to binding specificity and function of CARs comprised by modified human T cells in accordance with Example 1.

FIG. 4 presents data related to DG03-28z anti-Aβ CAR modified Treg-mediated suppression of T cell proliferation in accordance with Example 2.

FIG. 5 presents data related to Aβ CAR Tregs stimulated with oligomeric Aβ in vitro for 24 hours and the production of IL-10 in accordance with Example 3.

FIG. 6 presents a schematic design of an anti-mutSOD1-CD28-CD3ζ CAR in accordance with Example 4.

FIG. 7A-FIG. 7D presents data demonstrating suppression of effector T cell proliferation (FIG. 7A); expression of intracellular FoxP3 (FIG. 7B); and purification for tCD19⁺ cells (FIG. 7C-FIG. D) in accordance with an alternative Treg isolation method consisting of CD4⁺CD127⁻ T cells isolated using negative selection, followed by a positive selection of CD25^{hi} cells to isolate CD4⁺CD127⁻CD25^{hi}Treg cells, and stimulation with anti-CD3, anti-CD28, anti-CD2 multimers as described in Example 4.

FIG. 8 presents data related to three exemplary anti-mutSOD1 CARs (named DG05, DG06, and DG07) and their antigen specific activity against the G93A mutated form of SOD1 in accordance with Example 4. A negative control anti-B7H6 CAR does not respond to mutated SOD1.

FIG. 9 presents data related to the phenotype validation of Tregs isolated from human PBMCs in accordance with Example 4. CD3, FoxP3, CD4, truncated CD19 (tCD19), and the CAR scFv with protein L labeling were detected on most day 17 Tregs transduced on days 10 and 11 with an anti-mutSOD1 CAR (DG05-28z).

FIG. 10 presents data related to the phenotype validation of Tregs isolated from human PBMCs in accordance with Example 4. Scatter plot, CD4 and FoxP3 on most day 17 Tregs transduced on days 10 and 11 with an anti-mutSOD1 CAR (DG05-28z) or anti-Aβ CAR (DG03-28z).

FIG. 11 presents data demonstrating antigen-specific activity of exemplary anti-mutSOD1 CARs in accordance with Example 5. IL-10 production in response to plate-bound mutSOD1 or soluble oligomerized Aβ₁₋₄₂ by day 17 Tregs transduced on days 10 and 11 with an anti-mutSOD1 CAR (DG05-28z) or anti-Aβ CAR (DG03-28z).

FIG. 12 presents data demonstrating antigen-specific activity of exemplary anti-mutSOD1 CARs in accordance with Example 5. IFN-γ production in response to biotinylated wt or mut SOD1 linked to plate-bound streptavidin (SA) by human T effector cells transduced to express anti-mutSOD1 CARs (named DG05, DG06, and DG07) or negative control anti-B7H6 CAR.

FIG. 13 presents data demonstrating DG05-28z anti-mutSOD1 CAR modified Treg-mediated suppression of T cell proliferation in accordance with Example 5.

FIG. 14 presents a schematic design of an anti-α-synuclein-CD28-CD3ζ CAR in accordance with Example 6.

FIG. 15 presents data demonstrating the functional activity of exemplary modified Tregs in accordance with Example 7. IL-10 production in response to plate-bound α-synuclein, or to soluble oligomerized α-synuclein or Aβ₁₋₄₂ by day 17 Tregs transduced on days 10 and 11 with an anti-α-synuclein CAR (DG10-28z) or anti-Aβ CAR (DG03-28z).

FIG. 16A-FIG. 16B present data demonstrating expression (FIG. 16A) and function (FIG. 16B) of anti-mSOD1 CARs with different co-stimulatory signaling domains and with or without the CD3zeta (3ζ) stimulatory domain in ex vivo expanded and CAR transduced human Tregs in accordance with Example 8.

FIG. 17A-FIG. 17B present data demonstrating expression (FIG. 17A) and function (FIG. 17B) of anti-Aβ CARs with different co-stimulatory signaling domains and with or without the CD3zeta (3ζ) stimulatory domain in ex vivo expanded and CAR transduced human Tregs in accordance with Example 9.

FIG. 18 presents data demonstrating the antigen-specific activity of anti-mutSOD1 CARs as demonstrated by assays evaluating the cell surface expression of GITR, PD-1, and CTLA-4 and the production of IL-10 in accordance with Example 10. With regard to each of the six plots related to cell surface expression markers: the numerical values reported for each correspond to, from top to bottom: media; beads (no Ag); and mSOD1 Beads.

FIG. 19A-FIG. 19B present data demonstrating the functional activity of modified Tregs targeting ALS by assays comprising co-culturing said modified Tregs with spinal cord tissue explants derived from transgenic mice expressing human mSOD1 (FIG. 19A), and co-culturing modified Tregs with spinal, liver, or lung tissue explants derived from transgenic mice expressing human mSOD1 (FIG. 19B) in accordance with Example 11. Spinal cord tissues were collected from non-transgenic mice or mSOD1 transgenic mice at different stages of disease development: 13 weeks (see FIG. 19A: pre-paralysis), 14 weeks (see FIG. 19A: clinical onset), 16 weeks (see FIG. 19A: paralysis), or 18 weeks (disease end-stage weeks defined as 15% weight loss and hind-limb paralysis; see FIG. 19B). Liver and lung were also collected from mSOD1 transgenic mice at disease end-stage (see FIG. 19B).

FIG. 20A-FIG. 20B present data demonstrating the functional activity of modified Tregs targeting Alzheimer's disease, wherein said modified Tregs were exposed to oligomerized Aβ and then the mRNA levels of IL-10 and IL-4 (FIG. 20A) and protein secretion levels of IL-10 and IL-4 (FIG. 20B) were monitored in accordance with Example 12.

FIG. 21A-FIG. 21C present data demonstrating the antigen-specific anti-inflammatory activity of anti-mutSOD1 CARs in accordance with Example 13. FIG. 21A presents data related to an assay in which inhibition of PMA-stimulated superoxide generation was evaluated in accordance with Example 13. FIG. 21B presents data related to an assay in which inhibition of Zymosan-stimulated superoxide generation was evaluated in accordance with Example 13. FIG. 21C presents data related to an assay in which inhibition of TNF-α generation was evaluated in accordance with Example 13.

FIG. 22A-FIG. 22C present data demonstrating the antigen-specific anti-inflammatory activity of anti-Aβ CARs in accordance with Example 14. FIG. 22A presents data related to an assay in which inhibition of PMA-stimulated superoxide generation was evaluated in accordance with Example 14. FIG. 22B presents data related to an assay in which inhibition of Zymosan-stimulated superoxide generation was evaluated in accordance with Example 14. FIG. 22C presents data related to an assay in which inhibition of IL-6 generation was evaluated in accordance with Example 14.

FIG. 23A-FIG. 23B present data demonstrating the cytoprotective activity of neurodegenerative disease-modifying molecule (NDMM) expressed in human Tregs in accordance with Example 15. FIG. 23A presents data related to an assay in which NDMM constructs for Nrf2 (Keap1 inhibitor peptide) and human catalase were evaluated in accordance with Example 15. FIG. 23B presents data related to an assay in which NDMM constructs for brain derived neurotrophic factor (BDNF), and insulin growth factor-1 (IGF-1) were evaluated in accordance with Example 15.

FIG. 24A-FIG. 24E present data demonstrating that anti-mSOD1 human CAR Tregs are detected in spinal cord tissue of ALS mice and demonstrate CAR signaling activity. Transgenic NSG mice expressing human SOD1*G93A (mSOD1) develop weight loss (A) and ALS-like limb paralysis (B) after 11 weeks of age. Human CD52 mRNA (normalized to mouse β-actin) measured by real time qPCR is detected in spleen (C) and spinal cords (D) of preclinical 11.5-week-old mSOD1.NSG mice two days after intravenous injection of 10×10⁶ anti-mSOD1 DG05-28-3z (signaling) or DG05-28tm (non-signaling) human CAR Tregs. *, p<0.05, ***, p<0.001 by Newman-Keuls Multiple Comparison Test relative to baseline detection limit in tissues from vehicle-injected mice. Abundance of human CCL3 mRNA (normalized to human CD52 reference mRNA), an activation marker, is higher in spinal cords from mice injected with human Tregs with DG05-28-3z (signaling) CAR relative to DG05-28tm (non-signaling) CAR (E).

FIG. 25A-FIG. 25C contain the results of staining experiments detecting expression of CAR and BDNF or msCD19 polypeptides in CAR Tregs engineered to express human BDNF or msCD19 and a CAR construct.

FIG. 26 contains the amino acid sequence of the mature and signal peptide of BDNF (SEQ ID NO: 261).

FIG. 27A-FIG. 27B contain experiments showing that human Tregs transduced with vIL-10 (BCRF-1) secrete vIL-10 and potently inhibit IL-6 production in LPS-Stimulated macrophages compared to controls.

FIG. 28A-FIG. 28D contain experiments showing that human Tregs transduced with CAR and vIL-10 (BCRF-1) secrete vIL-10 and retain CAR function.

### DETAILED DESCRIPTION

The present disclosure generally relates to the construction of nucleic acid constructs which encode CARs, especially those which target a protein that is aberrantly expressed in the CNS of a subject with a neurodegenerative disease or condition and/or which encode specific molecules that prevent or inhibit oxidative/inflammatory activity at CNS sites and/or which encode molecules which promote neuronal growth/survival or which promote T cell function. The present disclosure further generally relates to the use of these nucleic acid constructs in the preparation of recombinant or modified cells, in particular recombinant or modified Tregs, preferably human Tregs which are engineered to express such CARs and/or other molecules expressed by such constructs. In exemplary embodiments, these recombinant or modified Tregs may be engineered to express one or more CARs, wherein said one or more CARs may target different proteins and/or molecular markers associated with the pathology of particular neurodegenerative diseases and conditions. These CARs optionally may further comprise a costimulation signaling or T cell signaling moiety such as CD28-CD3ζ DAP10-CD3ζ, CD44-CD3ζ, CD28 or CD3ζ or another costimulatory signaling or T cell signaling moiety. Modified Tregs according to the invention may further optionally be engineered to express one or more neurodegenerative disease modifying molecules (NDMMs) which may be on the same or different nucleic acid construct as the CAR or may be expressed on modified Tregs which do not comprise a CAR. The present disclosure specifically contemplates modified Tregs expressing one or more specific CARs targeting a neurodegenerative disease and/or neurodegenerative disease modifying molecules (NDMMs), pharmaceutical compositions comprising said modified Tregs, and methods of making and using these modified Tregs. The present disclosure also provides methods for treating a neurodegenerative disease, disorder, or condition, a subject, such as but not limited to Alzheimer's disease, Parkinson's disease, Amyotrophic lateral sclerosis (ALS), and neuroinflammation using these modified Tregs.

Another specific aspect of the present disclosure relates to the construction and use of modified Tregs comprising exogenously introduced polynucleotides encoding specific types of neurodegenerative disease modifying molecules (“NDMMs”) such as an anti-oxidants, nerve growth factors and/or non-classical neurotrophic factors. The disclosure also provides vectors for generating such modified Tregs, pharmaceutical compositions comprising such modified Tregs which express one or more CARs and/or one or more NDMMs, and methods of making and using modified Tregs expressing a combination of one or more CARs and one or more NDMMs in the treatment of specific neurodegenerative diseases.

### Definitions

As used herein, the terms “neurodegenerative disease”, “neurodegenerative disorder”, and “neurodegenerative condition” generally refer to any disease, disorder, and/or condition that affects the neurons (sometimes referred to as “nerve cells”), such as neurons of a brain and/or neurons of a nervous system which is associated with the degeneration or loss of neural cells. Often, neurodegenerative diseases may result in progressive degeneration and/or death of nerve cells. In general neurodegeneration is the progressive loss of structure or function of neurons, including the death of neurons. Neurodegenerative diseases may cause problems with movement (called ataxias), or mental or cognitive functioning (called dementias). Frequently neurodegeneration is associated with neuroinflammation and indeed the onset, progression or cause of many debilitating neurodegenerative diseases is thought to involve neuroinflammation. Therefore, it is to be understood that the terms neurodegenerative disease and neurodegenerative disorder and the like encompass neural diseases which are characterized by neuroinflammation. Sometimes in such diseases activated microglia may produce inflammatory cytokines that contribute to widespread inflammation and may lead to and/or result in a neurodegenerative condition and/or disease. Furthermore, some neurodegenerative diseases and/or conditions are associated with microglia cell over-activation, increased numbers of microglia cells, production of inflammatory proteins and/or inflammatory activities, and/or neuronal death. Examples of such neurodegenerative diseases include by way of example Alzheimer's disease and other dementias, Parkinson's disease and other Parkinson's disease related disorders, prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathies; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis. In exemplary embodiments, a neurodegenerative disease may comprise Alzheimer's disease, Parkinson's disease, and/or ALS. In exemplary embodiments, modified Tregs cells as described herein may be used in a method of treating these and other neurodegenerative diseases.

As used herein, the term “neuroinflammation” generally refers to inflammation of the nervous tissue. Sometimes, activated microglia may produce inflammatory cytokines that contribute to widespread inflammation and may lead to and/or result in a neurodegenerative condition and/or disease. In some instances, neuroinflammation may be initiated in response to a variety of cues, including infection, traumatic brain injury, toxic metabolites, and/or autoimmunity. In the central nervous system (CNS), including the brain and spinal cord, microglia are the resident innate immune cells that are activated in response to these cues, and generally generate reactive oxygen species and release signals to recruit peripheral immune cells for an inflammatory response. Cytokines may also be present at the sites of and/or may cause neuroinflammation, and in some instances they may be produced by microglia or macrophages. In exemplary embodiments, neuroinflammation may be associated with and/or may arise during a neurodegenerative disease, e.g., Alzheimer's disease, ALS, and Parkinson's disease.

The term “inflammation” refers to a broad physiological response mediated by various cell types, proteins, humoral factors, and tissues. While inflammation can send signals within a body to help the immune system eliminate pathogens or undesired conditions, inappropriate levels or altered types of inflammation can cause numerous physiological or immunological problems within the body. Such inflammation can be directly responsible for the pathology of various diseases including autoimmune diseases, fibrotic diseases, chronic infections, and allergies (Laria, A. et al., “The macrophages in rheumatic diseases”, J Inflamm Res. 2016 Feb. 9; 9: p. 1-11; Wynn, T. A., and Ramalingam, T. R., “Mechanisms of fibrosis: fibrotic translation for fibrotic diseases”, Nat Med, 2012 Jul. 6; 18(7): p. 1028-40; Yang, Z. P., Kuo, C. C., and Grayston, J. T, “Systemic dissemination of Chlamydia pneumoniae following intranasal inoculation in mice”, J Infect Dis. 1995 March; 171(3): p. 736-8; Jian, Z., and Zhu, L., “Update on the role of alternatively activated macrophages in asthma”, J Asthma Allergy, 2016 Jun. 3; 9: p. 101-7). Inflammation can also indirectly exacerbate the symptoms of many diseases, or play an assisting role in the pathogenesis, for example in cancers, obesity, metabolic diseases, and cardiovascular diseases, such as atherosclerosis (Coussens, L. M., and Werb, Z., “Inflammation and Cancer”. Nature. 2002 Dec. 19-26; 420(6917): p. 860-7; Monteiro, R., and Azevedo, I., “Chronic inflammation in obesity and the metabolic syndrome”, Mediators Inflamm. 2010; 2010; Libby, P., “Inflammation and cardiovascular disease mechanisms”, Am J Clin Nutr. 2006 February; 83(2): p. 456S-460S).

The term “neurodegenerative disease-modifying molecule” or “NDMM” as used herein generally refers to a molecule capable of altering (reducing, ameliorating or preventing) the symptoms, progression or onset of a neurodegenerative disease, disorder, or condition. Representative neurodegenerative conditions include by way of example Alzheimer's disease, ALS, Parkinson's disease, and other neuroinflammatory conditions. Examples of such NDMM molecules include, but are not limited to including, IL-37, IL-12, TNF-α, IFN-γ, CCL2, TNFAIP3, and other molecules capable of altering the expression level, activation status, or function of a disease-associated protein. In exemplary embodiments, an NDMM may comprise one or more cytokines. In other exemplary embodiments, an NDMM may comprise molecules that prevent oxidative/inflammatory activity. In other exemplary embodiments, an NDMM may comprise molecules that promote neuronal growth and/or survival. In exemplary embodiments, an NDMM may be expressed by modified Tregs according to the invention, e.g., modified Tregs comprising one or more CARs, as discussed in further detail herein. Furthermore, an NDMM may comprise one or more pro-neuronal factors, one or more anti-oxidants, one or more nerve growth factors, and/or one or more non-classical neurotrophic factors. Examples of pro-neuronal factors include, but are not limited to including, interleukin-1 receptor antagonist (IL-1ra); interleukin-6 (IL-6); activated protein C (APC); thrombomodulin; tissue plasminogen activator (tPA); Protein deglycase DJ-1; tissue inhibitor of metalloproteinases (TIMPs). Examples of antioxidants include, but are not limited to including, HO-1, Ferritin, Glutathione reductase, Glutathione peroxidase, Ferritin (H), Metallothionein I, Thioredoxin, Thioredoxin reductase, Peroxiredoxin MSP23, Cu/Zn superoxide dismutase, Catalase, NRF2 activity, peroxiredoxins (Prxs); activity-dependent neuroprotector homeobox (ADNP); phycocyanin; neuroglobin. Examples of nerve growth factors include, but are not limited to, classic neurotrophins such as brain-derived neurotrophic factor (BDNF), ciliary neurotrophic factor (CNTF), and glial cell-line derived neurotrophic factor (GDNF). Non-limiting examples of non-classical neurotrophic factors include insulin-like growth factor-1 (IGF-1), vascular endothelial growth factor (VEGF), Fibroblast Growth Factors (FGF), Hepatocyte Growth Factor (HGF), Bone Morphogenetic Proteins (BMPs), Erythropoietin (EPO), Thrombopoietin (TPO), and Granulocyte-colony stimulating factor (G-CSF). In some embodiments, NDMM expression may be controlled by an inducible promoter system, e.g., using one known in the art, and/or expression of the NDMM may be regulated by CAR-triggered transcriptional control.

As used herein, a “5′ cap” (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

The term “allogeneic” or “donor-derived” generally refers to any material derived from a different animal of the same species as the individual to whom the material is to be introduced or transplanted. Two or more individuals are said to be allogeneic to one another when the genes at one or more loci are not identical. In some aspects, allogeneic material from individuals of the same species may be sufficiently dissimilar genetically to interact antigenically.

The term “antibody” or “Ab,” as used herein, refers to an immunoglobulin molecule which specifically binds with an antigen. In some embodiments, the antigen may be a molecule expressed or aberrantly expressed by neurons in subjects comprising a neurodegenerative disease and/or condition. Examples of such diseases and conditions include, but are not limited to including, Alzheimer's disease, Parkinson's disease, and ALS. Further non-limiting examples include prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathies; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis. Antibodies can be intact immunoglobulins derived from natural sources or from recombinant sources and can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The term “antibody fragment” or “Ab fragment” refers to a portion of an intact antibody and refers to the antigenic determining variable regions of an intact antibody. Examples of antibody fragments include, but are not limited to, fragment antigen binding (Fab) fragments, F(ab′)₂ fragments, Fab′ fragments, Fv fragments, recombinant IgG (rIgG) fragments, single chain antibody fragments, including single chain variable fragments (scFv), single domain antibodies (e.g., sdAb, sdFv, nanobody) fragments, diabodies, and multispecific antibodies formed from antibody fragments. In exemplary embodiments, the antibody fragment may be an scFv.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired response. Moreover, a skilled artisan will understand that an antigen need not be encoded by a “gene” at all. It is readily apparent that an antigen can be generated, synthesized, or can be derived from a biological sample, or might be a macromolecule besides a polypeptide. Such a biological sample can include, but is not limited to a tissue sample, a neurological tissue sample, an inflamed tissue sample, a cell, or a fluid with other biological components. In some embodiments, the antigen is a molecule expressed in a neurodegenerative disease or condition, e.g., Alzheimer's disease, Parkinson's disease, and ALS. In exemplary embodiments, an antigen may be a form of any one or more of the following that may be associated with a neurodegenerative disease or condition: amyloid-beta 1-42, alpha-synuclein, superoxide dismutase-1 (SOD-1), hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; Leucine-rich repeat serine/threonine-protein kinase 2 (LRRK2). In some embodiments, an antigen may be a form of any one or more of the following that may be associated with a neurodegenerative disease or condition: Huntington with tandem glutamine repeats; prion proteins; transthyretin; dentatorubral pallidoluysian atrophy (DRPLA) protein; androgen receptor; ataxins; P/Q-type calcium channel α1A subunit; TATA-box-binding protein; glial fibrillary acidic protein; DNA excision repair protein ERCC-6; survival motor neuron protein; cystatin C.

The term “antigen binding domain” or “AB domain” refers to one or more extracellular domains of a chimeric antigen receptor (CAR) which have specificity for a particular antigen.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “autologous” or refers to any material derived from the same individual to whom it is later to be re-introduced.

The term “bind” refers to an attractive interaction between two molecules that results in a stable association in which the molecules are in close proximity to each other. The result of molecular binding is sometimes the formation of a molecular complex in which the attractive forces holding the components together are generally non-covalent, and thus are normally energetically weaker than covalent bonds.

The term “CD28” refers to the protein Cluster of Differentiation 28, one of the proteins expressed on T cells that provide co-stimulatory signals required for T cell activation and survival. Mouse CD28 protein may have at least 85, 90, 95, 96, 97, 98, 99 or 100% identity to NCBI Reference No: NP_031668.3 or a fragment thereof that has stimulatory activity. Human CD28 protein may have at least 85, 90, 95, 96, 97, 98, 99 or 100% identity to NCBI Reference No: NP_006130 or a fragment thereof that has stimulatory activity.

The term “CD3 zeta,” or alternatively, “zeta,” “ζ,” “zeta chain,” “CD3-zeta,” “CD3z,” “TCR-zeta,” “CD247,” or “CD3ζ” is a protein encoded by the CD247 gene on chromosome 1, with gene location 1 H2.3; 1 73.14 cM, in mice, and by the CD247 gene on chromosome 1, with gene location 1q24.2, in humans. CD3ζ together with T cell receptor (TCR) and CD3 (a protein complex composed of a CD3γ, a CD3 δ and two CD3ε), forms the TCR complex. Mouse CD3 ζ may have an amino acid sequence provided as NP_001106864.1, NP_001106863.1, NP_001106862.1, or NP_112439.1, or the equivalent residues from a non-mouse species, e.g., human, rodent, monkey, ape and the like. Human CD3ζ may have an amino acid sequence provided as NP_000725 or NP_932170, or the equivalent residues from a non-human species, e.g., mouse, rodent, monkey, ape and the like.

The term “CD3 zeta intracellular signaling domain,” or alternatively “CD3 zeta ICS domain” or a “CD3zICS,” is defined as the amino acid residues from the cytoplasmic domain of the CD3 zeta chain, or functional derivatives thereof, that are sufficient to functionally transmit an initial signal necessary for T cell activation.

The term “4-1BB” or “BB” refers to a member of the TNFR superfamily with an amino acid sequence provided as GenBank Acc. No. AAA53133.1, or the equivalent residues from a non-human species, e.g., mouse, rodent, monkey, ape and the like. In one aspect, the “4-1BB costimulatory domain” is the sequence provided as SEQ ID NO: 12 or the equivalent residues from a non-human species, e.g., mouse, rodent, monkey, ape and the like and/or the sequence may be encoded by the nucleic acid of SEQ ID NO: 212.

The term “Chimeric Antigen Receptor” or alternatively a “CAR” refers to a set of polypeptides, typically two in the simplest embodiments, which when expressed in an immune effector and/or regulatory cell, provides the cell with specificity for a target cell, and optionally promotes intracellular signal generation. CARs according to the invention will in general comprise a receptor or ligand binding moiety, e.g., one which targets a protein aberrantly expressed in subjects comprising a neurodegenerative disorder and optionally may comprise one or more costimulatory signaling or T cell signaling domains such as CD28, 4-1BB, CD3ζ, DAP10-CD3ζ CD44-CD3ζ, CD28-CD3ζ, or 4-1BB-CD3ζ. In some embodiments, a CAR comprises at least an extracellular antigen binding domain (AB domain), a transmembrane domain (TM domain) and a cytoplasmic signaling domain (also referred to herein as “an intracellular signaling domain (ICS domain) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an AB domain to an ICS domain. In some aspects, the stimulatory molecule is the zeta chain associated with the T cell receptor complex. In some aspects, the cytoplasmic portion of a CAR further comprises a costimulatory domain (CS domain) comprising one or more functional signaling domains derived from at least one costimulatory molecule as defined below. In some aspects, the costimulatory molecule is chosen from the costimulatory molecules described herein, e.g., 4-1BB (i.e., CD137), CD44, DAP10 and/or CD28. In some aspects, the CAR comprises a chimeric fusion protein comprising an extracellular AB domain, a TM domain and an ICS domain comprising a functional signaling domain derived from a stimulatory molecule. In some aspects, the CAR comprises a chimeric fusion protein comprising an extracellular AB domain, a TM domain, an ICS domain comprising a functional signaling domain derived from a stimulatory molecule, and a CS domain comprising a functional signaling domain derived from a costimulatory molecule. In some aspects, the CAR comprises a chimeric fusion protein comprising an extracellular AB domain, a TM domain, an ICS domain comprising a functional signaling domain derived from a stimulatory molecule, and two CS domains each of the two comprising a functional signaling domain derived from a costimulatory molecule(s) that is/are same with or different from each other. In some aspects, the CAR comprises a chimeric fusion protein comprising an extracellular AB domain, a TM domain, an ICS domain comprising a functional signaling domain derived from a stimulatory molecule, and at least two CS domains each comprising a functional signaling domain derived from a costimulatory molecule(s) that is/are same with or different from each other. In some aspects, the CAR comprises an optional leader sequence at the amino-terminus (N-ter) of the CAR fusion protein. In some aspects, the CAR further comprises a leader sequence at the N-terminus of the extracellular antigen binding domain, wherein the leader sequence is optionally cleaved from the antigen binding domain (e.g., an scFv) during cellular processing and localization of the CAR to the cellular membrane.

The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The term “costimulatory molecule” or “T cell signaling moiety” herein refers to a cognate binding partner on a T cell that specifically binds with a costimulatory ligand, thereby mediating a costimulatory response by the T cell, such as, but not limited to, proliferation or the expression of specific cytokines. Costimulatory molecules are cell surface molecules other than antigen receptors or their ligands that contribute to an efficient immune response. Costimulatory molecules include, but are not limited to a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R β, IL2R γ, IL7R α, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a ligand that specifically binds with CD83. In embodiments wherein a CAR comprises one or more CS domains, wherein each CS domain comprises a functional signaling domain derived from a costimulatory molecule. In some embodiments, the encoded CS domain is that of 4-1BB, CD28, or DAP10.

The term “cytokines” refers to a broad category of small proteins that are involved in cell signaling. Generally, their release has some effect on the behavior of cells around them. Cytokines may be involved in autocrine signaling, paracrine signaling and/or endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, epithelial cells, and various stromal cells. “Chemokines” are a family of cytokines generally involved in mediating chemotaxis.

An “effective amount” or “an amount effective to treat” refers to a dose that is adequate to prevent or treat a disease, condition, or disorder in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side effects that might accompany the administration of a particular active, and the desired physiological effect. It will be appreciated by one of skill in the art that various diseases or disorders could require prolonged treatment involving multiple administrations, perhaps using one or more modified Tregs in each or various rounds of administration.

The term “hinge”, “spacer”, or “linker” refers to an amino acid sequence of variable length typically encoded between two or more domains or portions of a polypeptide construct to confer flexibility, improved spatial organization, proximity, etc.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hogeboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al., “Monoclonal Antibodies and Cancer Therapy”, Alan R. Liss, p. 77, 1985; Boerner et al., J. Immunol., 147 (1):86-95, 1991; and U.S. Pat. No. 5,750,373.

An “iCAR” is a chimeric antigen receptor which contains inhibitory receptor signaling domains. These domains may be based, for example, on protectin D1 (PD1) or CTLA-4 (CD152). In some embodiments, the modified Tregs as discussed herein may be further transduced to express an iCAR. As used herein, “immune cell” refers to a cell of hematopoietic origin functionally involved in the initiation and/or execution of innate and/or adaptive immune response.

The term “internal ribosome entry site” or “IRES” refers to a cis-acting RNA sequence that mediates internal entry of the 40S ribosomal subunit on some eukaryotic and viral messenger RNAs. IRES allows for translation initiation in a 5′ cap independent manner during protein synthesis, thus enabling co-expression of two proteins from a single mRNA. Further details and variations of IRES sequences may be found in Bonnal et al., Nucleic Acids Res. 2003 Jan. 1; 31(1): 427-428.

An “intracellular signaling domain” or “ICS domain” as the term is used herein, refers to an intracellular portion of a molecule. The intracellular signaling domain generates a signal that promotes an immune regulatory and/or effector function of the cell transduced with a nucleic acid sequence comprising a CAR, e.g., a modified Treg comprising one or more CARs. Examples of immune effector function include cytolytic activity and helper activity, including the secretion of cytokines. Example of immune regulatory function, e.g., in a modified Treg, include, but are not limited to including, ICS domains include, but are not limited to including, CD28-CD3zeta; 4-1BB-CD3 zeta; Dap10-CD3zeta; CD44-CD3zeta; CTLA-4-CD3zeta; CD28; Dap10; 4-1BB; 3-zeta. Further examples include an ICS domain of a TCR/CD3 complex protein, an Fc receptor subunit, an IL-2 receptor subunit, CD3 zeta, FcR γ, FcR β, CD3γ, CD3 δ, CD3ε, CD5, CD22, CD79a, CD79b, CD66d, CD278 (ICOS), Fc ε RI, DAP10, or DAP12.

The term “DAP10” refers to a protein, which in humans is encoded by the HSCT gene. It may also be referred to as HCST, KAP10, PIK3AP, or hematopoietic cell signal transducer. In some embodiments, DAP10 may have the sequence provided in Genbank Accession No.: Q9UBK5.1.

An “isolated” biological component (such as an isolated chimeric antigen receptor or cell or vector or protein or nucleic acid) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

The term “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises one or more repeats of the amino acid sequence unit Gly-Gly-Gly-Gly-Ser (SEQ ID NO: 13). In one embodiment, the flexible polypeptide linker includes, but is not limited to, (Gly₄Ser)₃, which is also referred to as G4S X3 (SEQ ID NO: 13). Such a linker may be encoded for example, by the nucleic acid sequence (SEQ ID NO: 213).

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

A “pharmaceutically acceptable carrier” or “excipient” refers to compounds or materials conventionally used in immunogenic compositions during formulation and/or to permit storage.

The term “promoter”, as used herein, is defined as a DNA sequence recognized by the synthetic machinery of the cell, or introduced synthetic machinery, required to initiate the specific transcription of a polynucleotide sequence.

The term “recombinant” means a moiety, e.g., a polynucleotide with semi-synthetic or synthetic origin which either does not occur in nature or is linked to another polynucleotide in an arrangement not found in nature or it may refer to a cell which is modified to express or not express a polynucleotide normally not expressed or expressed by a corresponding unmodified cell.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. The linker may comprise portions of the framework sequences.

The term “sequence identity” or “sequence homology” are used interchangeably herein and both refer to the sequence similarity of different polypeptides or nucleic acids. In general the invention contemplates polypeptide or nucleic acids or constructs containing same having at least 90% or greater sequence homology or identity to any one or more of the polypeptide or nucleic acid sequences disclosed herein, more typically polypeptide or nucleic acid sequences having at least 95% or greater sequence homology to any one or more of the polypeptide or nucleic acid sequences disclosed herein or possessing at least 98% or greater sequence homology or sequence identity, or at least 99% or greater sequence homology or identity to any one or more of the polypeptide or nucleic acid sequences set forth herein. Methods for determining homology between nucleic acid and amino acid sequences are well known to those of ordinary skill in the art. Generally such homologous nucleic acids or polypeptides will be selected or designed so as to improve or so as to not adversely impact the desired properties of the specific polypeptide or nucleic acid or construct containing same.

A “signal peptide” (also referred to as a signal sequence, targeting signal, localization signal, localization sequence, transit peptide, leader sequence or leader peptide) is a short peptide present at the N-terminus of the majority of newly synthesized proteins that are destined towards the secretory pathway. The core of the signal peptide may contain a long stretch of hydrophobic amino acids. The signal peptide may or may not be cleaved from the mature polypeptide.

A “leader sequence” as used herein, also referred to as “signal peptide,” “signal sequence,” “targeting signal,” “localization signal,” “localization sequence,” “transit peptide,” or “leader peptide” in the art, is a short peptide present at the N-terminus of the majority of newly synthesized proteins that are destined towards the secretary pathway. The core of the signal peptide may contain a long stretch of hydrophobic amino acids. The signal peptide may or may not be cleaved from the mature polypeptide.

The “ribosome skip sequence” refers to an amino acid sequence that, when translated, causes cleavage of a nascent polyprotein on the ribosome, allowing for co-expression of multiple genes. In one aspect, the ribosome skip sequence may be the T2A sequence and comprises the amino acid sequence of SEQ ID NO: 14 or nucleotide sequence encoding such, such as SEQ ID NO: 214. Alternatively, any other 2A sequences may be used. Examples of other 2A sequences may be found elsewhere in the literature of the relevant art (for example, see Kim, J. H., et al., “High cleavage efficiency of a 2A peptide derived from porcine teschovirus-1 in human cell lines, zebrafish and mice” PLoS One. 2011; 6(4)).

The term “signaling domain” refers to the functional portion of a protein which acts by transmitting information within the cell to regulate cellular activity via defined signaling pathways by generating second messengers or functioning as effectors by responding to such messengers. Examples of signaling domains include, but are not limited to including, CD28-CD3ζ; 4-1BB-CD3ζ; Dap10-CD3ζ; CD44-CD3ζ; CTLA-4-CD3ζ; CD28; Dap10; 4-1BB; and CD3-ζ.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with antigenic peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Examples of an ITAM containing cytoplasmic signaling sequence include, but are not limited to, those derived from CD3ζ (amino acid sequence of SEQ ID NO: 16, which may be encoded by SEQ ID NO: 216), common FcRy (FCER1G), FcγRIIa, FcRβ (FcεR1b), CD3γ, CD3δ, CD3Σ, CD79a, CD79b, DAP10, and DAP12. In exemplary embodiments, the intracellular signaling domain in any one or more CARs comprised by a modified Treg may comprise an intracellular signaling sequence, e.g., a primary signaling sequence of CD3ζ. Alternatively, equivalent residues from a non-human or mouse species, e.g., rodent, monkey, ape and the like, may be utilized.

The term “subject” is intended to include living organisms in which an immune response can be elicited (e.g., mammals, human). The subject may have a disease or may be healthy. The subject may also be referred to as “patient” in the art.

The term “suicide mechanism” or “suicide gene” as used herein refers to a mechanism by which CAR-expressing cells of present invention may be eradicated from a subject administered with CAR-expressing cells. The suicide mechanism may be driven by, for example, inducible caspase 9 (Budde et al., PLoS One 2013 8(12):82742), codon-optimized CD20 (Marin et al., Hum. Gene Ther. Meth. 2012 23(6)376-86), CD34, a truncated EGFR (Wang X, Chang W-C, Wong C W, et al. A transgene-encoded cell surface polypeptide for selection, in vivo tracking, and ablation of engineered cells. Blood. 2011; 118(5):1255-1263. doi:10.1182/blood-2011-02-337360), a truncated CD19, or polypeptide RQR8 (Philip et al, and WO2013153391A, which is hereby incorporated herein by reference). In some embodiments, the suicide mechanism may be included and utilized in modified Tregs discussed herein to optimize the length for the modified Tregs to stay in the system of a subject or the amount of the modified Tregs, to reduce or minimize the toxicity and/or to maximize the benefit of said modified Tregs.

The term “target cell” as used herein refers to a cell expressing the target molecule of a CAR comprised by a modified Treg on the cell surface. In some embodiments, the target cell is a microglia cell. In some embodiments, the target cell is a neuron. In some embodiments, the target cell is a cell type that has a particular role in the pathology of a neurodegenerative disease and/or condition and/or neuroinflammation. In some embodiments, the target cell is a cell type that has a particular role in the pathology of a disease such as but not limited to Alzheimer's disease, Parkinson's disease, ALS, and any of the other neurodegenerative diseases and conditions discussed herein.

The term “target molecule” as used herein refers to a molecule that is targeted by a CAR or a cell which expresses same such as a modified Treg, e.g., a modified Treg comprising one or more CARs, of the present disclosure. The AB domain of a CAR comprised by a modified Treg of the present disclosure may have a binding affinity for the target molecule. In some embodiments, the target molecule is a form of amyloid-beta 1-42 associated with a neurodegenerative disease or condition. In some embodiments, the target molecule is a form of alpha-synuclein associated with a neurodegenerative disease or condition. In some embodiments, the target molecule is a form of superoxide dismutase-1 (SOD-1) associated with a neurodegenerative disease or condition. In some embodiments, the target molecule may be, but is not limited to being, forms of any of the following associated with a neurodegenerative disease and/or condition: hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; Leucine-rich repeat serine/threonine-protein kinase 2 (LRRK2), Huntington with tandem glutamine repeats; prion proteins; transthyretin; dentatorubral pallidoluysian atrophy (DRPLA) protein; androgen receptor; ataxins; P/Q-type calcium channel α1A subunit; TATA-box-binding protein; glial fibrillary acidic protein; DNA excision repair protein ERCC-6; survival motor neuron protein; and cystatin C. In some embodiments, the target molecule may be a molecule associated with any of the following non-limiting list of neurodegenerative diseases: Alzheimer's disease, Parkinson's disease, ALS, prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathies; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis.

The term “transfected,” “transformed,” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “transmembrane domain” or “TM domain”, what is implied is any three-dimensional protein structure which is thermodynamically stable in a membrane. This may be a single a helix, a transmembrane β barrel, a β-helix of gramicidin A, or any other structure. Transmembrane helices are usually about 20 amino acids in length. Typically, the transmembrane domain denotes a single transmembrane α helix of a transmembrane protein, also known as an integral protein.

As used herein, the terms “treat,” “treatment,” or “treating” generally refers to a clinical procedure for reducing or ameliorating the onset, progression, severity, and/or duration of a disease and/or condition, or for ameliorating one or more symptoms (preferably, one or more discernible symptoms) of a disease and/or condition. The disease may be, for example, a neurodegenerative disease or condition. In some embodiments, the effect of the “treatment” may be evaluated by the amelioration of at least one measurable physical parameter of a disease, resulting from the administration of one or more therapies (e.g., one or more therapeutic agents such as a modified Treg as described herein). The parameter may be, for example, gene expression profiles, the mass of disease-affected tissues, inflammation-associated markers, neurodegenerative disease-associated markers, the presence or absence of certain cytokines or chemokines or other disease-associated molecules, and may not necessarily discernible by the patient. In other embodiments “treat”, “treatment,” or “treating” may result in the inhibition of the progression of a disease and/or condition, either physically by, e.g., stabilization of a discernible symptom, physiologically by, e.g., stabilization of a physical parameter, or both. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease and/or condition in a mammal. Furthermore, the treatment or prevention provided by the methods described herein can include treatment or prevention of one or more conditions or symptoms of the disease being treated or prevented. Also, for purposes herein, “prevention” can encompass delaying the onset of the disease, or a symptom or condition thereof.

The term “xenogeneic” refers to a graft derived from an animal of a different species.

As used herein, the term “recombinant Tregs” or “modified Tregs” generally refers to a regulatory T cell that has been altered relative to its native state, e.g., genetically modified. For example, in exemplary embodiments, a modified Treg may be engineered to express one or more CARs. Additionally, exemplary modified Tregs may be engineered to express one or more NDMMs. Modified Tregs according to the invention may be used to treat various diseases in exemplary embodiments. For example, modified Tregs may be used in methods of treating specific neurodegenerative diseases, conditions, or disorders. Exemplary neurodegenerative diseases, conditions, and/or disorders that may be treated with modified Tregs as disclosed herein include by way of example ALS, Alzheimer's disease, and Parkinson's disease. Furthermore, in exemplary embodiments the modified Tregs of the present disclosure may be used to treat neuroinflammation in the CNS. In some embodiments, the modified Tregs of the present disclosure may be used to treat any of the following non-limiting list of neurodegenerative diseases and/or conditions: Alzheimer's disease, Parkinson's disease, ALS, prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathies; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis.

Modified Tregs, and Compositions and Methods of Use Thereof

The present disclosure generally relates to modified Tregs, e.g., Tregs engineered to express one or more chimeric antigen receptors (“CARs”) and/or one or more neurodegenerative disease modifying molecules, compositions comprising said modified Tregs and methods of using said modified Tregs and compositions containing, in particular for treating a disease, disorder, or condition, e.g., neurodegenerative diseases, disorders, or conditions. In exemplary embodiments, these modified Tregs are engineered to express on their surface at least one moiety, e.g., an antibody and typically an scFv which recognizes a protein the expression of which is associated with a specific neurodegenerative condition, e.g., specific molecular markers of a particular neurodegenerative disease or condition, such as, for example, proteins and/or molecular markers associated with neuroinflammation, ALS, Alzheimer's disease, and/or Parkinson's disease. In other exemplary embodiments, modified Tregs are engineered to express or further express specific molecules that prevent oxidative/inflammatory activity and/or which promote neuronal growth, function and/or survival such as anti-oxidants, nerve growth factors and non-classical neurotrophic growth factors. In exemplary embodiments, modified Tregs according to the invention will maintain Treg phenotype and/or maintain at least one Treg effector function, and ideally will retain substantially all effector functions possessed by unmodified Tregs. In some exemplary embodiments modified Tregs according to the invention may migrate or traffic to the site of neurodegeneration and/or neuroinflammation. In further exemplary embodiments, modified Tregs according to the invention may lead to an anti-inflammatory activity at a site of neurodegeneration and/or neuroinflammation. Said activities may comprise any activities which are associated with neurodegeneration and/or neuroinflammation, such as, for example, (1) microglia cell over-activation, (2) production of inflammatory proteins/activities, and (3) neuronal death. Exemplary methods of treatment comprising modified Tregs may result in reduced disease progression and/or may repair and/or improve function in a patient in need thereof. In some embodiments, Tregs may be isolated from donor PBMCs and expanded for use, e.g., clinical use. In some exemplary embodiments the donor cells may be allogeneic. In other exemplary embodiments the donor PBMCs used to derive Tregs may be isolated from the same subject who is to be treated. In some exemplary embodiments the isolated Tregs may be modified to reduce or eliminate the expression or functionality of the endogenous TCR. In some exemplary embodiments the isolated Tregs may be combined with Tregs isolated from different donors which donors may be MHC compatible or incompatible. In some embodiments, Tregs may be expanded by up to a 550-fold increase in number, in some instances in a two week period of culturing. In other exemplary embodiments, said Tregs will comprise a purity of 90% or greater. In other exemplary embodiments, modified Tregs may cross the blood-brain barrier (BBB).

In some embodiments, Tregs may be isolated, expanded, and transduced as follows: cells may be isolated from human PBMCs via a two-step negative and positive selection protocol. First, CD4⁺ cells are isolated using negative selection, followed by a positive selection of CD25hi⁺ cells to isolate CD4⁺CD25hi⁺ Treg cells. The isolated CD4⁺CD25hi⁺Tregs may be activated with anti-CD3, anti-CD28, anti-CD2 multimers or anti-CD3, anti-CD28 multimers (STEMCELL ImmunoCult) with human IL-2 (300 U/ml to 500 U/ml) over two weeks in culture in Treg growth medium. On day 9, Treg cells can be cryopreserved for use at a later date, so we can test the same donor and preparation on multiple occasions to assess assay variability.

CARs

In further exemplary aspects, modified Tregs may be modified to express one or more CARs, preferably expressed on their cell surface. Exemplary CARs according to the invention may comprise a ligand binding moiety such as a receptor or an antibody, e.g., an scFv which recognizes proteins and/or other molecular markers associated with diseases and/or conditions which are to be treated using the Tregs, for example, particular neurodegenerative diseases and/or neuroinflammation. In some embodiments of the invention these CARs may further comprise one or more costimulatory signaling or T cell signaling moieties or domains such as are identified herein and exemplified in the working examples or others which are generally known in the art. Exemplary neurodegenerative diseases characterized by the expression of aberrant proteins or aberrantly expressed proteins which are associated with disease pathology that may be recognized by CARs expressed by modified Tregs according to the invention include by way of example ALS, Alzheimer's disease, and Parkinson's disease. When expressed in modified Tregs, one or more CARs as described herein may trigger effector responses, such as, for example, cytokine expression. In exemplary embodiments, the CAR may trigger effector responses in the presence of one or more specific antigens. Moreover, in some aspects, modified Tregs may comprise one or more CARs, and said one or more CARs may trigger IL-10 production upon stimulation, e.g., stimulation by a specific antigen. In further exemplary embodiments, modified Tregs may comprise one or more CARs, and said modified Tregs may retain their ability to suppress other T cells. In some embodiments, modified Tregs may comprise one or more CARs, and said modified Tregs may be in subsets of T cells with known regulatory and/or anti-inflammatory activity, such as, for example, FOXP3⁺ Tregs. In exemplary embodiments, modified Tregs may comprise one or more CARs, wherein said one or more CARs comprise single chain variable fragments (scFv) that may be targeted to a protein and/or molecular marker of a disease, transmembrane signaling domain, and cytoplasmic signaling domain. In exemplary embodiments, modified Tregs comprising one or more CARs targeted to a disease of interest may result in greater inhibition of inflammatory cytokine production, higher expression of anti-inflammatory cytokines, greater protection against neuroinflammation-mediated motor neuron death, and improve survival as compared to control treatments.

In some embodiments, a modified Treg may comprise one or more CARs as described herein, wherein the sequence that encodes said CARs may comprise a signal sequence. It is to be understood that said signal sequence may or may not be cleaved during expression of said CAR. In some embodiments, a modified Treg may comprise one or more CARs that comprise a sequence of SEQ ID NO: 1 (DG01), which may optionally be encoded by the nucleic acid sequence of SEQ ID NO: 201), and/or a construct comprising at least 90%, at least 95%, at least 98%, or at least 99% sequence identity to the aforementioned construct. In some embodiments, DG01 may target a form of amyloid-beta associated with a neurodegenerative disease or condition. In some embodiments, a modified Treg may comprise one or more CARs that comprise a sequence of SEQ ID NO: 2 (DG02), which may optionally be encoded by the nucleic acid sequence of SEQ ID NO: 202, and/or a construct comprising at least 90%, at least 95%, at least 98%, or at least 99% sequence identity to the aforementioned construct. In some embodiments, DG02 may target a form of amyloid-beta associated with a neurodegenerative disease or condition In some embodiments, a modified Treg may comprise one or more CARs that comprise a sequence of SEQ ID NO: 3 (DG03), which may optionally be encoded by the nucleic acid sequence of SEQ ID NO: 203, and/or a construct comprising at least 90%, at least 95%, at least 98%, or at least 99% sequence identity to the aforementioned construct. In some embodiments, DG03 may target a form of amyloid-beta associated with a neurodegenerative disease or condition. In some embodiments, a modified Treg may comprise one or more CARs that comprise a sequence of SEQ ID NO: 4 (DG04), which may optionally be encoded by the nucleic acid sequence of SEQ ID NO: 204, and/or a construct comprising at least 90%, at least 95%, at least 98%, or at least 99% sequence identity to the aforementioned construct. In some embodiments, DG04 may target a form of amyloid-beta associated with a neurodegenerative disease or condition. In some embodiments, a modified Treg may comprise one or more CARs that comprise a sequence of SEQ ID NO: 5 (DG05), which may optionally be encoded by the nucleic acid sequence of SEQ ID NO: 205, and/or a construct comprising at least 90%, at least 95%, at least 98%, or at least 99% sequence identity to the aforementioned construct. In some embodiments, DG05 may target a form of human superoxide mutase associated with a neurodegenerative disease or condition. In some embodiments, a modified Treg may comprise one or more CARs that comprise a sequence of SEQ ID NO: 6 (DG06), which may optionally be encoded by the nucleic acid sequence of SEQ ID NO: 206, and/or a construct comprising at least 90%, at least 95%, at least 98%, or at least 99% sequence identity to the aforementioned construct. In some embodiments, DG06 may target a form of superoxide mutase associated with a neurodegenerative disease or condition. In some embodiments, a modified Treg may comprise one or more CARs that comprise a sequence of SEQ ID NO: 7 (DG07), which may optionally be encoded by the nucleic acid sequence of SEQ ID NO: 207, and/or a construct comprising at least 90%, at least 95%, at least 98%, or at least 99% sequence identity to the aforementioned construct. In some embodiments, DG07 may target a form of superoxide mutase associated with a neurodegenerative disease or condition. In some embodiments, a modified Treg may comprise one or more CARs that comprise a sequence of SEQ ID NO: 8 (DG08), which may optionally be encoded by the nucleic acid sequence of SEQ ID NO: 208, and/or a construct comprising at least 90%, at least 95%, at least 98%, or at least 99% sequence identity to the aforementioned construct. In some embodiments, DG08 may target a form of alpha-synuclein associated with a neurodegenerative disease or condition. In some embodiments, a modified Treg may comprise one or more CARs that comprise a sequence of SEQ ID NO: 9 (DG09), which may optionally be encoded by the nucleic acid sequence of SEQ ID NO: 209, and/or a construct comprising at least 90%, at least 95%, at least 98%, or at least 99% sequence identity to the aforementioned construct. In some embodiments, DG09 may target a form of alpha-synuclein associated with a neurodegenerative disease or condition. In some embodiments, a modified Treg may comprise one or more CARs that comprise a sequence of SEQ ID NO: 10 (DG010), which may optionally be encoded by the nucleic acid sequence of SEQ ID NO: 210, and/or a construct comprising at least 90%, at least 95%, at least 98%, or at least 99% sequence identity to the aforementioned construct. In some embodiments, DG10 may target a form of alpha-synuclein associated with a neurodegenerative disease or condition. In some embodiments, a modified Treg may comprise one or more CARs that comprise a sequence of SEQ ID NO: 11 (DG11), which may optionally be encoded by the nucleic acid sequence of SEQ ID NO: 211, and/or a construct comprising at least 90%, at least 95%, at least 98%, or at least 99% sequence identity to the aforementioned construct. In some embodiments, DG11 may target a form of alpha-synuclein associated with a neurodegenerative disease or condition.

In exemplary embodiments, a CAR may target a protein or a mutant version of a protein comprising a sequence as set forth in Table 1:

TABLE 1

| TARGET MOLECULE | SEQUENCE |
|---|---|
| Human amyloid beta, | SEQ ID NO: 17 |
| isoform APP770 | |
| (identifier: P05067-1) | |
| human superoxide | SEQ ID NO: 18 |
| dismutase (human | |
| superoxide dismutase, | |
| identifier: P00441-1, | |
| wild type sequence) | |
| human alpha-synuclein, | SEQ ID NO: 19 |
| Isoform 1 | |
| (identifier: P37840-1) | |

In some embodiments, a CAR may target a molecular marker, e.g., a protein, associated with any one or more of the following neurodegenerative diseases: Alzheimer's disease, Parkinson's disease, ALS, prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathies; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis.

In some exemplary embodiments, a CAR comprised by a modified Treg may target amyloid beta 1-42. In other exemplary embodiments, a CAR comprised by a modified Treg may target superoxide dismutase-1 (SOD-1). In other exemplary embodiments, a CAR comprised by a modified Treg may target alpha-synuclein. In other exemplary embodiments, a CAR comprised by a modified Treg may target any of the following non-limiting list: hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; Leucine-rich repeat serine/threonine-protein kinase 2 (LRRK2). In some embodiments, a CAR comprised by a modified Treg may target any one or more of the following: Huntington with tandem glutamine repeats; prion proteins; transthyretin; dentatorubral pallidoluysian atrophy (DRPLA) protein; androgen receptor; ataxins; P/Q-type calcium channel α1A subunit; TATA-box-binding protein; glial fibrillary acidic protein; DNA excision repair protein ERCC-6; survival motor neuron protein; cystatin C.

In other exemplary embodiments, modified Tregs may comprise one or more CARs and may further comprise one or more signaling domains which may be encoded by nucleic acids which are comprised on the same or different nucleic acid construct as the nucleic acids encoding the one or more CARs. Said signaling domains may activate different effector pathways and/or survival pathways in said modified Tregs and optionally may affect cytokine expression, e.g., trigger IL-10 expression in the presence of a target antigen such as an aberrantly expressed protein expressed in the CNS at a site of neurodegeneration.

In some exemplary embodiments, modified Tregs may have no signaling, or T cell signaling may be used (i.e. an antigen tether as CAR, e.g., CD28 transmembrane only). In other exemplary embodiments, one or more CARs expressed by the modified Tregs may comprise signaling domain combinations (costimulation signaling domains) including by way of example: CD28-CD3ζ; 4-1BB-CD3ζ; Dap10-CD3ζ; CD44-CD3ζ; CTLA-4-CD3ζ; CD28; Dap10; 4-1BB; CD3-ζ. In further exemplary embodiments, a costimulation signaling domain expressed by the modified Tregs may comprise CD28-CD3ζ; DAP10-CD3ζ; CD44-CD3ζ; 4-1BB-CD3ζ; CD28; or CD3-ζ.

Moreover, specific exemplary embodiments of the present invention relate to modified Tregs that comprise one or CARs which comprise single chain variable fragments that were derived from antibodies specific to proteins and/or other molecular markers associated with diseases and/or conditions associated with diseases, such as, for example, neurodegenerative diseases and/or neuroinflammation including but not limited to specific scFv antibody sequences which are disclosed herein.

In some embodiments, a modified Treg may comprise CAR DG05-28Z (SEQ ID NO: 24, which may optionally be encoded by SEQ ID NO: 224), and/or a construct comprising at least 90%, at least 95%, at least 98%, or at least 99% sequence identity to the aforementioned construct. In some embodiments, a modified Treg may comprise CAR DG06-28Z (SEQ ID NO: 25, which may optionally be encoded by SEQ ID NO: 225)), and/or a construct comprising at least 90%, at least 95%, at least 98%, or at least 99% sequence identity to the aforementioned construct. In some embodiments, a modified Treg may comprise CAR DG07-28Z (SEQ ID NO: 26, which may optionally be encoded by SEQ ID NO: 226), and/or a construct comprising at least 90%, at least 95%, at least 98%, or at least 99% sequence identity to the aforementioned construct. In some embodiments, a modified Treg may comprise CAR DG10-28Z (SEQ ID NO: 29, which may optionally be encoded by SEQ ID NO: 229), and/or a construct comprising at least 90%, at least 95%, at least 98%, or at least 99% sequence identity to the aforementioned construct. In some embodiments, a modified Treg may comprise DG01-CD28-CD3ζ (SEQ ID NO: 20, which may optionally be encoded by SEQ ID NO: 220), DG02-CD28-CD3ζ (SEQ ID NO: 21, which may optionally be encoded by SEQ ID NO: 221), DG03-CD28-CD3ζ (SEQ ID NO: 22, which may optionally be encoded by SEQ ID NO: 222), DG04-CD28-CD3ζ (SEQ ID NO: 23, which may optionally be encoded by SEQ ID NO: 223), and/or a construct comprising at least 90%, at least 95%, at least 98%, or at least 99% sequence identity to any one or more of the aforementioned constructs. In some embodiments, a modified Treg may comprise DG05-CD28-CD3ζ (SEQ ID NO: 24, which may optionally be encoded by SEQ ID NO: 224), DG06-CD28-CD3ζ (SEQ ID NO: 25, which may optionally be encoded by SEQ ID NO: 225), DG07-CD28-CD3ζ (SEQ ID NO: 26, which may optionally be encoded by SEQ ID NO: 226), and/or a construct comprising at least 90%, at least 95%, at least 98%, or at least 99% sequence identity to any one or more of the aforementioned constructs. In some embodiments, a modified Treg may comprise DG08-CD28-CD3ζ (SEQ ID NO: 27, which may optionally be encoded by SEQ ID NO: 227), DG09-CD28-CD3ζ (SEQ ID NO: 28, which may optionally be encoded by SEQ ID NO: 228), DG10-CD28-CD3ζ (SEQ ID NO: 29, which may optionally be encoded by SEQ ID NO: 229), DG11-CD28-CD3ζ (SEQ ID NO: 30, which may optionally be encoded by SEQ ID NO: 230), and/or a construct comprising at least 90%, at least 95%, at least 98%, or at least 99% sequence identity to any one or more of the aforementioned constructs. In some embodiments, a modified Treg may comprise DG05-CD28-CD3ζ (also referred to as DG05-28-3ζ) (SEQ ID NO: 24, which may optionally be encoded by SEQ ID NO: 224), and/or a construct comprising at least 90%, at least 95%, at least 98%, or at least 99% sequence identity to the aforementioned construct. In some embodiments, a modified Treg may comprise DG05-CD28tm-DAP10-CD3ζ (also referred to as DG05-28tm-10-3ζ) (SEQ ID NO: 40, which may optionally be encoded by SEQ ID NO: 240), and/or a construct comprising at least 90%, at least 95%, at least 98%, or at least 99% sequence identity to the aforementioned construct. In some embodiments, a modified Treg may comprise DG05-CD28tm-CD44-CD3ζ (also referred to as DG05-28tm-44-3ζ) (SEQ ID NO: 41, which may optionally be encoded by SEQ ID NO: 241), and/or a construct comprising at least 90%, at least 95%, at least 98%, or at least 99% sequence identity to the aforementioned construct. In some embodiments, a modified Treg may comprise DG05-CD28tm-CD3ζ (also referred to as DG05-28tm-3ζ) (SEQ ID NO: 42, which may optionally be encoded by SEQ ID NO: 242), and/or a construct comprising at least 90%, at least 95%, at least 98%, or at least 99% sequence identity to the aforementioned construct. In some embodiments, a modified Treg may comprise DG05-CD28 (also referred to as DG05-28) (SEQ ID NO: 43, which may optionally be encoded by SEQ ID NO: 243), and/or a construct comprising at least 90%, at least 95%, at least 98%, or at least 99% sequence identity to the aforementioned construct. In some embodiments, a modified Treg may comprise DG05-CD28tm (also referred to as DG05-28tm) (SEQ ID NO: 44, which may optionally be encoded by SEQ ID NO: 244), and/or a construct comprising at least 90%, at least 95%, at least 98%, or at least 99% sequence identity to the aforementioned construct. In some embodiments, a modified Treg may comprise DG03-CD28-CD3ζ (also referred to as DG03-28-3ζ) (SEQ ID NO: 22, which may optionally be encoded by SEQ ID NO: 222), and/or a construct comprising at least 90%, at least 95%, at least 98%, or at least 99% sequence identity to the aforementioned construct. In some embodiments, a modified Treg may comprise DG03-CD28tm-DAP10-CD3ζ (also referred to as DG03-28tm-10-3ζ) (SEQ ID NO: 45, which may optionally be encoded by SEQ ID NO: 245), and/or a construct comprising at least 90%, at least 95%, at least 98%, or at least 99% sequence identity to the aforementioned construct. In some embodiments, a modified Treg may comprise DG03-CD28tm-CD44-CD3ζ (also referred to as DG03-28tm-44-3ζ) (SEQ ID NO: 46, which may optionally be encoded by SEQ ID NO: 246), and/or a construct comprising at least 90%, at least 95%, at least 98%, or at least 99% sequence identity to the aforementioned construct. In some embodiments, a modified Treg may comprise DG03-CD28tm-4-1-BB-CD3ζ (also referred to as DG03-28tm-BB-3ζ) (SEQ ID NO: 47, which may optionally be encoded by SEQ ID NO: 247), and/or a construct comprising at least 90%, at least 95%, at least 98%, or at least 99% sequence identity to the aforementioned construct. In some embodiments, a modified Treg may comprise DG03-CD28tm-CD3ζ (also referred to as DG03-28tm-3ζ) (SEQ ID NO: 48, which may optionally be encoded by SEQ ID NO: 248), and/or a construct comprising at least 90%, at least 95%, at least 98%, or at least 99% sequence identity to the aforementioned construct. In some embodiments, a modified Treg may comprise DG03-CD28 (also referred to as DG03-28) (SEQ ID NO: 49, which may optionally be encoded by SEQ ID NO: 249), and/or a construct comprising at least 90%, at least 95%, at least 98%, or at least 99% sequence identity to the aforementioned construct. In some embodiments, a modified Treg may comprise DG03-CD28tm (also referred to as DG03-28tm) (SEQ ID NO: 50, which may optionally be encoded by SEQ ID NO: 250), and/or a construct comprising at least 90%, at least 95%, at least 98%, or at least 99% sequence identity to the aforementioned construct.

In further exemplary embodiments, modified Tregs according to the invention may express one or more CARs, e.g., which target proteins aberrantly expressed at a site of CNS neurodegeneration and may further be modified to express one or more NDMMs. Alternatively modified Tregs may be generated which express one or more NDMMs which do not express a CAR. These modified NDMM expressing Tregs optionally may be combined with modified Tregs which express one or more CARs. Said NDMMs may comprise molecules that prevent oxidative and/or inflammatory activity. In further exemplary embodiments, said NDMMs when expressed in Tregs may activate neuronal growth and/or survival. Exemplary NDMMs may comprise pro-neuronal factors, anti-oxidants, nerve growth factors, and/or non-classical neurotrophic factors. Exemplary anti-oxidants include, but are not limited to including, HO-1, Ferritin, Glutathione reductase, Glutathione peroxidase, Ferritin (H), Metallothionein I, Thioredoxin, Thioredoxin reductase, Peroxiredoxin MSP23, Cu/Zn superoxide dismutase, Catalase, NRF2 activity, peroxiredoxins (Prxs); activity-dependent neuroprotector homeobox (ADNP); phycocyanin; neuroglobin. Exemplary pro-neuronal factors include, but are not limited to including: interleukin-1 receptor antagonist (IL-1ra); interleukin-6 (IL-6); activated protein C (APC); thrombomodulin; tissue plasminogen activator (tPA); Protein deglycase DJ-1; tissue inhibitor of metalloproteinases (TIMPs). Exemplary nerve growth factors include, but are not limited to including, classic neurotrophins: Brain-derived neurotrophic factor (BDNF), Ciliary neurotrophic factor (CNTF), Glial cell-line derived neurotrophic factor (GDNF). Exemplary non-classical neurotrophic factors include, but are not limited to including, Insulin-like growth factor-1 (IGF-1), Vascular endothelial growth factor, VEGF), Fibroblast Growth Factors (FGF), Hepatocyte Growth Factor (HGF), Bone Morphogenetic Proteins (BMPs), Erythropoietin (EPO), Thrombopoietin (TPO), Granulocyte-colony stimulating factor (G-CSF). In some exemplary embodiments, NDMMs which are expressed by modified Tregs according to the invention may be controlled by a constitutive promoter. In other exemplary embodiments, NDMMs which are expressed by modified Tregs according to the invention may be controlled by an inducible promoter system. The selection of suitable constitutive and inducible promoters is well within the skill in the art and many such promoters are known and readily available. Furthermore, expression of said NDMMs by said modified Tregs may be regulated by CAR-triggered transcriptional control. In some embodiments of the present invention, CAR-expressing modified Tregs as described herein may further comprise exogenously introduced polynucleotides encoding one or more NDMMs. In some embodiments, the exogenously introduced polynucleotides encoding an NDMM and the CAR construct may be introduced into the cell using a single vector. When one vector is used for both a CAR and an NDMM, the CAR and the NDMM may be encoded in the vector under the same promoter in cis. In such cases, the CAR and NDMM constructs may be separated by a sequence that allows generation of two separate translation products, for example the IRES sequence or T2A sequence (encoded by SEQ ID NO: 214).

In some embodiments, a CAR-expressing modified Treg may express an NDMM which may be human catalase. In some embodiments, a CAR-expressing modified Treg may express an NDMM which may be Neh2 domain of human Nrf2. In some embodiments, a CAR-expressing modified Treg may express an NDMM which may be human BDNF. In some embodiments, a CAR-expressing modified Treg may express an NDMM which may be human IGF-1. In some embodiments, a modified Treg may comprise a construct for expression of the NDMM Nrf2 (Keap1 inhibitor peptide) (SEQ ID NO: 51, which may optionally be encoded by SEQ ID NO: 251), and/or a construct comprising at least 90%, at least 95%, at least 98%, or at least 99% sequence identity to the aforementioned construct. In some embodiments, a modified Treg may comprise a construct for expression of the NDMM human catalase (SEQ ID NO: 52, which may optionally be encoded by SEQ ID NO: 252), and/or a construct comprising at least 90%, at least 95%, at least 98%, or at least 99% sequence identity to the aforementioned construct. In some embodiments, a modified Treg may comprise a construct for expression of the NDMM BDNF (SEQ ID NO: 53, which may optionally be encoded by SEQ ID NO: 253), and/or a construct comprising at least 90%, at least 95%, at least 98%, or at least 99% sequence identity to the aforementioned construct. In some embodiments, a modified Treg may comprise a construct for expression of the NDMM IGF-1 (SEQ ID NO: 54, which may optionally be encoded by SEQ ID NO: 254), and/or a construct comprising at least 90%, at least 95%, at least 98%, or at least 99% sequence identity to the aforementioned construct.

Alternatively, a CAR construct and NDMM construct may be contained in separate vectors for transfecting or transducing cells using two or more different vectors.

Exemplary modified Tregs may comprise modified Tregs targeting proteins and/or molecular markers of Parkinson's disease. In exemplary embodiments, modified Tregs targeted to Parkinson's disease may comprise one or more CARs, wherein said one or more CARs may target α-synuclein fibrils. In some embodiments, a CAR targeted to alpha-synuclein may comprise a sequence of DG08 (SEQ ID NO: 8), and/or a construct comprising at least 90%, at least 95%, at least 98%, or at least 99% sequence identity to the aforementioned construct. In some embodiments, a CAR targeted to alpha-synuclein may comprise a sequence of DG09 (SEQ ID NO: 9), and/or a construct comprising at least 90%, at least 95%, at least 98%, or at least 99% sequence identity to the aforementioned construct. In some embodiments, a CAR targeted to alpha-synuclein may comprise a sequence of DG10 (SEQ ID NO: 10), and/or a construct comprising at least 90%, at least 95%, at least 98%, or at least 99% sequence identity to the aforementioned construct. In some embodiments, a CAR targeted to alpha-synuclein may comprise a sequence of DG11 (SEQ ID NO: 11), and/or a construct comprising at least 90%, at least 95%, at least 98%, or at least 99% sequence identity to the aforementioned construct. In some embodiments, a modified Treg may comprise DG08-CD28-CD3ζ (SEQ ID NO: 27), DG09-CD28-CD3ζ (SEQ ID NO: 28), DG10-CD28-CD3ζ (SEQ ID NO: 29), DG11-CD28-CD3ζ (SEQ ID NO: 30), and/or a construct comprising at least 90%, at least 95%, at least 98%, or at least 99% sequence identity to any one or more of the aforementioned constructs, wherein each of the constructs is targeted to alpha-synuclein. In further exemplary embodiments, modified Tregs, such as modified Tregs comprising one or more CARs, may target one or more neurotoxic inflammatory mediators, e.g., neurotoxic inflammatory mediations produced by activated microglia. Said modified Tregs may decrease and/or inhibit microglia activation. In exemplary embodiments, modified Tregs comprise targeted anti-inflammatory and neuroprotective therapeutic activity at the disease site of dopamine neuron degeneration in PD. In some embodiments, modified Tregs may mediate their function only at the site where α-synuclein fibrils are present.

In exemplary embodiments, modified Tregs may comprise one or more CARs and/or one or more NDMMs targeted to Parkinson's disease, wherein said one or more CARs comprise single chain variable fragments such as V_{H} and V_{L} amino acid sequences of human and mouse monoclonal antibodies against human α-synuclein fibrils (such as, for example, amino acid sequences derived from clones NI 202.3G12, NI 202.12F4, NI 202.21D11, and mAb49/G). In exemplary embodiments, modified Tregs may comprise said scFV and further comprise a construct comprising CD28-CD3ζ CAR, i.e., scFv-CD28-CD3ζ CAR, wherein said scFv is specific for human α-synuclein fibrils. Constructs comprising said scFv may comprise V_{H}+V_{L} and V_{L}+V_{H} arrangements. In exemplary embodiments, a vector may comprise said scFv-CD28-CD3ζ CAR construct, and may further comprise a separate truncated (non-signaling) human CD19 (tCD19). Said tCD19 may be used as a transduction marker, such as for cell monitoring and/or cell purification purposes. In exemplary embodiments, a vector comprising any of the sequences described herein may comprise a retroviral expression vector, e.g., pSFG.

Furthermore, in exemplary embodiments, modified Tregs may comprise one or more CARs and/or one or more NDMMs targeted to ALS. Said modified Tregs may target mutSOD1, e.g., said modified Tregs may comprise one or more CARs targeted to mutSOD1. In some embodiments, a CAR targeted to mutSOD1 may comprise a sequence of DG05 (SEQ ID NO: 5), and/or a construct comprising at least 90%, at least 95%, at least 98%, or at least 99% sequence identity to the aforementioned construct. In some embodiments, a CAR targeted to mutSOD1 may comprise a sequence of DG06 (SEQ ID NO: 6), and/or a construct comprising at least 90%, at least 95%, at least 98%, or at least 99% sequence identity to the aforementioned construct. In some embodiments, a CAR targeted to mutSOD1 may comprise a sequence of DG07 (SEQ ID NO: 7), and/or a construct comprising at least 90%, at least 95%, at least 98%, or at least 99% sequence identity to the aforementioned construct. In some embodiments, a modified Treg may comprise DG05-CD28-CD3ζ (SEQ ID NO: 24), DG06-CD28-CD3ζ (SEQ ID NO: 25), DG07-CD28-CD3ζ (SEQ ID NO: 26), and/or a construct comprising at least 90%, at least 95%, at least 98%, or at least 99% sequence identity to any one or more of the aforementioned constructs, wherein each of the constructs is targeted to mutSOD1. In some embodiments, a modified Treg may comprise DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs, wherein each of the constructs is targeted to mutSOD1.

In some embodiments, modified Tregs may comprise CARs targeted to mutSOD1, and in some exemplary embodiments an scFv of said CARs may be expressed extracellularly with the C-terminus of the V_{L} fused to human CD28 hinge, transmembrane, and cytoplasmic domain, followed by a human CD3ζ cytoplasmic domain to create an anti-mutSOD1-CD28-CD3ζ CAR. In some embodiments, an scFv of a CAR comprised by a modified Treg may be constructed by linking heavy chain variable region and light chain variable region with a linker, such as, for example, a (G₄S)₃ linker. In some embodiments, the C-terminus of a V_{L} of a CAR of a modified Treg may be fused with human CD28 hinge, transmembrane, and cytoplasmic domain, and may be followed by a human CD3ζ cytoplasmic domain. Said CAR of said modified Treg may be an anti-mutSOD1 CAR. Said CARs may trigger both primary and costimulation signaling upon antigen binding, e.g., binding of mutSOD1. In some embodiments, a CAR costimulating domain may comprise, but not limited to one comprising, CD3ζ alone, 4-1BB, or CD28 or it may comprise CD28-CD3ζ DAP10-CD3ζ or CD44-CD3ζ. In some embodiments a truncated (non-signaling) human CD19 (tCD19) may also expressed in the same vector as said CARS, such as by using a 2A co-expression system, said tCD19 may serve as a way to track and purify transduced T cells. In some embodiments, modified Tregs targeted to ALS may enter the spinal cord when administered to a patient in need of treatment. In exemplary embodiments, modified Tregs may comprise markers such as, for example, VLA4, LFA-1, CCR6, or CXCR3. In some exemplary embodiments, modified CARs targeted to ALS, e.g., modified Tregs comprising one or more anti-mutSOD1 CARs, and optionally will retain a Treg phenotype and/or elicit at least some Treg effector functions when expressing one or more CARs and/or one or more NDMMs. In exemplary embodiments, modified Tregs may express IL-10 in response to an ALS protein and/or molecular marker of disease.

In some exemplary embodiments, modified Tregs according to the invention may secrete anti-inflammatory cytokines, which may result in inhibition of activated microglia and/or macrophages. Said secretion may occur as a result of stimulation of one or more CARs comprised by said modified Tregs by an ALS protein and/or disease associated marker, such as mutSOD1. In some embodiments, said cytokines may comprise IL-10, IL-4, or TGF-β. In some exemplary embodiments, modified Tregs may reduce and/or prevent production of neurotoxic free radicals and inflammatory cytokines by microglia. In some exemplary embodiments, modified Tregs may be used in methods of treating ALS, and said methods may result in one or more of the following as compared to a control treatment: less macrophage mediated motor neuron death; less IL-1β, TNF-α, nitric oxide and/or free radicals (superoxide anion); and greater amounts of IL-10, IL-4, and TGF-β.

In some exemplary embodiments, modified Tregs may comprise CARs targeted to the short isoform of C9orf72 (sC9orf72), which, like mutSOD1, may be expressed on or near motor neurons in ALS. In exemplary embodiments, modified Tregs may comprise CARs targeted to sC9orf72, wherein said CAR may comprise a human scFV against sC9orf72. In some specific exemplary embodiments, CARs targeted to sC9orf27 may comprise anti-sC9orf72 CARs using V_{H} and V_{L} sequences from a unique human αsC9orf72. In some embodiments, said V_{L} c-terminus of each αsC9orf72 scFv may be fused with human CD28 hinge, transmembrane, and cytoplasmic domain, followed by a human CD3ζ cytoplasmic domain to create an anti-sC9orf72 CAR that may be comprised by a modified Treg. In some embodiments, a non-signaling, truncated human CD19 (tCD19) can serve as a transduction marker on a vector comprising said CARs. In exemplary embodiments, modified Tregs targeted to sC9orf72 may inhibit microglia mediated motor neuron degeneration; decrease IL-1β, TNF-α, nitric oxide; and/or increase IL-10. IL-4, and TGF-β, such as when administered to a patient in need of treatment.

In some exemplary embodiments, modified Tregs may comprise modified Tregs targeted to ALS, such as modified Tregs comprising anti-mutSOD1 CARs, and said modified Tregs may enter the spinal cord parenchyma, recognize accumulated spinal mutSOD1 protein, and react by producing anti-inflammatory mediators. Said modified Tregs may decrease expression of inflammatory mediators (e.g. CCL2, CCL3, CCL4, TNF-α, IL1β, NOX2) and increase expression of anti-inflammatory mediators (e.g. IL-10, IL-4, and TGF-β) when administered to a patient in need of treatment. Furthermore, modified Tregs targeted to ALS, such as modified Tregs comprising anti-mutSOD1 CARs, may inhibit persistent and/or neurotoxic inflammation around motor neurons when used in methods of treatment of ALS.

Furthermore, in other exemplary embodiments, modified Tregs may be targeted to proteins and/or molecular markers associated with Alzheimer's disease. In some exemplary embodiments, said modified Tregs may comprise one or more CARs targeted to said proteins and/or markers. In some embodiments, said protein and/or marker may comprise amyloid-beta (Aβ), in particular oligomeric Aβ, and/or intraneuronal tangles of twisted tau protein fibers. In some embodiments, a CAR targeted to amyloid beta may comprise a sequence of DG01 (SEQ ID NO: 1), and/or a construct comprising at least 90%, at least 95%, at least 98%, or at least 99% sequence identity to the aforementioned construct. In some embodiments, a CAR targeted to amyloid beta may comprise a sequence of DG02 (SEQ ID NO: 2), and/or a construct comprising at least 90%, at least 95%, at least 98%, or at least 99% sequence identity to the aforementioned construct. In some embodiments, a CAR targeted to amyloid beta may comprise a sequence of DG03 (SEQ ID NO: 3), and/or a construct comprising at least 90%, at least 95%, at least 98%, or at least 99% sequence identity to the aforementioned construct. In some embodiments, a CAR targeted to amyloid beta may comprise a sequence of DG04 (SEQ ID NO: 4), and/or a construct comprising at least 90%, at least 95%, at least 98%, or at least 99% sequence identity to the aforementioned construct. In some embodiments, a modified Treg may comprise DG01-CD28-CD3ζ (SEQ ID NO: 20), DG02-CD28-CD3ζ (SEQ ID NO: 21), DG03-CD28-CD3ζ (SEQ ID NO: 22), DG04-CD28-CD3ζ (SEQ ID NO: 23), and/or a construct comprising at least 90%, at least 95%, at least 98%, or at least 99% sequence identity to any one or more of the aforementioned constructs, wherein each of the constructs is targeted to amyloid-beta. In some embodiments, a modified Treg may comprise DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); and/or DG03-CD28tm (also referred to as DG03-28tm) (SEQ ID NO: 50), and/or a construct comprising at least 90%, at least 95%, at least 98%, or at least 99% sequence identity to any one or more of the aforementioned constructs, wherein each of the constructs is targeted to amyloid-beta.

In exemplary embodiments, CARs comprised by modified Tregs may be targeted to Aβ peptides, and may comprise anti-Aβ. CARs using single chain variable fragment (scFv) sequences from antibodies, e.g., human and/or humanized antibodies, with different binding specificities to Aβ, e.g., oligomeric Aβ. In exemplary embodiments, said scFvs may be fused to human CD28 hinge, transmembrane, and cytoplasmic domains, followed by a human CD3ζ cytoplasmic domain. Said CARs may trigger both primary (CD3ζ) and co-stimulatory (CD28) signaling upon antigen binding and cross-linking. In some embodiments, a truncated (non-signaling) CD19 (tCD19) may also expressed in the same vector comprising said CARs, such as by using a T2A co-expression system, and it may serve as a means to track and purify transduced T cells. Modified Tregs comprising anti-Aβ. CARs may suppress proliferation of CD3-activated allogeneic CD8⁺ T cells in some embodiments. Furthermore, in some embodiments, when activated with oligomeric Aβ, modified Tregs comprising anti-Aβ. CARs may produce anti-inflammatory cytokines, e.g., IL-10. Furthermore, said modified Tregs may inhibit production of pro-inflammatory mediators and may enhance phagocytic capacity of activated microglia or macrophages, such as by secreting IL-10, TGF-β, and IL-4 anti-inflammatory cytokines for example. In some exemplary embodiments, modified Tregs according to the invention which express a CAR specific for oligomeric Aβ may have targeted anti-inflammatory activity and neuroprotective effects in regions where oligomeric Aβ may accumulate. In some exemplary embodiments, e.g., modified Tregs which express a CAR specific for oligomeric Aβ may migrate to the hippocampus, wherein oligomeric Aβ may accumulate.

Moreover, in some exemplary embodiments, modified Tregs comprising anti-CARs may traffic and accumulate to brain regions of Aβ deposits and neuroinflammation, wherein such regions may include sites of Aβ deposits in the hippocampus and frontal cortex. In some embodiments, modified Tregs comprising CARs targeting Alzheimer's may accumulate in said brain regions and may lead to increased expression of human anti-inflammatory cytokines IL-10, TGF-β, and IL4 in said regions. These anti-inflammatory cytokines may lead to decreased expression of pro-inflammatory mediators and the numbers of microglia. In some exemplary embodiments, modified Tregs comprising CARs targeting Alzheimer's disease may improve memory function in a patient treated with said modified Tregs.

Specific features of and/or that may be comprised by modified Tregs and/or specific features that may be comprised by targets of modified Tregs are discussed in greater detail below.

CARs

In exemplary embodiments, modified Tregs may comprise one or more CARs targeted to a neurodegenerative disease or condition, as discussed above and below. In some exemplary embodiments, one or more CARs may comprise an AB domain that binds to a target molecule which is associated with a neurodegenerative disease or condition. AB domains are discussed further below.

Antigen Binding (AB) Domain

The AB domain may be derived from a polypeptide that binds to a target molecule. In some embodiments, the polypeptide may be a receptor or a portion of a receptor that binds to a target molecule. In another embodiment, the AB domain may be derived from a ligand that binds to the target molecule.

In another embodiment, the AB domain may be derived from an antibody (Ab) or antigen-binding fragment thereof that binds to a target molecule. Examples of an Ab or antigen-binding fragment thereof include, but are not limited to, a monoclonal Ab, a monospecific Ab, a polyspecific Ab, a humanized Ab, a tetrameric Ab, a tetravalent Ab, a multispecific Ab, a single chain Ab, a domain-specific Ab, a single-domain Ab (dAb), a domain-deleted Ab, an scFc fusion protein, a chimeric Ab, a synthetic Ab, a recombinant Ab, a hybrid Ab, a mutated Ab, CDR-grafted Ab, an Ab fragment comprising a fragment antigen-binding (Fab), an F(ab′)₂, an Fab′ fragment, an variable fragment (Fv), a single-chain antibody fragment, a single-chain Fv (scFv) fragment, an Fd fragment, a dAb fragment, a diabody, a nanobody, a bivalent nanobody, a shark variable IgNAR domain, a VHH Ab, a camelid Ab, and a minibody. In a particular embodiment, the AB domain comprises a single-chain antibody fragments comprising a variable heavy chain region and/or a variable light chain region, such as scFv. In another particular embodiment, the AB domain comprises a nanobody.

Single-domain Abs are Ab fragments comprising all or a portion of the heavy chain variable domain or all or a portion of the light chain variable domain of an antibody. In certain embodiments, a single-domain antibody is a human single-domain antibody

Antibody fragments can be made by various techniques, including but not limited to proteolytic digestion of an intact Ab as well as production by recombinant host cells. In some embodiments, the antibodies are recombinantly produced fragments, such as fragments comprising arrangements that do not naturally occur, such as those with two or more Ab regions or chains joined by synthetic linkers, such as peptide linkers, and/or that may not be produced by enzyme digestion of a naturally occurring intact Ab. In some aspects, the Ab fragments are scFvs. In some aspects, the Ab fragments are nanobodies.

In some aspects, the AB domain may be derived from an Ab or an antigen-binding fragment thereof that has one or more specified functional features, such as binding properties, including binding to particular epitopes, such as epitopes that are similar to or overlap with those of other Abs.

In some embodiments, the AB comprises an scFv comprising CDR sequences of an Ab specific to the target molecule. CDRs may be determined using conventional methods. The precise amino acid sequence boundaries of a given CDR or FR can be readily determined using any of a number of well-known schemes, including those described by Kabat et al. (1991), “Sequences of Proteins of Immunological Interest,” 5^{th} Ed. Public Health Service, National Institutes of Health, Bethesda, Md. (“Kabat” numbering scheme), Al-Lazikani et al., “(1997) J Mol. Biol. 273, 927-948 (“Chothia” numbering scheme), MacCallum et al., J Mol. Biol. 262:732-745 (1996), “Antibody-antigen interactions: Contact analysis and binding site topography,” J. Mol. Biol. 262, 732-745 (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme).

In some embodiments, the sequence comprising the AB domain further comprises a leader sequence or signal sequence. In some embodiments where the AB domain comprises an scFv, the leader sequence may be positioned at the amino terminus of the scFv. In some embodiments where the heavy chain variable region is N-terminal, the leader sequence may be positioned at the amino terminus of the heavy chain variable region. In some embodiments where the light chain variable region is N-terminal, the leader sequence may be positioned at the amino terminus of the light chain variable region. The leader sequence may comprise any suitable leader sequence. In some embodiments of the invention, the amino acid sequence of the leader sequence may comprise a sequence as set forth in SEQ ID NO: 31, or a sequence encoded by the nucleic acid sequence as set forth in SEQ ID NO: 231. In the mature form of the isolated cells of the invention, the leader sequence may not be present.

Hinge

In some embodiments, a modified Treg may comprise one or more CARs, and said CARs may comprise a hinge sequence between the AB domain and a TM domain. One of the ordinary skill in the art will appreciate that a hinge sequence is a short sequence of amino acids that facilitates flexibility (see, e.g. Woof et al., Nat. Rev. Immunol., 4(2): 89-99 (2004)). The hinge sequence can be any suitable sequence derived or obtained from any suitable molecule. In some embodiments, the length of the hinge sequence may be optimized based on the desired length of the extracellular portion of a CAR, which may be based on the location of the epitope within the target molecule. For example, if the epitope is in the membrane proximal region within the target molecule, longer hinges may be optimal.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In some embodiments, the hinge includes at least a portion of an IgG1, an IgG2, an IgG3, an IgG4, an IgE, an IgM, or an IgA immunoglobulin Fc region that falls within its CH2 and CH3 domains. In some embodiments, the hinge may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the hinge to an Fc receptor (FcR). In some aspects, the modified immunoglobulin Fc region may be designed with one or more mutations which result in one ore more amino acid substitutions, modifications, or deletions that cause impaired binding of the hinge to one or more FcR including, but not limited to, FcγR1, FcγR2A, FcγR2B1, Fcγ2B2, Fcγ3A, Fcγ3B, FcεRI, FcεR2, FcαRI, Fcα/μR, or FcRn.

In some aspects, a portion of the immunoglobulin constant region serves as a hinge between the AB domain, for example scFv or nanobody, and the TM domain. The hinge can be of a length that provides for increased responsiveness of the CAR-expressing cell following antigen binding, as compared to in the absence of the hinge. In some examples, the hinge is at or about 12 amino acids in length or is no more than 12 amino acids in length. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. Exemplary hinges include a CD28 hinge, IgG4 hinge alone, IgG4 hinge linked to CH2 and CH3 domains, or IgG4 hinge linked to the CH3 domain. Exemplary hinges include, but are not limited to, those described in Hudecek et al. (2013) Clin. Cancer Res., 19:3153, international patent application publication number WO2014031687, U.S. Pat. No. 8,822,647 or published App. No. US2014/0271635.

In some embodiments, the hinge sequence is derived from CD8a molecule, a DAP10 molecule, a CD8a molecule, or a CD28 molecule. In a preferred embodiment, the hinge sequence is derived from CD28. In one embodiment, the hinge comprises the amino acid sequence of human CD28 hinge (SEQ ID NO: 32) or the sequence encoded by SEQ ID NO: 232. In some embodiments, the hinge has an amino acid sequence at least 85%, 86%, 87%, 88%, 89%, 90%, 91%, 92%, 93%, 94%, 95%, 96%, 97%, 98%, 99%, or 100% identical to SEQ ID NO: 32. In some embodiments, the hinge comprises the amino acid sequence of mouse CD28 hinge (SEQ ID NO: 33) or the sequence encoded by SEQ ID NO: 233. In some embodiments, the hinge has an amino acid sequence at least 85%, 86%, 87%, 88%, 89%, 90%, 91%, 92%, 93%, 94%, 95%, 96%, 97%, 98%, 99%, or 100% identical to SEQ ID NO: 33. In one embodiment, the hinge comprises the amino acid sequence of human CD8A hinge (SEQ ID NO: 34) or the sequence encoded by SEQ ID NO: 234. In some embodiments, the hinge has an amino acid sequence at least 85%, 86%, 87%, 88%, 89%, 90%, 91%, 92%, 93%, 94%, 95%, 96%, 97%, 98%, 99%, or 100% identical to SEQ ID NO: 34. In one embodiment, the hinge comprises the amino acid sequence of human DAP10 hinge (SEQ ID NO: 35) or the sequence encoded by SEQ ID NO: 235. In some embodiments, the hinge has an amino acid sequence at least 85%, 86%, 87%, 88%, 89%, 90%, 91%, 92%, 93%, 94%, 95%, 96%, 97%, 98%, 99%, or 100% identical to SEQ ID NO: 35.

Transmembrane (TM) Domain

In some embodiments, a modified Treg may comprise one or more CARs, and said CARs may comprise a TM domain. With respect to the TM domain, a CAR can be designed to comprise a TM domain that is fused to the AB domain of the CAR. A hinge sequence may be inserted between the AB domain and the TM domain. In some embodiments, a TM domain that naturally is associated with one of the domains in the CAR is used. In some instances, the TM domain can be selected or modified by amino acid substitution to avoid binding of such domains to the transmembrane domains of the same or different surface membrane proteins to minimize interactions with other members of the receptor complex.

A TM domain may be derived either from a natural or from a synthetic source. Where the source is natural, the domain may be derived from any membrane-bound or transmembrane protein. Typically, the TM domain denotes a single transmembrane α helix of a transmembrane protein, also known as an integral protein. TM domains of particular use in this invention may be derived from (i.e. comprise at least the transmembrane region(s) of) CD28, CD3ε, CD4, CD5, CD8, CD9, CD16, CD22, CD33, CD37, CD45, CD64, CD80, CD86, CD134, CD137, CD154, Dap10, CD44, CTLA-4, TCRα, TCRβ, or CD3 zeta (SEQ ID NO: 16, which may be encoded by SEQ ID NO: 216) and/or TM domains containing functional variants thereof such as those retaining a substantial portion of the structural, e.g., transmembrane, properties thereof.

Alternatively the TM domain may be synthetic, in which case the TM domain will comprise predominantly hydrophobic residues such as leucine and valine. Preferably a triplet of phenylalanine, tryptophan and valine will be found at each end of a synthetic TM domain. A TM domain of the invention is thermodynamically stable in a membrane. It may be a single a helix, a transmembrane 13 barrel, a (3-helix of gramicidin A, or any other structure. Transmembrane helices are usually about 20 amino acids in length.

In some preferred embodiments, the TM domain in a CAR may be derived from the TM region of CD28. In one embodiment, the TM domain comprises the amino acid sequence of human CD28 TM (SEQ ID NO: 36) or the sequence encoded by SEQ ID NO: 236. In some embodiments, the TM domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 36. In one embodiment, the TM domain comprises the amino acid sequence of mouse CD28 TM (SEQ ID NO: 37) or the sequence encoded by SEQ ID NO: 237. In some embodiments, the TM domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 37. In one embodiment, the TM domain comprises the amino acid sequence of human CD8A TM (SEQ ID NO: 38) or the sequence encoded by SEQ ID NO: 238. In some embodiments, the TM domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 38. In one embodiment, the TM domain comprises the amino acid sequence of human DAP10 TM (SEQ ID NO: 39) or the sequence encoded by SEQ ID NO: 239. In some embodiments, the TM domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 39.

Optionally, a short oligo- or polypeptide spacer, preferably between 2 and 10 amino acids in length may form the linkage between the TM domain and the ICS domain(s) of a CAR. A glycine-serine doublet may provide a suitable spacer.

Intracellular Signaling (ICS) Domain and Costimulatory (CS) Domain

In some embodiments, a modified Treg may comprise one or more CARs, and said CARs may comprise a ICS and/or CS domain. The ICS domain or otherwise the cytoplasmic domain of a CAR may trigger or elicit activation of at least one of the normal functions of the cell in which the CAR has been placed, for example, the secretion of cytokines. Thus, the term “intracellular signaling domain” or “ICS domain” refers to the portion of a protein which transduces a functional signal and directs the cell to perform a specialized function. While usually the entire ICS domain may be employed, in many cases it is not necessary to use the entire chain. To the extent that a truncated portion of the intracellular signaling domain is used, such truncated portion may be used in place of the intact chain as long as it transduces the function signal. The term “intracellular signaling domain” or “ICS domain” is thus meant to include any truncated portion of the ICS domain sufficient to transduce a function signal.

Signals generated through one ICS domain alone may be insufficient for full activation of a cell, and a secondary or costimulatory signal may also be required. In such cases, a costimulatory domain (CS domain) may be included in the cytoplasmic portion of a CAR. A CS domain is a domain that transduces such a secondary or costimulatory signal. Optionally, a CAR may comprise two or more CS domains. The CS domain(s) may be placed upstream of the ICS domain or downstream of the ICS domain.

For example, T cell activation can be said to be mediated by two distinct classes of cytoplasmic signaling sequence: those that initiate antigen-dependent primary activation through the T cell receptor (TCR) (primary cytoplasmic signaling sequences) and those that act in an antigen-independent manner to provide a secondary or costimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Such a cytoplasmic signaling sequence may be contained in the ICS or the CS domain of a CAR.

Examples of ITAM-containing primary cytoplasmic signaling sequences that are of particular use may include those derived from an ICS domain of a lymphocyte receptor chain, a TCR/CD3 complex protein, an Fc receptor subunit, an IL-2 receptor subunit, CD3ζFcRγ, FcRβ, CD3γ, CD3δ, CD3ε, CD5, CD22, CD66d, CD79a, CD79b, CD278 (ICOS), FcεRI, DAP10, and DAP12.

In some embodiments, an ICS domain in a CAR may comprise a cytoplasmic signaling sequence derived from CD3 zeta. In some embodiments, the ICS domain comprises the amino acid sequence of SEQ ID NO: 16), or the sequence encoded by SEQ ID NO: 216. In some embodiments, the ICS domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 16.

Various CS domains have been reported to confer differing properties (Gacerez et al. J. Cell. Physiol. 231: 2590-2598, 2016). For example, the 4-1BB CS domain has been reported to exhibit enhanced persistence in some in vivo xenograph models (Milone et al. Mol Ther 2009; 17:1453-1464; Song et al. Cancer Res 2011; 71:4617-4627). Additionally, these different CS domains produce different cytokine profiles, which in turn, may produce different effects on target cell-mediated cytotoxicity and the disease microenvironment.

Examples of co-stimulatory molecules include MHC class I molecules, TNF receptor proteins, immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CD5, CRTAM, DAP10, GADS, GITR, HVEM (LIGHTR), IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like.

The ICS domain and the CS domain(s) of a CAR may be linked to each other in a random or specified order. Optionally, a short oligo- or polypeptide linker, preferably between 2 and 10 amino acids in length may form the linkage. A glycine-serine doublet provides a particularly suitable linker.

Further Modifications

In some embodiments, a modified Treg may comprise one or more CARs, and said CARs may comprise further modifications. In some embodiments, one or more CARs, nucleotide sequences encoding the same, vectors encoding the same, and cells comprising nucleotide sequences encoding said CARs may be further modified, engineered, optimized, or appended in order to provide or select for various features. These features may include, but are not limited to, efficacy, persistence, target specificity, reduced immunogenicity, multi-targeting, enhanced immune response, expansion, growth, reduced off-target effects, reduced subject toxicity, detection, selection, targeting, and the like. For example, the cells may be engineered to express another CAR, or to have a suicide mechanism, and may be modified to remove or modify expression of an endogenous receptor or molecule such as a TCR and/or MHC molecule.

In some embodiments, a vector or nucleic acid sequence encoding a CAR further encodes other genes. The vector or nucleic acid sequence may be constructed to allow for the co-expression of multiple genes using a multitude of techniques including co-transfection of two or more plasmids, the use of multiple or bidirectional promoters, or the creation of bicistronic or multicistronic vectors. The construction of multicistronic vectors may include the encoding of IRES elements or 2A peptides, such as T2A, P2A (which amino acid sequence may comprise SEQ ID NO: 15, which may be encoded by SEQ ID NO: 215), E2A, or F2A (for example, see Kim, J. H., et al., “High cleavage efficiency of a 2A peptide derived from porcine teschovirus-1 in human cell lines, zebrafish and mice”, PLoS One. 2011; 6(4)). In some embodiments, the nucleic acid sequence or vector encoding a CAR further encodes tCD19 with the use of a T2A ribosomal skip sequence. In one embodiment, the T2A ribosomal skip sequence comprises the nucleic acid sequence as set forth in SEQ ID NO: 214. In one embodiment, the T2A ribosomal skip sequence encodes the amino acid sequence of SEQ ID NO: 14.

CARs comprised by modified Tregs according to the invention optionally may be further modified to improve efficacy against cells expressing the target molecule. In some embodiments, the improved efficacy may be measured by a decrease in microglial cell activation, a decrease in inflammatory response, and/or a decrease in neuronal death. In some embodiments, modified Tregs comprising one or more CARs may further comprise more than a CAR. Additional CARs may or may not be specific for the target molecule of the first CAR. In some embodiments, the one or more additional CARs may act as inhibitory or activating CARs. In some aspects, a CAR of some embodiments is the stimulatory or activating CAR; in other aspects, it is the costimulatory CAR. In some embodiments, modified Tregs may further include inhibitory CARs (iCARs, see Fedorov et al., Sci. Transl. Medicine, 2013 December; 5(215): 215ra172), such as a CAR recognizing an antigen other than the target molecule of the first CAR, whereby an activating signal delivered through the first CAR is modified or altered by binding of the inhibitory CAR to its ligand, e.g., to reduce off-target effects.

Vectors or Constructs

The present disclosure also provides vectors or constructs such as plasmids or retroviral constructs in which a DNA may be inserted such as one encoding a CAR. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs may typically achieved by operably linking a nucleic acid encoding a CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, γ-retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Various promoter sequences may be used, including, but not limited to the immediate early cytomegalovirus (CMV) promoter, the CMV-actin-globin hybrid (CAG) promotor, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Inducible promoters are also contemplated for use. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionein promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

In some embodiments, the selectable marker gene comprises a nucleic acid sequence encoding truncated CD19 (trCD19).

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Transduction of Tregs

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means

Treg Cells

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and disease sites. In some embodiments, any number of T cell lines available and known to those skilled in the art, may be used. In some embodiments, cells can be derived from a healthy donor or from a patient diagnosed with a neurodegenerative disease or condition.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom

In some aspects, the sample from which the cells are derived or isolated is blood or a blood-derived sample, or is or is derived from an apheresis product. Exemplary samples include whole blood, peripheral blood mononuclear cells (PBMCs), leukocytes, bone marrow, thymus, tissue biopsy, neural tissue, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

Cell Purification

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺/Mg⁺⁺ free PBS. In certain embodiments, components of a blood cell sample are removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, the surface maker is trCD19. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population

In some embodiments, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, Tregs may be isolated, expanded, and transduced as follows: cells may be isolated from human PBMCs via a two-step negative and positive selection protocol. First, CD4⁺ cells are isolated using negative selection, followed by a positive selection of CD25hi⁺ cells to isolate CD4⁺CD25hi⁺ Treg cells. The isolated CD4⁺CD25hi⁺ Tregs may be activated with anti-CD3, anti-CD28, anti-CD2 multimers or anti-CD3, anti-CD28 multimers (STEMCELL ImmunoCult) with human IL-2 (300 U/ml to 500 U/ml) over two weeks in culture in Treg growth medium. On day 9, Treg cells can be cryopreserved for use at a later date, so we can test the same donor and preparation on multiple occasions to assess assay variability.

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 disclose other examples of such particles.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the magnetically responsive particles are coated in primary antibodies or other binding partners, secondary antibodies, lectins, enzymes, or streptavidin. In some embodiments, the magnetic particles are attached to cells via a coating of primary antibodies specific for one or more markers. In some embodiments, the cells, rather than the beads, are labeled with a primary antibody or binding partner, and then cell-type specific secondary antibody- or other binding partner (e.g., streptavidin)-coated magnetic particles, are added. In some embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In some embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the methods include density-based cell separation methods, such as the preparation of white blood cells from peripheral blood by lysing the red blood cells and centrifugation through a Percoll or Ficoll gradient.

In any of the aforementioned separation steps, the separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

Cell Preparation and Expansion

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The cells discussed herein can be activated and expanded, either prior to or after genetic modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Tregs can be expanded in vitro or in vivo. In some embodiments, the isolated cells of the invention can be expanded by co-culturing with tissue or cells. The cells can also be expanded in vivo, for example in the subject's blood after administrating the cell into the subject.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° Celsius at a rate of 1 degree per minute and stored in the vapor phase of a liquid nitrogen storage tank.

Methods Using Modified Tregs According to the Invention

In exemplary embodiments, treatment of neurodegenerative diseases, conditions, and/or disorders may comprise administration of an effective amount of one or more modified Tregs as disclosed herein. In exemplary embodiments, treatment of a neurodegenerative disease by administering one or more types of modified Tregs according to the invention (e.g., wherein such Tregs may include those which express different CARS and/or NDDMs) may result in a decrease in inflammation, modulation of microglial cell activity, and/or decreased neuronal damage at the sites where the protein and/or molecular marker is expressed (i.e. diseased tissue). In exemplary embodiments, modified Tregs according to the invention may be administered to a patient in need of treatment, wherein said modified Tregs may be administered by intravenous injection, subcutaneous injection, intracavitary injection, intraventricular injection, intracranial injection, or intrathecally injection. Exemplary treatment methods generally comprise the administration of an effective amount of one or more modified Tregs, wherein such treatment comprising said modified Tregs may modulate local inflammation or neuronal survival. Said modulation may occur, in some embodiments, by expression of specific molecules, e.g., NDMMs, e.g., anti-oxidants, e.g., neuronal growth and/or survival factors.

In exemplary embodiments, modified Tregs according to the invention, e.g., modified Tregs comprising one or more CARs, may be used in a method of treating Parkinson's disease. Said modified Tregs targeted to Parkinson's disease may comprise one or more CARs, wherein said one or more CARs may target α-synuclein fibrils. In some embodiments, a CAR targeted to alpha-synuclein may comprise a sequence of DG08 (SEQ ID NO: 8). In some embodiments, a CAR targeted to alpha-synuclein may comprise a sequence of DG09 (SEQ ID NO: 9). In some embodiments, a CAR targeted to alpha-synuclein may comprise a sequence of DG10 (SEQ ID NO: 10). In some embodiments, a CAR targeted to alpha-synuclein may comprise a sequence of DG11 (SEQ ID NO: 11). In some embodiments, a modified Treg may comprise DG08-CD28-CD3ζ (SEQ ID NO: 27), DG09-CD28-CD3ζ (SEQ ID NO: 28), DG10-CD28-CD3ζ (SEQ ID NO: 29), and/or DG11-CD28-CD3ζ (SEQ ID NO: 30), wherein each construct is targeted to alpha-synuclein. In further exemplary embodiments, modified Tregs, such as modified Tregs comprising one or more CARs, may be used to treat Parkinson's disease and may target one or more neurotoxic inflammatory mediators, e.g., neurotoxic inflammatory mediations produced by activated microglia. Said modified Tregs may decrease and/or inhibit microglia activation. In exemplary embodiments, modified Tregs targeting Parkinson's disease may comprise targeted anti-inflammatory and neuroprotective therapeutic activity at the disease site of dopamine neuron degeneration in PD. In some embodiments, modified Tregs targeting Parkinson's may mediate their function only at the site where α-synuclein fibrils are present.

Furthermore, modified Tregs targeting Parkinson's disease may be used in methods of treating Parkinson's disease, wherein said modified Tregs may comprise one or more CARs and/or one or more NDMMs targeted to Parkinson's disease, and further wherein said one or more CARs comprise single chain variable fragments such as V_{H} and V_{L} amino acid sequences of human and mouse monoclonal antibodies against human α-synuclein fibrils (such as, for example, amino acid sequences derived from clones NI 202.3G12, NI 202.12F4, NI 202.21D11, and mAb49/G). In exemplary embodiments, modified Tregs targeting Parkinson's disease may comprise said scFV and further comprise a construct comprising CD28-CD3ζ CAR, i.e., scFv-CD28-CD3ζ CAR, wherein said scFv is specific for human α-synuclein fibrils. Constructs comprising said scFv may comprise V_{H}+V_{L} and V_{L}+V_{H} arrangements.

In exemplary embodiments, modified Tregs according to the invention, e.g., modified Tregs comprising one or more CARs, may be used in a method of treating ALS. Said modified Tregs targeted to Parkinson's disease may comprise one or more CARs and/or one or more NDMMs targeted to ALS. Said modified Tregs may target mutSOD1, e.g., said modified Tregs may comprise one or more CARs targeted to mutSOD1. In some embodiments, a CAR targeted to mutSOD1 may comprise a sequence of DG05 (SEQ ID NO: 5). In some embodiments, a CAR targeted to mutSOD1 may comprise a sequence of DG06 (SEQ ID NO: 6). In some embodiments, a CAR targeted to mutSOD1 may comprise a sequence of DG07 (SEQ ID NO: 7). In some embodiments, a modified Treg may comprise DG05-CD28-CD3ζ (SEQ ID NO: 24), DG06-CD28-CD3ζ (SEQ ID NO: 25), and/or DG07-CD28-CD3ζ (SEQ ID NO: 26), wherein each construct is targeted to mutSOD1. In some embodiments, a modified Treg may comprise DG05-CD28-CD3ζ (also referred to as DG05-28-3ζ) (SEQ ID NO: 24); DG05-CD28tm-DAP10-CD3ζ (also referred to as DG05-28tm-10-(SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); and/or DG05-CD28tm (also referred to as DG05-28tm) (SEQ ID NO: 44), wherein each of the constructs is targeted to mutSOD1. In some embodiments, modified Tregs may become activated at sites of mutSOD1-producing motor neurons and/or sites of inflammation thereby resulting in reduced inflammation at the disease site in methods comprising treatment of ALS comprising use of modified Tregs.

In some embodiments, one or more modified Tregs may comprise any one or more of DG05-28-3ζ; DG05-28tm-10-3ζ; DG05-28tm-44-3ζ; DG05-28tm-3ζ; and/or DG05-28, and said modified Tregs may produce IL-10 in response to mSOD1 antigen. In some embodiments, one or more modified Tregs may comprise any one or more of DG05-28-3ζ; DG05-28tm-10-3ζ; DG05-28tm-44-3ζ; DG05-28tm-3ζ; and/or DG05-28, and said modified Tregs may produce increased levels of IL-10 in response to mSOD1 antigen as compared to modified Tregs not exposed to mSOD1. In some embodiments, one or more modified Tregs may comprise DG05-CD28-CD3ζ, and said modified Tregs may comprise increased expression of cell surface markers such as GITR, PD-1, and/or CTLA-4 in response to mSOD1 as compared to modified Tregs not exposed to mSOD1. In some embodiments, one or more modified Tregs may comprise DG05-CD28-CD3ζ, and said modified Tregs may produce IL-10 in response to mSOD1 antigen, e.g., mSOD1 antigen that may be found in spinal cord tissue, as compared to modified Tregs not exposed to mSOD1 antigen. In some embodiments, one or more modified Tregs may comprise DG05-CD28-CD3ζ, and said modified Tregs, when stimulated with mSOD1 antigen and/or anti-CD3 antibody, may inhibit superoxide generation as compared to modified Tregs that were not stimulated with mSOD1 antigen or anti-CD3 antibody. In some embodiments, one or more modified Tregs may comprise DG05-CD28-CD3ζ, and said modified Tregs, when stimulated with mSOD1 antigen, may inhibit TNF-α production as compared to modified Tregs not stimulated with mSOD1 antigen.

In some embodiments, modified Tregs targeting ALS may be used in methods of treating ALS and may comprise CARs targeted to mutSOD1, and in some exemplary embodiments an scFv of one or more of said CARs may be expressed extracellularly with the C-terminus of the V_{L} fused to human CD28 hinge, transmembrane, and cytoplasmic domain, followed by a human CD3ζ cytoplasmic domain to create an anti-mutSOD1-CD28-CD3ζ CAR. In some embodiments, an scFv of a CAR comprised by a modified Treg according to the invention targeting ALS may be constructed by linking heavy chain variable region and light chain variable region with a linker, such as, for example, a (G₄S)₃ linker. In some embodiments, the C-terminus of a V_{L} of a CAR of a modified Treg targeting ALS may be fused with human CD28 hinge, transmembrane, and cytoplasmic domain, and may be followed by a human CD3ζ cytoplasmic domain. Said CAR of said modified Treg targeting ALS may e.g., be an anti-mutSOD1 CAR. Said CARs may trigger both primary and costimulation signaling upon antigen binding, e.g., binding of mutSOD1. In some embodiments, a CAR expressed by the Treg may comprise a costimulating domain including but not limited to comprising, CD3ζ alone, 4-1BB, or CD28. In some embodiments a truncated (non-signaling) human CD19 (tCD19) may also expressed in the same vector as said CARs, such as by using a 2A co-expression system, said tCD19 may serve as a way to track and purify transduced T cells. In some embodiments, methods of treating ALS may comprise use of modified Tregs targeted to ALS that may enter the spinal cord when administered to a patient in need of treatment. In some exemplary embodiments, modified Tregs targeted to ALS may comprise markers such as, for example, VLA4, LFA-1, CCR6, CXCR3 or other proteins which promote neuron survival and/or functionality and/or prolong T cell function. In exemplary embodiments, modified CARs targeted to ALS, e.g., modified Tregs comprising one or more anti-mutSOD1 CARs, may preserve a Treg phenotype when expressing one or more CARs and/or one or more NDMMs. In exemplary embodiments, modified Tregs may express IL-10 in response to an ALS protein and/or molecular marker of disease when used in methods of treating ALS.

Furthermore, methods of treating ALS according to the invention may comprise use of modified Tregs which secrete anti-inflammatory cytokines, thereby resulting in an inhibition of activated microglia and/or macrophages. Said secretion may occur as a result of stimulation of one or more CARs expressed by said modified Tregs for an ALS protein and/or disease associated marker, such as mutSOD1. In some embodiments, said cytokines may comprise IL-10, IL-4, TGF-β. In exemplary embodiments, modified Tregs may reduce and/or prevent production of neurotoxic free radicals and inflammatory cytokines by microglia when used in methods of treating ALS. In some exemplary embodiments, modified Tregs may be used in methods of treating ALS, and said methods may result in one or more of the following as compared to a control treatment: less macrophage mediated motor neuron death; less IL-1β, TNF-α, and nitric oxide; and greater amounts of IL-10, IL-4, and TGF-β. In some embodiments, a method of treating ALS may comprise use of modified Tregs targeting C9orf72 (sC9orf72), and may achieve similar results and may be used in a similar manner as to modified Tregs targeting mutSOD1.

Moreover, some methods of treating ALS according to the invention may comprise use of modified Tregs, wherein said modified Tregs may comprise anti-mutSOD1 CARs, and said modified Tregs may enter the spinal cord parenchyma, recognize accumulated spinal mutSOD1 protein, and react by producing anti-inflammatory mediators. Said modified Tregs may decrease expression of inflammatory mediators (e.g. CCL2, CCL3, CCL4, TNF-α, IL1β, NOX2) and increase expression of anti-inflammatory mediators (e.g. IL-10, IL-4, and TGF-β) when administered to a patient in need of treatment. Furthermore, modified Tregs targeted to ALS, such as modified Tregs comprising anti-mutSOD1 CARs, may inhibit persistent and/or neurotoxic inflammation around motor neurons when used in methods of treating ALS.

In some exemplary embodiments, modified Tregs, e.g., modified Tregs comprising one or more CARs, may be used in methods of treating Alzheimer's disease. Said modified Tregs may be targeted to proteins and/or molecular markers associated with Alzheimer's disease. In exemplary embodiments, said modified Tregs may comprise one or more CARs targeted to said proteins and/or markers. In some embodiments, said protein and/or marker may comprise amyloid-beta (Aβ), in particular oligomeric Aβ, and/or intraneuronal tangles of twisted tau protein fibers. In some embodiments, a CAR targeted to amyloid beta may comprise a sequence of DG01 (SEQ ID NO: 1). In some embodiments, a CAR targeted to amyloid beta may comprise a sequence of DG02 (SEQ ID NO: 2). In some embodiments, a CAR targeted to amyloid beta may comprise a sequence of DG03 (SEQ ID NO: 3). In some embodiments, a CAR targeted to amyloid beta may comprise a sequence of DG04 (SEQ ID NO: 4). In some embodiments, a modified Treg may comprise DG01-CD28-CD3ζ (SEQ ID NO: 20), DG02-CD28-CD3ζ (SEQ ID NO: 21), DG03-CD28-CD3ζ (SEQ ID NO: 22), and/or DG04-CD28-CD3ζ (SEQ ID NO: 23), wherein each construct is targeted to amyloid-beta. In some embodiments, a modified Treg may comprise DG03-CD28-CD3 (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); and/or DG03-CD28tm (also referred to as DG03-28tm) (SEQ ID NO: 50), wherein each of the constructs is targeted to amyloid-beta. In exemplary embodiments, CARs comprised by modified Tregs may be targeted to Aβ peptides, and may comprise anti-Aβ CARs using single chain variable fragment (scFv) sequences from antibodies, e.g., human and/or humanized antibodies, with different binding specificities to Aβ, e.g., oligomeric Aβ. In some exemplary embodiments, said scFvs may fused to human CD28 hinge, transmembrane, and cytoplasmic domains, followed by a human CD3ζ cytoplasmic domain. Said CARs may trigger both primary (CD3ζ) and co-stimulatory (CD28) signaling upon antigen binding and cross-linking. In some embodiments, a truncated (non-signaling) CD19 (tCD19) may also expressed in the same vector comprising said CARs, such as by using a T2A co-expression system, and it may serve as a means to track and purify transduced T cells. Modified Tregs comprising anti-Aβ CARs used in methods of treating Alzheimer's disease may suppress proliferation of CD3-activated allogeneic CD8+ T cells in some embodiments. Furthermore, in some embodiments, when activated with oligomeric Aβ, modified Tregs comprising anti-Aβ CARs may produce anti-inflammatory cytokines, e.g., IL-10 when used in methods of treating Alzheimer's disease. Furthermore, said modified Tregs may inhibit production of pro-inflammatory mediators and may enhance phagocytic capacity of activated microglia or macrophages such as by secreting IL-10, TGF-β, and IL-4 anti-inflammatory cytokines for example when used in methods of treating Alzheimer's disease. In some exemplary embodiments, modified Tregs comprising expression of a CAR specific for oligomeric Aβ may have targeted anti-inflammatory activity and neuroprotective effects in regions where oligomeric Aβ may accumulate when used in methods of treating Alzheimer's disease. In some exemplary embodiments, modified Tregs according to the invention may migrate to the hippocampus, wherein oligomeric Aβ may accumulate, when used in methods of treating Alzheimer's disease.

In some embodiments, one or more modified Tregs may comprise any one or more of DG03-28-3ζ; DG03-28tm-10-3ζ; DG03-28tm-44-3ζ; and/or DG03-28tm-CD3ζ, and said modified Tregs may produce IL-10 in response to Aβ antigen, as compared to modified Tregs that were not exposed to said Aβ antigen. In some embodiments, one or more modified Tregs may comprise DG03-CD28-CD3ζ, and said modified Tregs may produce increased levels of IL-10 and/or IL-4 in response to Aβ antigen, which may, for example, be measured by mRNA levels of IL-10 and/or IL-4, and/or be measured by and ELISA assay, as compared to modified Tregs that were not exposed to said Aβ antigen. In some embodiments, one or more modified Tregs may comprise DG03-CD28-CD3ζ, and said modified Tregs may, when stimulated with Aβ antigen and/or anti-CD3 antibody, may inhibit superoxide generation as compared to modified Tregs that were not stimulated with Aβ antigen or anti-CD3 antibody. In some embodiments, one or more modified Tregs may comprise DG03-CD28-CD3ζ, and said modified Tregs, when stimulated with Aβ antigen and/or anti-CD3 antibody, may inhibit IL-6 production as compared to modified Tregs that were not stimulated with Aβ antigen or anti-CD3 antibody.

Moreover, in some exemplary embodiments, modified Tregs that may be used in methods of treating Alzheimer's disease may comprise anti-Aβ CARs and may traffic and accumulate to brain regions of Aβ deposits and neuroinflammation, wherein such regions may include sites of Aβ deposits in the hippocampus and frontal cortex. In some embodiments, modified Tregs comprising CARs targeting Alzheimer's may accumulate in said brain regions and may lead to increased expression of human anti-inflammatory cytokines IL-10, TGF-β, and IL4 in said regions when used in methods of treating Alzheimer's disease. These anti-inflammatory cytokines may lead to a decrease expression of pro-inflammatory mediators and the numbers of microglia. In exemplary embodiments, modified Tregs comprising CARs targeting Alzheimer's disease may improve memory function in a patient treated with said modified Tregs.

In some embodiments, modified Tregs may comprise a construct for expression of the NDMM Nrf2 (Keap1 inhibitor peptide), and said modified Tregs may demonstrate cytoprotective activity, such as, for example, protection of cells from hydrogen peroxide toxicity as compared to methods of treatment that do not comprise use of said modified Tregs. In some embodiments, a modified Treg may comprise a construct for expression of the NDMM human catalase and said modified Tregs may demonstrate cytoprotective activity, such as, for example, protection of cells from hydrogen peroxide toxicity as compared to methods of treatment that do not comprise use of said modified Tregs. In some embodiments, a modified Treg may comprise a construct for expression of the NDMM BDNF and said modified Tregs may demonstrate cytoprotective activity, such as, for example, protection of cells from hydrogen peroxide toxicity as compared to methods of treatment that do not comprise use of said modified Tregs. In some embodiments, a modified Treg may comprise a construct for expression of the NDMM IGF-1 and said modified Tregs may demonstrate cytoprotective activity, such as, for example, protection of cells from hydrogen peroxide toxicity as compared to methods of treatment that do not comprise use of said modified Tregs.

Further therapeutic applications of modified Tregs according to the invention are discussed in detail below.

Therapeutic Applications

Isolated cells obtained by the methods described above, or cell lines derived from such isolated cells, can be used as a medicament in the treatment of a disease, disorder, or condition in a subject. In some embodiments, such a medicament can be used for treating a neurodegenerative disease or condition. In some embodiments, said neurodegenerative disease or condition may be Parkinson's disease, Alzheimer's disease, or ALS.

Cell Origin

For purposes of the inventive methods, wherein host cells or populations of cells are administered, the cells can be cells that are xenogeneic, allogeneic or autologous to the subject. Generally, the cells are autologous or allogeneic compared to the treated subject. In instances wherein the cells are allogeneic, preferably the cells are MHC or HLA histocompatible relative to the subject to be treated and/or are modified to impair or eliminate expression or functionality of the cells' endogenous TCRs and/or MHCs. In some instances, allogeneic Tregs may be preferred, especially if the Tregs of the subject to be treated are diseased and/or possess some property that renders them less than ideal for therapeutic use. In some instances, allogeneic Tregs may be preferred, especially if the Tregs are obtained from healthy donors as they may better migrate or traffic to desired sites, i.e., sites of neurodegeneration or neuroinflammation within the CNS.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

Subject

The subject referred to herein may be any living subject. In a preferred embodiment, the subject is a mammal. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

In some embodiments, the subject, to whom the cells, cell populations, or compositions are administered is a primate, preferably a human. In some embodiments, the primate is a monkey or an ape. The subject can be male or female and can be any suitable age, including infant, juvenile, adolescent, adult, and geriatric subjects. In some examples, the patient or subject is a validated animal model for disease, adoptive cell therapy, and/or for assessing toxic outcomes, such as cytokine release syndrome (CRS).

In some embodiments, the subject has persistent or relapsed disease, e.g., following treatment with another immunotherapy and/or other therapy. In some embodiments, the administration effectively treats the subject despite the subject having become resistant to another therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the methods include administration of modified Tregs comprising one or more CARs and/or one or more NDMMs or a composition containing the cells to a subject, tissue, or cell, such as one having, at risk for, or suspected of having a neurodegenerative disease or condition. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by reducing, inhibiting, or inactivating microglia cells, reducing inflammation and/or neuroinflammation, and/or decreasing neuronal death.

Functional Activity

In some embodiments, the present disclosure includes a type of cellular therapy wherein isolated cells, e.g., Tregs, are genetically modified to express one or more CARs and/or one or more NDMMs against a target molecule which is expressed in a neurodegenerative disease or condition, and a modified Treg cell is infused into a subject in need thereof. Examples of such target molecules include amyloid beta 1-42, superoxide dismutase-1 (SOD-1), alpha-synuclein, hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; Leucine-rich repeat serine/threonine-protein kinase 2 (LRRK2), Huntington with tandem glutamine repeats; prion proteins; transthyretin; dentatorubral pallidoluysian atrophy (DRPLA) protein; androgen receptor; ataxins; P/Q-type calcium channel α1A subunit; TATA-box-binding protein; glial fibrillary acidic protein; DNA excision repair protein ERCC-6; survival motor neuron protein; cystatin C. Such administration can decrease neurodegeneration and/or neuroinflammation in a target molecule specific manner.

In some embodiments, the modified Tregs can undergo in vivo expansion and can persist for an extended amount of time.

Once the cells (modified Tregs) are administered to a subject (e.g., a human), the biological activity of the engineered cell populations in some aspects is measured by any of a number of known methods. Parameters to assess include specific binding of an engineered or natural Treg cell or other immune cell to antigen, in vivo, e.g., by imaging, or ex vivo, e.g., by ELISA or flow cytometry.

In some aspects the biological activity is measured by assessing clinical outcome, such as the reduction in disease symptoms, such as symptoms associated a neurodegenerative disease or condition, e.g., Alzheimer's disease, ALS and Parkinson's disease.

Targets

The Tregs of the present disclosure, which may comprise one or more CARs and/or one or more NDMMs, may be used to treat, prevent, or diagnose any conditions, disorders, or diseases involving the expression of target molecules described herein (e.g., alpha-synuclein, amyloid beta, or mutSOD1). For example, the invention also contemplates a method of treating or preventing neurodegenerative diseases or conditions that may include: Alzheimer's disease, Parkinson's disease, ALS, prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathies; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis. The contemplated method comprises administering modified Tregs that optionally may comprise one or more CARs and/or one or more NDMMs according to the present disclosure.

Modes of Administration

The compositions of the present invention may be administered in a number of ways depending upon whether local or systemic treatment is desired. In the case of adoptive cell therapy, methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

Such administration may be topical, parenteral, or enteral. The compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In some embodiments, parenteral administration of the compositions of the present invention comprises subcutaneous or intraperitoneal administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal. Preferably, the formulated composition comprising modified Tregs is suitable for administration via injection.

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, aerosols, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In some embodiments of the present disclosure the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising populations of modified Tregs of the present disclosure may include pharmaceutically acceptable excipient(s). Excipients included in the formulations will have different purposes depending, for example, on the modified Treg, the subpopulation of modified Tregs used, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising populations of the modified Tregs of the present disclosure may typically have been prepared and cultured in the absence of any non-human components, such as animal serum (e.g., bovine serum albumin).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

Dosing

The pharmaceutical composition in some embodiments contains modified Tregs of the present disclosure, e.g., Tregs comprising one or more CARs and/or one or more NDMMs, in amounts effective to treat or prevent the disease or condition, e.g., a neurodegenerative disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of modified Tregs, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection into the disease site.

For purposes of the present disclosure, the amount or dose of modified Tregs administered should be sufficient to effect a therapeutic or prophylactic response in the subject or animal over a reasonable time frame. For example, the dose of modified Tregs should be sufficient to bind to antigen, or detect, treat or prevent disease in a period of from about 2 hours or longer, e.g., about 12 to about 24 or more hours, from the time of administration. In certain embodiments, the time period could be even longer. The dose will be determined by the efficacy of the particular modified Treg and the condition of the animal (e.g., human), as well as the body weight of the animal (e.g., human) to be treated.

In some embodiments, modified Tregs according to the invention are administered as part of a combination treatment, such as simultaneously with or sequentially with, in any order, another therapeutic intervention known in the art. For example modified Treg cells according to the invention in some embodiments are co-administered with one or more additional therapeutic agents such as an antibody, nucleic acid or small molecule or in combination with another therapeutic intervention, either simultaneously or sequentially in any order. In some contexts, the cells are co-administered with another moiety which promotes the ability of the cells to cross the BBB. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after to the one or more additional therapeutic agents.

### EXAMPLES

### Example 1: Treg Isolation, Expansion and CAR Transduction for Modified Tregs Targeting Alzheimer's Disease

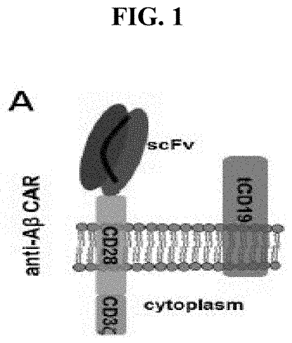

In the present Example, modified Tregs targeting Alzheimer's disease comprised extracellular scFvs fused to human CD28 hinge, transmembrane, and cytoplasmic domains, followed by a human CD3ζ cytoplasmic domain (FIG. 1). CARs targeting Alzheimer's disease include DG01 (SEQ ID NO: 1), DG02 (SEQ ID NO: 2), DG03 (SEQ ID NO: 3), and DG04 (SEQ ID NO: 4), DG01-CD28-CD3ζ (SEQ ID NO: 20), DG02-CD28-CD3ζ (SEQ ID NO: 21), DG03-CD28-CD3ζ (SEQ ID NO: 22), and DG04-CD28-CD3ζ (SEQ ID NO: 23). Modified Tregs targeting Alzheimer's disease were prepared as follows.

CD4+CD25+ Tregs were isolated from human PBMCs in a two-step cell isolation process. First, human CD4+ cells were isolated by negative-selection using MOJOSORT™ Human CD4 T Cell Isolation Kit (Biolegend) and a EASYSEP™ Magnet (StemCell) according to manufacturer's instructions. Second, CD25hi cells were enriched from the CD4+-isolated cells by positive-selection using anti-human CD25 MicroBeads II (Miltenyi) and MS Columns with MiniMACS™ Separator magnet (Miltenyi). The CD4+CD25hi cells were cultured in 24-well non-tissue culture plates at 1×10⁶ cells/mL in Treg growth medium supplemented with 10% heat-inactivated human AB serum (Sigma). Treg growth media was either (1) X-Vivo-15 or (2) RPMI supplemented with 10 mM HEPES, 0.1 mM non-essential amino acids, 1 mM sodium pyruvate, 100 U/mL penicillin, 100 μg/mL streptomycin and 50 μM 2-ME. Cell incubation condition was humidified 37° C. and 5% CO₂. Cells were stimulated with 25 μL/1×10⁶ cells of ImmunoCult™ Human CD3/CD28 T Cell Activator (StemCell) on days 0 and 9 in culture. In some instances, it is possible to use the CD3/CD28/CD2 T cell activator instead. Treg growth media was supplemented with 300 to 500 U/mL human IL-2 (Tecin from Roche, kindly provided by the NIH) starting on culture day 2. Cultured cells were transferred to 25 cm² tissue culture-treated flasks on day 5. Cells were transduced over two days with retroviral CAR constructs on day 10 and 11 in culture. 24-well non-tissue culture plate wells were pre-coated with RetroNectin® (Takara Bio USA, Inc.) according to manufacturer's instructions and then day 10 cultured cells were added at 0.3×10⁶ cells/well in 0.3 mL of Treg growth media. Retroviral supernatant was added at 0.7 mL/well and plates were centrifuged at 1500 rcf at 30° C. for 1 h, and then incubated overnight. On the next day, 0.5 mL culture supernatant was replaced with 0.5 mL retroviral supernatant with 500 U/mL IL-2 and cells were re-centrifuged and then incubated overnight. The next day cells were transferred to 25 cm² tissue culture-treated flasks at 1×10⁶ cells/mL. On day 13 of culture, a sample of cells was evaluated for transduction efficiency by measuring the percentage of the co-transduced truncated CD19 on cells by flow cytometry. If cells were less than 30% transduced, then cells can be enriched using anti-CD19-PE and EasySep™ Release PE Positive Selection Kit (StemCell) according to manufacturer's instructions. Cells were cultured with fresh Treg growth media added every two days until day 17.

The flow cytometry step proceeded as follows.

Cell phenotyping was determined by staining with specific antibodies: FITC-conjugated anti-human CD3 (clone OKT3; Biolegend), FITC anti-human CD4 (clone OKT4; Biolegend), PE anti-human CD127 (clone A019DS; Biolegend), PE anti-human CD25 (clone (clone M-A251; Biolegend), PE anti-human CD19 (clone SJ25C1; Biolegend), or PE anti-mouse CD19 (clone 6D5; Biolegend). For intracellular staining, eBioscience™ Foxp3/Transcription Factor Staining Buffer Set was used with APC anti-human Fox3 (clone PCH101; Invitrogen) or APC rat IgG2a isotype control (clone eBR2a; Invitrogen). For direct labeling of the CAR scFv, biotinylated protein L (1 μg/mL; GenScript) followed by streptavidin-PE (Biolegend) was used. Other reagents to label scFv-based CARs can include FITC anti-human IgG, F(ab′)₂-specific (Jackson ImmunoResearch), PE AffiniPure F(ab′)2 Fragment anti-human IgG (Jackson ImmunoResearch), or PE AffiniPure F(ab′)₂ Fragment anti-mouse IgG (Jackson ImmunoResearch). Cells were analyzed using an ACCURI™ C6 flow cytometer (BD Biosciences, Ann Arbor, Mich., USA).

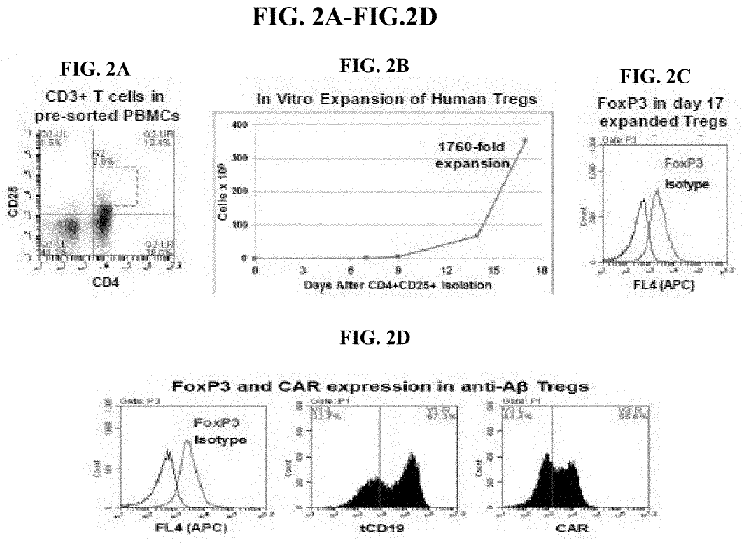

FIG. 2A-FIG. 2D presents data related to the in vitro expansion and phenotype validation of Tregs isolated from human PBMCs as discussed above. FIG. 2A demonstrates that CD4⁺CD25hi Tregs (R2 box) represented a small percentage of total T cells in human PBMCs prior to CD4 and CD25 enrichment isolation. FIG. 2B demonstrates that CD4⁺CD25hi isolated Tregs expanded 1760-fold after 17 days in culture using the present Treg expansion protocol. FIG. 2C demonstrates that Day 17 Tregs expressed intracellular FoxP3. FIG. 2D demonstrates that FoxP3, truncated CD19 (tCD19), and the CAR scFv were detected on most day 17 Tregs transduced on days 10 and 11.

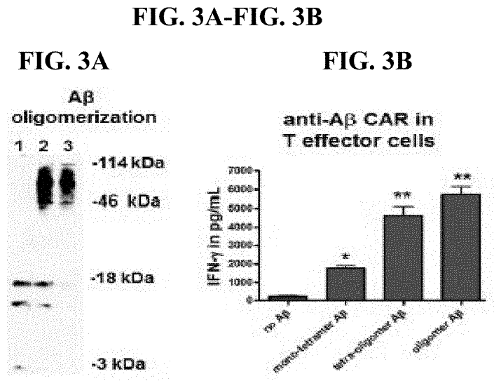

CARs targeting amyloid beta were functionally validated as follows. Oligomerization of Aβ₁₋₄₂ peptides can occur over the course of seven days incubation in PBS, and in the present example resulted in increasing ratios of larger oligomers as a function of time, as demonstrated by FIG. 3A. To test CAR antigen-specific activity, total effector T cells (CD4⁺ and CD8⁺ Teff cells) transduced to express DG03-28ζ anti-Aβ CAR (SEQ ID NO: 22) were used. Higher secretion of IFN-γ was found when these CART cells were exposed to Aβ oligomers with a higher ratio of larger aggregates, whereas little response occurs without antigen (FIG. 3B). This demonstrated that the CAR retains binding specificity to Aβ and that the oligomerized antigen activated CAR T cells (FIG. 3B).

### Example 2: Treg Suppression Assay Using Exemplary Modified Tregs Targeting Alzheimer's Disease

Modified Tregs comprising CARs, as described in Example 1, were plated in a 96-well V bottom plate in Treg growth medium at serial dilutions between 12,500 and 400,000 cells/0.1 mL/well. Allogeneic PBMCs were pre-labeled using CellTrace™ CFSE Cell Proliferation Kit (Invitrogen) according to manufacturer's instructions. CSFE-labeled PBMCs were stimulated with or without anti-CD3 (1 ug/mL; clone HIT3a; Biolegend) and added to plated Tregs or to wells with no Tregs. After 72 h, cells were blocked using human Cohns fraction (1 mg/mL; Sigma), and then stained with APC anti-CD8 (clone RTA-T8; Biolegend) for flow cytometer analysis of CD8⁺ cells expression of CSFE. The CFSE can be reduced with each cell division, so dividing cells can have a lower CFSE value. The % suppression of CD8⁺ T cell proliferation was determined compared to control wells of stimulated PBMCs with anti-CD3 mAbs.

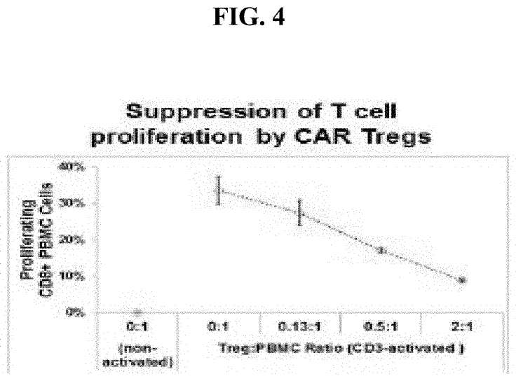

Results of the above Treg suppression assay are presented in FIG. 4. FIG. 4 demonstrates that increasing ratios of day 17 anti-Aβ CAR Tregs comprising DG03-28z suppressed the proliferation of CSFE-labeled CD8⁺ cells in co-cultured CD3-stimulated allogeneic PBMCs.

### Example 3: Antigen-Stimulation Modified Tregs Targeting Alzheimer's Disease

For stimulation with soluble antigen, day 17 CAR Tregs were plated in non-coated 96-well tissue culture plates at 50,000 cell/well in Treg growth media without IL-2. Aβ₁₋₄₂ or α-synuclein was pre-oligomerized by incubating at 40 μM in PBS at 37° C. for 1 week with daily agitation. Oligomerized Aβ₁₋₄₂ was added to Tregs in 0.1 mL Treg growth media and cell free supernatant was collected for human IL-10 ELISA (Biolegend) at 24 h and 72 h. For stimulation with plate-bound antigens, 96-well ELISA plates were coated with Aβ₁₋₄₂, wtSOD1, mutSOD1, or α-synuclein in PBS overnight at 4° C. Plates were rinsed three times with PBS and day 17 CAR Tregs were added to the antigen-coated 96-well ELISA plates at 50,000 cell/well in Treg growth media without IL-2. Cell free supernatant was collected for human IL-10 ELISA (Biolegend) at 24 h and 72 h. Alternatively, streptavidin in PBS was coated on 96-well ELISA plates overnight at 4° C. and biotinylated antigens were applied after rinsing unbound streptavidin. Another alternative method for antigen stimulation can be to use nanometer- or micrometer-sized polystyrene beads coated with the antigen e.g. biotinylated antigen linked to streptavidin-conjugated beads.

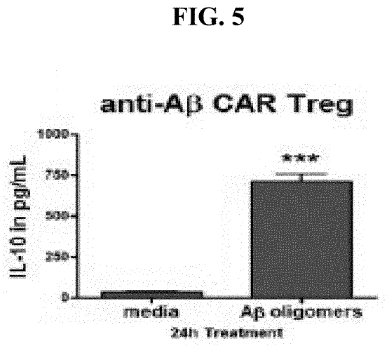

FIG. 5 demonstrates that anti-Aβ CAR Tregs comprising DG03-28z stimulated with oligomeric Aβ in vitro for 24 hours produced IL-10. P<0.001 by Student's t-test.

### Example 4: Treg Isolation, Expansion and CAR Transduction for Modified Tregs Targeting ALS

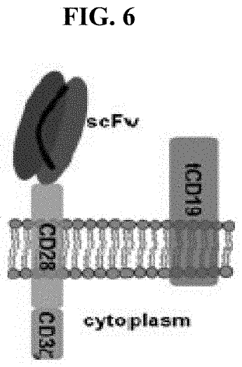

In the present example, modified Tregs targeting ALS comprised anti-mutSOD1 CARs using human variable heavy (V_{H}) and light (V_{L}) chain sequences. The anti-mutSOD1 scFv were expressed extracellularly with the C-terminus of the V_{L} fused to human CD28 hinge, transmembrane, and cytoplasmic domain, followed by a human CD3ζ cytoplasmic domain to create an anti-mutSOD1-CD28-CD3ζ CAR (FIG. 6). This CAR will trigger both primary and costimulation signaling upon antigen binding. A truncated (non-signaling) human or mouse CD19 (tCD19) is also expressed in the same vector using a 2A co-expression system and it serves as a way to track and purify transduced T cells. Modified Tregs targeting ALS are prepared as follows. CARs targeting ALS include DG05 (SEQ ID NO: 5), DG06 (SEQ ID NO: 6), DG07 (SEQ ID NO: 7), DG05-CD28-CD3ζ (SEQ ID NO: 24), DG06-CD28-CD3ζ (SEQ ID NO: 25), and DG07-CD28-CD3ζ (SEQ ID NO: 26).

CD4+CD25+ Tregs were isolated from human PBMCs in a two-step cell isolation process. First, human CD4+ cells were isolated by negative-selection using MOJOSORT™ Human CD4 T Cell Isolation Kit (Biolegend) and a EASYSEP™ Magnet (StemCell) according to manufacturer's instructions. Second, CD25hi cells were enriched from the CD4+-isolated cells by positive-selection using anti-human CD25 MicroBeads II (Miltenyi) and MS Columns with MiniMACS™ Separator magnet (Miltenyi). The CD4+CD25hi cells were cultured in 24-well non-tissue culture plates at 1×10⁶ cells/mL in Treg growth medium supplemented with 10% heat-inactivated human AB serum (Sigma). Treg growth media was either (1) X-Vivo-15 or (2) RPMI supplemented with 10 mM HEPES, 0.1 mM non-essential amino acids, 1 mM sodium pyruvate, 100 U/mL penicillin, 100 μg/mL streptomycin and 50 μM 2-ME. Cell incubation condition was humidified 37° C. and 5% CO₂. Cells were stimulated with 25 μL/1×10⁶ cells of ImmunoCult™ Human CD3/CD28 T Cell Activator (StemCell) on days 0 and 9 in culture. In some instances, it is possible to use the CD3/CD28/CD2 T cell activator instead. Treg growth media was supplemented with 300 to 500 U/mL human IL-2 (Tecin from Roche, kindly provided by the NIH) starting on culture day 2. Cultured cells were transferred to 25 cm² tissue culture-treated flasks on day 5. Cells were transduced over two days with retroviral CAR constructs on day 10 and 11 in culture. 24-well non-tissue culture plate wells were pre-coated with RetroNectin® (Takara Bio USA, Inc.) according to manufacturer's instructions and then day 10 cultured cells were added at 0.3×10⁶ cells/well in 0.3 mL of Treg growth media. Retroviral supernatant was added at 0.7 mL/well and plates were centrifuged at 1500 rcf at 30° C. for 1 h, and then incubated overnight. On the next day, 0.5 mL culture supernatant was replaced with 0.5 mL retroviral supernatant with 500 U/mL IL-2 and cells were re-centrifuged and then incubated overnight. The next day cells were transferred to 25 cm² tissue culture-treated flasks at 1×10⁶ cells/mL. On day 13 of culture, a sample of cells was evaluated for transduction efficiency by measuring the percentage of the co-transduced truncated CD19 on cells by flow cytometry. If cells were less than 30% transduced, then cells can be enriched using anti-CD19-PE and EasySep™ Release PE Positive Selection Kit (StemCell) according to manufacturer's instructions. Cells were cultured with fresh Treg growth media added every two days until day 17.

The flow cytometry step proceeded as follows.

Cell phenotyping was determined by staining with specific antibodies: FITC-conjugated anti-human CD3 (clone OKT3; Biolegend), FITC anti-human CD4 (clone OKT4; Biolegend), PE anti-human CD127 (clone A019DS; Biolegend), PE anti-human CD25 (clone (clone M-A251; Biolegend), PE anti-human CD19 (clone SJ25C1; Biolegend), or PE anti-mouse CD19 (clone 6D5; Biolegend). For intracellular staining, eBioscience™ Foxp3/Transcription Factor Staining Buffer Set was used with APC anti-human Fox3 (clone PCH101; Invitrogen) or APC rat IgG2a isotype control (clone eBR2a; Invitrogen). For direct labeling of the CAR scFv, biotinylated protein L (1 μg/mL; GenScript) followed by streptavidin-PE (Biolegend) was used. Other reagents to label scFv-based CARs can include FITC anti-human IgG, F(ab′)₂-specific (Jackson ImmunoResearch), PE AffiniPure F(ab′)2 Fragment anti-human IgG (Jackson ImmunoResearch), or PE AffiniPure F(ab′)₂ Fragment anti-mouse IgG (Jackson ImmunoResearch). Cells were analyzed using an ACCURI™ C6 flow cytometer (BD Biosciences, Ann Arbor, Mich., USA).

Alternatively, for control CAR TZ47-28z CAR, transduction proceeded as follows. Cells were isolated from human PBMCs via a two-step negative and positive selection protocol. First, CD4⁺CD127⁻ T cells were isolated using negative selection, followed by a positive selection of CD25⁺ cells to isolate CD4⁺CD127⁻CD25⁺ Treg cells. The isolated CD4⁺CD25⁺CD127⁻ Tregs were activated with anti-CD3ζ, anti-CD28, anti-CD2 coated beads (STEMCELL ImmunoCult) with human IL-2 over two weeks in culture. On day 9, Treg cells were cryopreserved for use at a later date, so the same donor and preparation can be tested on multiple occasions to assess assay variability. Using this protocol, it was possible to generate 5×10⁸ Treg cells (CD4⁺, CD127⁻, CD25⁺, FoxP3⁺) from an initial 1×10⁸ whole PBMCs.

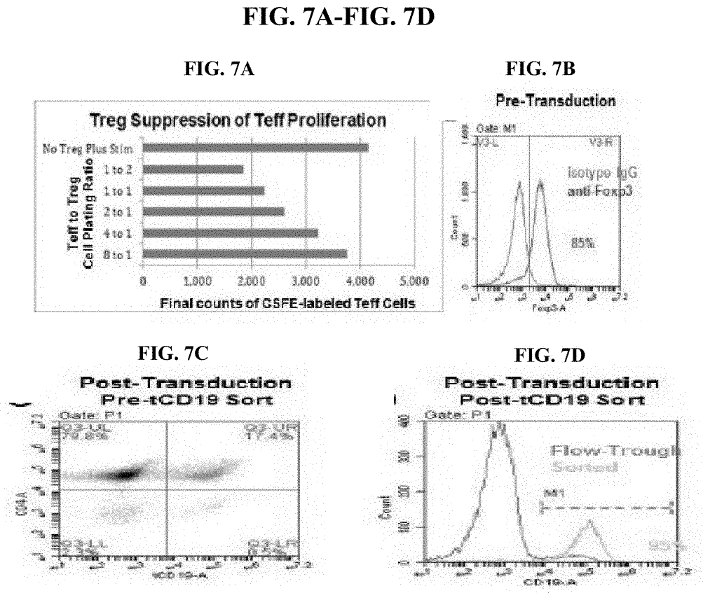

For CAR transduction, thawed 9-day-expanded Treg cells were stimulated for 48 hours with Immunocult activator beads. Tregs were transduced by spin inoculation with anti-mutSOD1 CARs or a negative control CAR and tCD19 or tCD19 vector alone in Rectronection-coated plates (Clontech Laboratories, Inc.). Transduced tCD19⁺ cells were purified using magnetic bead selection (StemCell) three days after transduction. With this method it was shown that expanded Tregs transduced with an anti-B7H6 CAR and tCD19 retained a Treg phenotype: suppression of effector T cell proliferation (FIG. 7A), and the majority of cells expressed intracellular FoxP3 (FIG. 7B). Purification for tCD19⁺ cells resulted in a purity of up to 95% (FIG. 7C-FIG. 7D). Regarding FIG. 7A, CD4⁺CD127⁻ CD25⁺ cells isolated from human PBMCs and expanded in vitro had Treg-like function as shown by suppression of CD3-stimulated proliferation of CSFE-labeled T effector cells (Teff). Regarding FIG. 7B, CD4⁺CD127⁻CD25⁺ isolated and expanded cells were positive for intracellular Treg marker FoxP3. Regarding FIG. 7C-FIG. 7D, following transduction of isolated Tregs with truncated CD19 (tCD19) retrovirus, 17% of CD4⁺ cells expressed tCD19 (FIG. 7C), and after tCD19 purification 95% of sorted cells were tCD19⁺ (FIG. 7D).

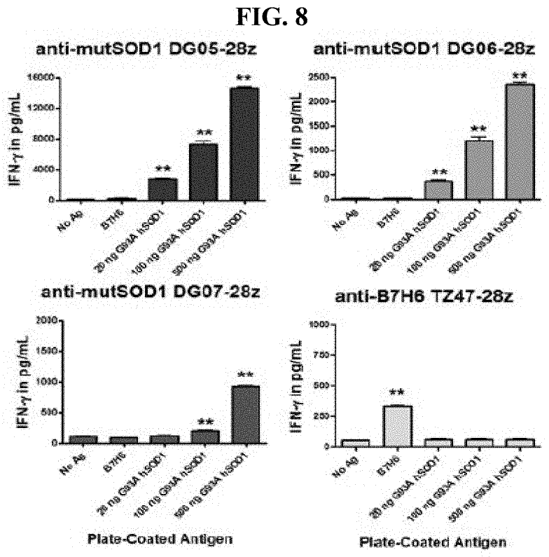

Using total effector T cells (CD4⁺ and CD8⁺ T cells)—derived from human PBMCs stimulated with 40 ng/mL soluble OKT3 and 100 u/mL IL-2, spin transduced on Retronectin-coated wells on days 2 and 3 in culture, and used on day 8 in culture—three anti-mutSOD1 CARs (named DG05, DG06, and DG07) showed antigen specific activity against the G93A mutated form of SOD1, which was similar to anti-B7H6 (TZ47) CAR T cells targeting B7H6 (FIG. 8). The two human antibodies from which anti-mutSOD1 CARs DG05 and DG06 were developed were selective for human SOD1 with ALS mutations.

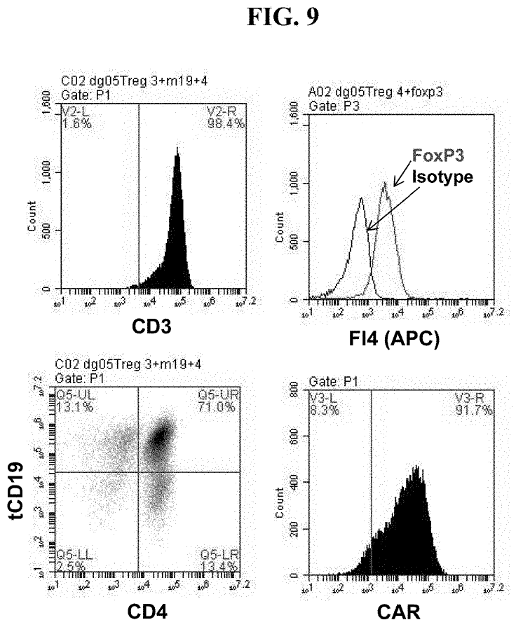

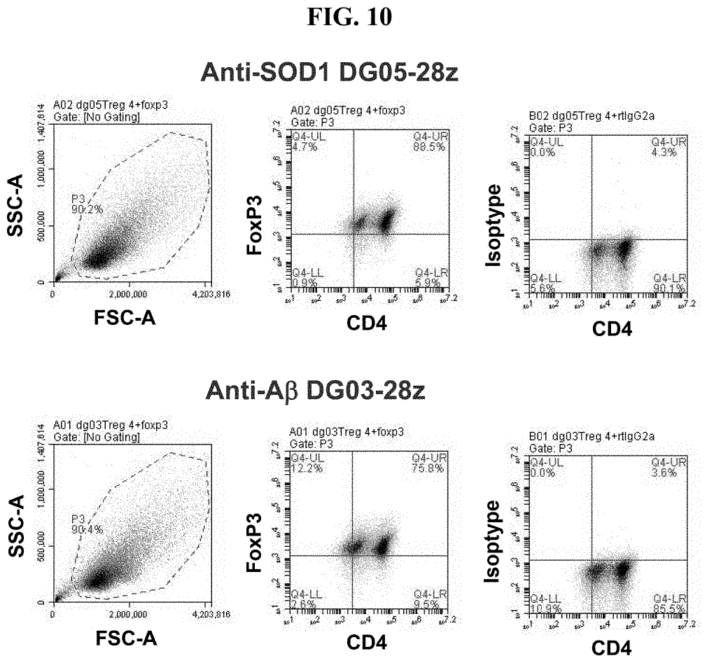

FIG. 9 presents data related to the phenotype validation of Tregs isolated from human PBMCs as discussed above. FIG. 9 demonstrates that DG05-28Z (SEQ ID NO: 24) expressed in human Tregs tested positive for the following markers using a flow cytometry-based assay: FoxP3; CD4; CD3; transduction marker truncated CD19 (tCD19); and CAR by labeling with biotinylated protein L and streptavidin-PE. Furthermore, modified Tregs expressing anti-mutSOD1 CAR DG05-28z had a similar phenotype to Tregs expressing CAR targeting a different target (DG03-28z (SEQ ID NO: 22)) (FIG. 10).

### Example 5: Functional Activity of Modified Tregs Targeting ALS

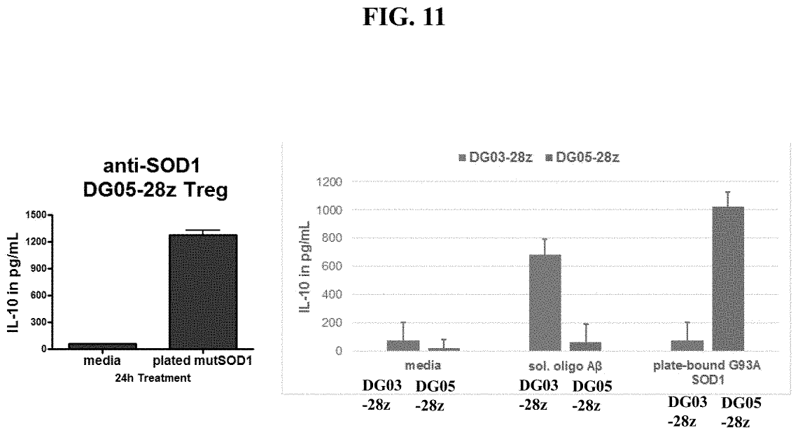

IL-10 ELISA data using anti-mutSOD1 CAR DG05-28z (SEQ ID NO: 24) expressed in human Tregs are presented below. FIG. 11 demonstrates that anti-mutSOD1 CAR Tregs DG05-28z produced IL-10 when cultured in wells with mutSOD1 coated on the well surface (plate-bound) versus media alone. Furthermore, FIG. 11 demonstrates that anti-mutSOD1 CAR Tregs did not produce IL-10 in response to soluble oligomerized Aβ₁₋₄₂, whereas anti-Aβ CAR Tregs did produce IL-10 in response to soluble oligomerized Aβ₁₋₄₂.

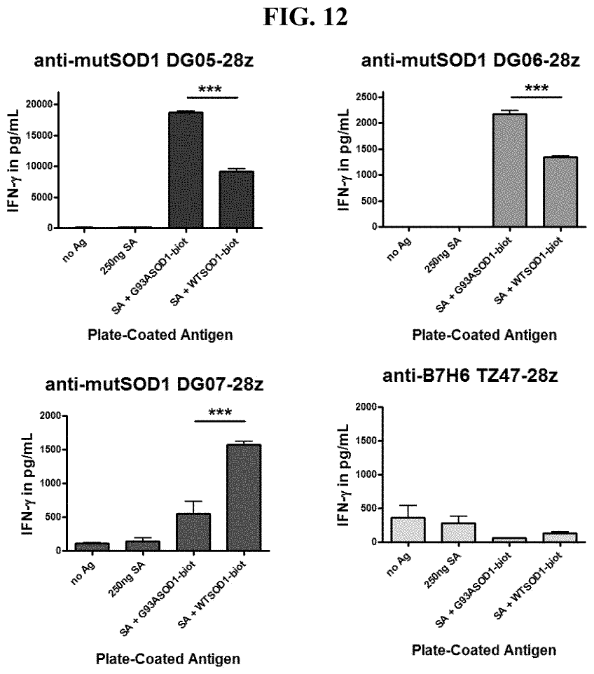

FIG. 12 demonstrates that DG05-28z (SEQ ID NO: 24) and DG06-28z (SEQ ID NO: 25) anti-mutSOD1 CARs expressed in human effector T cells produced IFN-gamma in response to biotinylated mutSOD1 relative to wtSOD1 that was linked to plate-bound streptavidin as described above. A third anti-mutSOD1 CAR called DG07-28z (SEQ ID NO: 26) produced a higher amount of IFN-γ in response to wtSOD1 as compared to mutSOD1 when expressed in human T effector cells.

The IL-10 production assays involving modified Tregs expressing anti-mutSOD1 CAR DG05-28z demonstrated that it is reasonable to expect that anti-SOD1 CAR Tregs can respond to mutant and wild-type forms of SOD1, but only when they were aggregated or bound on a surface (e.g. tissue culture plate). Based on data of DG05-28z and DG06-28z anti-mutSOD1 CARs expressed in human T effector cells as described above, it is reasonable to expect that CARs can be more selective to aggregated or plate-bound mutSOD1 relative to wtSOD1.

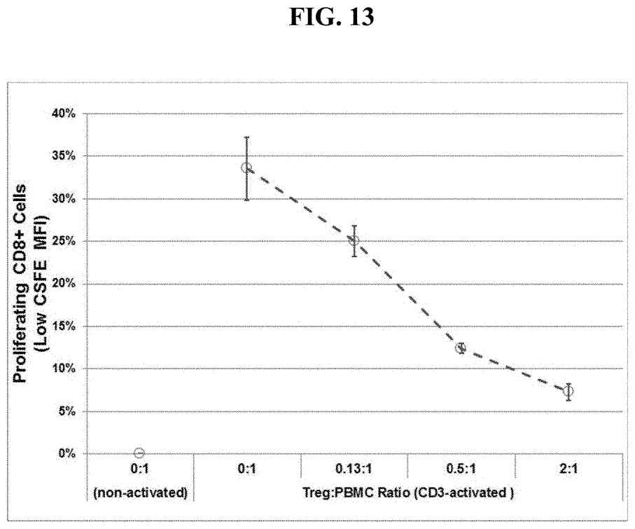

Modified Treg regulator function was evaluated by in vitro proliferation inhibition of T effector cells. The source of Teff was allogeneic PBMCs that were CSFE-labeled. Modified Tregs comprising CARs were plated in a 96-well V bottom plate in Treg growth medium at serial dilutions between 12,500 and 400,000 cells/0.1 mL/well. Allogeneic PBMCs were pre-labeled using CellTrace™ CFSE Cell Proliferation Kit (Invitrogen) according to manufacturer's instructions. CSFE-labeled PBMCs were stimulated with or without anti-CD3 (1 ug/mL; clone HIT3a; Biolegend) and added to plated Tregs or to wells with no Tregs. After 72 h, cells were blocked using human Cohns fraction (1 mg/mL; Sigma), and then stained with APC anti-CD8 (clone RTA-T8; Biolegend) for flow cytometer analysis of CD8⁺ cells expression of CSFE. The CFSE can be reduced with each cell division, so dividing cells can have a lower CFSE value. The % suppression of CD8⁺ T cell proliferation was determined compared to control wells of stimulated PBMCs with anti-CD3 mAbs. Proliferation of the CD8 subset of Teff in the CSFE-labeled PBMCs was measured after 72 h or co-cultured with anti-mutSOD1 CAR Tregs. The CAR Tregs were co-cultured with PBMCs at Treg:PBMC ratios of 0.13:1, 0.5:1, and at 2:1 (FIG. 13).

### Example 6: Treg Isolation, Expansion and CAR Transduction for Modified Tregs Targeting Parkinson's Disease

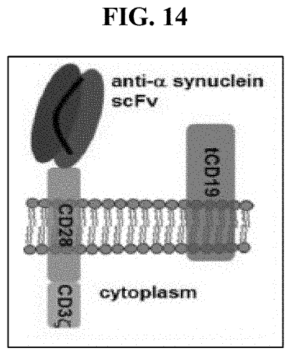

In the present example, modified Tregs targeting Parkinson's disease comprised anti-α-synuclein CARs using human variable heavy (V_{H}) and light (V_{L}) chain sequences. The anti-α-synuclein scFv was expressed extracellularly with the C-terminus of the V_{L} fused to human CD28 hinge, transmembrane, and cytoplasmic domain, followed by a human CD3ζ cytoplasmic domain to create an anti-α-synuclein-CD28-CD3ζ CAR (FIG. 14). This CAR will trigger both primary and costimulation signaling upon antigen binding. A truncated (non-signaling) human CD19 (tCD19) is also expressed in the same vector using a 2A co-expression system and it serves as a way to track and purify transduced T cells. CARs targeting Parkinson's disease include DG08 (SEQ ID NO: 8), DG09 (SEQ ID NO: 9), DG10 (SEQ ID NO: 10), DG11 (SEQ ID NO: 11), DG08-CD28-CD3ζ (SEQ ID NO: 27), DG09-CD28-CD3ζ (SEQ ID NO: 28), DG10-CD28-CD3ζ (SEQ ID NO: 29), and DG11-CD28-CD3ζ (SEQ ID NO: 30). Modified Tregs targeting Parkinson's disease were prepared as follows.

CD4+CD25+ Tregs were isolated from human PBMCs in a two-step cell isolation process. First, human CD4+ cells were isolated by negative-selection using MOJOSORT™ Human CD4 T Cell Isolation Kit (Biolegend) and a EASYSEP™ Magnet (StemCell) according to manufacturer's instructions. Second, CD25hi cells were enriched from the CD4+-isolated cells by positive-selection using anti-human CD25 MicroBeads II (Miltenyi) and MS Columns with MiniMACS™ Separator magnet (Miltenyi). The CD4+CD25hi cells were cultured in 24-well non-tissue culture plates at 1×10⁶ cells/mL in Treg growth medium supplemented with 10% heat-inactivated human AB serum (Sigma). Treg growth media was either (1) X-Vivo-15 or (2) RPMI supplemented with 10 mM HEPES, 0.1 mM non-essential amino acids, 1 mM sodium pyruvate, 100 U/mL penicillin, 100 μg/mL streptomycin and 50 μM 2-ME. Cell incubation condition was humidified 37° C. and 5% CO₂. Cells were stimulated with 25 μL/1×10⁶ cells of ImmunoCult™ Human CD3/CD28 T Cell Activator (StemCell) on days 0 and 9 in culture. In some instances, it is possible to use the CD3/CD28/CD2 T cell activator instead. Treg growth media was supplemented with 300 to 500 U/mL human IL-2 (Tecin from Roche, kindly provided by the NIH) starting on culture day 2. Cultured cells were transferred to 25 cm² tissue culture-treated flasks on day 5. Cells were transduced over two days with retroviral CAR constructs on day 10 and 11 in culture. 24-well non-tissue culture plate wells were pre-coated with RetroNectin® (Takara Bio USA, Inc.) according to manufacturer's instructions and then day 10 cultured cells were added at 0.3×10⁶ cells/well in 0.3 mL of Treg growth media. Retroviral supernatant was added at 0.7 mL/well and plates were centrifuged at 1500 rcf at 30° C. for 1 h, and then incubated overnight. On the next day, 0.5 mL culture supernatant was replaced with 0.5 mL retroviral supernatant with 500 U/mL IL-2 and cells were re-centrifuged and then incubated overnight. The next day cells were transferred to 25 cm² tissue culture-treated flasks at 1×10⁶ cells/mL. On day 13 of culture, a sample of cells was evaluated for transduction efficiency by measuring the percentage of the co-transduced truncated CD19 on cells by flow cytometry. If cells were less than 30% transduced, then cells can be enriched using anti-CD19-PE and EasySep™ Release PE Positive Selection Kit (StemCell) according to manufacturer's instructions. Cells were cultured with fresh Treg growth media added every two days until day 17.

### Example 7: Functional Activity of Modified Tregs Targeting Parkinson's Disease

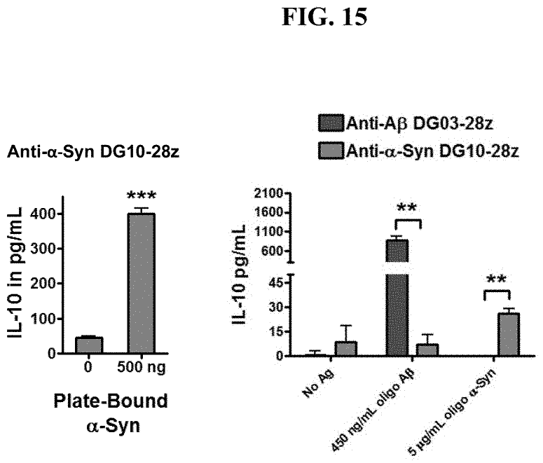

The functional activity of modified Tregs targeting Parkinson's disease was evaluated by culturing modified Tregs in wells with α-synuclein coated on the well surface (plate-bound) and with soluble oligomerized α-synuclein versus media alone For stimulation with soluble antigen, day 17 CAR Tregs were plated in non-coated 96-well tissue culture plates at 50,000 cell/well in Treg growth media without IL-2. Aβ₁₋₄₂ or α-synuclein was pre-oligomerized by incubating at 40 μM in PBS at 37° C. for 1 week with daily agitation. Oligomerized α-synuclein was added to Tregs in 0.1 mL Treg growth media and cell free supernatant was collected for human IL-10 ELISA (Biolegend) at 24 h and 72 h. For stimulation with plate-bound antigens, 96-well ELISA plates were coated with Aβ₁₋₄₂, wtSOD1, mutSOD1, or α-synuclein in PBS overnight at 4° C. Plates were rinsed three times with PBS and day 17 CAR Tregs were added to the antigen-coated 96-well ELISA plates at 50,000 cell/well in Treg growth media without IL-2. Cell free supernatant was collected for human IL-10 ELISA (Biolegend) at 24 h and 72 h. Alternatively, streptavidin in PBS was coated on 96-well ELISA plates overnight at 4° C. and biotinylated antigens were applied after rinsing unbound streptavidin. Another alternative method for antigen stimulation can be to use nanometer- or micrometer-sized polystyrene beads coated with the antigen e.g. biotinylated antigen linked to streptavidin-conjugated beads. FIG. 15 presents data that demonstrated anti-α-synuclein CAR Tregs produced IL-10 when cultured in wells with α-synuclein coated on the well surface (plate-bound) and with soluble oligomerized α-synuclein versus media alone. Additionally, as presented in FIG. 15, anti-α-synuclein CAR Tregs did not produce IL-10 in response to soluble oligomerized Aβ₁₋₄₂, whereas anti-Aβ CAR Tregs did produce IL-10 in response to soluble oligomerized Aβ₁₋₄₂, thus, demonstrating antigen specificity for the CARs on Tregs.

### Example 8: Expression and Functional Activity of Modified Tregs Targeting ALS

In the present example, expression and function of anti-mSOD1 CARs with different costimulatory signaling domains, and also with or without the CD3-zeta (“CD3ζ”) stimulatory domain, were evaluated in ex vivo expanded and CAR transduced human Tregs. The anti-mSOD1 scFv DG05 and CD28 transmembrane domain (“28tm”) were in all constructs, and mouse truncated CD19 was in the vector with the CAR on the same construct. The CD3-zeta (“CD3ζ”) domain was joined to co-stimulatory domains CD28 (“28”), DAP10 (“10”), or CD44 (“44”) to produce the following anti-mSOD1 CARs: DG05-28-3ζ (SEQ ID NO: 24); DG05-28tm-10-3 (SEQ ID NO: 40), and DG05-28tm-44-3ζ (SEQ ID NO: 41). Additionally, an anti-mSOD1 CAR comprising CD28 transmembrane domain (“CD28tm”) with no stimulatory or co-stimulatory domain was produced (DG05-28tm, SEQ ID NO: 44); an anti-mSOD1 CAR comprising CD28 transmembrane domain with a 3ζ domain and without a CD28 co-stimulatory domain was produced (DG05-28tm-3ζ, SEQ ID NO: 42); and an anti-mSOD1 CAR comprising a CD28 co-stimulatory domain with no 3ζ domain was produced (DG05-28, SEQ ID NO: 43).

The modified Tregs of the present example were isolated, expanded, and transduced as generally described in Example 4. More specifically, Day 17 CAR Tregs were prepared from human PBMCs as described in Example 4). PG13 retrovirus with the following anti-mSOD1 CARs was used: DG05-28-3 ζ (SEQ ID NO: 24); DG05-28tm-10-3ζ (SEQ ID NO: 40); DG05-28tm-44-3 ζ (SEQ ID NO: 41), DG05-28tm-3 ζ (SEQ ID NO: 42), DG05-28 (SEQ ID NO: 43), and DG05-28tm (SEQ ID NO: 44).

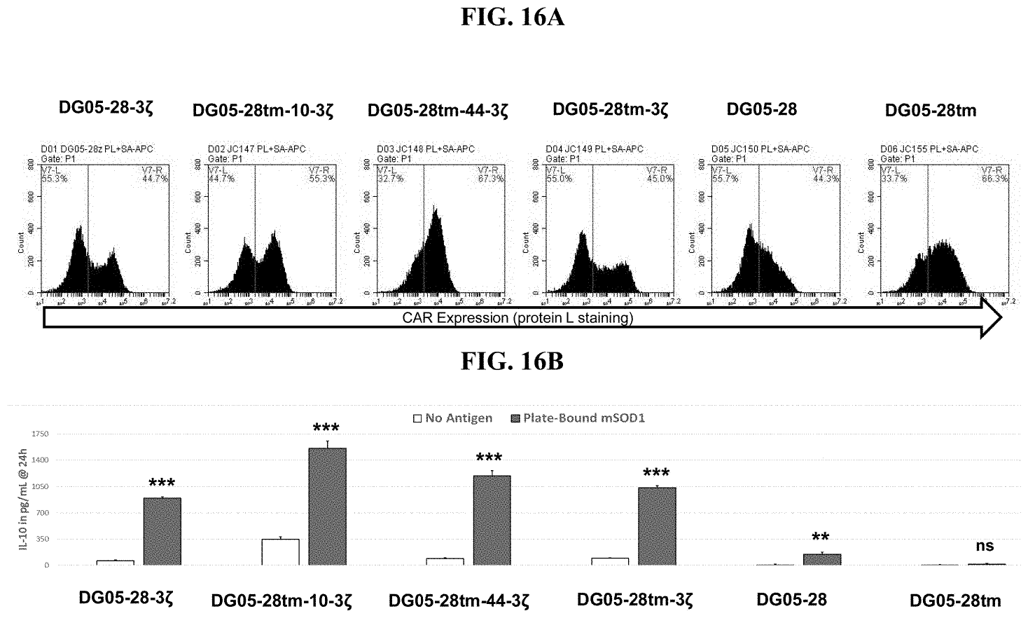

The flow cytometry assays of the present example proceeded as follows. The percentage of CAR-transduced Tregs was measured by direct labeling of the CAR scFv with biotinylated protein L (1 μg/mL; GenScript, Piscataway, N.J., USA) followed by streptavidin-PE (BioLegend, San Diego, Calif., USA). Cells were analyzed using an ACCURI™ C6 flow cytometer (BD Biosciences, Ann Arbor, Mich., USA). Cells stained with streptavidin-PE only were used to threshold for non-specific background signal (see FIG. 16A, red vertical line of the six flow cytometry panels).

Assays of the present example that comprised CAR Treg activation by plate-bound antigen were performed as follows. 96-well ELISA plates were coated with 50 μL/well of 10 μg/mL of purified mutSOD1 protein in PBS overnight at 4° C. Plates were rinsed three times with PBS and then blocked with 0.1 mL/well of X-VIVO™-15+10% human sera. Media was added to control wells that were not coated with mSOD1 protein. CAR Tregs were added at 50,000 cells/0.2 mL/well and incubated for 6 h. Cells were transferred to 96 well cell culture plate wells and incubated for another 18 h. Cell-free media was collected for human IL-10 ELISA (BioLegend, San Diego, Calif., USA).

Referring now to FIG. 16A, the flow cytometry results, which utilized protein L staining, demonstrated expression of each of the CAR constructs DG05-28-3ζ; DG05-28tm-10-3ζ; DG05-28tm-44-3ζ; DG05-28tm-3ζ; DG05-28; and DG05-28tm on human Tregs.

Referring now to FIG. 16B, DG05-28-3ζ; DG05-28tm-10-3ζ; DG05-28tm-44-3ζ; DG05-28tm-3ζ; DG05-28, but not DG05-28tm, produced IL-10 in response to mSOD1 antigen (ELISA). Regarding FIG. 16B, ns=not significant; **=p<0.01; ***=p<0.001 by student t-test (n=3).

### Example 9: Functional Activity of Modified Tregs Targeting Alzheimer's Disease

In the present example, expression and function of anti-Aβ CARs with different costimulatory signaling domains, and with or without the CD3-zeta (3ζ) stimulatory domain, were evaluated in ex vivo expanded and CAR transduced human Tregs. The anti-Aβ scFv DG03 and CD28 transmembrane (“28tm”) domain were in all constructs, and mouse truncated CD19 was also expressed with the CAR on the same construct. The CD3zeta (“3ζ”) was joined to co-stimulatory domains from CD28 (“28”), DAP10 (“10”), CD44 (“44”), 4-1BB (“BB”) to produce the following anti-Aβ CARs: DG03-28-3ζ (SEQ ID NO: 22); DG03-28tm-10-3ζ (SEQ ID NO: 45); DG03-28tm-44-3ζ (SEQ ID NO: 46); and DG03-28tm-BB-3ζ (SEQ ID NO: 47). Additionally, an anti-Aβ CAR comprising a CD28 transmembrane domain with no stimulatory or co-stimulatory domains was produced (DG03-28tm (SEQ ID NO: 50)); an anti-Aβ CAR comprising a CD28 transmembrane domain with a CD3ζ domain and without a costimulatory domain was produced (DG03-28tm-3ζ (SEQ ID NO: 48)); and an anti-Aβ CAR comprising a CD28 transmembrane domain and a CD28 costimulatory domain but without a CD3ζ domain was produced (DG03-28 (SEQ ID NO: 49)).

The modified Tregs of the present example were isolated, expanded, and transduced as generally described in Example 1. More specifically, Day 17 CAR Tregs were prepared from human PBMCs as described in Example 1. PG13 retrovirus with the following anti-Aβ CARs was used: DG03-28-3ζ (SEQ ID NO: 22), DG03-28tm-10-3ζ (SEQ ID NO: 45), DG03-28tm-44-3ζ (SEQ ID NO: 46), DG03-28tm-BB-3ζ (SEQ ID NO: 47), DG03-28tm-3ζ (SEQ ID NO: 48), DG03-28 (SEQ ID NO: 49), and DG03-28tm (SEQ ID NO: 50).

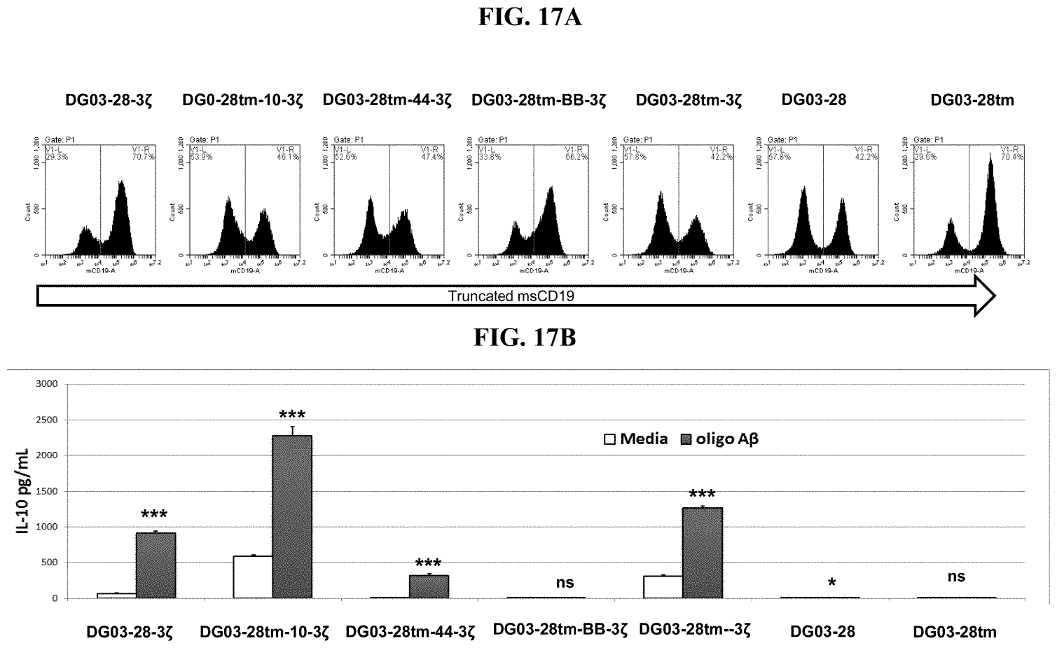

The flow cytometry assays of the present example proceeded as follows. The percentage of CAR-transduced Tregs was measured by indirect labeling of the co-expressed mouse truncated CD19 with PE anti-mouse CD19 (BioLegend, San Diego, Calif., USA). Cells were analyzed using an ACCURI™ C6 flow cytometer (BD Biosciences, Ann Arbor, Mich., USA). Non-stained cells were used to set the threshold for non-specific background signal (see FIG. 17A, red vertical line of the seven flow cytometry panels).

The oligomerized Aβ of the present example was prepared as follows. Aβ1-42 peptide in PBS at 200 μM was incubated for seven days at 37° C. in PBS. Oligomerized Aβ was diluted to 40 μM, aliquoted, and stored at −20° C.

Assays of the present example that comprised CAR Treg activation by plate-bound oligomerized Aβ proceeded as follows. CAR Tregs were plated in tissue culture-treated 96-well plates at 50,000 cells/0.2 mL/well in X-VIVO™-15+10% human sera with or without 100 nM Aβ and incubated for 24 h. Cell-free media was collected for human IL-10 ELISA (BioLegend, San Diego, Calif., USA).

Referring now to FIG. 17A, the flow cytometry results, which utilized msCD19 staining, demonstrated expression of each of the CAR constructs DG03-28-3ζ, DG03-28tm-10-3ζ; DG03-28tm-44-3ζ; DG03-28tm-BB-3ζ; DG03-28tm-3ζ; DG03-28, and DG03-28tm on human Tregs.

Referring now to FIG. 17B, DG03-28-3ζ; DG03-28tm-10-3ζ; DG03-28tm-44-3ζ; and DG03-28tm-CD3ζ, but not DG03-28tm-BB-3ζ; DG03-28; and DG03-28tm; produced IL-10 in response to plate-bound oligomerized Aβ antigen (ELISA). Regarding FIG. 17B, ns=not significant; *=p<0.05; ***=p<0.001 by student t-test (n=3).

### Example 10: Functional Activity of Modified Tregs Targeting ALS

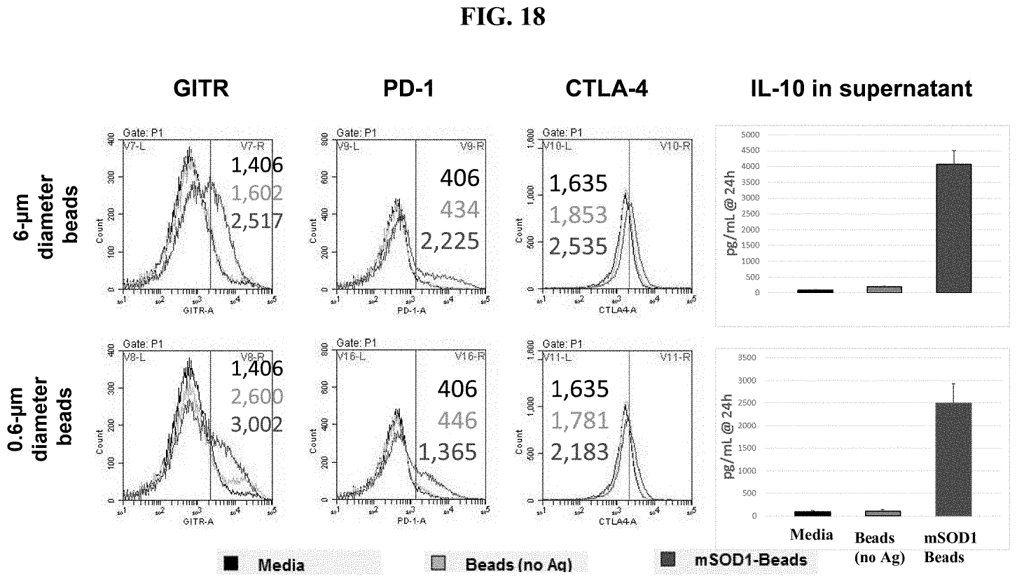

In the present example, the antigen-specific activity of anti-mutSOD1 CARs were evaluated in general accordance with the procedures described in Example 5. Ex vivo expanded and CAR transduced human Tregs engineered to express the anti-mSOD1 CAR DG05-28-3ζ (SEQ ID NO: 24) were co-cultured with 6.0-μm or 0.6-μm diameter polystyrene beads (used to mimic aggregates of mSOD1 that develop in CNS of ALS patients) pre-coated with mSOD1, and the effects on cell surface expression of GITR, PD-1, and CTLA-4 were monitored (see FIG. 18). Additionally, production of IL-10 was evaluated by performing an ELISA assay (see FIG. 18). The anti-mSOD1 CAR targeting ALS included modified Tregs comprising DG05-28-CD3ζ (SEQ ID NO: 24).

Modified Treg isolation, expansion, and CAR transduction proceeded as generally described in Example 4. More specifically, CAR Tregs were prepared from human PBMCs as described in Example 4, and PG13 retrovirus with the anti-mSOD1 CAR DG05-28-3ζ was used for transduction on Days 10 and 11.

CAR Treg cryopreservation and recovery after thawing proceeded as follows for the present example. Day 16 CAR Tregs were cryopreserved in cryovials at 20×10⁶ cells/mL in solution of 90% heat-inactivated FBS and 10% DMSO in MR. FROSTY™ Freezing Container at −80° C. for 24 h and then transferred to liquid nitrogen. Cells were thawed and rinsed in PBS, resuspended to 2×10⁶ cells/mL in X-VIVO™-15+10% human sera+500 u/mL IL-2, and incubated for 24 h. Cells were centrifuged to pellet cells, resuspended in 4 mL X-VIVO™-15 media and centrifuged over 4 mL of LYMPHOPREP™ separation media at 800×g for 20 min. Cells at the interphase were collected and rinsed in X-VIVO™-15 and pelleted. Cells were resuspended to 1×10⁶ cells/mL in X-VIVO™-15+10% human sera (no IL-2).

Preparation of antigen-coated polystyrene beads for use with the bead-based assays of the present example proceeded as follows. Purified biotinylated, His-tagged human mutant G93A SOD1 protein (mSOD1) was provided as a gift by Dr. Roos (University of Chicago Medical Center, Chicago, Ill., USA). SPHERO™ Streptavidin Coated Particles 6 μm-diameter polystyrene (Spherotech cat. #SVP-60-5, Lake Forest, Ill., USA) and SPHERO™ Rabbit anti-6×His Coated Particles 0.6 μm-diameter polystyrene (Spherotech cat. #HISP-05-2, Lake Forest, Ill., USA) were rinsed in PBS with sterile 1% heat-inactivated FBS. Beads were pelleted by microcentrifugation at 12,000×g for 2 min. Beads were resuspended in 1% FBS with or without 2 μg/mL mSOD1 and incubated at RT for 1.5 h. Cells were pelleted, rinsed in 1% FBS, and resuspended in X-VIVO™-15+10% human sera.

The bead-based assays of the present example that comprised CAR Treg activation by bead-bound antigen proceeded as follows. CAR Tregs were plated in tissue culture-treated 12-well plates at 1.6×10⁶ cells/0.8 mL/well in X-VIVO™-15+10% human sera. The non-coated and mSOD1-coated 6 μm-diameter polystyrene beads were added at bead:cell ratio of 4:1. The non-coated and mSOD1-coated 0.6 μm-diameter polystyrene beads were added at a bead:cell ratio of 100:1. No beads were added to some wells as media-only controls. After 24 h incubation cell-free media was collected for human IL-10 ELISA (BioLegend, San Diego, Calif., USA). Cells were collected for flow cytometry to assess cell surface markers. Cells were labeled with APC anti-human GITR (clone 621; BioLegend, San Diego, Calif., USA), APC anti-human PD-1 (clone EH12.2H7; BioLegend, San Diego, Calif., USA), and PE anti-human CTLA-4 (clone BNI3; BioLegend, San Diego, Calif., USA). Mean fluorescence intensity (MFI) was measured and histograms prepared using an ACCURI™ C6 flow cytometer (BD Biosciences, Ann Arbor, Mich., USA). Non-stained cells were used to set the threshold for non-specific background signal (see FIG. 18, vertical line of each of the six flow cytometry plots).

Referring now to FIG. 18, the results demonstrated that the modified Tregs co-cultured with 6.0 um or 0.6 um beads pre-coated with mSOD1 increased cell surface expression of GITR, PD-1, and CTLA-4 (flow cytometry after 24 h; mean MFI shown) (see FIG. 18, six flow cytometry plots).

Moreover, the IL-10 ELISA assay of the present example further demonstrated the responsiveness of the modified human Tregs to mSOD1, as the modified human Tregs responded to the mSOD1 coated beads as demonstrated by the increase in IL-10 production relative to beads not coated with antigen or to media alone (see FIG. 18, two IL-10 in supernatant graphs).

### Example 11: Functional Activity of Modified Tregs Targeting ALS

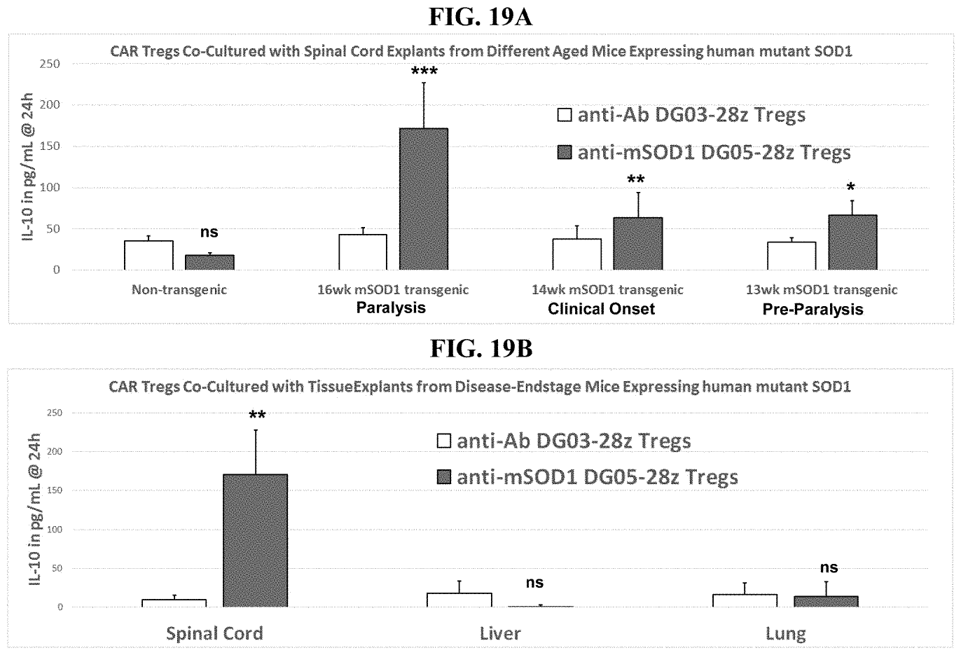

In the present example, the functional activity of modified Tregs targeting ALS was evaluated by co-culturing modified Tregs with spinal cord tissue explants derived from transgenic mice expressing human mSOD1. Additionally, the functional activity of modified Tregs targeting ALS was evaluated by co-culturing modified Tregs with spinal, liver, or lung tissue explants derived from transgenic mice expressing human mSOD1. The modified Tregs included modified Tregs comprising DG05-CD28-CD3ζ (referred to as DG05-28z in FIG. 19A and FIG. 19B) (SEQ ID NO: 24), and, as a negative control, modified Tregs comprising an anti-Aβ CAR (referred to as DG03-28z in FIG. 19A and FIG. 19B) (DG03-CD28-CD3ζ (SEQ ID NO: 22)).

Modified Treg isolation, expansion, and CAR transduction proceeded as generally described in Example 4. More specifically, CAR Tregs were prepared from human PBMCs as described in Example 4. PG13 retrovirus with the anti-mSOD1 CAR DG05-CD28-CD3ζ or anti-Aβ CAR DG03-CD28-CD3ζ was used for transductions on Days 10 and 11.

CAR Treg cryopreservation and recovery after thaw proceeded as follows. Day 16 CAR Tregs were cryopreserved in cryovials at 20×10⁶ cells/mL in solution of 90% heat-inactivated FBS and 10% DMSO in Mr. FROSTY™ Freezing Container at −80° C. for 24 h and then transferred to liquid nitrogen. Cells were thawed and rinsed in PBS, resuspended to 2×10⁶ cells/mL in X-VIVO™-15+10% human sera+500 u/mL IL-2, and incubated for 24 h. Cells were centrifuged to pellet cells, resuspended in 4 mL X-VIVO™-15 media and centrifuged over 4 mL of LYMPHOPREP™ separation media at 800×g for 20 min. Cells at the interphase were collected and rinsed in X-VIVO™-15 and pelleted. Cells were resuspended to 1×10⁶ cells/mL in X-VIVO™-15+10% human sera (no IL-2).

CAR Treg activation by mouse tissue explants proceeded as follows for the present example. B6SJL.SOD1-G93A (stock #002726, The Jackson Laboratory, Bar Harbor, Me., USA) and non-transgenic littermates were used in the present example. The mSOD1 transgenic mice were monitored for weight loss and limb paralysis. Spinal cord tissues were collected from non-transgenic mice or mSOD1 transgenic mice at different stages of disease development: 13 weeks (pre-paralysis), 14 weeks (clinical onset), 16 weeks (paralysis), or 18 weeks (disease end-stage weeks defined as 15% weight loss and hind-limb paralysis). Liver and lung were also collected from mSOD1 transgenic mice at disease end-stage. Tissues were transferred to ice-cold X-VIVO™-15 media, cut into 2 mm×2 mm pieces, placed in wells of 96-well ELISA plate without media for 5 min., then CAR Tregs were added at 50,000 cells/well in 0.2 mL of X-VIVO™-15+10% human sera (no IL-2). After 24 h incubation cell-free media was collected for human IL-10 ELISA (BioLegend, San Diego, Calif., USA).

Referring now to FIG. 19A, DG05-CD28-CD3ζ, which targeted mSOD1, stimulated production of IL10, whereas Tregs expressing the negative control CAR (DG03-CD28-CD3ζ) did not stimulate production of IL-10 when co-cultured with spinal cord explants (ns=not significant; *=p<0.05; **=p<0.01; ***=p<0.001 by student t-test (n=8 for FIG. 19A)).

Referring now to FIG. 19B, DG05-CD28-CD3ζ, which targeted mSOD1, stimulated production of IL10 when co-cultured with spinal cord tissue, but not when co-cultured with liver or lung tissue, whereas Tregs expressing the negative control CAR (DG03-CD28-CD3ζ) did not stimulate production of IL-10 when co-cultured with any of spinal cord, liver, or lung tissue (ns=not significant; *=p<0.05; **=p<0.01; ***=p<0.001 by student t-test (n=3 for FIG. 19B)).

### Example 12: Functional Activity of Modified Tregs Targeting Alzheimer's Disease

In the present example, the functional activity of modified Tregs targeting Alzheimer's disease was evaluated by exposing said modified Tregs to oligomerized Aβ and monitoring mRNA and protein secretion levels of IL-10 and IL-4. The modified Tregs included modified Tregs comprising the anti-Aβ CAR DG03-CD28-CD3ζ (SEQ ID NO: 22).

Modified Treg isolation, expansion, and CAR transduction proceeded as generally described in Example 1. More specifically, Day 17 CAR Tregs were prepared from human PBMCs as described in Example 1. PG13 retrovirus with the anti-Aβ CAR DG03-CD28-CD3ζ was used for transductions on Days 10 and 11.

Preparation of oligomerized Aβ for use with the present example proceeded as follows. Aβ1-42 peptide in PBS at 200 μM was incubated for seven days at 37° C. in PBS. Oligomerized Aβ was diluted to 40 μM, aliquoted, and stored at −20° C.

CAR Treg activation by oligomerized Aβ proceeded as follows for the present example. CAR Tregs were plated in tissue culture-treated 96-well plates at 50,000 cells/0.2 mL/well in X-VIVO™-15+10% human sera with or without 100 nM Aβ and incubated for 24 h. Cell-free media was collected for human IL-4 and human IL-10 ELISAs (BioLegend, San Diego, Calif., USA).

Qualitative RT-PCR proceeded as follows for the present example. CAR Tregs were plated in tissue culture-treated 12-well plates at 1.0×10⁶ cells/1.0 mL/well in X-VIVO™-15+10% human sera and incubated overnight. Oligomerized Aβ (100 nM) was added the next day with some wells left untreated (no antigen). Cells were collected 7.5 h after addition of oligomerized Aβ to the cells, which were then pelleted, and then the RNA extracted using TRIzol Reagent (Invitrogen, ThermoFisher Scientific, Waltham, Mass., USA). Eluted RNA was quantified by spectrophotometry and 1 ug was reverse-transcribed using QSCRIPT™ cDNA SuperMix (Quanta Biosciences, Beverly, Mass., USA). Reaction mixtures contained 2 ng cDNA, 200 nM dNTPs, 400 nM primers, 1× Standard Taq Buffer (New England BioLabs), and 0.625 U of TAQ polymerase (BioLabs) in a total reaction volume of 25 μl. The sequences of the primer used were as follows: human β-actin (101-bp product): forward, 5′-GGC CGA GGA CTT TGA TTG C-3′ (SEQ ID NO: 255); reverse, 5′-TGG GGT GGC TTT TAG GAT GG-3′ (SEQ ID NO: 256); human IL-4 (148-bp product): forward, 5′-GCT TCC CCC TCT GTT CTT CC-3′ (SEQ ID NO: 257); reverse, 5′-GAT GTC TGT TAC GGT CAA CTC G-3′ (SEQ ID NO: 258); and human IL-10 (82-bp product): forward, 5′-TCA AGG CGC ATG TGA ACT CC-3′ (SEQ ID NO: 259); reverse, 5′-CAG GGA AGA AAT CGA TGA CAG C-3′ (SEQ ID NO: 260). Samples were placed in a 2720 Thermal Cycler (Applied BioSystems, Foster City, Calif., USA) at 95° C. for 2 min followed sequentially by a cyclic phase at 95° C. for 30 s, 60° C. for 30 s, and then 68° C. for 35 s for 35 cycles. Amplification products were electrophoresed on a 1.5% agarose gel containing 1×SYBR™ Safe DNA Gel Stain (Invitrogen, Thermo Fisher Scientific, Waltham, Mass., USA) at 105 V for 35 min. Bands were visualized and imaged at 302 nm using Alpha Imager EP gel documentation system (Alpha Innotech, San Leandro, Calif., USA).

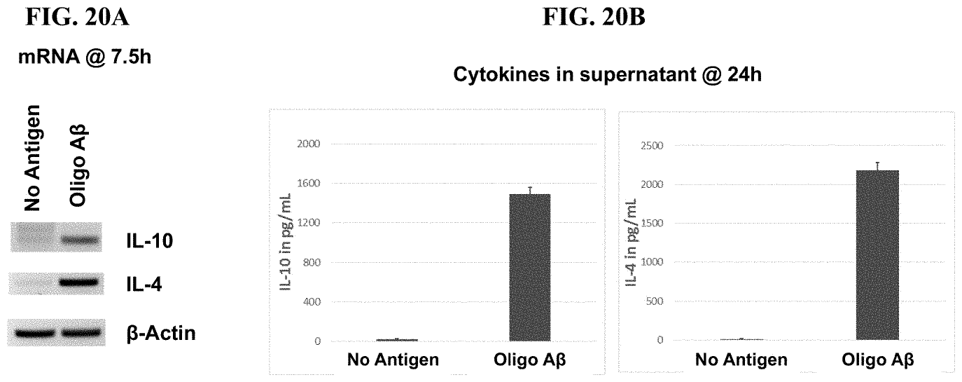

Referring now to FIG. 20A, the modified Tregs exposed to oligomerized demonstrated an increase in IL-10 and IL-4 mRNA levels as compared to the modified Tregs which were not exposed to oligomerized Aβ as monitored by RT-PCR gel staining. β-actin staining served as a control (see FIG. 20A).

Referring now to FIG. 20B, the modified Tregs exposed to oligomerized demonstrated an increase in IL-10 and IL-4 production as compared to modified Tregs which were not exposed to oligomerized Aβ as monitored by ELISA.

### Example 13: Functional Activity of Modified Tregs Targeting ALS

In the present example, the antigen-specific anti-inflammatory activity of anti-mutSOD1 CARs was evaluated in the assays described below. The modified Tregs included modified Tregs comprising anti-mSOD1 CAR DG05-CD28-CD3ζ (SEQ ID NO: 24).

Modified Treg isolation, expansion, and CAR transduction proceeded as generally described in Example 4. More specifically, CAR Tregs were prepared from human PBMCs as described in Example 4. PG13 retrovirus with the anti-mSOD1 CAR DG05-CD28-CD3ζ was used for transduction on Days 10 and 11.

CAR Treg cryopreservation and recovery after thaw proceeded as follows for the present example. Day 16 CAR Tregs were cryopreserved in a cryovials at 20×10⁶ cells/mL in solution of 90% heat-inactivated FBS and 10% DMSO in Mr. FROSTY™ Freezing Container at −80° C. for 24 h and then transferred to liquid nitrogen. Cells were thawed and rinsed in PBS, resuspended to 2×10⁶ cells/mL in X-VIVO™-15+10% human sera+500 u/mL IL-2, and incubated for 24 h. Cells were centrifuged to pellet cells, resuspended in 4 mL X-VIVO™-15 media and centrifuged over 4 mL of LYMPHOPREP™ separation media at 800×g for 20 min. Cells at the interphase were collected and rinsed in X-VIVO™-15 and pelleted. Cells were resuspended to 1×10⁶ cells/mL in X-VIVO™-15+10% human sera (no IL-2).

Pre-activation of the CAR Tregs of the present example proceeded as follows. 96-well ELISA plates were coated with 50 μL/well of 10 μg/mL of purified mutSOD1 protein or 5 ug/mL OKT3 antibody (anti-CD3) in PBS overnight at 4° C. Control wells for non-activated CAR Tregs were left uncoated. Plates were rinsed three times with PBS and then blocked with 0.1 mL/well of X-VIVO™-15+10% human sera. CAR Tregs were added at 100,000 cells/0.2 mL/well and incubated for 6 h. Cells were then collected and diluted to 500,000 CAR Tregs/mL in X-VIVO™-15+10% human sera.

Co-culture of the pre-activated CAR Tregs and monocyte/macrophages of the present example proceeded as follows. Monocytes were isolated by CD14-negative selection (StemCell 19359) from human PBMCs (different donor from CAR Tregs) and incubated for 6 h at 50,000 cells/0.1 mL/well in a 96-well tissue culture-treated white luminometer plate (CORNING® COSTAR® 3719). Non-Activated, mSOD1 pre-activated, or anti-CD3 pre-activated CAR Tregs were added at 40,000 cells/0.1 mL/well. Co-cultured cells were incubated in a final volume of 0.2 mL/well for 2 days.

Stimulation of the CAR Treg/macrophage co-cultures of the present example proceeded as follows. After 2 days of incubation, CAR Treg/macrophage co-cultures were stimulated by adding either phorbol myristate acetate (PMA; 40 nM), zymosan particles (20 μg/mL), or lipopolysaccharide (LPS; 20 ng/mL). To measure superoxide generation, lucigenin (5 μM) was added with PMA or zymosan and lucigenin-mediated bioluminescence was measured on a Centro LB 960 Microplate Luminometer (2 s exposure) at 7 timepoints over 6 h. To measure TNF-α by ELISA (BioLegend, San Diego, Calif., USA), cell-free media was collected 6 h after adding LPS.

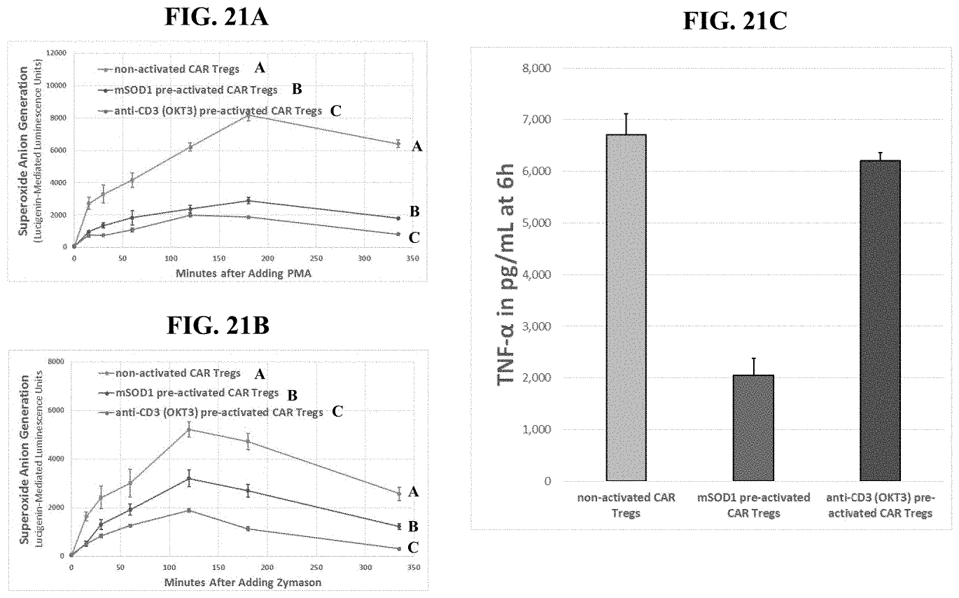

Referring now to FIG. 21A, modified Tregs comprising DG05-CD28-CD3ζ which were pre-stimulated with either mSOD1 or anti-CD3 antibody demonstrated inhibition of PMA-stimulated superoxide generation (as measured by Lucigenin-mediated luminescence). Regarding FIG. 21A, ns=not significant; **=p<0.01 relative to non-activated CAR Tregs by Dunnett's multiple comparison test (n=3).

Referring now to FIG. 21B, modified Tregs comprising DG05-CD28-CD3ζ which were pre-stimulated with either mSOD1 or anti-CD3 antibody demonstrated inhibition of zymason-stimulated superoxide generation (as measured by Lucigenin-mediated luminescence). Regarding FIG. 21B, ns=not significant; **=p<0.01 relative to non-activated CAR Tregs by Dunnett's multiple comparison test (n=3).

Referring now to FIG. 21C, modified Tregs comprising DG05-CD28-CD3ζ which were pre-stimulated with mSOD1 demonstrated inhibition of LPS-stimulated TNF-α production as measured by ELISA at 24 h, whereas modified Tregs comprising DG05-CD28-CD3ζ which were pre-stimulated with anti-CD3 antibody did not demonstrate inhibition of LPS-stimulated TNF-α production. Regarding FIG. 21C, ns=not significant; **=p<0.01 relative to non-activated CAR Tregs by Dunnett's multiple comparison test (n=3).

### Example 14: Functional Activity of Modified Tregs Targeting Alzheimer's Disease

In the present example, the antigen-specific anti-inflammatory activity of anti-Aβ CARS was evaluated in the assays described below. The modified Tregs included modified Tregs comprising anti-Aβ. CAR DG03-CD28-CD3ζ (SEQ ID NO: 22).

Modified Treg isolation, expansion, and CAR transduction proceeded as generally described in Example 1. More specifically, CAR Tregs were prepared from human PBMCs as described in Example 1. PG13 retrovirus with the anti-Aβ. CAR DG03-CD28-CD3ζ was used for transduction on Days 10 and 11.

Modified CAR Treg cryopreservation and recovery after thaw proceeded as follows for the present example. Day 16 CAR Tregs were cryopreserved in a cryovials at 20×10⁶ cells/mL in solution of 90% heat-inactivated FBS and 10% DMSO in Mr. FROSTY™ Freezing Container at −80° C. for 24 h and then transferred to liquid nitrogen. Cells were thawed and rinsed in PBS, resuspended to 2×10⁶ cells/mL in X-VIVO™-15+10% human sera+500 u/mL IL-2, and incubated for 24 h. Cells were centrifuged to pellet cells, resuspended in 4 mL X-VIVO™-15 media and centrifuged over 4 mL of LYMPHOPREP™ separation media at 800×g for 20 min. Cells at the interphase were collected and rinsed in X-VIVO™-15 and pelleted. Cells were resuspended to 1×10⁶ cells/mL in X-VIVO™-15+10% human sera (no IL-2).

Pre-activation of the modified CAR Tregs of the present example proceeded as follows. 96-well ELISA plates were coated with 50 μL/well of 3 μg/mL of Aβ1-42 peptide or 5 ug/mL OKT3 antibody (anti-CD3) in PBS overnight at 4° C. Control wells for non-activated CAR Tregs were left uncoated. Plates were rinsed three times with PBS and then blocked with 0.1 mL/well of X-VIVO™-15+10% human sera. CAR Tregs were added at 100,000 cells/0.2 mL/well and incubated for 6 h. Cells were then collected and diluted to 500,000 CAR Tregs/mL in X-VIVO™-15+10% human sera.

Co-culture of the pre-activated modified CAR Tregs and monocytes/macrophages of the present example proceeded as follows. Monocytes were isolated by CD14-negative selection (BioLegend 480060, San Diego, Calif., USA) from human PBMCs (different donor from CAR Tregs) and incubated for 6 h at 50,000 cells/0.1 mL/well in a 96-well tissue culture-treated white luminometer plate (CORNING® COSTAR® 3719). Non-activated, Aβ pre-activated, or anti-CD3 pre-activated CAR Tregs were added at 50,000 cells/0.1 mL/well. Co-cultured cells were incubated in a final volume of 0.2 mL/well for 2 days.

Stimulation of the modified CAR Treg/macrophage co-cultures of the present example proceeded as follows. After 2 days of incubation, CAR Treg/macrophage co-cultures were stimulated by adding either phorbol myristate acetate (PMA; 40 nM), zymosan particles (20 μg/mL), or lipopolysaccharide (LPS; 20 ng/mL). To measure superoxide generation, lucigenin (5 μM) was added with PMA or zymosan and lucigenin-mediated bioluminescence was measured on a Centro LB 960 Microplate Luminometer (2 s exposure) at 6 timepoints over 6 h. To measure IL-6 by ELISA (BioLegend, San Diego, Calif., USA), cell-free media was collected 24 h after adding LPS.

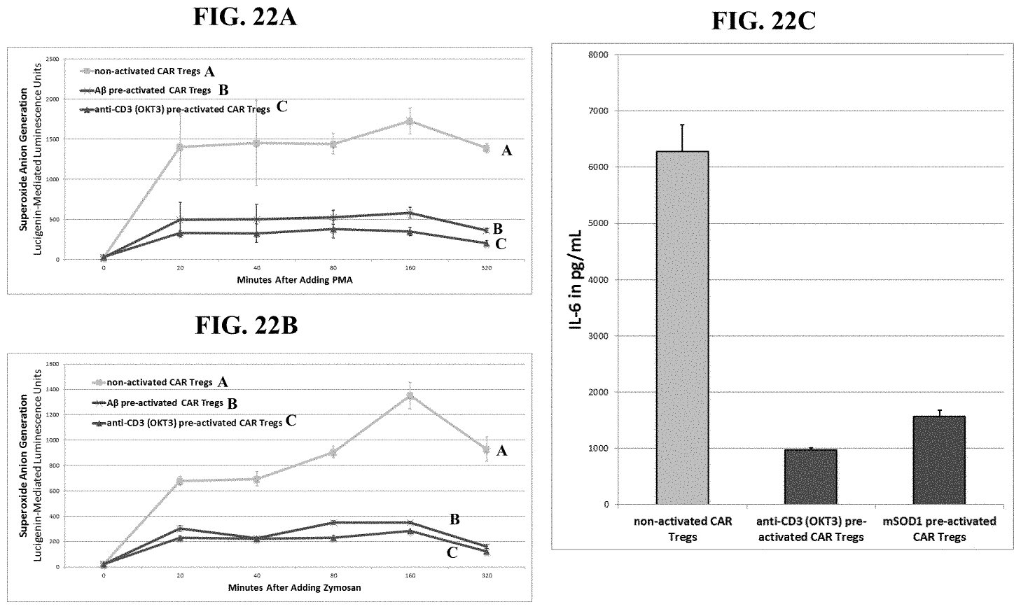

Referring now to FIG. 22A, modified Tregs comprising DG03-CD28-CD3ζ which were pre-stimulated with either plate bound Aβ antigen or with anti-CD3 antibody inhibited PMA stimulated superoxide generation (as measured by Lucigenin-mediated luminescence). Regarding FIG. 22A, **=p<0.01 relative to non-activated CAR Tregs by Dunnett's multiple comparison test (n=3).

Referring now to FIG. 22B, modified Tregs comprising DG03-CD28-CD3ζ which were pre-stimulated with either plate-bound Aβ antigen or with anti-CD3 antibody inhibited Zymosan-stimulated superoxide generation (as measured by Lucigenin-mediated luminescence). Regarding FIG. 22B, **=p<0.01 relative to non-activated CAR Tregs by Dunnett's multiple comparison test (n=3).

Referring now to FIG. 22C, modified Tregs comprising DG03-CD28-CD3ζ which were pre-stimulated with either plate-bound Aβ antigen or with anti-CD3 antibody inhibited LPS-stimulated IL-6 production as measured by ELISA at 24 h. Regarding FIG. 22C, **=p<0.01 relative to non-activated CAR Tregs by Dunnett's multiple comparison test (n=3).

### Example 15: Functional Activity of Neurodegenerative Disease-Modifying Molecule (NDMM) Tregs Engineered to Express Anti-Oxidants or Growth Factors

In the present example, the cytoprotective activity of neurodegenerative disease-modifying molecules (NDMMs) expressed in human Tregs was evaluated.

Isolation, expansion, and transduction of the NDMM-Tregs of the present example proceeded as follows. NDMM-engineered Tregs were prepared from human PBMCs as described in Example 1; Example 4; and Example 6. Instead of CAR constructs, the PG13 retrovirus used for Treg transduction on Days 10 and 11 were with NDMM constructs for Nrf2 (Keap1 inhibitor peptide) (SEQ ID NO: 51), human catalase (SEQ ID NO: 52), brain derived neurotrophic factor (BDNF) (SEQ ID NO: 53), and insulin growth factor-1 (IGF-1) (SEQ ID NO: 54). The NDMM constructs co-expressed truncated mouse CD19 to monitor transduction efficiency. Mock-transduced Tregs (no PG13 retrovirus) were used as controls.

Flow cytometry of the NDMM Tregs of the present example proceeded as follows. The percentage of NDMM-transduced Tregs was measured by indirect labeling of the co-expressed mouse truncated CD19 with PE anti-mouse CD19 (BioLegend, San Diego, Calif., USA). Cells were analyzed using an ACCURI™ C6 flow cytometer (BD Biosciences, Ann Arbor, Mich., USA). Non-stained cells were used to set the threshold for non-specific background signal.

Cryopreservation and recovery after thaw of the NDMM Tregs of the present example proceeded as follows. Day 17 CAR Tregs were cryopreserved in a cryovials at 20×10⁶ cells/mL in solution of 90% heat-inactivated FBS and 10% DMSO in Mr. FROSTY™ Freezing Container at −80° C. for 24 h and then transferred to liquid nitrogen. Cells were thawed and rinsed in PBS, resuspended to 2×10⁶ cells/mL in X-VIVO™-15+10% human sera+500 u/mL IL-2, and incubated for 24 h. Cells were centrifuged to pellet cells, resuspended in 4 mL X-VIVO™-15 media and centrifuged over 4 mL of LYMPHOPREP separation media at 800×g for 20 min. Cells at the interphase were collected and rinsed in X-VIVO™-15 and pelleted. Cells were resuspended to 2×10⁶ cells/mL in incomplete Dulbecco's High Glucose Modified Eagles Medium (DMEM; Hyclone 30022) supplemented with 10% heat-inactivated FBS, MEM nonessential amino acid solution, HEPES (10 mM), and penicillin/streptomycin solution.

Co-culture of NDMM-Tregs and luciferase-expressing SH-SYSY neuronal cells of the present example proceed as follows. The human neurobalstoma cell line SH-SYSY, previously transduced to express luciferase (Luc), was grown to 60%-90% confluency in complete DMEM supplemented with 10% heat-inactivated FBS, MEM nonessential amino acid solution, HEPES (10 mM), sodium pyruvate (1 mM), 2-merceptoethanol (50 μM), and penicillin/streptomycin solution. Luc-SH-SYSY cells were collected by trypsinization, pelleted by centrifugation at 500×g for 5 min, resuspended in incomplete DMEM (without sodium pyruvate and 2-merceptoethanol), and plated at 10,000 cells/0.1 mL/well in 96-well tissue culture-treated white luminometer plate (CORNING® COSTAR® 3719). After 3 h incubation, NDMM- or mock-Tregs in incomplete DMEM media were added to Luc-SH-SYSY cells at 100,000 Tregs/0.05 mL/well (final volume of 0.15 mL/well). Co-cultured cells were incubated for 24 h.

The hydrogen peroxide toxicity assay of the present example was performed as follows. After 24 h of incubation of NDMM-Treg/luc-SH-SYSY co-cultures, 50 μL of incomplete DMEM media with final concentrations of hydrogen peroxide (H₂O₂) at 0, 20, 40, 60, 80, and 320 μM was added. H₂O₂-exposed co-cultures were incubated for 24 h and then the relative number of surviving luc-SH-SYSY neuronal cells was measured by bioluminescence. Luciferin (50 μg/mL) was added to each well, incubated for 30 min, and bioluminescence was measured on a Centro LB 960 Microplate Luminometer (2 s exposure).

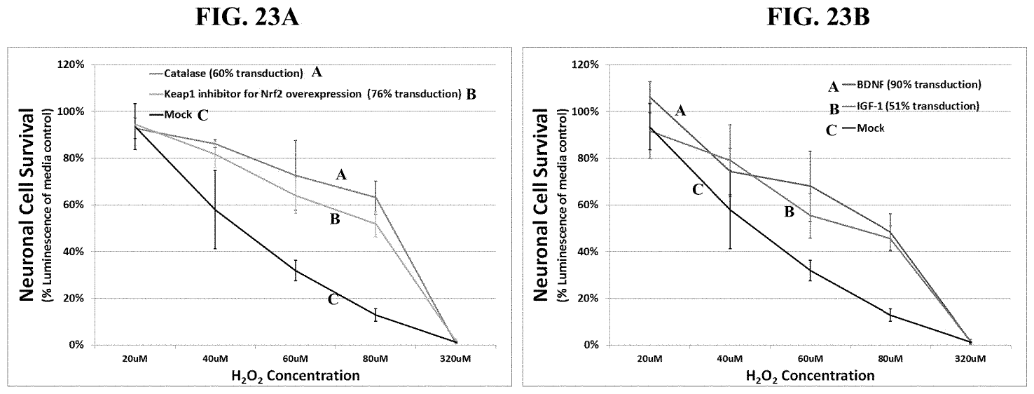

Referring now to FIG. 23A, ex vivo expanded and transduced human Tregs engineered to express antioxidants (Keap1 inhibitor peptide or Catalase) protected human SH-SYSY neuronal cells (luciferase-expressing) from hydrogen peroxide toxicity. Regarding FIG. 23A, ns=not significant; *=p<0.05; **=p<0.01 relative to mock-transduced Tregs by Dunnett's multiple comparison test (n=3).

Referring now to FIG. 23B, ex vivo expanded and transduced human Tregs engineered to express growth factors (BDNF or IGF-1) protected human SH-SYSY neuronal cells (luciferase-expressing) from hydrogen peroxide toxicity. Regarding FIG. 23B, ns=not significant; *=p<0.05; **=p<0.01 relative to mock-transduced Tregs by Dunnett's multiple comparison test (n=3).

### Example 16: Evaluation of Human Anti-mSOD1 CAR Tregs in an ALS Mouse Model

To evaluate human anti-mSOD1 CAR Tregs in an ALS mouse model, the development of a transgenic ALS mouse strain that does not reject transplanted xenogeneic cells was required. Human SOD1*G93A (mSOD1) mice (stock #004435) and NOD-scid IL2Rgamma^{null} (NSG) mice, which lacking T, B, NK cells, were purchased from The Jackson Laboratory and maintained at Dartmouth's CMR animal facility. Male B6-mSOD1 mice were mated with female NSG mice. The heterozygous mSOD1 mice, confirmed by genotyping for human SOD1, of the F1 generation carrying the X-chromosomal IL2Rgamma^{null} mutation were used for the next backcrossing to female NSG mice. This 1^{st} back-crossed generation (BC1) produced IL2Rgamma^{null} mice with some being scid and mSOD1-positive (mSOD1.NSG) mice. The SCID/mSOD1 positive mice were confirmed by the lack of mouse CD4 cells in blood as determined by flow cytometry and genotyping for human SOD1. These mSOD1.NSG BC1 mice were used for experiments and to breed with female NSG mice to generate the 2^{nd} back-crossed generation (BC2), also used for experimentation. We anticipate that the mSOD1 Tg mouse model backcrossed onto NSG mice a will result in mice which develop the illness.

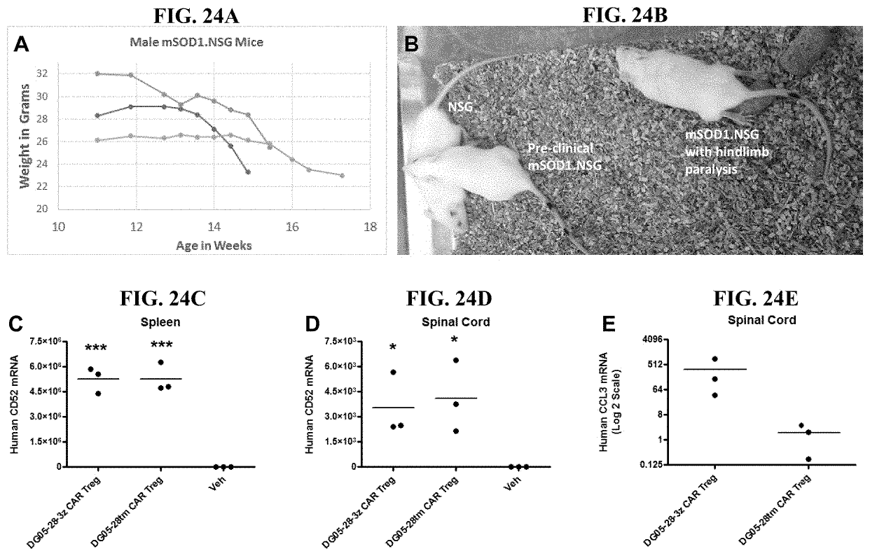

Development of motor neuron disease, which occurred in all mSOD1.NSG mice to date, was monitored by loss of weight, loss of paw grip strength, and by observation of limb paralysis (FIGS. 24 A and 24B).

CD4+CD25+ Tregs were isolated from human PBMCs in a two-step cell isolation process. First, human CD4+ cells were isolated by negative-selection using MOJOSORT™ Human CD4 T Cell Isolation Kit (Biolegend) and an EASYSEP™ Magnet (StemCell) according to manufacturer's instructions. Second, CD25hi cells were enriched from the CD4+-isolated cells by positive-selection using anti-human CD25 MicroBeads II (Miltenyi) and MS Columns with MiniMACS™ Separator magnet (Miltenyi). The CD4+CD25hi cells were cultured in 24-well non-tissue culture plates at 1×10⁶ cells/mL in Treg growth medium (X-Vivo-15 supplemented with 10% heat-inactivated human AB serum (Sigma)). Cell incubation condition was humidified 37° C. and 5% CO₂. Cells were stimulated with 25 μL/1×10⁶ cells of ImmunoCult™ Human CD3/CD28 T Cell Activator (StemCell) on days 0 and 9 in culture. Treg growth media was supplemented with 500 U/mL human IL-2 (Tecin from Roche, kindly provided by the NIH) starting on culture day 2. Cultured cells were transferred to 25 cm² tissue culture-treated flasks on day 5. Cells were transduced over two days with retroviral CAR constructs DG05-28-3z or DG05-28tm on day 10 and day 11 of culture. 24-well non-tissue culture plate wells were pre-coated with RetroNectin® (Takara Bio USA, Inc.) according to manufacturer's instructions and then day 10 cultured cells were added at 0.3×10⁶ cells/well in 0.3 mL of Treg growth media. Retroviral supernatant was added at 0.7 mL/well and plates were centrifuged at 1500 rcf at 30° C. for 1 h, and then incubated overnight. On the next day, 0.5 mL culture supernatant was replaced with 0.5 mL retroviral supernatant with 500 U/mL IL-2 and cells were re-centrifuged and then incubated overnight. The next day cells were transferred to 25 cm² tissue culture-treated flasks at 1×10⁶ cells/mL. On day 13 of culture, a sample of cells was validated for transduction efficiency by measuring the percentage of the co-transduced truncated CD19 on cells by flow cytometry. Cells were cultured with fresh Treg growth media with fresh IL-2 added every two days until day 17. DG05-28-3z or DG05-28tm CAR Tregs were rinsed three times and resuspended in X-VIVO 15 without serum to 25×10⁶ cell/mL and administered at 0.4 mL/mouse by tail vein intravenous injection into 11-12-week-old preclinical mSOD1.NSG mice. Three mice were injected with 10×10⁶ DG05-28-3z CAR Tregs, three mice were injected with 10×10⁶ DG05-28tm CAR Tregs, and three mice were injected with vehicle. Two days after injection, mice with euthanized by CO₂ inhalation and tissues harvested.

Freshly isolated spleens and spinal cords were immediately incubated in RNAlater solution (Qiagen) for 1 day at 4° C. and then stored at −80° C. before RNA was extracted. RNA was extracted using TRIzol Reagent (Invitrogen, ThermoFisher Scientific, Waltham, Mass., USA). Eluted RNA was quantified by spectrophotometry and 1 ug was reverse-transcribed using QSCRIPT™ cDNA SuperMix (Quanta Biosciences, Beverly, Mass., USA). Quantitative real-time PCR was performed using PerfeCTa SYBR Green FastMix (Quanta Biosciences), 20-80 ng sample cDNA, and 300 nM of a RT-PCR primer set (IDT, San Jose, Calif.). The sequences of the primer used were as follows: mouse β-actin (141-bp product): forward, 5′-GGC TGT ATT CCC CTC CAT C-3; reverse, 5′-ATG CCA TGT TCA ATG GGG TA-3; human CD52 (124 bp product): forward, 5′-AGC CCT GAG ATC ACC TAA A-3; reverse, 5′-GAG TCC AGT TTG TAT CTG TAC C-3; human CCL3 (151-bp product): forward, 5′-AGT TCT CTG CAT CAC TTG CTG-3; reverse, 5′-CGG CTT CGC TTG GTT AGG AA-3′. CFX96 Touch Real-Time PCR Detection System (Bio-Rad) was used with the following parameters: initial denaturation (95° C./2 min) was followed by cycles of denaturation (95° C./2 s) and primer annealing (60° C./30 s). A melt curve was performed on all samples for quality control. The relative quantity of gene expression was analyzed by the 2-(ΔΔCt) method with normalization to the reference gene mouse β-actin or human CD52.

Injected human Tregs were detected in the spleens two days after injection by real time qPCR detection of human CD52 mRNA (FIG. 24 C). Human CD52 mRNA was also detected in the targeted spinal cords (FIG. 24 D). CCL3 mRNA and protein expression is increased in human and mouse Tregs after activation, and CCL3 is important in Treg regulatory function (Patterson S J, Pesenacker A M, Wang A Y, Gillies J, Mojibian M, Morishita K, Tan R, Kieffer T J, Verchere C B, Panagiotopoulos C, Levings MK., “T regulatory cell chemokine production mediates pathogenic T cell attraction and suppression”, J Clin Invest. 2016 Mar. 1; 126(3):1039-51). Higher abundance of human CCL3 mRNA is found in spinal cords from mice injected with DG05-28-3z CAR Tregs in comparison to the spinal cords from mice injected with the DG05-28tm CAR Tregs that lack a CAR signaling domain (FIG. 24 E).

In summary human Tregs are found in spleen and spinal cord (measured by human CD52 mRNA) in mSODTg/NSG mice injected iv with these CAR Tregs. CD52 mRNA is highly expressed in human Tregs and the primers are specific for human.

In the mice injected with a CAR containing CD28 and CD3z signaling domains, the presence of human CCL3 was detected which is significant as this cytokine is produced by Tregs and is believed to be important for Treg function. In the foregoing experiments it should be noted that two CARs were used which both recognize human mSOD1—one with a typical CD28/CD3z signaling domain and one with just a CD28Tm section (i.e., no signaling domain), which does not stimulate the cells but may tether them to sites of Ag.

Taken together these data provide further evidence that intravenously injected human CAR Tregs can access the spinal cords in mSOD1.NSG mice and become activated in vivo via CAR-mediated signaling. This further corroborates that the administration of human CAR expressing Tregs according to the invention may provide an effective means of treating human subjects with neurodegenerative diseases and conditions, e.g., ALS.

### Example 17: Expression of BDNF and mCD19 CARs in Tregs

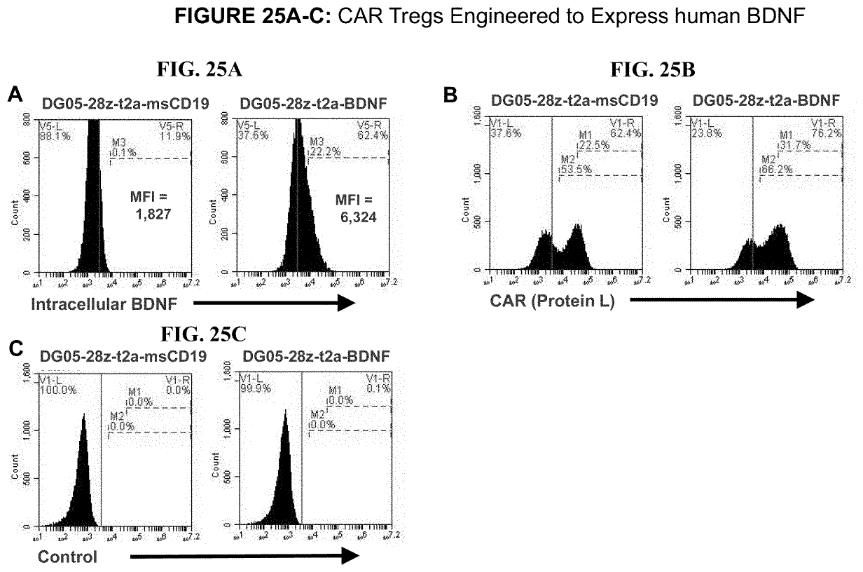

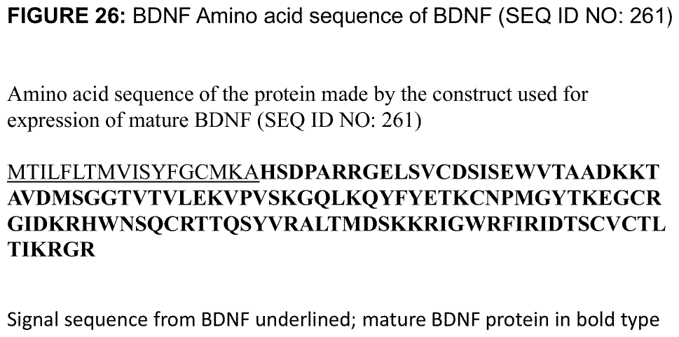

A CAR-T2a-BDNF construct according to the invention comprising the full length BDNF coding and signal sequence (see FIG. 26) was transduced into human Tregs. As shown in the flow cytometry staining experiments in FIG. 25A-C these transduced human Tregs expressed the CAR and BDNF (complete BDNF protein). In these experiments the transduced cells were permeabilized with permeabilization reagents (eBioscience) and then stained with anti-human BDNF-PE (Miltenyi Biotech). Panel A shows intracellular staining for the BDNF (A); Panel B shows protein L staining for the CAR (which polypeptide binds to scFv portion of the CAR) (B), and Panel C is a negative staining control which shows staining results for Streptavidin PE Streptavidin-PE (Biolegend) alone (C). These results are also compared these to CAR-T2a-mCD19 as a positive control for BDNF expression. As shown in the figure the Tregs expressed the polypeptides encoded by both constructs comparably.

### Example 18: Expression of vIL-10 (BCRF-1) in Human Tregs

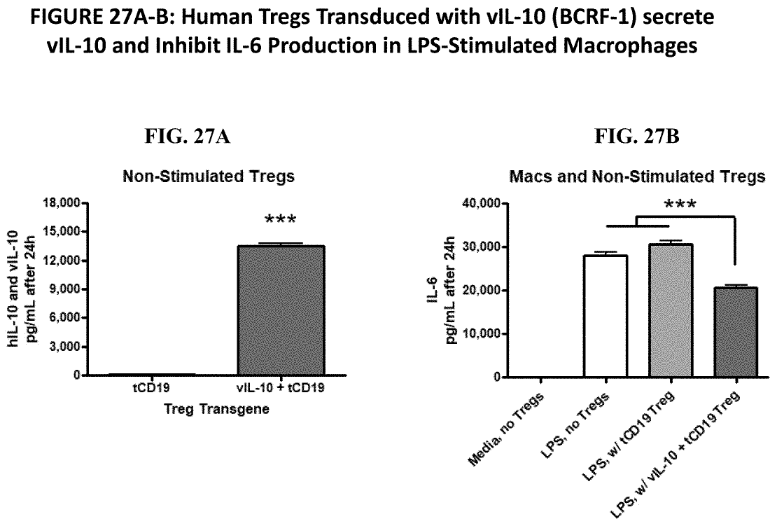

As shown in the experiments in FIGS. 27A and B human Tregs were transduced with vIL-10 (BCRF-1). As can be seen from the data in the figure the transduced cells potently secreted vIL-10 and elicited anti-inflammatory activity based on their potent inhibition of IL-6 expression in LPS-stimulated macrophages.

### Example 19: Human Tregs Transduced with CAR and vIL-10 (BCRF-1) Secrete vIL-10 and Retain CAR Function

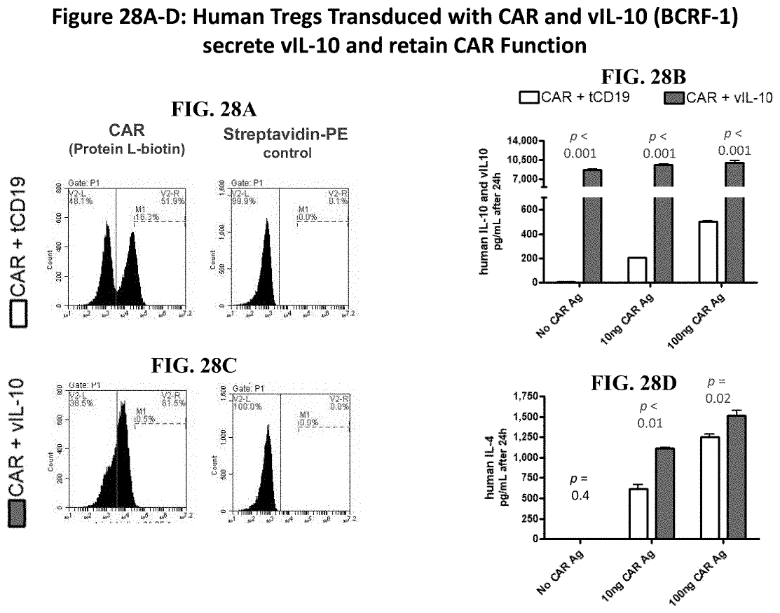

As shown in the experiments in FIG. 28A-D Tregs were also transduced with a CAR construct according to the invention and a nucleic acid sequence encoding vIL-10 (BCRF-1). The resultant transduced Tregs were cultured for 24 hrs in wells with no antigen (Ag), 10 ng Ag, or 100 ng Ag per well. After 24 hours, cytokine amounts were determined by ELISA. Student's t-test was used to compare the results between groups. As can be seen from the results in the figure the transduced Tregs secrete vIL-10 and retain CAR function.

In the preceding procedures, various steps have been described. It will, however, be evident that various modifications and changes may be made thereto, and additional procedures may be implemented, without departing from the broader scope of the exemplary procedures as set forth in the claims that follow.

APPENDIX A
AMINO ACID AND NUCLEIC ACID SEQUENCES

DG01 SCFV (SEQ ID NO: 1)
MEWTWVFLFLLSVTAGVHSQVQLVESGGGVVQPGRSLRLSCAASGFAFSSYGMHW
VRQAPGKGLEWVAVIWFDGTKKYYTDSVKGRFTISRDNSKNTLYLQMNTLRAEDT
AVYYCARDRGIGARRGPYYMDVWGKGTTVTVSSAGGGGSGGGGSGGGGSDIQMT
QSPSSLSASVGDRVTITCRASQSISSYLNWYQQKPGKAPKLLIYAASSLQSGVPSRFSG
SGSGTDFTLTISSLQPEDFATYYCQQSYSTPLTFGGGTKVEIK
DG02 SCFV (SEQ ID NO: 2)
MEWTWVFLFLLSVTAGVHSEVQLLESGGGLVQPGGSLRLSCAASGFTFSNYGMSW
VRQAPGKGLEWVASIRSGGGRTYYSDNVKGRFTISRDNSKNTLYLQMNSLRAEDTA
VYYCVRYDHYSGSSDYWGQGTLVTVSSAGGGGSGGGGSGGGGSDVVMTQSPLSLP
VTPGEPASISCKSSQSLLDSDGKTYLNWLLQKPGQSPQRLIYLVSKLDSGVPDRFSGS
GSGTDFTLKISRVEAEDVGVYYCWQGTHFPRTFGQGTKVEIK
DG03 SCFV (SEQ ID NO: 3)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIK
DG04 SCFV (SEQ ID NO: 4)
MEWTWVFLFLLSVTAGVHSQVELVESGGGLVQPGGSLRLSCAASGFTFSSYAMSWV
RQAPGKGLEWVSAINASGTRTYYADSVKGRFTISRDNSKNTLYLQMNSLRAEDTAV
YYCARGKGNTHKPYGYVRYFDVWGQGTLVTVSSAGGGGSGGGGSGGGGSDIVLTQ
SPATLSLSPGERATLSCRASQSVSSSYLAWYQQKPGQAPRLLIYGASSRATGVPARFS
GSGSGTDFTLTISSLEPEDFATYYCLQIYNMPITFGQGTKVEIK
DG05 SCFV (SEQ ID NO: 5)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIK
DG06 SCFV (SEQ ID NO: 6)
MEWTWVFLFLLSVTAGVHSQVQLVESGGGVVQPGRSLRLSCAASGFTFSNYGIHWV
RQAPGKGLEWVAIIWHDGSNSYYVDSVKGRFTISRDNSKNTLYLQMNSLRAEDTAV
YFCARIIGGAFDIWGQGTMVTVSSAGGGGSGGGGSGGGGSDIQMTQSPSSLSASVGD
RVTITCRASQGISSWLAWYQQKPEKAPKSLIYAASSLQSGVPSRFSGSGSGTDFTLTIS
SLQPEDFATYYCQQYNSYPITFGQGTRLEIK
DG07 SCFV (SEQ ID NO: 7)
MEWTWVFLFLLSVTAGVHSEVQLVESGGGLVQPGGSLRLSCAASGFSISGYWMSW
VRQAPGKGLEWVANIKQDGGEKYYGDSVKGRFTISRDNAKNSLYLQMNSLRAEDT
AVYYCVMAGGLDYWGQGTLVTVSSAGGGGSGGGGSGGGGSEIVLTQSPATLSLSPG
ERATLSCRASQSVSSYLAWYQQKPGQAPRLLIYDASNRATGIPARFSGSGSGTDFTLT
ISSLEPEDFAVYYCQQRSNWYTFGQGTKLEIK
DG08 SCFV (SEQ ID NO: 8)
MEWTWVFLFLLSVTAGVHSQVQLVQSGAEVKKPGASVRLSCRASGYNFIDFHIHWV
RQAPGEGLEWMGWSNPQSGNSSSAQRFQGRVTMTTDTSMSAAYMDLNWLTLDDT
AVYYCTRPHDGAGNYRFDTWGQGTLVTVSSAGGGGSGGGGSGGGGSSYELTQPPS
VSVAPGQTARITCSGDALPKHYAHWYQQKPGQVPIVVIYKDTERPSGIPERFSGSTSG
TTVTLTISGVQAEDEAHYYCQSADVSSTYVVFGGGTKLTVL
DG09 SCFV (SEQ ID NO: 9)
MEWTWVFLFLLSVTAGVHSEVQLVESGGGLVEPGGSLRLSCAVSGFDFEKAWMSW
VRQAPGQGLQWVARIKSTADGGTTSYAAPVEGRFIISRDDSRNMLYLQMNSLKTED
TAVYYCTSAHWGQGTLVTVSSAGGGGSGGGGSGGGGSSYELTQPPSVSVSPGQTAR
ITCSGEALPMQFAHWYQQRPGKAPVIVVYKDSERPSGVPERFSGSSSGTTATLTITGV
QAEDEADYYCQSPDSTNTYEVFGGGTKLTVL
DG10 SCFV (SEQ ID NO: 10)
MEWTWVFLFLLSVTAGVHSQVQLVQSGAEVKKPGASVKVSCKASGYTFTNYAMH
WVRQAPGQRLEWMGWINAGNGKRKYSQKFQDRVTINRDTSASTIYMELSSLGSEDT
AVYYCAREEDHAGSGSYLSMDVWGQGSTVTVSSAGGGGSGGGGSGGGGSDIVMT
QSPDSLAVSLGERATINCKSSQNVLYSSNNKNYLAWYQQKPGHPPKLLIYWASTRES
GVPDRFSGSGSGTDFTLTITSLQTEDVAVYYCQQYYSSPLTFGGGTKVEIK
DG11 SCFV (SEQ ID NO: 11)
MEWTWVFLFLLSVTAGVHSEVQLVETGGGLVQPKGSLKLSCATSGFTFNTYAMNW
VRQAPGKGLEWVARIRTKSNDYATYYADSVKGRITISRDDSQSMLYLQMNNLKTED
TAMYYCVRVGYRPYAMDYWGQGTSVTVSSAGGGGSGGGGSGGGGSDVLMTQTPL
SLPVSLGDQASISCRSSQNIVHSNGNTYLEWYLQKPGQSPTLLIYKVSNRFSGVPDRF
SGSGSGTDFTLKISRVEAEDLGVYYCFQGSHVPLTFGAGTKLELK
4-1BB CO-STIMULATORY DOMAIN (SEQ ID NO: 12)
KRGRKKLLYIFKQPFMRPVQTTQEEDGCSCRFPEEEEGGCEL
(G4S)3 LINKER (SEQ ID NO: 13)
GGGGSGGGGSGGGGS
T2A (SEQ ID NO: 14)
RAKRSGSGEGRGSLITCGDVEENPGP
P2A (SEQ ID NO: 15)
RAKRSGSGATNFSLLKQAGDVEENPGP
CD3Z (SEQ ID NO: 16)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALP
PR
HUMAN AMYLOID BETA, ISOFORM APP770 (IDENTIFIER: P05067-1) (SEQ ID NO: 17)
MLPGLALLLLAAWTARALEVPTDGNAGLLAEPQIAMFCGRLNMHMNVQNGKWDS
DPSGTKTCIDTKEGILQYCQEVYPELQITNVVEANQPVTIQNWCKRGRKQCKTHPHF
VIPYRCLVGEFVSDALLVPDKCKFLHQERMDVCETHLHWHTVAKETCSEKSTNLHD
YGMLLPCGIDKFRGVEFVCCPLAEESDNVDSADAEEDDSDVWWGGADTDYADGSE
DKVVEVAEEEEVAEVEEEEADDDEDDEDGDEVEEEAEEPYEEATERTTSIATTTTTT
TESVEEVVREVCSEQAETGPCRAMISRWYFDVTEGKCAPFFYGGCGGNRNNFDTEE
YCMAVCGSAMSQSLLKTTQEPLARDPVKLPTTAASTPDAVDKYLETPGDENEHAHF
QKAKERLEAKHRERMSQVMREWEEAERQAKNLPKADKKAVIQHFQEKVESLEQEA
ANERQQLVETHMARVEAMLNDRRRLALENYITALQAVPPRPRHVFNMLKKYVRAE
QKDRQHTLKHFEHVRMVDPKKAAQIRSQVMTHLRVIYERMNQSLSLLYNVPAVAE
EIQDEVDELLQKEQNYSDDVLANMISEPRISYGNDALMPSLTETKTTVELLPVNGEFS
LDDLQPWHSFGADSVPANTENEVEPVDARPAADRGLTTRPGSGLTNIKTEEISEVKM
DAEFRHDSGYEVHHQKLVFFAEDVGSNKGAIIGLMVGGVVIATVIVITLVMLKKKQ
YTSIHHGVVEVDAAVTPEERHLSKMQQNGYENPTYKFFEQMQN
HUMAN SUPEROXIDE DISMUTASE, IDENTIFIER: P00441-1 (SEQ ID NO: 18)
MATKAVCVLKGDGPVQGIINFEQKESNGPVKVWGSIKGLTEGLHGFHVHEFGDN
TAGCTSAGPHFNPLSRKHGGPKDEERHVGDLGNVTADKDGVADVSIEDSVISL SGDH
CIIGRTLVVHEKADDLGKGGNEESTKTGNAGSRLACGVIGIAQ
HUMAN ALPHA-SYNUCLEIN, ISOFORM 1 (IDENTIFIER: P37840-1) (SEQ ID NO: 19)
MDVFMKGLSKAKEGVVAAAEKTKQGVAEAAGKTKEGVLYVGSKTKEGVVHGVAT
VAEKTKEQVTNVGGAVVTGVTAVAQKTVEGAGSIAAATGFVKKDQLGKNEEGAPQ
EGILEDMPVDPDNEAYEMPSEEGYQDYEPEA
DG01.28.Z CAR (SEQ ID NO: 20)
MEWTWVFLFLLSVTAGVHSQVQLVESGGGVVQPGRSLRLSCAASGFAFSSYGMHW
VRQAPGKGLEWVAVIWFDGTKKYYTDSVKGRFTISRDNSKNTLYLQMNTLRAEDT
AVYYCARDRGIGARRGPYYMDVWGKGTTVTVSSAGGGGSGGGGSGGGGSDIQMT
QSPSSLSASVGDRVTITCRASQSISSYLNWYQQKPGKAPKLLIYAASSLQSGVPSRFSG
SGSGTDFTLTISSLQPEDFATYYCQQSYSTPLTFGGGTKVEIKASVKGKHLCPSPLFPG
PSKPFWVLVVVGGVLACYSLLVTVAFIIFWVRSKRSRLLHSDYMNMTPRRPGPTRK
HYQPYAPPRDFAAYRSKLRVKFSRSADAPAYQQGQNQLYNELNLGRREEYDVLDK
RRGRDPEMGGKPRRKNPQEGLYNELQKDKMAEAYSEIGMKGERRRGKGHDGLYQ
GLSTATKDTYDALHMQALPP
DG02.28.Z CAR (SEQ ID NO: 21)
MEWTWVFLFLLSVTAGVHSEVQLLESGGGLVQPGGSLRLSCAASGFTFSNYGMSW
VRQAPGKGLEWVASIRSGGGRTYYSDNVKGRFTISRDNSKNTLYLQMNSLRAEDTA
VYYCVRYDHYSGSSDYWGQGTLVTVSSAGGGGSGGGGSGGGGSDVVMTQSPLSLP
VTPGEPASISCKSSQSLLDSDGKTYLNWLLQKPGQSPQRLIYLVSKLDSGVPDRFSGS
GSGTDFTLKISRVEAEDVGVYYCWQGTHFPRTFGQGTKVEIKASVKGKHLCPSPLFP
GPSKPFWVLVVVGGVLACYSLLVTVAFIIFWVRSKRSRLLHSDYMNMTPRRPGPTR
KHYQPYAPPRDFAAYRSKLRVKFSRSADAPAYQQGQNQLYNELNLGRREEYDVLD
KRRGRDPEMGGKPRRKNPQEGLYNELQKDKMAEAYSEIGMKGERRRGKGHDGLY
QGLSTATKDTYDALHMQALPP
DG03.28.Z CAR (SEQ ID NO: 22)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWVRSKRSRLLHSDYMNMTPRRPGPTRKHYQPYAPPR
DFAAYRSKLRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTY
DALHMQALPP
DG04.28.Z CAR (SEQ ID NO: 23)
MEWTWVFLFLLSVTAGVHSQVELVESGGGLVQPGGSLRLSCAASGFTFSSYAMSWV
RQAPGKGLEWVSAINASGTRTYYADSVKGRFTISRDNSKNTLYLQMNSLRAEDTAV
YYCARGKGNTHKPYGYVRYFDVWGQGTLVTVSSAGGGGSGGGGSGGGGSDIVLTQ
SPATLSLSPGERATLSCRASQSVSSSYLAWYQQKPGQAPRLLIYGASSRATGVPARFS
GSGSGTDFTLTISSLEPEDFATYYCLQIYNMPITFGQGTKVEIKASVKGKHLCPSPLFP
GPSKPFWVLVVVGGVLACYSLLVTVAFIIFWVRSKRSRLLHSDYMNMTPRRPGPTR
KHYQPYAPPRDFAAYRSKLRVKFSRSADAPAYQQGQNQLYNELNLGRREEYDVLD
KRRGRDPEMGGKPRRKNPQEGLYNELQKDKMAEAYSEIGMKGERRRGKGHDGLY
QGLSTATKDTYDALHMQALPP
DG05.28.Z CAR (SEQ ID NO: 24)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIKASVKGKHLCPSPLFPGPSKPFWV
LVVVGGVLACYSLLVTVAFIIFWVRSKRSRLLHSDYMNMTPRRPGPTRKHYQPYAPP
RDFAAYRSKLRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDT
YDALHMQALPP
DG06.28.Z CAR (SEQ ID NO: 25)
MEWTWVFLFLLSVTAGVHSQVQLVESGGGVVQPGRSLRLSCAASGFTFSNYGIHWV
RQAPGKGLEWVAIIWHDGSNSYYVDSVKGRFTISRDNSKNTLYLQMNSLRAEDTAV
YFCARIIGGAFDIWGQGTMVTVSSAGGGGSGGGGSGGGGSDIQMTQSPSSLSASVGD
RVTITCRASQGISSWLAWYQQKPEKAPKSLIYAASSLQSGVPSRFSGSGSGTDFTLTIS
SLQPEDFATYYCQQYNSYPITFGQGTRLEIKASVKGKHLCPSPLFPGPSKPFWVLVVV
GGVLACYSLLVTVAFIIFWVRSKRSRLLHSDYMNMTPRRPGPTRKHYQPYAPPRDFA
AYRSKLRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDAL
HMQALPP
DG07.28.Z CAR (SEQ ID NO: 26)
MEWTWVFLFLLSVTAGVHSEVQLVESGGGLVQPGGSLRLSCAASGFSISGYWMSW
VRQAPGKGLEWVANIKQDGGEKYYGDSVKGRFTISRDNAKNSLYLQMNSLRAEDT
AVYYCVMAGGLDYWGQGTLVTVSSAGGGGSGGGGSGGGGSEIVLTQSPATLSLSPG
ERATLSCRASQSVSSYLAWYQQKPGQAPRLLIYDASNRATGIPARFSGSGSGTDFTLT
ISSLEPEDFAVYYCQQRSNWYTFGQGTKLEIKASVKGKHLCPSPLFPGPSKPFWVLV
VVGGVLACYSLLVTVAFIIFWVRSKRSRLLHSDYMNMTPRRPGPTRKHYQPYAPPR
DFAAYRSKLRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTY
DALHMQALPP
DG08.28.Z CAR (SEQ ID NO: 27)
MEWTWVFLFLLSVTAGVHSQVQLVQSGAEVKKPGASVRLSCRASGYNFIDFHIHWV
RQAPGEGLEWMGWSNPQSGNSSSAQRFQGRVTMTTDTSMSAAYMDLNWLTLDDT
AVYYCTRPHDGAGNYRFDTWGQGTLVTVSSAGGGGSGGGGSGGGGSSYELTQPPS
VSVAPGQTARITCSGDALPKHYAHWYQQKPGQVPIVVIYKDTERPSGIPERFSGSTSG
TTVTLTISGVQAEDEAHYYCQSADVSSTYVVFGGGTKLTVLASVKGKHLCPSPLFPG
PSKPFWVLVVVGGVLACYSLLVTVAFIIFWVRSKRSRLLHSDYMNMTPRRPGPTRK
HYQPYAPPRDFAAYRSKLRVKFSRSADAPAYQQGQNQLYNELNLGRREEYDVLDK
RRGRDPEMGGKPRRKNPQEGLYNELQKDKMAEAYSEIGMKGERRRGKGHDGLYQ
GLSTATKDTYDALHMQALPP
DG09.28.Z CAR (SEQ ID NO: 28)
MEWTWVFLFLLSVTAGVHSEVQLVESGGGLVEPGGSLRLSCAVSGFDFEKAWMSW
VRQAPGQGLQWVARIKSTADGGTTSYAAPVEGRFIISRDDSRNMLYLQMNSLKTED
TAVYYCTSAHWGQGTLVTVSSAGGGGSGGGGSGGGGSSYELTQPPSVSVSPGQTAR
ITCSGEALPMQFAHWYQQRPGKAPVIVVYKDSERPSGVPERFSGSSSGTTATLTITGV
QAEDEADYYCQSPDSTNTYEVFGGGTKLTVLASVKGKHLCPSPLFPGPSKPFWVLV
VVGGVLACYSLLVTVAFIIFWVRSKRSRLLHSDYMNMTPRRPGPTRKHYQPYAPPR
DFAAYRSKLRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTY
DALHMQALPP
DG10.28.Z CAR (SEQ ID NO: 29)
MEWTWVFLFLLSVTAGVHSQVQLVQSGAEVKKPGASVKVSCKASGYTFTNYAMH
WVRQAPGQRLEWMGWINAGNGKRKYSQKFQDRVTINRDTSASTIYMELSSLGSEDT
AVYYCAREEDHAGSGSYLSMDVWGQGSTVTVSSAGGGGSGGGGSGGGGSDIVMT
QSPDSLAVSLGERATINCKSSQNVLYSSNNKNYLAWYQQKPGHPPKLLIYWASTRES
GVPDRFSGSGSGTDFTLTITSLQTEDVAVYYCQQYYSSPLTFGGGTKVEIKASVKGK
HLCPSPLFPGPSKPFWVLVVVGGVLACYSLLVTVAFIIFWVRSKRSRLLHSDYMNMT
PRRPGPTRKHYQPYAPPRDFAAYRSKLRVKFSRSADAPAYQQGQNQLYNELNLGRR
EEYDVLDKRRGRDPEMGGKPRRKNPQEGLYNELQKDKMAEAYSEIGMKGERRRGK
GHDGLYQGLSTATKDTYDALHMQALPP
DG11.28.Z CAR (SEQ ID NO: 30)
MEWTWVFLFLLSVTAGVHSEVQLVETGGGLVQPKGSLKLSCATSGFTFNTYAMNW
VRQAPGKGLEWVARIRTKSNDYATYYADSVKGRITISRDDSQSMLYLQMNNLKTED
TAMYYCVRVGYRPYAMDYWGQGTSVTVSSAGGGGSGGGGSGGGGSDVLMTQTPL
SLPVSLGDQASISCRSSQNIVHSNGNTYLEWYLQKPGQSPTLLIYKVSNRFSGVPDRF
SGSGSGTDFTLKISRVEAEDLGVYYCFQGSHVPLTFGAGTKLELKASVKGKHLCPSP
LFPGPSKPFWVLVVVGGVLACYSLLVTVAFIIFWVRSKRSRLLHSDYMNMTPRRPGP
TRKHYQPYAPPRDFAAYRSKLRVKFSRSADAPAYQQGQNQLYNELNLGRREEYDVL
DKRRGRDPEMGGKPRRKNPQEGLYNELQKDKMAEAYSEIGMKGERRRGKGHDGL
YQGLSTATKDTYDALHMQALPP
SIGNAL SEQUENCE (SEQ ID NO: 31)
MEWTWVFLFLLSVTAGVHS
HUMAN CD28 HINGE (SEQ ID NO: 32)
VKGKHLCPSPLFPGPSKP
MOUSE CD28 HINGE (SEQ ID NO: 33)
IKEKHLCHTQSSPKL
HUMAN CD8A HINGE (SEQ ID NO: 34)
TTTPAPRPPTPAPTIASQPLSLRPEACRPAAGGAVHTRGLDFACD
HUMAN DAP10 HINGE (SEQ ID NO: 35)
QTTPGERSSLPAFYPGTSGSCSGCGSLSLP
HUMAN CD28 TM (SEQ ID NO: 36)
FWVLVVVGGVLACYSLLVTVAFIIFWV
MOUSE CD28 TM (SEQ ID NO: 37)
FWALVVVAGVLFCYGLLVTVALCVIWT
HUMAN CD8A TM (SEQ ID NO: 38)
IYIWAPLAGTCGVLLLSLVITLYC
HUMAN DAP10 TM (SEQ ID NO: 39)
LLAGLVAADAVASLLIVGAVF
DG05-CD28tm-DAP10-CD3ζ (SEQ ID NO: 40)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIKASVKGKHLCPSPLFPGPSKPFWV
LVVVGGVLACYSLLVTVAFIIFWVRSKRSLCARPRRSPAQEDGKVYINMPGRGKLRV
KFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
DG05-CD28tm-CD44-CD3ζ (SEQ ID NO: 41)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIKASVKGKHLCPSPLFPGPSKPFWV
LVVVGGVLACYSLLVTVAFIIFWVSRRRCGQKKKLVINSGNGAVEDRKPSGLNGEAS
KSQEMVHLVNKESSETPDQFMTADETRNLQNVDMKIGVRVKFSRSADAPAYQQGQ
NQLYNELNLGRREEYDVLDKRRGRDPEMGGKPRRKNPQEGLYNELQKDKMAEAYS
EIGMKGERRRGKGHDGLYQGLSTATKDTYDALHMQALPPR
DG05-CD28tm-CD3ζ (SEQ ID NO: 42)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIKASVKGKHLCPSPLFPGPSKPFWV
LVVVGGVLACYSLLVTVAFIIFWVRSKRSRRVKFSRSADAPAYQQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPR
DG05-CD28 (SEQ ID NO: 43)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIKASVKGKHLCPSPLFPGPSKPFWV
LVVVGGVLACYSLLVTVAFIIFWVRSKRSRLLHSDYMNMTPRRPGPTRKHYQPYAPP
RDFAAYRS
DG05-CD28tm (SEQ ID NO: 44)
MEWTWVFLFLLSVTAGVHSEVQLVQSGGGLVKPGGSLRLSCAGSGFTFSSYSMHWL
RQAPGKGLEWVSAIGTAGGTYYADSVKGRFTISRDNAKNSLYLQMNSLRAEDTAVY
YCAREYFFGSGNYGYWGQGTLVTVSSAGGGGSGGGGSGGGGSEIVLTQSPATLSLS
PGERATLSCRASQSVSSYLAWYQQKPGQAPRLLIYDASNRATGIPARFSGSGSGTDFT
LTISSLEPEDFAVYYCQQRSNWPPTFGQGTKVEIKASVKGKHLCPSPLFPGPSKPFWV
LVVVGGVLACYSLLVTVAFIIFWVRSKRSRLLHSD
DG03-CD28tm-DAP10-CD3ζ (SEQ ID NO: 45)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWVRSKRSLCARPRRSPAQEDGKVYINMPGRGKLRVKF
SRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
DG03-CD28tm-CD44-CD3ζ (SEQ ID NO: 46)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWVSRRRCGQKKKLVINSGNGAVEDRKPSGLNGEASKS
QEMVHLVNKESSETPDQFMTADETRNLQNVDMKIGVRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
DG03-CD28tm-4-1-BB-CD3ζ (SEQ ID NO: 47)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWVRSKRSLEKRGRKKLLYIFKQPFMRPVQTTQEEDGC
SCRFPEEEEGGCELKLRVKFSRSADAPAYQQGQNQLYNELNLGRREEYDVLDKRRG
RDPEMGGKPRRKNPQEGLYNELQKDKMAEAYSEIGMKGERRRGKGHDGLYQGLST
ATKDTYDALHMQALPPR
DG03-CD28tm-CD3ζ (SEQ ID NO: 48)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWVRSKRSRRVKFSRSADAPAYQQGQNQLYNELNLGR
REEYDVLDKRRGRDPEMGGKPRRKNPQEGLYNELQKDKMAEAYSEIGMKGERRRG
KGHDGLYQGLSTATKDTYDALHMQALPPR
DG03-CD28 (SEQ ID NO: 49)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWVRSKRSRLLHSDYMNMTPRRPGPTRKHYQPYAPPR
DFAAYRS
DG03-CD28tm (SEQ ID NO: 50)
MEWTWVFLFLLSVTAGVHSEVQLVESGGGLVQPGGSLRLSCAASGFTFSSYGMSWV
RQAPGKGLELVASINSNGGSTYYPDSVKGRFTISRDNAKNSLYLQMNSLRAEDTAVY
YCASGDYWGQGTTVTVSSAGGGGSGGGGSGGGGSDIVMTQSPLSLPVTPGEPASISC
RSSQSLVYSNGDTYLHWYLQKPGQSPQLLIYKVSNRFSGVPDRFSGSGSGTDFTLKIS
RVEAEDVGVYYCSQSTHVPWTFGQGTKVEIKASVKGKHLCPSPLFPGPSKPFWVLV
VVGGVLACYSLLVTVAFIIFWV
Construct for expression of the NDMM Nrf2 (Keap1 inhibitor peptide) (SEQ ID NO: 51)
MMDLELPPPGLPSQQDMDLIDILWRQDIDLGVSREVFDFSQRRKEYELEKQKKLEKE
RQEQLQKEQEKAFFAQLQLDEETGEFLPIQPAQ
Construct for expression of the NDMM human catalase (SEQ ID NO: 52)
MADSRDPASDQMQHWKEQRAAQKADVLTTGAGNPVGDKLNVITVGPRGPLLVQD
VVFTDEMAHFDRERIPERVVHAKGAGAFGYFEVTHDITKYSKAKVFEHIGKKTPIAV
RFSTVAGESGSADTVRDPRGFAVKFYTEDGNWDLVGNNTPIFFIRDPILFPSFIHSQKR
NPQTHLKDPDMVWDFWSLRPESLHQVSFLFSDRGIPDGHRHMNGYGSHTFKLVNA
NGEAVYCKFHYKTDQGIKNLSVEDAARLSQEDPDYGIRDLFNAIATGKYPSWTFYIQ
VMTFNQAETFPFNPFDLTKVWPHKDYPLIPVGKLVLNRNPVNYFAEVEQIAFDPSNM
PPGIEASPDKMLQGRLFAYPDTHRHRLGPNYLHIPVNCPYRARVANYQRDGPMCMQ
DNQGGAPNYYPNSFGAPEQQPSALEHSIQYSGEVRRFNTANDDNVTQVRAFYVNVL
NEEQRKRLCENIAGHLKDAQIFIQKKAVKNFTEVHPDYGSHIQALLDKYNAEKPKNA
IHTFVQSGSHLAAREKANL
Construct for expression of the NDMM BDNF (SEQ ID NO: 53)
MTILFLTMVISYFGCMKAAPMKEANIRGQGGLAYPGVRTHGTLESVNGPKAGSRGL
TSLADTFEHVIEELLDEDQKVRPNEENNKDADLYTSRVMLSSQVPLEPPLLFLLEEYK
NYLDAANMSMRVRRHSDPARRGELSVCDSISEWVTAADKKTAVDMSGGTVTVLEK
VPVSKGQLKQYFYETKCNPMGYTKEGCRGIDKRHWNSQCRTTQSYVRALTMDSKK
RIGWRFIRIDTSCVCTLTIKRGR
Construct for expression of the NDMM IGF-1 (SEQ ID NO: 54)
MGKISSLPTQLFKCCFCDFLKVKMHTMSSSHLFYLALCLLTFTSSATAGPETLCGAEL
VDALQFVCGDRGFYFNKPTGYGSSSRRAPQTGIVDECCFRSCDLRRLEMYCAPLKPA
KSARSVRAQRHTDMPKTQKYQPPSTNKNTKSQRRKGWPKTHPGGEQKEGTEASLQI
RGKKKEQRREIGSRNAECRGKKGK
DG01 SCFV (SEQ ID NO: 201)
atggaatgga cctgggtgtt cctgtttctg ctgtccgtga ccgctggcgt gcacagccag gtgcagctgg tggaaagcgg
cggaggagtc gtgcagcctg gcagaagcct gaggctgagc tgtgccgcca gcggcttcgc cttcagctcc tacggcatgc
actgggtgag acaggcccct ggcaagggac tggagtgggt ggctgtgatc tggttcgacg gcaccaagaa gtactacacc
gacagcgtca agggcaggtt caccatctcc agggacaata gcaagaatac cctgtacctc caaatgaaca ccctgagggc
cgaggacacc gccgtgtatt actgcgccag ggatagggga atcggcgcca ggagaggccc ctactacatg gacgtgtggg
gcaagggcac aacagtgacc gtttcttctg ctggaggagg aggttctgga ggaggaggaa gcggaggagg aggctccgac
atccagatga cacagtcccc cagctccctg tccgccagcg tgggcgatag agtgaccatc acctgcaggg ccagccagag
catctccagc tacctgaact ggtatcaaca gaagcccggc aaagccccca aactgctgat ctacgctgcc agcagcctgc
agagcggcgt gccttccaga ttcagcggct ccggcagcgg caccgatttc acactgacca tctccagcct gcagcccgag
gacttcgcca cctactactg ccagcagagc tacagcaccc ccctgacctt tggcggaggc accaaggtgg agatcaaa
DG02 SCFV (SEQ ID NO: 202)
atggaatgga cctgggtgtt tctgttcctg ctgagcgtga cagccggcgt gcacagcgag gtgcagctgc tggagagcgg
aggaggactg gtgcaacccg gcggaagcct cagactgagc tgtgccgcca gcggcttcac cttcagcaac tatggcatga
gctgggtgag gcaggcccct ggcaaaggcc tcgaatgggt ggcttccatt aggagcggcg gcggcaggac ctattacagc
gacaacgtga agggcaggtt caccatctcc agggacaata gcaagaacac cctgtacctg cagatgaact ccctgagggc
cgaggatacc gccgtgtact actgcgtgag gtacgaccac tacagcggct ccagcgacta ttggggacag ggcaccctgg
tgacagtgtc cagcgccgga ggaggcggca gcggcggcgg cggcagcggc ggcggcggaa gcgatgtggt
gatgacccag tcccccctga gcctgcctgt gacacctgga gagcccgcca gcatcagctg taagagcagc cagagcctcc
tggacagcga cggcaaaacc tacctgaact ggctcctgca gaagcccgga caaagccccc agaggctgat ctacctggtg
agcaaactgg acagcggcgt gcctgacaga ttctccggct ccggcagcgg caccgacttc acactgaaga tcagcagagt
ggaggctgag gacgtgggcg tctactactg ctggcagggc acccacttcc ccaggacctt cggccaggga accaaggtgg
agatcaag
DG03 SCFV (SEQ ID NO: 203)
atggaatgga cctgggtgtt cctgtttctg ctctccgtga ccgccggagt gcacagcgag gtgcagctgg tcgaaagcgg
cggaggactg gtgcagcctg gcggcagcct gagactgagc tgtgccgcct ccggcttcac ctttagcagc tacggaatgt
cctgggtgag acaggctcct ggcaagggcc tggaactggt ggccagcatc aatagcaacg gcggcagcac ctactaccct
gatagcgtga agggcaggtt caccatctcc agggacaacg ccaagaacag cctgtacctg cagatgaaca gcctcagggc
cgaggacaca gccgtgtact actgcgccag cggcgactat tggggacagg gaacaaccgt gaccgtcagc agcgccggcg
gcggcggcag cggcggcggc ggcagcggcg gcggcggctc cgatatcgtg atgacccaga gccccctgtc cctgcctgtc
acacctggcg aacccgccag cattagctgc aggtccagcc agagcctggt gtacagcaat ggcgacacct acctgcactg
gtacctgcag aagcctggcc agagccccca gctgctgatc tacaaggtga gcaacaggtt ctccggagtg cctgacaggt
tcagcggctc cggcagcgga accgatttca ccctcaagat cagcagagtg gaggccgagg acgtgggcgt ctactattgt
agccagagca cccacgtgcc ctggaccttt ggccagggca ccaaggtgga gatcaaa
DG04 SCFV (SEQ ID NO: 204)
atggaatgga cctgggtgtt tctgttcctc ctgagcgtga ccgccggagt gcacagccaa gtggagctgg tggagagcgg
aggaggactg gtgcagcctg gaggctccct gaggctgagc tgtgctgcca gcggcttcac cttcagctcc tatgctatga
gctgggtgag acaggcccct ggcaaaggcc tggagtgggt gagcgccatc aacgcctccg gcaccaggac ctactatgcc
gactccgtga agggcaggtt caccatctcc agggacaaca gcaagaacac cctgtacctg cagatgaaca gcctgagggc
tgaagacacc gccgtgtact actgtgccag gggcaagggc aacacacaca agccctatgg ctacgtgaga tacttcgacg
tgtggggaca gggcaccctg gtgacagtga gcagcgccgg aggaggaggt tctggaggag gaggaagcgg
cggaggagga agcgacatcg tgctgacaca atcccccgcc acactgtccc tgtcccctgg cgagagggcc acactgagct
gcagggccag ccagagcgtg tcctcctcct acctggcctg gtaccagcag aaacctggcc aggcccccag gctgctgatc
tatggcgcca gcagcagagc cacaggagtg cctgccagat ttagcggcag cggcagcggc accgacttta ccctgaccat
ttccagcctg gagcccgagg acttcgccac ctactactgc ctgcagatct acaacatgcc tatcaccttc ggccagggca
caaaagtgga aatcaag
DG05 SCFV (SEQ ID NO: 205)
atggaatgga cctgggtgtt tctgttcctc ctgtccgtga ccgccggagt gcactccgaa gtgcagctgg tgcagtccgg
cggaggactg gtgaaacccg gaggaagcct cagactgagc tgcgccggca gcggctttac cttctccagc tactccatgc
actggctgag acaggcccct ggcaagggcc tggaatgggt cagcgccatc ggcaccgccg gaggcacata ctatgccgac
agcgtgaagg gcaggttcac catcagcagg gacaacgcca agaacagcct gtacctgcag atgaactctc tgagggccga
ggataccgct gtgtactact gcgccaggga gtacttcttt ggcagcggca actacggata ctggggccag ggcaccctgg
tgacagtgag ctccgccgga ggaggaggaa gcggaggagg cggaagcgga ggaggcggca gcgaaatcgt
gctgacccag agccctgcca ccctgagcct gagccctggc gaaagggcca ccctgagctg cagagccagc cagagcgtga
gcagctacct ggcctggtac cagcagaagc ccggacaggc ccccagactg ctgatctacg acgccagcaa cagagccacc
ggcattcccg ccagattctc cggcagcggc agcggaaccg acttcacact gaccatcagc tccttagaac ccgaggactt
cgccgtgtac tactgtcagc agagaagcaa ctggcctccc accttcggcc agggcacaaa ggtggagatc aag
DG06 SCFV (SEQ ID NO: 206)
atggaatgga cctgggtgtt cctgttcctc ctgagcgtga ccgctggcgt ccacagccag gtgcagctgg tggaaagcgg
aggcggagtg gtgcagcctg gaaggtccct cagactgagc tgcgctgcca gcggcttcac cttcagcaac tacggcatcc
actgggtgag acaagccccc ggcaaaggcc tggagtgggt ggccatcatc tggcacgacg gcagcaactc ctactacgtg
gactccgtga agggcaggtt cacaatcagc agagacaaca gcaagaatac cctgtacctg cagatgaaca gcctcagggc
cgaagatacc gccgtgtact tctgcgccag gatcatcggc ggcgcctttg acatttgggg ccaaggcact atggttaccg
tgagcagcgc tggcggaggc ggcagcggcg gcggcggcag cggcggcggc ggaagcgaca tccagatgac
ccagagccct tccagcctca gcgcctccgt gggagacaga gtgaccatca cctgcagggc cagccagggc atctccagct
ggctggcctg gtaccagcag aagcctgaga aagcccccaa gagcctgatc tacgctgcct ccagcctgca gtccggcgtg
ccttccagat tctccggcag cggcagcggc accgacttta ccctgaccat ttccagcctg caacccgagg acttcgccac
ctactactgc cagcagtaca acagctaccc catcaccttt ggccagggca ccagactgga gatcaag
DG07 SCFV (SEQ ID NO: 207)
atggaatgga cctgggtgtt cctgttcctg ctgtccgtga ccgccggagt ccacagcgag gtgcagctgg tggaaagcgg
cggaggactg gtgcagcctg gaggcagcct gaggctgagc tgtgctgcca gcggcttctc catcagcggc tactggatgt
cctgggtgag gcaggcccct ggaaagggct tagaatgggt ggccaacatc aaacaggacg gcggcgagaa gtactacgga
gacagcgtca agggcagatt caccatcagc agggacaacg ccaagaacag cctgtacctg cagatgaaca gcctgagggc
cgaggacacc gctgtgtact actgcgtgat ggccggaggc ctggattatt ggggccaggg cacactggtg acagtgagca
gcgccggcgg cggcggcagc ggcggcggcg gcagcggcgg cggcggcagc gagatcgtgc tgacccagag
ccctgccaca ctgagcctga gccccggcga aagagccacc ctcagctgca gggccagcca gagcgtgagc agctacctgg
cctggtacca gcagaagccc ggacaggccc ctaggctgct gatctacgat gccagcaaca gagccaccgg catccctgct
aggttcagcg gttctggcag cggcaccgac ttcaccctga ccatcagcag cctggagcct gaggacttcg ctgtctacta
ctgccagcag aggagcaact ggtatacctt cggccaggga accaagctgg agatcaag
DG08 SCFV (SEQ ID NO: 208)
atggaatgga cctgggtgtt cctgttcctg ctcagcgtga ccgccggagt gcactcccag gtgcagctgg tgcagagcgg
cgccgaagtg aaaaagcccg gcgccagcgt gagactctcc tgtagagcca gcggctacaa cttcatcgac ttccacatcc
actgggtgag acaggcccct ggagagggcc tggagtggat gggctggagc aacccccaga gcggcaatag
cagcagcgcc cagaggttcc agggcagagt gaccatgacc accgatacct ccatgagcgc cgcctacatg gacctgaact
ggctgaccct ggacgacacc gccgtgtact actgcaccag gcctcacgac ggcgctggca actacaggtt cgacacctgg
ggacagggaa ccctggtgac agtcagcagc gcgggaggtg gtggtagcgg tggtgggggt tccggtggag gtggcagttc
atatgagctt acacaacccc caagtgtgag cgtggctcct ggccagacag ccaggatcac ctgcagcgga gacgccctgc
ccaaacacta cgcccactgg tatcagcaga aacccggcca ggtgcccatc gtggtgatct acaaggacac cgagagacct
agcggcatcc ccgagagatt cagcggcagc accagcggca ccacagtcac cctgaccatc tccggcgtgc aggccgagga
tgaagcccac tactactgcc aaagcgccga cgtgtcctcc acctatgtgg tgttcggcgg cggcaccaag ctgaccgtcc tc
DG09 SCFV (SEQ ID NO: 209)
atggagtgga cttgggtatt cctgtttttg ttgtccgtaa ctgctggggt acactcagag gtgcagctgg tagagtctgg
aggggggctc gtcgaaccag gcggctctct caggctttcc tgtgccgtaa gcgggtttga attgagaaa gcatggatgt
cctgggtaag gcaagctcca gggcagggac tccagtgggt agcgcggata aagtcaacag ctgatggcgg aaccacctct
tatgcagcac cggttgaggg aaggttcatc atctcacgag acgattcccg caacatgttg tatctgcaga tgaacagttt
gaaaactgaa gacacagctg tttactactg tacttcagcg cattggggac agggtactct tgtgacggtc tctagcgccg
gggggggagg ctctggaggg gggggttcag ggggtggtgg cagctcctat gagctgactc aaccgccttc agtaagcgta
agccctggtc agaccgctag aataacctgt agtggagagg ccctgccgat gcaattcgcc cactggtatc agcagaggcc
tggaaaagcc ccagtgattg tcgtttacaa agattccgaa cgccctagcg gggttcccga acgctttagc ggtagttcaa
gcgggacaac agcaaccctt acgataaccg gtgtacaagc ggaagacgaa gcggattact attgccaatc acctgatagt
acaaatactt atgaggtatt tggcggggga acgaagttga ctgtactg
DG10 SCFV (SEQ ID NO: 210)
atggaatgga cctgggtgtt cctcttcctc ctgtccgtca ccgctggcgt gcacagccag gtgcagctgg tccagagcgg
agccgaggtg aaaaagcccg gcgcctccgt gaaggtcagc tgcaaggcct ccggctacac cttcaccaac tacgccatgc
actgggtgag acaggcccct ggccagagac tggagtggat gggctggatc aacgccggca acggcaagag aaagtacagc
cagaagtttc aggacagggt gaccatcaac agggacacca gcgcctccac catctacatg gagctgtcca gcctgggcag
cgaggatacc gccgtgtact actgtgccag agaggaggat cacgctggca gcggcagcta cctgagcatg gacgtctggg
gacagggcag caccgtgaca gtgagcagcg ctggaggcgg cggctccggc ggcggaggaa gcggaggcgg
aggctccgac atcgtgatga cccagtcccc cgatagcctg gctgtgagcc tgggcgagag ggccacaatc aactgtaaga
gcagccagaa cgtgctgtac tcctccaaca acaagaacta cctggcctgg taccagcaga aacctggcca tccccccaag
ctgctgatct actgggccag caccagggag agcggagtgc ctgacaggtt tagcggcagc ggcagcggca cagactttac
cctgaccatc acctccctgc agaccgagga cgtggccgtg tactattgcc agcagtacta cagctcccct ctgaccttcg
gcggcggcac caaagtggag atcaaa
DG11 SCFV (SEQ ID NO: 211)
atggagtgga cttgggtatt ccattcctc ctctccgtga cagcgggtgt gcactctgaa gtacaacttg tagagaccgg
tgggggattg gtgcaaccca agggttccct gaaactctca tgtgctacct ctggttttac tttcaacacc tatgcaatga
attgggttag gcaagcaccc ggtaaaggac ttgagtgggt ggcacggata cgcactaaga gtaatgacta tgctacgtac
tacgcagact ccgtaaaagg ccggatcacc atatctcgag acgatagcca gtctatgctg tatcttcaaa tgaacaacct
caaaacggaa gatacggcga tgtattactg cgtgcgagtt ggttataggc cttatgctat ggattactgg ggacagggca
cgtctgtcac ggtaagttct gccggagggg ggggcagcgg aggaggagga tctggcggag ggggctccga tgtccttatg
acacagactc ccctcagttt gcccgtgtcc ttgggggacc aggcttctat atcatgccgc agttcccaaa atatcgtcca
ttcaaatggc aatacttacc ttgagtggta tttgcagaag cctggacaga gcccgacgct tctgatctat aaggtaagca
acaggttcag tggtgtaccc gacagattta gtggaagtgg gtccggaact gatttcactc ttaagattag tcgggtagag
gctgaagacc ttggggtgta ttattgcttt caagggagtc acgtccctct tacatttggt gctgggacta agttggagct gaag
4-1BB CO-STIMULATORY DOMAIN (SEQ ID NO: 212)
aaacggggca gaaagaaact cctgtatata ttcaaacaac catttatgag accagtacaa actactcaag aggaagatgg
ctgtagctgc cgatttccag aagaagaaga aggaggatgt gaactg
(G4S)3 LINKER (SEQ ID NO: 213)
ggcggaggcg gatcaggagg aggaggatca ggcggaggag gatca
T2A (SEQ ID NO: 214)
agagccaaaa ggtctggctc cggtgagggc agaggaagtc ttataacatg cggtgacgtg gaggagaatc ccggccct
P2A (SEQ ID NO: 215)
agagccaaaa ggtccggaag cggcgccacc aacttcagcc tgctgaagca ggccggcgac gtggaagaga atcctggccc
C
CD3Z (SEQ ID NO: 216)
agagtgaagt tcagcaggag cgcagacgcc cccgcgtacc agcagggcca gaaccagctc tataacgagc tcaatctagg
acgaagagag gagtacgatg ttttggacaa gagacgtggc cgggaccctg agatgggggg aaagccgaga aggaagaacc
ctcaggaagg cctgtacaat gaactgcaga aagataagat ggcggaggcc tacagtgaga ttgggatgaa aggcgagcgc
cggaggggca aggggcacga tggcctttac cagggtctca gtacagccac caaggacacc tacgacgccc ttcacatgca
ggccctgccc cctcgc
DG01.28.Z CAR (SEQ ID NO: 220)
atggaatgga cctgggtgtt cctgtttctg ctgtccgtga ccgctggcgt gcacagccag gtgcagctgg tggaaagcgg
cggaggagtc gtgcagcctg gcagaagcct gaggctgagc tgtgccgcca gcggcttcgc cttcagctcc tacggcatgc
actgggtgag acaggcccct ggcaagggac tggagtgggt ggctgtgatc tggttcgacg gcaccaagaa gtactacacc
gacagcgtca agggcaggtt caccatctcc agggacaata gcaagaatac cctgtacctc caaatgaaca ccctgagggc
cgaggacacc gccgtgtatt actgcgccag ggatagggga atcggcgcca ggagaggccc ctactacatg gacgtgtggg
gcaagggcac aacagtgacc gtttcttctg ctggaggagg aggttctgga ggaggaggaa gcggaggagg aggctccgac
atccagatga cacagtcccc cagctccctg tccgccagcg tgggcgatag agtgaccatc acctgcaggg ccagccagag
catctccagc tacctgaact ggtatcaaca gaagcccggc aaagccccca aactgctgat ctacgctgcc agcagcctgc
agagcggcgt gccttccaga ttcagcggct ccggcagcgg caccgatttc acactgacca tctccagcct gcagcccgag
gacttcgcca cctactactg ccagcagagc tacagcaccc ccctgacctt tggcggaggc accaaggtgg agatcaaagc
tagcgtgaaa gggaaacacc tttgtccaag tcccctattt cccggacctt ctaagccctt ttgggtgctg gtggtggttg
gtggagtcct ggcttgctat agcttgctag taacagtggc ctttattatt ttctgggtga ggagtaagag gagcaggctc
ctgcacagtg actacatgaa catgactccc cgccgccccg ggcccacccg caagcattac cagccctatg ccccaccacg
cgacttcgca gcctatcgct ccaagcttag agtgaagttc agcaggagcg cagacgcccc cgcgtaccag cagggccaga
accagctcta taacgagctc aatctaggac gaagagagga gtacgatgtt ttggacaaga gacgtggccg ggaccctgag
atggggggaa agccgagaag gaagaaccct caggaaggcc tgtacaatga actgcagaaa gataagatgg cggaggccta
cagtgagatt gggatgaaag gcgagcgccg gaggggcaag gggcacgatg gcctttacca gggtctcagt acagccacca
aggacaccta cgacgccctt cacatgcagg ccctgccccc t
DG02.28.Z CAR (SEQ ID NO: 221)
atggaatgga cctgggtgtt tctgttcctg ctgagcgtga cagccggcgt gcacagcgag gtgcagctgc tggagagcgg
aggaggactg gtgcaacccg gcggaagcct cagactgagc tgtgccgcca gcggcttcac cttcagcaac tatggcatga
gctgggtgag gcaggcccct ggcaaaggcc tcgaatgggt ggcttccatt aggagcggcg gcggcaggac ctattacagc
gacaacgtga agggcaggtt caccatctcc agggacaata gcaagaacac cctgtacctg cagatgaact ccctgagggc
cgaggatacc gccgtgtact actgcgtgag gtacgaccac tacagcggct ccagcgacta ttggggacag ggcaccctgg
tgacagtgtc cagcgccgga ggaggcggca gcggcggcgg cggcagcggc ggcggcggaa gcgatgtggt
gatgacccag tcccccctga gcctgcctgt gacacctgga gagcccgcca gcatcagctg taagagcagc cagagcctcc
tggacagcga cggcaaaacc tacctgaact ggctcctgca gaagcccgga caaagccccc agaggctgat ctacctggtg
agcaaactgg acagcggcgt gcctgacaga ttctccggct ccggcagcgg caccgacttc acactgaaga tcagcagagt
ggaggctgag gacgtgggcg tctactactg ctggcagggc acccacttcc ccaggacctt cggccaggga accaaggtgg
agatcaaggc tagcgtgaaa gggaaacacc tttgtccaag tcccctattt cccggacctt ctaagccctt ttgggtgctg
gtggtggttg gtggagtcct ggcttgctat agcttgctag taacagtggc ctttattatt ttctgggtga ggagtaagag
gagcaggctc ctgcacagtg actacatgaa catgactccc cgccgccccg ggcccacccg caagcattac cagccctatg
ccccaccacg cgacttcgca gcctatcgct ccaagcttag agtgaagttc agcaggagcg cagacgcccc cgcgtaccag
cagggccaga accagctcta taacgagctc aatctaggac gaagagagga gtacgatgtt ttggacaaga gacgtggccg
ggaccctgag atggggggaa agccgagaag gaagaaccct caggaaggcc tgtacaatga actgcagaaa gataagatgg
cggaggccta cagtgagatt gggatgaaag gcgagcgccg gaggggcaag gggcacgatg gcctttacca gggtctcagt
acagccacca aggacaccta cgacgccctt cacatgcagg ccctgccccc t
DG03.28.Z CAR (SEQ ID NO: 222)
atggaatgga cctgggtgtt cctgtttctg ctctccgtga ccgccggagt gcacagcgag gtgcagctgg tcgaaagcgg
cggaggactg gtgcagcctg gcggcagcct gagactgagc tgtgccgcct ccggcttcac ctttagcagc tacggaatgt
cctgggtgag acaggctcct ggcaagggcc tggaactggt ggccagcatc aatagcaacg gcggcagcac ctactaccct
gatagcgtga agggcaggtt caccatctcc agggacaacg ccaagaacag cctgtacctg cagatgaaca gcctcagggc
cgaggacaca gccgtgtact actgcgccag cggcgactat tggggacagg gaacaaccgt gaccgtcagc agcgccggcg
gcggcggcag cggcggcggc ggcagcggcg gcggcggctc cgatatcgtg atgacccaga gccccctgtc cctgcctgtc
acacctggcg aacccgccag cattagctgc aggtccagcc agagcctggt gtacagcaat ggcgacacct acctgcactg
gtacctgcag aagcctggcc agagccccca gctgctgatc tacaaggtga gcaacaggtt ctccggagtg cctgacaggt
tcagcggctc cggcagcgga accgatttca ccctcaagat cagcagagtg gaggccgagg acgtgggcgt ctactattgt
agccagagca cccacgtgcc ctggaccttt ggccagggca ccaaggtgga gatcaaagct agcgtgaaag ggaaacacct
ttgtccaagt cccctatttc ccggaccttc taagcccttt tgggtgctgg tggtggttgg tggagtcctg gcttgctata
gcttgctagt aacagtggcc tttattattt tctgggtgag gagtaagagg agcaggctcc tgcacagtga ctacatgaac
atgactcccc gccgccccgg gcccacccgc aagcattacc agccctatgc cccaccacgc gacttcgcag cctatcgctc
caagcttaga gtgaagttca gcaggagcgc agacgccccc gcgtaccagc agggccagaa ccagctctat aacgagctca
atctaggacg aagagaggag tacgatgttt tggacaagag acgtggccgg gaccctgaga tggggggaaa gccgagaagg
aagaaccctc aggaaggcct gtacaatgaa ctgcagaaag ataagatggc ggaggcctac agtgagattg ggatgaaagg
cgagcgccgg aggggcaagg ggcacgatgg cctttaccag ggtctcagta cagccaccaa ggacacctac gacgcccttc
acatgcaggc cctgccccct
DG04.28.Z CAR (SEQ ID NO: 223)
atggaatgga cctgggtgtt tctgttcctc ctgagcgtga ccgccggagt gcacagccaa gtggagctgg tggagagcgg
aggaggactg gtgcagcctg gaggctccct gaggctgagc tgtgctgcca gcggcttcac cttcagctcc tatgctatga
gctgggtgag acaggcccct ggcaaaggcc tggagtgggt gagcgccatc aacgcctccg gcaccaggac ctactatgcc
gactccgtga agggcaggtt caccatctcc agggacaaca gcaagaacac cctgtacctg cagatgaaca gcctgagggc
tgaagacacc gccgtgtact actgtgccag gggcaagggc aacacacaca agccctatgg ctacgtgaga tacttcgacg
tgtggggaca gggcaccctg gtgacagtga gcagcgccgg aggaggaggt tctggaggag gaggaagcgg
cggaggagga agcgacatcg tgctgacaca atcccccgcc acactgtccc tgtcccctgg cgagagggcc acactgagct
gcagggccag ccagagcgtg tcctcctcct acctggcctg gtaccagcag aaacctggcc aggcccccag gctgctgatc
tatggcgcca gcagcagagc cacaggagtg cctgccagat ttagcggcag cggcagcggc accgacttta ccctgaccat
ttccagcctg gagcccgagg acttcgccac ctactactgc ctgcagatct acaacatgcc tatcaccttc ggccagggca
caaaagtgga aatcaaggct agcgtgaaag ggaaacacct ttgtccaagt cccctatttc ccggaccttc taagcccttt
tgggtgctgg tggtggttgg tggagtcctg gcttgctata gcttgctagt aacagtggcc tttattattt tctgggtgag
gagtaagagg agcaggctcc tgcacagtga ctacatgaac atgactcccc gccgccccgg gcccacccgc aagcattacc
agccctatgc cccaccacgc gacttcgcag cctatcgctc caagcttaga gtgaagttca gcaggagcgc agacgccccc
gcgtaccagc agggccagaa ccagctctat aacgagctca atctaggacg aagagaggag tacgatgttt tggacaagag
acgtggccgg gaccctgaga tggggggaaa gccgagaagg aagaaccctc aggaaggcct gtacaatgaa ctgcagaaag
ataagatggc ggaggcctac agtgagattg ggatgaaagg cgagcgccgg aggggcaagg ggcacgatgg cctttaccag
ggtctcagta cagccaccaa ggacacctac gacgcccttc acatgcaggc cctgccccct
DG05.28.Z CAR (SEQ ID NO: 224)
atggaatgga cctgggtgtt tctgttcctc ctgtccgtga ccgccggagt gcactccgaa gtgcagctgg tgcagtccgg
cggaggactg gtgaaacccg gaggaagcct cagactgagc tgcgccggca gcggctttac cttctccagc tactccatgc
actggctgag acaggcccct ggcaagggcc tggaatgggt cagcgccatc ggcaccgccg gaggcacata ctatgccgac
agcgtgaagg gcaggttcac catcagcagg gacaacgcca agaacagcct gtacctgcag atgaactctc tgagggccga
ggataccgct gtgtactact gcgccaggga gtacttcttt ggcagcggca actacggata ctggggccag ggcaccctgg
tgacagtgag ctccgccgga ggaggaggaa gcggaggagg cggaagcgga ggaggcggca gcgaaatcgt
gctgacccag agccctgcca ccctgagcct gagccctggc gaaagggcca ccctgagctg cagagccagc cagagcgtga
gcagctacct ggcctggtac cagcagaagc ccggacaggc ccccagactg ctgatctacg acgccagcaa cagagccacc
ggcattcccg ccagattctc cggcagcggc agcggaaccg acttcacact gaccatcagc tccttagaac ccgaggactt
cgccgtgtac tactgtcagc agagaagcaa ctggcctccc accttcggcc agggcacaaa ggtggagatc aaggctagcg
tgaaagggaa acacctttgt ccaagtcccc tatttcccgg accttctaag cccttttggg tgctggtggt ggttggtgga
gtcctggctt gctatagctt gctagtaaca gtggccttta ttttttactg ggtgaggagt aagaggagca ggctcctgca
cagtgactac atgaacatga ctccccgccg ccccgggccc acccgcaagc attaccagcc ctatgcccca ccacgcgact
tcgcagccta tcgctccaag cttagagtga agttcagcag gagcgcagac gcccccgcgt accagcaggg ccagaaccag
ctctataacg agctcaatct aggacgaaga gaggagtacg atgttttgga caagagacgt ggccgggacc ctgagatggg
gggaaagccg agaaggaaga accctcagga aggcctgtac aatgaactgc agaaagataa gatggcggag gcctacagtg
agattgggat gaaaggcgag cgccggaggg gcaaggggca cgatggcctt taccagggtc tcagtacagc caccaaggac
acctacgacg cccttcacat gcaggccctg ccccct
DG06.28.Z CAR (SEQ ID NO: 225)
atggaatgga cctgggtgtt cctgttcctc ctgagcgtga ccgctggcgt ccacagccag gtgcagctgg tggaaagcgg
aggcggagtg gtgcagcctg gaaggtccct cagactgagc tgcgctgcca gcggcttcac cttcagcaac tacggcatcc
actgggtgag acaagccccc ggcaaaggcc tggagtgggt ggccatcatc tggcacgacg gcagcaactc ctactacgtg
gactccgtga agggcaggtt cacaatcagc agagacaaca gcaagaatac cctgtacctg cagatgaaca gcctcagggc
cgaagatacc gccgtgtact tctgcgccag gatcatcggc ggcgcctttg acatttgggg ccaaggcact atggttaccg
tgagcagcgc tggcggaggc ggcagcggcg gcggcggcag cggcggcggc ggaagcgaca tccagatgac
ccagagccct tccagcctca gcgcctccgt gggagacaga gtgaccatca cctgcagggc cagccagggc atctccagct
ggctggcctg gtaccagcag aagcctgaga aagcccccaa gagcctgatc tacgctgcct ccagcctgca gtccggcgtg
ccttccagat tctccggcag cggcagcggc accgacttta ccctgaccat ttccagcctg caacccgagg acttcgccac
ctactactgc cagcagtaca acagctaccc catcaccttt ggccagggca ccagactgga gatcaaggct agcgtgaaag
ggaaacacct ttgtccaagt cccctatttc ccggaccttc taagcccttt tgggtgctgg tggtggttgg tggagtcctg
gcttgctata gcttgctagt aacagtggcc tttattattt tctgggtgag gagtaagagg agcaggctcc tgcacagtga
ctacatgaac atgactcccc gccgccccgg gcccacccgc aagcattacc agccctatgc cccaccacgc gacttcgcag
cctatcgctc caagcttaga gtgaagttca gcaggagcgc agacgccccc gcgtaccagc agggccagaa ccagctctat
aacgagctca atctaggacg aagagaggag tacgatgttt tggacaagag acgtggccgg gaccctgaga tggggggaaa
gccgagaagg aagaaccctc aggaaggcct gtacaatgaa ctgcagaaag ataagatggc ggaggcctac agtgagattg
ggatgaaagg cgagcgccgg aggggcaagg ggcacgatgg cctttaccag ggtctcagta cagccaccaa ggacacctac
gacgcccttc acatgcaggc cctgccccct
DG07.28.Z CAR (SEQ ID NO: 226)
atggaatgga cctgggtgtt cctgttcctg ctgtccgtga ccgccggagt ccacagcgag gtgcagctgg tggaaagcgg
cggaggactg gtgcagcctg gaggcagcct gaggctgagc tgtgctgcca gcggcttctc catcagcggc tactggatgt
cctgggtgag gcaggcccct ggaaagggct tagaatgggt ggccaacatc aaacaggacg gcggcgagaa gtactacgga
gacagcgtca agggcagatt caccatcagc agggacaacg ccaagaacag cctgtacctg cagatgaaca gcctgagggc
cgaggacacc gctgtgtact actgcgtgat ggccggaggc ctggattatt ggggccaggg cacactggtg acagtgagca
gcgccggcgg cggcggcagc ggcggcggcg gcagcggcgg cggcggcagc gagatcgtgc tgacccagag
ccctgccaca ctgagcctga gccccggcga aagagccacc ctcagctgca gggccagcca gagcgtgagc agctacctgg
cctggtacca gcagaagccc ggacaggccc ctaggctgct gatctacgat gccagcaaca gagccaccgg catccctgct
aggttcagcg gttctggcag cggcaccgac ttcaccctga ccatcagcag cctggagcct gaggacttcg ctgtctacta
ctgccagcag aggagcaact ggtatacctt cggccaggga accaagctgg agatcaaggc tagcgtgaaa gggaaacacc
tttgtccaag tcccctattt cccggacctt ctaagccctt ttgggtgctg gtggtggttg gtggagtcct ggcttgctat
agcttgctag taacagtggc ctttattatt ttctgggtga ggagtaagag gagcaggctc ctgcacagtg actacatgaa
catgactccc cgccgccccg ggcccacccg caagcattac cagccctatg ccccaccacg cgacttcgca gcctatcgct
ccaagcttag agtgaagttc agcaggagcg cagacgcccc cgcgtaccag cagggccaga accagctcta taacgagctc
aatctaggac gaagagagga gtacgatgtt ttggacaaga gacgtggccg ggaccctgag atggggggaa agccgagaag
gaagaaccct caggaaggcc tgtacaatga actgcagaaa gataagatgg cggaggccta cagtgagatt gggatgaaag
gcgagcgccg gaggggcaag gggcacgatg gcctttacca gggtctcagt acagccacca aggacaccta cgacgccctt
cacatgcagg ccctgccccc t
DG08.28.Z CAR (SEQ ID NO: 227)
atggaatgga cctgggtgtt cctgttcctg ctcagcgtga ccgccggagt gcactcccag gtgcagctgg tgcagagcgg
cgccgaagtg aaaaagcccg gcgccagcgt gagactctcc tgtagagcca gcggctacaa cttcatcgac ttccacatcc
actgggtgag acaggcccct ggagagggcc tggagtggat gggctggagc aacccccaga gcggcaatag
cagcagcgcc cagaggttcc agggcagagt gaccatgacc accgatacct ccatgagcgc cgcctacatg gacctgaact
ggctgaccct ggacgacacc gccgtgtact actgcaccag gcctcacgac ggcgctggca actacaggtt cgacacctgg
ggacagggaa ccctggtgac agtcagcagc gcgggaggtg gtggtagcgg tggtgggggt tccggtggag gtggcagttc
atatgagctt acacaacccc caagtgtgag cgtggctcct ggccagacag ccaggatcac ctgcagcgga gacgccctgc
ccaaacacta cgcccactgg tatcagcaga aacccggcca ggtgcccatc gtggtgatct acaaggacac cgagagacct
agcggcatcc ccgagagatt cagcggcagc accagcggca ccacagtcac cctgaccatc tccggcgtgc aggccgagga
tgaagcccac tactactgcc aaagcgccga cgtgtcctcc acctatgtgg tgttcggcgg cggcaccaag ctgaccgtcc
tcgctagcgt gaaagggaaa cacctttgtc caagtcccct atttcccgga ccttctaagc ccttttgggt gctggtggtg
gttggtggag tcctggcttg ctatagcttg ctagtaacag tggcctttat tattttctgg gtgaggagta agaggagcag
gctcctgcac agtgactaca tgaacatgac tccccgccgc cccgggccca cccgcaagca ttaccagccc tatgccccac
cacgcgactt cgcagcctat cgctccaagc ttagagtgaa gttcagcagg agcgcagacg cccccgcgta ccagcagggc
cagaaccagc tctataacga gctcaatcta ggacgaagag aggagtacga tgttttggac aagagacgtg gccgggaccc
tgagatgggg ggaaagccga gaaggaagaa ccctcaggaa ggcctgtaca atgaactgca gaaagataag atggcggagg
cctacagtga gattgggatg aaaggcgagc gccggagggg caaggggcac gatggccttt accagggtct cagtacagcc
accaaggaca cctacgacgc ccttcacatg caggccctgc cccct
DG09.28.Z CAR (SEQ ID NO: 228)
atggagtgga cttgggtatt cctgtttttg ttgtccgtaa ctgctggggt acactcagag gtgcagctgg tagagtctgg
aggggggctc gtcgaaccag gcggctctct caggctttcc tgtgccgtaa gcgggtttga ttttgagaaa gcatggatgt
cctgggtaag gcaagctcca gggcagggac tccagtgggt agcgcggata aagtcaacag ctgatggcgg aaccacctct
tatgcagcac cggttgaggg aaggttcatc atctcacgag acgattcccg caacatgttg tatctgcaga tgaacagttt
gaaaactgaa gacacagctg tttactactg tacttcagcg cattggggac agggtactct tgtgacggtc tctagcgccg
gggggggagg ctctggaggg gggggttcag ggggtggtgg cagctcctat gagctgactc aaccgccttc agtaagcgta
agccctggtc agaccgctag aataacctgt agtggagagg ccctgccgat gcaattcgcc cactggtatc agcagaggcc
tggaaaagcc ccagtgattg tcgtttacaa agattccgaa cgccctagcg gggttcccga acgctttagc ggtagttcaa
gcgggacaac agcaaccctt acgataaccg gtgtacaagc ggaagacgaa gcggattact attgccaatc acctgatagt
acaaatactt atgaggtatt tggcggggga acgaagttga ctgtactggc tagcgtgaaa gggaaacacc tttgtccaag
tcccctattt cccggacctt ctaagccctt ttgggtgctg gtggtggttg gtggagtcct ggcttgctat agcttgctag
taacagtggc ctttattatt ttctgggtga ggagtaagag gagcaggctc ctgcacagtg actacatgaa catgactccc
cgccgccccg ggcccacccg caagcattac cagccctatg ccccaccacg cgacttcgca gcctatcgct ccaagcttag
agtgaagttc agcaggagcg cagacgcccc cgcgtaccag cagggccaga accagctcta taacgagctc aatctaggac
gaagagagga gtacgatgtt ttggacaaga gacgtggccg ggaccctgag atggggggaa agccgagaag gaagaaccct
caggaaggcc tgtacaatga actgcagaaa gataagatgg cggaggccta cagtgagatt gggatgaaag gcgagcgccg
gaggggcaag gggcacgatg gcctttacca gggtctcagt acagccacca aggacaccta cgacgccctt cacatgcagg
ccctgccccc t
DG10.28.Z CAR (SEQ ID NO: 229)
atggaatgga cctgggtgtt cctcttcctc ctgtccgtca ccgctggcgt gcacagccag gtgcagctgg tccagagcgg
agccgaggtg aaaaagcccg gcgcctccgt gaaggtcagc tgcaaggcct ccggctacac cttcaccaac tacgccatgc
actgggtgag acaggcccct ggccagagac tggagtggat gggctggatc aacgccggca acggcaagag aaagtacagc
cagaagtttc aggacagggt gaccatcaac agggacacca gcgcctccac catctacatg gagctgtcca gcctgggcag
cgaggatacc gccgtgtact actgtgccag agaggaggat cacgctggca gcggcagcta cctgagcatg gacgtctggg
gacagggcag caccgtgaca gtgagcagcg ctggaggcgg cggctccggc ggcggaggaa gcggaggcgg
aggctccgac atcgtgatga cccagtcccc cgatagcctg gctgtgagcc tgggcgagag ggccacaatc aactgtaaga
gcagccagaa cgtgctgtac tcctccaaca acaagaacta cctggcctgg taccagcaga aacctggcca tccccccaag
ctgctgatct actgggccag caccagggag agcggagtgc ctgacaggtt tagcggcagc ggcagcggca cagactttac
cctgaccatc acctccctgc agaccgagga cgtggccgtg tactattgcc agcagtacta cagctcccct ctgaccttcg
gcggcggcac caaagtggag atcaaagcta gcgtgaaagg gaaacacctt tgtccaagtc ccctatttcc cggaccttct
aagccctttt gggtgctggt ggtggttggt ggagtcctgg cttgctatag cttgctagta acagtggcct ttattatttt
ctgggtgagg agtaagagga gcaggctcct gcacagtgac tacatgaaca tgactccccg ccgccccggg cccacccgca
agcattacca gccctatgcc ccaccacgcg acttcgcagc ctatcgctcc aagcttagag tgaagttcag caggagcgca
gacgcccccg cgtaccagca gggccagaac cagctctata acgagctcaa tctaggacga agagaggagt acgatgtttt
ggacaagaga cgtggccggg accctgagat ggggggaaag ccgagaagga agaaccctca ggaaggcctg
tacaatgaac tgcagaaaga taagatggcg gaggcctaca gtgagattgg gatgaaaggc gagcgccgga ggggcaaggg
gcacgatggc ctttaccagg gtctcagtac agccaccaag gacacctacg acgcccttca catgcaggcc ctgccccct
DG11.28.Z CAR (SEQ ID NO: 230)
atggagtgga cttgggtatt ccttttcctc ctctccgtga cagcgggtgt gcactctgaa gtacaacttg tagagaccgg
tgggggattg gtgcaaccca agggttccct gaaactctca tgtgctacct ctggttttac tttcaacacc tatgcaatga
attgggttag gcaagcaccc ggtaaaggac ttgagtgggt ggcacggata cgcactaaga gtaatgacta tgctacgtac
tacgcagact ccgtaaaagg ccggatcacc atatctcgag acgatagcca gtctatgctg tatcttcaaa tgaacaacct
caaaacggaa gatacggcga tgtattactg cgtgcgagtt ggttataggc cttatgctat ggattactgg ggacagggca
cgtctgtcac ggtaagttct gccggagggg ggggcagcgg aggaggagga tctggcggag ggggctccga tgtccttatg
acacagactc ccctcagttt gcccgtgtcc ttgggggacc aggcttctat atcatgccgc agttcccaaa atatcgtcca
ttcaaatggc aatacttacc ttgagtggta tttgcagaag cctggacaga gcccgacgct tctgatctat aaggtaagca
acaggttcag tggtgtaccc gacagattta gtggaagtgg gtccggaact gatttcactc ttaagattag tcgggtagag
gctgaagacc ttggggtgta ttattgcttt caagggagtc acgtccctct tacatttggt gctgggacta agttggagct
gaaggctagc gtgaaaggga aacacctttg tccaagtccc ctatttcccg gaccttctaa gcccttttgg gtgctggtgg
tggttggtgg agtcctggct tgctatagct tgctagtaac agtggccttt attattttct gggtgaggag taagaggagc
aggctcctgc acagtgacta catgaacatg actccccgcc gccccgggcc cacccgcaag cattaccagc cctatgcccc
accacgcgac ttcgcagcct atcgctccaa gcttagagtg aagttcagca ggagcgcaga cgcccccgcg taccagcagg
gccagaacca gctctataac gagctcaatc taggacgaag agaggagtac gatgttttgg acaagagacg tggccgggac
cctgagatgg ggggaaagcc gagaaggaag aaccctcagg aaggcctgta caatgaactg cagaaagata agatggcgga
ggcctacagt gagattggga tgaaaggcga gcgccggagg ggcaaggggc acgatggcct ttaccagggt ctcagtacag
ccaccaagga cacctacgac gcccttcaca tgcaggccct gccccct
SIGNAL SEQUENCE (SEQ ID NO: 231)
atggaatgga cctgggtctt tctcttcctc ctgtcagtaa ctgcaggtgt ccac
HUMAN CD28 HINGE (SEQ ID NO: 232)
gtgaaaggga aacacctttg tccaagtccc ctatttcccg gaccttctaa gccc
MOUSE CD28 HINGE (SEQ ID NO: 233)
ataaaagaga aacatctttg tcatactcag tcatctccta agctg
HUMAN CD8A HINGE (SEQ ID NO: 234)
accacgacgc cagcgccgcg accaccaaca ccggcgccca ccatcgcgtc gcagcccctg tccctgcgcc cagaggcgtg
ccggccagcg gcggggggcg cagtgcacac gagggggctg gacttcgcct gtgat
HUMAN DAP10 HINGE (SEQ ID NO: 235)
cagacgaccc caggagagag atcatcactc cctgcctttt accctggcac ttcaggctcc tgttccggat gtgggtccct
ctctctgccg
HUMAN CD28 TM (SEQ ID NO: 236)
ttttgggtgc tggtggtggt tggtggagtc ctggcttgct atagcttgct agtaacagtg gcctttatta tatctgggt g
MOUSE CD28 TM (SEQ ID NO: 237)
ttttgggcac tggtcgtggt tgctggagtc ctgttttgtt atggcttgct agtgacagtg gctctttgtg ttatctggac a
HUMAN CD8A TM (SEQ ID NO: 238)
atctacatct gggcgccctt ggccgggact tgtggggtcc ttctcctgtc actggttatc accctttact gc
HUMAN DAP10 TM (SEQ ID NO: 239)
ctcctggcag gcctcgtggc tgctgatgcg gtggcatcgc tgctcatcgt gggggcggtg ttc
DG05-CD28tm-DAP10-CD3ζ (SEQ ID NO: 240)
ATGGAATGGACCTGGGTGTTTCTGTTCCTCCTGTCCGTGACCGCCGGAGTGCACT
CCGAAGTGCAGCTGGTGCAGTCCGGCGGAGGACTGGTGAAACCCGGAGGAAGCC
TCAGACTGAGCTGCGCCGGCAGCGGCTTTACCTTCTCCAGCTACTCCATGCACTG
GCTGAGACAGGCCCCTGGCAAGGGCCTGGAATGGGTCAGCGCCATCGGCACCGC
CGGAGGCACATACTATGCCGACAGCGTGAAGGGCAGGTTCACCATCAGCAGGGA
CAACGCCAAGAACAGCCTGTACCTGCAGATGAACTCTCTGAGGGCCGAGGATAC
CGCTGTGTACTACTGCGCCAGGGAGTACTTCTTTGGCAGCGGCAACTACGGATAC
TGGGGCCAGGGCACCCTGGTGACAGTGAGCTCCGCCGGAGGAGGAGGAAGCGG
AGGAGGCGGAAGCGGAGGAGGCGGCAGCGAAATCGTGCTGACCCAGAGCCCTG
CCACCCTGAGCCTGAGCCCTGGCGAAAGGGCCACCCTGAGCTGCAGAGCCAGCC
AGAGCGTGAGCAGCTACCTGGCCTGGTACCAGCAGAAGCCCGGACAGGCCCCCA
GACTGCTGATCTACGACGCCAGCAACAGAGCCACCGGCATTCCCGCCAGATTCTC
CGGCAGCGGCAGCGGAACCGACTTCACACTGACCATCAGCTCCTTAGAACCCGA
GGACTTCGCCGTGTACTACTGTCAGCAGAGAAGCAACTGGCCTCCCACCTTCGGC
CAGGGCACAAAGGTGGAGATCAAGGCTAGCGTGAAAGGGAAACACCTTTGTCCA
AGTCCCCTATTTCCCGGACCTTCTAAGCCCTTTTGGGTGCTGGTGGTGGTTGGTGG
AGTCCTGGCTTGCTATAGCTTGCTAGTAACAGTGGCCTTTATTATTTTCTGGGTGA
GGAGTAAGAGGAGCCTGTGCGCACGCCCACGCCGCAGCCCCGCCCAAGAAGATG
GCAAAGTCTACATCAACATGCCAGGCAGGGGCAAGCTTAGAGTGAAGTTCAGCA
GGAGCGCAGACGCCCCCGCGTACCAGCAGGGCCAGAACCAGCTCTATAACGAGC
TCAATCTAGGACGAAGAGAGGAGTACGATGTTTTGGACAAGAGACGTGGCCGGG
ACCCTGAGATGGGGGGAAAGCCGAGAAGGAAGAACCCTCAGGAAGGCCTGTAC
AATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTAC
AGCCACCAAGGACACCTACGACGCCCTTCACATGCAGGCCCTGCCCCCTCGC
DG05-CD28tm-CD44-CD3ζ (SEQ ID NO: 241)
ATGGAATGGACCTGGGTGTTTCTGTTCCTCCTGTCCGTGACCGCCGGAGTGCACT
CCGAAGTGCAGCTGGTGCAGTCCGGCGGAGGACTGGTGAAACCCGGAGGAAGCC
TCAGACTGAGCTGCGCCGGCAGCGGCTTTACCTTCTCCAGCTACTCCATGCACTG
GCTGAGACAGGCCCCTGGCAAGGGCCTGGAATGGGTCAGCGCCATCGGCACCGC
CGGAGGCACATACTATGCCGACAGCGTGAAGGGCAGGTTCACCATCAGCAGGGA
CAACGCCAAGAACAGCCTGTACCTGCAGATGAACTCTCTGAGGGCCGAGGATAC
CGCTGTGTACTACTGCGCCAGGGAGTACTTCTTTGGCAGCGGCAACTACGGATAC
TGGGGCCAGGGCACCCTGGTGACAGTGAGCTCCGCCGGAGGAGGAGGAAGCGG
AGGAGGCGGAAGCGGAGGAGGCGGCAGCGAAATCGTGCTGACCCAGAGCCCTG
CCACCCTGAGCCTGAGCCCTGGCGAAAGGGCCACCCTGAGCTGCAGAGCCAGCC
AGAGCGTGAGCAGCTACCTGGCCTGGTACCAGCAGAAGCCCGGACAGGCCCCCA
GACTGCTGATCTACGACGCCAGCAACAGAGCCACCGGCATTCCCGCCAGATTCTC
CGGCAGCGGCAGCGGAACCGACTTCACACTGACCATCAGCTCCTTAGAACCCGA
GGACTTCGCCGTGTACTACTGTCAGCAGAGAAGCAACTGGCCTCCCACCTTCGGC
CAGGGCACAAAGGTGGAGATCAAGGCTAGCGTGAAAGGGAAACACCTTTGTCCA
AGTCCCCTATTTCCCGGACCTTCTAAGCCCTTTTGGGTGCTGGTGGTGGTTGGTGG
AGTCCTGGCTTGCTATAGCTTGCTAGTAACAGTGGCCTTTATTATTTTCTGGGTGA
GTCGAAGAAGGTGTGGGCAGAAGAAAAAGCTAGTGATCAACAGTGGCAATGGA
GCTGTGGAGGACAGAAAGCCAAGTGGACTCAACGGAGAGGCCAGCAAGTCTCA
GGAAATGGTGCATTTGGTGAACAAGGAGTCGTCAGAAACTCCAGACCAGTTTAT
GACAGCTGATGAGACAAGGAACCTGCAGAATGTGGACATGAAGATTGGGGTGAG
AGTGAAGTTCAGCAGGAGCGCAGACGCCCCCGCGTACCAGCAGGGCCAGAACCA
GCTCTATAACGAGCTCAATCTAGGACGAAGAGAGGAGTACGATGTTTTGGACAA
GAGACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCC
TGCCCCCTCGC
DG05-CD28tm-CD3ζ (SEQ ID NO: 242)
ATGGAATGGACCTGGGTGTTTCTGTTCCTCCTGTCCGTGACCGCCGGAGTGCACT
CCGAAGTGCAGCTGGTGCAGTCCGGCGGAGGACTGGTGAAACCCGGAGGAAGCC
TCAGACTGAGCTGCGCCGGCAGCGGCTTTACCTTCTCCAGCTACTCCATGCACTG
GCTGAGACAGGCCCCTGGCAAGGGCCTGGAATGGGTCAGCGCCATCGGCACCGC
CGGAGGCACATACTATGCCGACAGCGTGAAGGGCAGGTTCACCATCAGCAGGGA
CAACGCCAAGAACAGCCTGTACCTGCAGATGAACTCTCTGAGGGCCGAGGATAC
CGCTGTGTACTACTGCGCCAGGGAGTACTTCTTTGGCAGCGGCAACTACGGATAC
TGGGGCCAGGGCACCCTGGTGACAGTGAGCTCCGCCGGAGGAGGAGGAAGCGG
AGGAGGCGGAAGCGGAGGAGGCGGCAGCGAAATCGTGCTGACCCAGAGCCCTG
CCACCCTGAGCCTGAGCCCTGGCGAAAGGGCCACCCTGAGCTGCAGAGCCAGCC
AGAGCGTGAGCAGCTACCTGGCCTGGTACCAGCAGAAGCCCGGACAGGCCCCCA
GACTGCTGATCTACGACGCCAGCAACAGAGCCACCGGCATTCCCGCCAGATTCTC
CGGCAGCGGCAGCGGAACCGACTTCACACTGACCATCAGCTCCTTAGAACCCGA
GGACTTCGCCGTGTACTACTGTCAGCAGAGAAGCAACTGGCCTCCCACCTTCGGC
CAGGGCACAAAGGTGGAGATCAAGGCTAGCGTGAAAGGGAAACACCTTTGTCCA
AGTCCCCTATTTCCCGGACCTTCTAAGCCCTTTTGGGTGCTGGTGGTGGTTGGTGG
AGTCCTGGCTTGCTATAGCTTGCTAGTAACAGTGGCCTTTATTATTTTCTGGGTGA
GGAGTAAGAGGAGCAGGAGAGTGAAGTTCAGCAGGAGCGCAGACGCCCCCGCG
TACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAG
CCGAGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACG
ACGCCCTTCACATGCAGGCCCTGCCCCCTCGC
DG05-CD28 (SEQ ID NO: 243)
ATGGAATGGACCTGGGTGTTTCTGTTCCTCCTGTCCGTGACCGCCGGAGTGCACT
CCGAAGTGCAGCTGGTGCAGTCCGGCGGAGGACTGGTGAAACCCGGAGGAAGCC
TCAGACTGAGCTGCGCCGGCAGCGGCTTTACCTTCTCCAGCTACTCCATGCACTG
GCTGAGACAGGCCCCTGGCAAGGGCCTGGAATGGGTCAGCGCCATCGGCACCGC
CGGAGGCACATACTATGCCGACAGCGTGAAGGGCAGGTTCACCATCAGCAGGGA
CAACGCCAAGAACAGCCTGTACCTGCAGATGAACTCTCTGAGGGCCGAGGATAC
CGCTGTGTACTACTGCGCCAGGGAGTACTTCTTTGGCAGCGGCAACTACGGATAC
TGGGGCCAGGGCACCCTGGTGACAGTGAGCTCCGCCGGAGGAGGAGGAAGCGG
AGGAGGCGGAAGCGGAGGAGGCGGCAGCGAAATCGTGCTGACCCAGAGCCCTG
CCACCCTGAGCCTGAGCCCTGGCGAAAGGGCCACCCTGAGCTGCAGAGCCAGCC
AGAGCGTGAGCAGCTACCTGGCCTGGTACCAGCAGAAGCCCGGACAGGCCCCCA
GACTGCTGATCTACGACGCCAGCAACAGAGCCACCGGCATTCCCGCCAGATTCTC
CGGCAGCGGCAGCGGAACCGACTTCACACTGACCATCAGCTCCTTAGAACCCGA
GGACTTCGCCGTGTACTACTGTCAGCAGAGAAGCAACTGGCCTCCCACCTTCGGC
CAGGGCACAAAGGTGGAGATCAAGGCTAGCGTGAAAGGGAAACACCTTTGTCCA
AGTCCCCTATTTCCCGGACCTTCTAAGCCCTTTTGGGTGCTGGTGGTGGTTGGTGG
AGTCCTGGCTTGCTATAGCTTGCTAGTAACAGTGGCCTTTATTATTTTCTGGGTGA
GGAGTAAGAGGAGCAGGCTCCTGCACAGTGACTACATGAACATGACTCCCCGCC
GCCCCGGGCCCACCCGCAAGCATTACCAGCCCTATGCCCCACCACGCGACTTCGC
AGCCTATCGCTCC
DG05-CD28tm (SEQ ID NO: 244)
ATGGAATGGACCTGGGTGTTTCTGTTCCTCCTGTCCGTGACCGCCGGAGTGCACT
CCGAAGTGCAGCTGGTGCAGTCCGGCGGAGGACTGGTGAAACCCGGAGGAAGCC
TCAGACTGAGCTGCGCCGGCAGCGGCTTTACCTTCTCCAGCTACTCCATGCACTG
GCTGAGACAGGCCCCTGGCAAGGGCCTGGAATGGGTCAGCGCCATCGGCACCGC
CGGAGGCACATACTATGCCGACAGCGTGAAGGGCAGGTTCACCATCAGCAGGGA
CAACGCCAAGAACAGCCTGTACCTGCAGATGAACTCTCTGAGGGCCGAGGATAC
CGCTGTGTACTACTGCGCCAGGGAGTACTTCTTTGGCAGCGGCAACTACGGATAC
TGGGGCCAGGGCACCCTGGTGACAGTGAGCTCCGCCGGAGGAGGAGGAAGCGG
AGGAGGCGGAAGCGGAGGAGGCGGCAGCGAAATCGTGCTGACCCAGAGCCCTG
CCACCCTGAGCCTGAGCCCTGGCGAAAGGGCCACCCTGAGCTGCAGAGCCAGCC
AGAGCGTGAGCAGCTACCTGGCCTGGTACCAGCAGAAGCCCGGACAGGCCCCCA
GACTGCTGATCTACGACGCCAGCAACAGAGCCACCGGCATTCCCGCCAGATTCTC
CGGCAGCGGCAGCGGAACCGACTTCACACTGACCATCAGCTCCTTAGAACCCGA
GGACTTCGCCGTGTACTACTGTCAGCAGAGAAGCAACTGGCCTCCCACCTTCGGC
CAGGGCACAAAGGTGGAGATCAAGGCTAGCGTGAAAGGGAAACACCTTTGTCCA
AGTCCCCTATTTCCCGGACCTTCTAAGCCCTTTTGGGTGCTGGTGGTGGTTGGTGG
AGTCCTGGCTTGCTATAGCTTGCTAGTAACAGTGGCCTTTATTATTTTCTGGGTGA
GGAGTAAGAGGAGCAGGCTCCTGCACAGTGAC
DG03-CD28tm-DAP10-CD3ζ (SEQ ID NO: 245)
ATGGAATGGACCTGGGTGTTCCTGTTTCTGCTCTCCGTGACCGCCGGAGTGCACA
GCGAGGTGCAGCTGGTCGAAAGCGGCGGAGGACTGGTGCAGCCTGGCGGCAGCC
TGAGACTGAGCTGTGCCGCCTCCGGCTTCACCTTTAGCAGCTACGGAATGTCCTG
GGTGAGACAGGCTCCTGGCAAGGGCCTGGAACTGGTGGCCAGCATCAATAGCAA
CGGCGGCAGCACCTACTACCCTGATAGCGTGAAGGGCAGGTTCACCATCTCCAG
GGACAACGCCAAGAACAGCCTGTACCTGCAGATGAACAGCCTCAGGGCCGAGGA
CACAGCCGTGTACTACTGCGCCAGCGGCGACTATTGGGGACAGGGAACAACCGT
GACCGTCAGCAGCGCCGGCGGCGGCGGCAGCGGCGGCGGCGGCAGCGGCGGCG
GCGGCTCCGATATCGTGATGACCCAGAGCCCCCTGTCCCTGCCTGTCACACCTGG
CGAACCCGCCAGCATTAGCTGCAGGTCCAGCCAGAGCCTGGTGTACAGCAATGG
CGACACCTACCTGCACTGGTACCTGCAGAAGCCTGGCCAGAGCCCCCAGCTGCT
GATCTACAAGGTGAGCAACAGGTTCTCCGGAGTGCCTGACAGGTTCAGCGGCTC
CGGCAGCGGAACCGATTTCACCCTCAAGATCAGCAGAGTGGAGGCCGAGGACGT
GGGCGTCTACTATTGTAGCCAGAGCACCCACGTGCCCTGGACCTTTGGCCAGGGC
ACCAAGGTGGAGATCAAAGCTAGCGTGAAAGGGAAACACCTTTGTCCAAGTCCC
CTATTTCCCGGACCTTCTAAGCCCTTTTGGGTGCTGGTGGTGGTTGGTGGAGTCCT
GGCTTGCTATAGCTTGCTAGTAACAGTGGCCTTTATTATTTTCTGGGTGAGGAGT
AAGAGGAGCCTGTGCGCACGCCCACGCCGCAGCCCCGCCCAAGAAGATGGCAAA
GTCTACATCAACATGCCAGGCAGGGGCAAGCTTAGAGTGAAGTTCAGCAGGAGC
GCAGACGCCCCCGCGTACCAGCAGGGCCAGAACCAGCTCTATAACGAGCTCAAT
CTAGGACGAAGAGAGGAGTACGATGTTTTGGACAAGAGACGTGGCCGGGACCCT
GAGATGGGGGGAAAGCCGAGAAGGAAGAACCCTCAGGAAGGCCTGTACAATGA
ACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCA
CCAAGGACACCTACGACGCCCTTCACATGCAGGCCCTGCCCCCTCGC
DG03-CD28tm-CD44-CD3ζ (SEQ ID NO: 246)
ATGGAATGGACCTGGGTGTTCCTGTTTCTGCTCTCCGTGACCGCCGGAGTGCACA
GCGAGGTGCAGCTGGTCGAAAGCGGCGGAGGACTGGTGCAGCCTGGCGGCAGCC
TGAGACTGAGCTGTGCCGCCTCCGGCTTCACCTTTAGCAGCTACGGAATGTCCTG
GGTGAGACAGGCTCCTGGCAAGGGCCTGGAACTGGTGGCCAGCATCAATAGCAA
CGGCGGCAGCACCTACTACCCTGATAGCGTGAAGGGCAGGTTCACCATCTCCAG
GGACAACGCCAAGAACAGCCTGTACCTGCAGATGAACAGCCTCAGGGCCGAGGA
CACAGCCGTGTACTACTGCGCCAGCGGCGACTATTGGGGACAGGGAACAACCGT
GACCGTCAGCAGCGCCGGCGGCGGCGGCAGCGGCGGCGGCGGCAGCGGCGGCG
GCGGCTCCGATATCGTGATGACCCAGAGCCCCCTGTCCCTGCCTGTCACACCTGG
CGAACCCGCCAGCATTAGCTGCAGGTCCAGCCAGAGCCTGGTGTACAGCAATGG
CGACACCTACCTGCACTGGTACCTGCAGAAGCCTGGCCAGAGCCCCCAGCTGCT
GATCTACAAGGTGAGCAACAGGTTCTCCGGAGTGCCTGACAGGTTCAGCGGCTC
CGGCAGCGGAACCGATTTCACCCTCAAGATCAGCAGAGTGGAGGCCGAGGACGT
GGGCGTCTACTATTGTAGCCAGAGCACCCACGTGCCCTGGACCTTTGGCCAGGGC
ACCAAGGTGGAGATCAAAGCTAGCGTGAAAGGGAAACACCTTTGTCCAAGTCCC
CTATTTCCCGGACCTTCTAAGCCCTTTTGGGTGCTGGTGGTGGTTGGTGGAGTCCT
GGCTTGCTATAGCTTGCTAGTAACAGTGGCCTTTATTATTTTCTGGGTGAGTCGAA
GAAGGTGTGGGCAGAAGAAAAAGCTAGTGATCAACAGTGGCAATGGAGCTGTG
GAGGACAGAAAGCCAAGTGGACTCAACGGAGAGGCCAGCAAGTCTCAGGAAAT
GGTGCATTTGGTGAACAAGGAGTCGTCAGAAACTCCAGACCAGTTTATGACAGC
TGATGAGACAAGGAACCTGCAGAATGTGGACATGAAGATTGGGGTGAGAGTGAA
GTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACG
TGGCCGGGACCCTGAGATGGGGGGAAAGCCGAGAAGGAAGAACCCTCAGGAAG
GCCTGTACAATGAACTGCAGAAAGATAAGATGGCGGAGGCCTACAGTGAGATTG
GGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCC
CTCGC
DG03-CD28tm-4-1-BB-CD3ζ (SEQ ID NO: 247)
ATGGAATGGACCTGGGTGTTCCTGTTTCTGCTCTCCGTGACCGCCGGAGTGCACA
GCGAGGTGCAGCTGGTCGAAAGCGGCGGAGGACTGGTGCAGCCTGGCGGCAGCC
TGAGACTGAGCTGTGCCGCCTCCGGCTTCACCTTTAGCAGCTACGGAATGTCCTG
GGTGAGACAGGCTCCTGGCAAGGGCCTGGAACTGGTGGCCAGCATCAATAGCAA
CGGCGGCAGCACCTACTACCCTGATAGCGTGAAGGGCAGGTTCACCATCTCCAG
GGACAACGCCAAGAACAGCCTGTACCTGCAGATGAACAGCCTCAGGGCCGAGGA
CACAGCCGTGTACTACTGCGCCAGCGGCGACTATTGGGGACAGGGAACAACCGT
GACCGTCAGCAGCGCCGGCGGCGGCGGCAGCGGCGGCGGCGGCAGCGGCGGCG
GCGGCTCCGATATCGTGATGACCCAGAGCCCCCTGTCCCTGCCTGTCACACCTGG
CGAACCCGCCAGCATTAGCTGCAGGTCCAGCCAGAGCCTGGTGTACAGCAATGG
CGACACCTACCTGCACTGGTACCTGCAGAAGCCTGGCCAGAGCCCCCAGCTGCT
GATCTACAAGGTGAGCAACAGGTTCTCCGGAGTGCCTGACAGGTTCAGCGGCTC
CGGCAGCGGAACCGATTTCACCCTCAAGATCAGCAGAGTGGAGGCCGAGGACGT
GGGCGTCTACTATTGTAGCCAGAGCACCCACGTGCCCTGGACCTTTGGCCAGGGC
ACCAAGGTGGAGATCAAAGCTAGCGTGAAAGGGAAACACCTTTGTCCAAGTCCC
CTATTTCCCGGACCTTCTAAGCCCTTTTGGGTGCTGGTGGTGGTTGGTGGAGTCCT
GGCTTGCTATAGCTTGCTAGTAACAGTGGCCTTTATTATTTTCTGGGTGAGGAGT
AAGAGGAGCCTCGAGAAACGGGGCAGAAAGAAACTCCTGTATATATTCAAACAA
CCATTTATGAGACCAGTACAAACTACTCAAGAGGAAGATGGCTGTAGCTGCCGA
TTTCCAGAAGAAGAAGAAGGAGGATGTGAACTGAAGCTTAGAGTGAAGTTCAGC
AGGAGCGCAGACGCCCCCGCGTACCAGCAGGGCCAGAACCAGCTCTATAACGAG
CTCAATCTAGGACGAAGAGAGGAGTACGATGTTTTGGACAAGAGACGTGGCCGG
GACCCTGAGATGGGGGGAAAGCCGAGAAGGAAGAACCCTCAGGAAGGCCTGTA
CAATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTAC
AGCCACCAAGGACACCTACGACGCCCTTCACATGCAGGCCCTGCCCCCTCGC
DG03-CD28tm-CD3ζ (SEQ ID NO: 248)
ATGGAATGGACCTGGGTGTTCCTGTTTCTGCTCTCCGTGACCGCCGGAGTGCACA
GCGAGGTGCAGCTGGTCGAAAGCGGCGGAGGACTGGTGCAGCCTGGCGGCAGCC
TGAGACTGAGCTGTGCCGCCTCCGGCTTCACCTTTAGCAGCTACGGAATGTCCTG
GGTGAGACAGGCTCCTGGCAAGGGCCTGGAACTGGTGGCCAGCATCAATAGCAA
CGGCGGCAGCACCTACTACCCTGATAGCGTGAAGGGCAGGTTCACCATCTCCAG
GGACAACGCCAAGAACAGCCTGTACCTGCAGATGAACAGCCTCAGGGCCGAGGA
CACAGCCGTGTACTACTGCGCCAGCGGCGACTATTGGGGACAGGGAACAACCGT
GACCGTCAGCAGCGCCGGCGGCGGCGGCAGCGGCGGCGGCGGCAGCGGCGGCG
GCGGCTCCGATATCGTGATGACCCAGAGCCCCCTGTCCCTGCCTGTCACACCTGG
CGAACCCGCCAGCATTAGCTGCAGGTCCAGCCAGAGCCTGGTGTACAGCAATGG
CGACACCTACCTGCACTGGTACCTGCAGAAGCCTGGCCAGAGCCCCCAGCTGCT
GATCTACAAGGTGAGCAACAGGTTCTCCGGAGTGCCTGACAGGTTCAGCGGCTC
CGGCAGCGGAACCGATTTCACCCTCAAGATCAGCAGAGTGGAGGCCGAGGACGT
GGGCGTCTACTATTGTAGCCAGAGCACCCACGTGCCCTGGACCTTTGGCCAGGGC
ACCAAGGTGGAGATCAAAGCTAGCGTGAAAGGGAAACACCTTTGTCCAAGTCCC
CTATTTCCCGGACCTTCTAAGCCCTTTTGGGTGCTGGTGGTGGTTGGTGGAGTCCT
GGCTTGCTATAGCTTGCTAGTAACAGTGGCCTTTATTATTTTCTGGGTGAGGAGT
AAGAGGAGCAGGAGAGTGAAGTTCAGCAGGAGCGCAGACGCCCCCGCGTACCA
GCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGAG
AAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCC
CTTCACATGCAGGCCCTGCCCCCTCGC
DG03-CD28 (SEQ ID NO: 249)
ATGGAATGGACCTGGGTGTTCCTGTTTCTGCTCTCCGTGACCGCCGGAGTGCACA
GCGAGGTGCAGCTGGTCGAAAGCGGCGGAGGACTGGTGCAGCCTGGCGGCAGCC
TGAGACTGAGCTGTGCCGCCTCCGGCTTCACCTTTAGCAGCTACGGAATGTCCTG
GGTGAGACAGGCTCCTGGCAAGGGCCTGGAACTGGTGGCCAGCATCAATAGCAA
CGGCGGCAGCACCTACTACCCTGATAGCGTGAAGGGCAGGTTCACCATCTCCAG
GGACAACGCCAAGAACAGCCTGTACCTGCAGATGAACAGCCTCAGGGCCGAGGA
CACAGCCGTGTACTACTGCGCCAGCGGCGACTATTGGGGACAGGGAACAACCGT
GACCGTCAGCAGCGCCGGCGGCGGCGGCAGCGGCGGCGGCGGCAGCGGCGGCG
GCGGCTCCGATATCGTGATGACCCAGAGCCCCCTGTCCCTGCCTGTCACACCTGG
CGAACCCGCCAGCATTAGCTGCAGGTCCAGCCAGAGCCTGGTGTACAGCAATGG
CGACACCTACCTGCACTGGTACCTGCAGAAGCCTGGCCAGAGCCCCCAGCTGCT
GATCTACAAGGTGAGCAACAGGTTCTCCGGAGTGCCTGACAGGTTCAGCGGCTC
CGGCAGCGGAACCGATTTCACCCTCAAGATCAGCAGAGTGGAGGCCGAGGACGT
GGGCGTCTACTATTGTAGCCAGAGCACCCACGTGCCCTGGACCTTTGGCCAGGGC
ACCAAGGTGGAGATCAAAGCTAGCGTGAAAGGGAAACACCTTTGTCCAAGTCCC
CTATTTCCCGGACCTTCTAAGCCCTTTTGGGTGCTGGTGGTGGTTGGTGGAGTCCT
GGCTTGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCT
ATCGCTCC
DG03-CD28tm (SEQ ID NO: 250)
ATGGAATGGACCTGGGTGTTCCTGTTTCTGCTCTCCGTGACCGCCGGAGTGCACA
GCGAGGTGCAGCTGGTCGAAAGCGGCGGAGGACTGGTGCAGCCTGGCGGCAGCC
TGAGACTGAGCTGTGCCGCCTCCGGCTTCACCTTTAGCAGCTACGGAATGTCCTG
GGTGAGACAGGCTCCTGGCAAGGGCCTGGAACTGGTGGCCAGCATCAATAGCAA
CGGCGGCAGCACCTACTACCCTGATAGCGTGAAGGGCAGGTTCACCATCTCCAG
GGACAACGCCAAGAACAGCCTGTACCTGCAGATGAACAGCCTCAGGGCCGAGGA
CACAGCCGTGTACTACTGCGCCAGCGGCGACTATTGGGGACAGGGAACAACCGT
GACCGTCAGCAGCGCCGGCGGCGGCGGCAGCGGCGGCGGCGGCAGCGGCGGCG
GCGGCTCCGATATCGTGATGACCCAGAGCCCCCTGTCCCTGCCTGTCACACCTGG
CGAACCCGCCAGCATTAGCTGCAGGTCCAGCCAGAGCCTGGTGTACAGCAATGG
CGACACCTACCTGCACTGGTACCTGCAGAAGCCTGGCCAGAGCCCCCAGCTGCT
GATCTACAAGGTGAGCAACAGGTTCTCCGGAGTGCCTGACAGGTTCAGCGGCTC
CGGCAGCGGAACCGATTTCACCCTCAAGATCAGCAGAGTGGAGGCCGAGGACGT
GGGCGTCTACTATTGTAGCCAGAGCACCCACGTGCCCTGGACCTTTGGCCAGGGC
ACCAAGGTGGAGATCAAAGCTAGCGTGAAAGGGAAACACCTTTGTCCAAGTCCC
CTATTTCCCGGACCTTCTAAGCCCTTTTGGGTGCTGGTGGTGGTTGGTGGAGTCCT
GGCTTGCTATAGCTTGCTAGTAACAGTGGCCTTTATTATTTTCTGGGTG
Construct for expression of the NDMM Nrf2 (Keap1 inhibitor peptide) (SEQ ID NO: 251)
ATGATGGATTTGGAACTTCCCCCCCCAGGGCTCCCATCCCAACAAGACATGGATC
TCATAGACATACTGTGGAGACAGGACATCGATCTGGGGGTCAGCCGCGAAGTTT
TCGACTTTTCACAAAGGCGGAAAGAATATGAATTGGAAAAGCAGAAAAAATTGG
AAAAAGAACGCCAGGAACAGCTTCAGAAGGAGCAGGAAAAAGCCTTTTTTGCCC
AGCTTCAGCTGGACGAGGAAACAGGGGAATTTCTCCCCATCCAACCAGCCCAG
Construct for expression of the NDMM human catalase (SEQ ID NO: 252)
ATGGCAGACAGTCGAGACCCTGCTagcGACCAAATGCAACATTGGAAAGAGCAAC
GGGCGGCCCAGAAAGCCGACGTTTTGACCACTGGGGCAGGTAATCCTGTTGGAG
ATAAGCTGAACGTcATcACGGTTGGACCCCGGGGACCGCTGCTCGTTCAAGACGT
GGTTTTTACGGATGAGATGGCCCATTTTGATCGAGAGAGGATACCAGAAAGGGT
TGTGCACGCTAAGGGCGCAGGTGCCTTCGGATATTTCGAGGTAACTCACGACATT
ACTAAGTATAGCAAGGCCAAGGTATTTGAACACATTGGCAAGAAGACGCCGATA
GCGGTCCGATTCAGTACAGTGGCGGGCGAGTCAGGTTCAGCCGATACCGTGAGA
GATCCGAGAGGATTTGCCGTGAAATTTTATACAGAGGACGGCAACTGGGACTTG
GTAGGAAACAATACCCCAATATTTTTCATAAGGGACCCAATCCTTTTTCCCAGCT
TTATTCATTCACAGAAGCGGAACCCACAAACGCACTTGAAAGATCCTGACATGGT
GTGGGATTTTTGGAGCTTGAGGCCAGAGAGCCTGCACCAAGTGAGCTTCTTGTTC
AGCGACAGAGGCATACCGGACGGTCATAGACACATGAACGGTTACGGTAGTCAC
ACCTTCAAACTGGTGAACGCCAACGGAGAGGCTGTCTATTGTAAGTTCCACTATA
AAACCGATCAAGGCATCAAAAACCTGAGCGTAGAGGACGCAGCCCGCCTTTCTC
AAGAAGATCCAGACTATGGGATCCGGGATCTCTTTAACGCCATAGCTACGGGTA
AATATCCCTCTTGGACGTTCTATATCCAGGTAATGACATTCAATCAAGCAGAGAC
TTTCCCCTTTAACCCGTTTGACCTTACTAAAGTATGGCCGCATAAGGACTACCCTC
TGATTCCCGTCGGCAAACTCGTGCTTAACAGGAATCCAGTCAACTATTTCGCAGA
AGTCGAGCAAATCGCCTTTGACCCTTCTAACATGCCGCCGGGAATCGAAGCGTCA
CCGGACAAGATGCTTCAAGGTCGGCTTTTCGCATACCCCGACACTCACCGACACA
GACTGGGTCCGAATTATCTTCACATACCTGTCAACTGCCCATATAGAGCACGCGT
TGCGAACTACCAGCGCGATGGTCCGATGTGCATGCAGGACAACCAGGGGGGGGC
ACCCAACTATTATCCAAATTCATTTGGGGCGCCGGAACAACAACCGTCAGCCCTT
GAACACTCCATCCAGTATTCTGGCGAAGTAAGACGCTTCAACACGGCTAATGATG
ACAACGTTACACAGGTTAGAGCGTTTTATGTGAACGTCTTGAACGAGGAACAAC
GGAAACGACTTTGCGAAAACATAGCGGGTCATTTGAAAGATGCTCAGATTTTTAT
CCAAAAAAAAGCCGTCAAAAATTTTACCGAAGTCCACCCCGATTACGGTTCACAT
ATTCAGGCCCTGTTGGATAAGTACAACGCGGAAAAGCCCAAGAATGCAATACAC
ACGTTTGTTCAGAGCGGGAGCCACCTCGCTGCTCGAGAGAAAGCAAATCTG
Construct for expression of the NDMM BDNF (SEQ ID NO: 253)
ATGACGATCCTGTTTCTGACAATGGTGATTAGCTATTTCGGATGTATGAAAGCCG
CCCCGATGAAGGAGGCCAATATCAGGGGACAAGGTGGGCTGGCTTATCCGGGCG
TAAGGACACACGGCACACTGGAGAGTGTGAACGGCCCGAAGGCCGGATCACGA
GGATTGACGAGCCTCGCAGATACGTTTGAGCATGTAATCGAAGAGCTCTTGGATG
AAGACCAAAAGGTCCGCCCCAATGAGGAGAACAACAAAGACGCAGACCTGTAC
ACATCACGAGTTATGCTGTCAAGTCAAGTGCCGCTCGAACCACCACTCCTCTTTC
TGCTGGAGGAGTACAAAAACTATTTGGACGCTGCTAACATGTCTATGCGAGTGCG
CAGACATAGTGACCCTGCCAGACGCGGTGAGCTTTCAGTCTGTGATTCTATAAGT
GAGTGGGTAACCGCAGCAGATAAGAAGACTGCCGTAGACATGTCAGGGGGAACT
GTGACTGTACTTGAAAAGGTTCCCGTTTCTAAAGGGCAGCTCAAACAGTATTTCT
ATGAAACAAAGTGTAATCCAATGGGGTACACCAAGGAAGGTTGCAGGGGAATCG
ACAAGCGACATTGGAACAGTCAATGTCGGACCACTCAGAGCTACGTCCGCGCTC
TCACGATGGATAGTAAGAAACGCATCGGGTGGAGATTCATCAGAATCGACACCT
CTTGCGTCTGTACTCTTACAATTAAGCGAGGGCGA
Construct for expression of the NDMM IGF-1 (SEQ ID NO: 254)
ATGGGGAAAATCTCCTCTCTCCCTACCCAGTTGTTCAAGTGTTGCTTTTGTGACTT
CTTGAAGGTAAAAATGCACACTATGTCATCCAGTCACCTTTTTTATTTGGCTCTGT
GCCTCCTCACATTCACCAGTTCAGCTACTGCCGGGCCTGAAACACTCTGCGGCGC
CGAACTCGTTGATGCGCTTCAATTCGTGTGTGGAGATAGGGGGTTTTACTTTAAC
AAGCCGACGGGTTATGGTAGCTCAAGTAGACGAGCGCCACAGACTGGAATAGTA
GATGAATGCTGTTTCCGCTCATGCGACCTTCGCAGATTGGAAATGTACTGCGCTC
CTCTTAAACCAGCAAAGAGTGCGCGGTCCGTGCGAGCCCAACGACATACCGATA
TGCCAAAAACCCAGAAATATCAGCCGCCGTCTACCAACAAGAACACCAAGAGTC
AGAGGAGAAAGGGTTGGCCCAAGACGCACCCGGGTGGCGAACAAAAAGAAGGT
ACTGAGGCAAGTTTGCAAATTCGAGGAAAGAAGAAAGAACAACGAAGAGAGAT
AGGTTCTCGCAATGCGGAATGTCGAGGCAAAAAAGGTAAG
Forward primer human β-actin (SEQ ID NO: 255)
GGC CGA GGA CTT TGA TTG C
Reverse primer human β-actin (SEQ ID NO: 256)
TGG GGT GGC TTT TAG GAT GG
Forward primer human IL-4 (SEQ ID NO: 257)
GCT TCC CCC TCT GTT CTT CC
Reverse primer human IL-4 (SEQ ID NO: 258)
GAT GTC TGT TAC GGT CAA CTC G
Forward primer human IL-10 (SEQ ID NO: 259)
TCA AGG CGC ATG TGA ACT CC
Reverse primer human IL-10 (SEQ ID NO: 260)
CAG GGA AGA AAT CGA TGA CAG C
Amino acid sequence of Mature BDNF and Signal Peptide (SEQ ID NO: 261)
MTILFLTMVISYFGCMKAHSDPARRGELSVCDSISEWVTAADKKTAVDMSGGTV
TVLEKVPVSKGQLKQYFYETKCNPMGYTKEGCRGIDKRHWNSQCRTTQSYVR
ALTMDSKKRIGWRFIRIDTSCVCTLTIKRGR

1. A method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising administering an effective amount of cells which are engineered to express a chimeric antigen receptor (“CAR”) which targets at least one (i) aberrant protein which is expressed at site(s) of neurodegeneration associated with a specific neurodegenerative disease and is associated with the pathology of said specific neurodegenerative disease or condition and/or (ii) a protein which is aberrantly expressed (e.g. overexpressed) at site(s) of neurodegeneration associated with a specific neurodegenerative disease and/or is associated with the pathology of said specific neurodegenerative disease or condition, or in addition the cells are engineered to express a neurodegenerative disease modifying molecule (NDMM), wherein said CAR and NDMM may be expressed by the same or different cells, wherein said CAR and/or NDMM expressing cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is associated with the expression of said aberrant or aberrantly expressed protein.

2. The method of claim 1, wherein:
(i) said site(s) of neurodegeneration are present in the central nervous system;
(ii) said site(s) of neurodegeneration are present in the peripheral nervous system;
(iii) said site(s) of neurodegeneration are present in the peripheral and the central nervous system;
(iv) said CAR-expressing cells comprise immune cells;
(v) said CAR-expressing immune cells comprise T cells or T cell progenitors,
(vi) said CAR-expressing immune cells comprise T regulatory cells (Tregs) such as FOXP3⁺ Tregs;
(vii) the administered cells comprise a CAR which recognizes at least one aberrant protein expressed at a site of neurodegeneration and optionally express viral IL-10;
(viii) the CAR comprises an scFv or ligand which recognizes at least one aberrant protein or aberrantly expressed protein expressed at a site of neurodegeneration;
(ix) the CAR comprised on said administered cells comprises DG01 (SEQ ID NO: 1), DG02 (SEQ ID NO: 2), DG03 (SEQ ID NO: 3), DG04 (SEQ ID NO: 4), DG05 (SEQ ID NO: 5), DG06 (SEQ ID NO: 6), and/or DG07 (SEQ ID NO: 7), DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs;
(x) the administered cells are engineered to express one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs;
(xi) the administered cells of (x), wherein each of said one or more constructs targets mutSOD1 and optionally encodes viral IL-10;
(xii) the administered cells are engineered to express one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (also referred to as DG03-28tm) (SEQ ID NO: 50), and/or a construct comprising at least 90%, at least 95%, at least 98%, or at least 99% sequence identity to any one or more of the aforementioned constructs;
(xiii) the administered cells of (x) wherein each of said one or more constructs targets amyloid beta;
(xiv) said CAR cells are further engineered to express at least one NDMM, e.g., a pro-neuronal factor or nerve growth factor, wherein said CAR and said NDMM are expressed by the same or different cells;
(xv) said administered cells are engineered to express NDMM Nrf2 (Keap1 inhibitor peptide) (SEQ ID NO: 51), and/or a construct comprising at least 90%, at least 95%, at least 98%, or at least 99% sequence identity to the aforementioned construct;
(xvi) said administered cells are engineered to express human catalase (SEQ ID NO: 52), and/or a construct comprising at least 90%, at least 95%, at least 98%, or at least 99% sequence identity to the aforementioned construct;
(xvii) said administered cells are engineered to express viral IL-10 and/or BDNF (SEQ ID NO: 53), and/or a construct comprising at least 90%, at least 95%, at least 98%, or at least 99% sequence identity to the aforementioned construct;
(xviii) said administered cells are engineered to express viral IL-10 and/or IGF-1 (SEQ ID NO: 54), and/or a construct comprising at least 90%, at least 95%, at least 98%, or at least 99% sequence identity to the aforementioned construct;
(xix) said cells are further engineered to express at least one anti-oxidative protein which inhibits or protects neurons from anti-oxidative stress and/or inhibits or prevents the death of neurons at the site of neurodegeneration, wherein said CAR and said anti-oxidative protein are expressed by the same or different cells;
(xx) said cells are further engineered to express at least one anti-oxidative protein which also promotes T cell function or T cell lifespan;
(xxi) the neurodegenerative disease or condition comprises at least one of Parkinson's disease, Alzheimer's disease, Prion disease, a Motor neurone disease (MND) such as amyotrophic lateral sclerosis (ALS), Huntington's disease (HD), Spinocerebellar ataxia (SCA), Spinal muscular atrophy (SMA), Friedreich's ataxia, Lewy body disease, epilepsy, encephalitis, hydrocephalus, stroke, chronic traumatic encephalopathy (CTE); a synucleinopathy; a tauopathy, a spongiform encephalopathy; familial amyloidotic polyneuropathy; Dutch hereditary cerebral hemorrhage with amyloidosis; congophilic angiopathy; corticobasal degeneration; Pick's disease; progressive supranuclear palsy; Creutzfeldt-Jacob disease; Gerstmann-Sträussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type L diffuse Lewy body disease; frontotemporal lobar degeneration; hereditary dentatorubral-pallidoluysian atrophy; Kennedy's disease; Alexander's disease; Cockayne syndrome; and Icelandic hereditary cerebral hemorrhage with amyloidosis;
(xxii) the neurodegenerative disease comprises Parkinson's disease;
(xxiii) the neurodegenerative disease comprises Alzheimer's disease;
(xxiv) the neurodegenerative disease comprises amyotrophic lateral sclerosis (ALS);
(xxv) the CAR binds to one or more of human amyloid beta, amyloid-beta 1-42, alpha-synuclein, superoxide dismutase-1 (SOD-1), hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; and Leucine-rich repeat serine/threonine-protein kinase 2 (LRRK2), Huntingtin with tandem glutamine repeats; prion proteins; transthyretin; dentatorubral pallidoluysian atrophy (DRPLA) protein; androgen receptor; an ataxin; P/Q-type calcium channel α1A subunit; TATA-box-binding protein; glial fibrillary acidic protein; DNA excision repair protein ERCC-6; survival motor neuron protein; and cystatin C;
(xxvi) the administered cells express at least one pro-neuronal factor, neurotrophic factor, or nerve growth factor selected from brain-derived neurotrophic factor (BDNF), ciliary neurotrophic factor (CNTF), glial cell-line derived neurotrophic factor (GDNF), interleukin-1 receptor antagonist (IL-1ra); interleukin-6 (IL-6); activated protein C (APC); thrombomodulin; tissue plasminogen activator (tPA); Protein deglycase DJ-1; a tissue inhibitor of metalloproteinases (TIMP), insulin-like growth factor-1 (IGF-1), vascular endothelial growth factor (VEGF), fibroblast growth factor (FGF), a bone morphogenetic protein (BMP), erythropoietin (EPO), thrombopoietin (TPO), and granulocyte-colony stimulating factor (G-CSF), optionally wherein said at least one pro-neuronal factor, neurotrophic factor, or nerve growth factor are expressed by the same cell as said CAR or by a different cell as said CAR;
(xxvii) administered cells express at least one anti-oxidative protein selected from superoxide dismutases such as human superoxide dismutase, Cu/Zn superoxide dismutase, HO-1, ferritin, glutathione reductase, glutathione peroxidase, ferritin (H), metallothionein I, thioredoxin, thioredoxin reductase, peroxiredoxins (Prxs) such as pereoxiredoxin MSP₂₃; activity-dependent neuroprotector homeobox (ADNP); phycocyanin; neuroglobin, catalase, and NRF2, optionally wherein said at least one anti-oxidative protein is expressed by the same cell as said CAR or by a different cell as said CAR;
(xxviii) the administered cells reduce or stabilize the amount of inflammation present at said site(s) of neurodegeneration;
(xxix) the administered cells inhibit or prevent at least one of:
(1) microglia cell over-activation wherein over-activation includes microglia which possess at least one activity or increase in an activity characteristic of activated microglia such as (a) a change in morphology, (b) migration to inflammatory sites, (c) production of neurotoxic or inflammatory cytokines such as IL-1, (d) interaction with neural plaques or β amyloid deposits, (e) synthesis of neurotoxic proteins, (f) secretion of proteases and/or reactive oxygen species, (g) induction of amyloid production by neighboring cells, (h) destruction of myelin;
(2) increased numbers of microglia;
(3) the production of inflammatory proteins or inflammatory activities at sites of neurodegeneration; and/or
(4) neuronal death or impaired neuronal function;
(xxx) the administered cells slow the onset of the neurodegenerative disease;
(xxxi) the administered cells slow the progression of the neurodegenerative disease;
(xxxii) the administered cells repair or increase neural cell function or slow the loss of neural cell function;
(xxxiii) the administered cells increase cognition or stabilize the loss of cognition in the treated subject;
(xxxiv) the administered cells reduce or slow the loss of neuromotor function and/or paralysis in the treated subject;
(xxxv) the administered cells reduce the number or size of neural lesions or plaques in the treated subject;
(xxxvi) the administered cells reduce the number of or severity of seizures in the treated subject;
(xxxvii) the administered cells express increased levels of IL-10, optionally viral IL-10, in response to mSOD1 antigen in the treated subject;
(xxxviii) the administered cells express increased levels of cell surface markers including one or more of GITR, PD-1 and/or CTLA-4 in response to mSOD1 antigen in the treated subject;
(xxxix) the administered cells inhibit superoxide generation in response to mSOD1 antigen and/or anti-CD3 in the treated subject;
(xxxx) the administered cells inhibit TNF-α production in response to mSOD1 antigen in the treated subject;
(xxxxi) the administered cells are engineered to express a construct comprising DG05 (SEQ ID NO: 5), and/or a construct comprising at least 90%, at least 95%, at least 98%, or at least 99% sequence identity to the aforementioned construct;
(xxxxii) the administered cells are engineered to express one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs;
(xxxxiii) the administered cells express increased levels of IL-10, optionally viral IL-10, and/or IL-4 in response to amyloid beta antigen in the treated subject;
(xxxxiv) the administered cells inhibit superoxide generation in response to amyloid beta antigen and/or anti-CD3 in the treated subject;
(xxxxv) the administered cells inhibit IL-6 production in response to amyloid beta antigen and/or anti-CD3 in the treated subject;
(xxxxvi) the administered cells protect cells of the treated subject from hydrogen peroxide toxicity;
(xxxxvii) the administered cells are engineered to express a construct comprising DG03 (SEQ ID NO: 3);
(xxxxviii) the administered cells are engineered to express one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs;
(xxxxix) the administered cells protect cells of the treated subject from hydrogen peroxide toxicity;
(l) the administered cells are engineered to express one or more of the following constructs: NDMM human catalase construct (SEQ ID NO: 52), NDMM Nrf2 (Keap1 inhibitor peptide) construct (SEQ ID NO: 51), NDMM BDNF construct (SEQ ID NO: 53), NDMM IGF-1 construct (SEQ ID NO: 54), and/or a construct comprising at least 90%, at least 95%, at least 98%, or at least 99% sequence identity to any one or more of the aforementioned constructs;
(li) wherein the cells are administered systemically or locally;
(lii) the cells when administered cross the blood brain barrier (BBB);
(liii) the cells are administered by injection, e.g. by a means selected from intravenous, subcutaneous, intracavitary, intraventricular, intracranial, and intrathecal;
(liv) the CAR comprises an scFv which binds to one or more of human amyloid beta, human superoxide dismutases, or human alpha-synuclein;
(lv) the CAR comprises an scFv which binds to one or more of human amyloid beta, human superoxide dismutases, or human alpha-synuclein having a sequence contained in Table 1;
(lvi) the administered cells further comprise a suicide gene, optionally expressed under the control of an inducible promoter;
(lvii) the CAR expressed by the administered cells comprises at least one signaling domain, e.g., a costimulatory domain, optionally selected from CD28-CD3ζ, 4-1BB-CD3ζ, DAP10-CD3ζ, CD44-CD3ζ, CTLA4-CD3ζ, CD28, DAP10, 4-1BB and CD3ζ;
(lviii) the CAR expressed by the administered cells comprises at least one signaling domain selected from CD28-CD3ζ, DAP10-CD3ζ, CD44-CD3ζ, CD28 and CD3ζ;
(lix) the cells express several CARs which recognize different proteins expressed at sites of neurodegeneration or the administered cells are comprise of cells which themselves express different CARs according to any of the foregoing.

3-59. (canceled)

60. A nucleic acid which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory domain, the expression of which are optionally controlled by the same or different inducible or constitutive promoters.

61. The nucleic acid of claim 60, wherein
(i) the protein associated with a neurodegenerative disease or condition is selected from human amyloid beta, amyloid-beta 1-42, alpha-synuclein, superoxide dismutase-1 (SOD-1), hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin; ataxin-2; parkin; Serine/threonine-protein kinase PINK1; and Leucine-rich repeat serine/threonine-protein kinase 2 (LRRK2), Huntingtin with tandem glutamine repeats; prion proteins; transthyretin; dentatorubral pallidoluysian atrophy (DRPLA) protein; androgen receptor; an ataxin; P/Q-type calcium channel α1A subunit; TATA-box-binding protein; glial fibrillary acidic protein; DNA excision repair protein ERCC-6; survival motor neuron protein; and cystatin C;
(ii) the protein associated with a neurodegenerative disease or condition comprises one or more of an amyloid protein, e.g, amyloid-beta protein, or a mutant superoxide dismutase 1, or human alpha-synuclein;
(iii) the nucleic acid further encodes human catalase and/or the Neh2 domain of human Nrf2;
(iv) the nucleic acid further encodes human IGF-1;
(v) the nucleic acid further encodes human BDNF;
(vi) the nucleic acid further encodes further encodes one or more of the following constructs: NDMM Nrf2 (Keap1 inhibitor peptide) (SEQ ID NO: 51); NDMM human catalase (SEQ ID NO: 52); NDMM BDNF (SEQ ID NO: 53); and/or NDMM IGF-1 (SEQ ID NO: 54), and/or a construct comprising at least 90%, at least 95%, at least 98%, or at least 99% sequence identity to any one or more of the aforementioned constructs;
(vii) the nucleic acid of any of the foregoing wherein a ligand binding domain in the CAR comprises a scFv;
(viii) the nucleic acid of any of the foregoing, wherein the CAR comprises at least one signaling domain e.g., a costimulatory domain, optionally selected from CD28-CD3ζ, 4-1BB-CD3ζ, DAP10-CD3ζ, CD44-CD3ζ, CTLA4-CD3ζ, CD28, DAP10, 4-1BB and CD3ζ;
(ix) the nucleic acid of any of the foregoing, which encodes one or more of the following: DG01 (SEQ ID NO: 1), DG02 (SEQ ID NO: 2), DG03 (SEQ ID NO: 3), DG04 (SEQ ID NO: 4), DG05 (SEQ ID NO: 5), DG06 (SEQ ID NO: 6), DG07 (SEQ ID NO: 7), DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs;
(x) the nucleic acid of any of the foregoing, which encodes one or more of the following: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs;
(xi) the nucleic acid of any of the foregoing, which encodes one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3) ID NO: 48); DG03-CD28 (also referred to as DG03-28) (SEQ ID NO: 49); DG03-CD28tm (also referred to as DG03-28tm) (SEQ ID NO: 50), and/or a construct comprising at least 90%, at least 95%, at least 98%, or at least 99% sequence identity to any one or more of the aforementioned constructs;
(xii) the nucleic acid of any of the foregoing, which is contained on a construct such as a plasmid or a virus, e.g., a retroviral construct;
(xiii) the nucleic acid of any of the foregoing, wherein the nucleic acid encoding the CAR is contained on a construct which further comprises a gene encoding a pro-neuronal factor and/or an anti-oxidant protein and/or a nerve growth factor or non-classical neurotrophic factor;
(xiv) the nucleic acid of any of the foregoing, wherein the nucleic acid encoding the CAR is contained on a construct which further comprises a nucleic acid encoding at least one anti-oxidative protein, e.g., one which prolongs T cell function and/or which is neuroprotective;
(xv) the nucleic acid of any of the foregoing, wherein the ligand binding moiety in the CAR binds to an aberrant protein or a protein which is aberrantly expressed at sites of neurodegeneration or neuroinflammation present in individuals comprising a neurodegenerative disease or condition selected from Parkinson's disease, Alzheimer's disease, Prion disease, a Motor neurone disease (MND) such as amyotrophic lateral sclerosis (ALS), Huntington's disease (HD), Spinocerebellar ataxia (SCA), Spinal muscular atrophy (SMA), Friedreich's ataxia, Lewy body disease, epilepsy, encephalitis, hydrocephalus, stroke, chronic traumatic encephalopathy (CTE); a synucleinopathy; a tauopathy, a spongiform encephalopathy; familial amyloidotic polyneuropathy; Dutch hereditary cerebral hemorrhage with amyloidosis; congophilic angiopathy; corticobasal degeneration; Pick's disease; progressive supranuclear palsy; Creutzfeldt-Jacob disease; Gerstmann-Sträussler-Schneiker syndrome; fatal familial insomnia; kuru, bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; and Icelandic hereditary cerebral hemorrhage with amyloidosis;
(xvi) the nucleic acid of any of the foregoing, wherein the ligand binding moiety in the CAR binds to an aberrant protein or a protein which is aberrantly expressed at sites of neurodegeneration or neuroinflammation present in individuals comprising Parkinson's disease;
(xvii) the nucleic acid of any of the foregoing, wherein the ligand binding moiety in the CAR binds to an aberrant protein or a protein which is aberrantly expressed at sites of neurodegeneration or neuroinflammation present in individuals comprising Alzheimer's disease;
(xviii) the nucleic acid of any of the foregoing, wherein the ligand binding moiety in the CAR binds to an aberrant protein or a protein which is aberrantly expressed at sites of neurodegeneration or neuroinflammation present in individuals comprising amyotrophic lateral sclerosis (ALS);
(xix) the nucleic acid of any of the foregoing, wherein a ligand binding moiety in the CAR binds to one or more of human amyloid beta, amyloid-beta 1-42, alpha-synuclein, superoxide dismutase-1 (SOD-1), hyperphosphorylated tau protein; TAR DNA-binding protein 43 (TDP-43): chromosome 9 open reading frame 72 (c9orf72); β-Synuclein; γ-Synuclein; RNA-binding protein fused in sarcoma (FUS); ubiquitin; ubiquilin-2, p62; optineurin, ataxin-2; parkin; Serine/threonine-protein kinase PINK1; and Leucine-rich repeat serine/threonine-protein kinase 2 (LRRK2), Huntingtin with tandem glutamine repeats; prion proteins; transthyretin; dentatorubral pallidoluysian atrophy (DRPLA) protein; androgen receptor; an ataxin; P/Q-type calcium channel α1A subunit; TATA-box-binding protein; glial fibrillary acidic protein; DNA excision repair protein ERCC-6; survival motor neuron protein; and cystatin C;
(xx) the nucleic acid of any of the foregoing, which comprises a nucleic acid encoding at least one pro-neuronal factor, neurotrophic factor, or nerve growth factor selected from brain-derived neurotrophic factor (BDNF), ciliary neurotrophic factor (CNTF), glial cell-line derived neurotrophic factor (GDNF), interleukin-1 receptor antagonist (IL-1ra); interleukin-6 (IL-6); activated protein C (APC); thrombomodulin; tissue plasminogen activator (tPA); Protein deglycase DJ-1; a tissue inhibitor of metalloproteinases (TIMP), insulin-like growth factor-1 (IGF-1), vascular endothelial growth factor (VEGF), fibroblast growth factor (FGF), a bone morphogenetic protein (BMP), erythropoietin (EPO), thrombopoietin (TPO), and granulocyte-colony stimulating factor (G-CSF);
(xxi) the nucleic acid of any of the foregoing, which comprises a nucleic acid encoding an anti-oxidative protein which is selected from superoxide dismutases such as human superoxide dismutase, Cu/Zn superoxide dismutase, HO-1, ferritin, glutathione reductase, glutathione peroxidase, ferritin (H), metallothionein I, thioredoxin, thioredoxin reductase, peroxiredoxins (Prxs) such as pereoxiredoxin MSP₂₃; activity-dependent neuroprotector homeobox (ADNP); phycocyanin; neuroglobin, catalase, and NRF2, or any combination of the foregoing.

62-81. (canceled)

82. A recombinant or engineered cell which comprises at least one nucleic acid encoding a CAR according to claim 60;

83. The cell of claim 82, which comprises:
(i) a human immune cell, e.g., a T cell or T cell progenitor or an NK cell;
(ii) a Treg cell, e.g., a FoxP3⁺ Treg cell;
(iii) does not express a functional TCR;
(iv) comprises a suicide gene optionally expressed under the control of an inducible promoter;
(v) is further engineered to express at least one anti-oxidant, nerve growth factor or non-classical neurotrophic factor the expression of which is optionally under the control of an inducible promoter;
(vi) when administered crosses the BBB;
(vii) possesses Treg effector functions;
(viii) expresses Treg surface markers and/or expresses IL-10 or a viral variant of IL-10;
(ix) at least one CAR is expressed on the surface thereof;
(x) at least one of the following constructs are expressed by said cell: DG01 (SEQ ID NO: 1), DG02 (SEQ ID NO: 2), DG03 (SEQ ID NO: 3), DG04 (SEQ ID NO: 4), DG05 (SEQ ID NO: 5), DG06 (SEQ ID NO: 6), and/or DG07 (SEQ ID NO: 7), DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs;
(xi) at least one of the following constructs are expressed by said cell: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs;
(xii) at least one of the following constructs are expressed by said cell: NDMM Nrf2 (Keap1 inhibitor peptide) (SEQ ID NO: 51); NDMM human catalase (SEQ ID NO: 52); NDMM BDNF (SEQ ID NO: 53); NDMM IGF-1 (SEQ ID NO: 54), and/or a construct comprising at least 90%, at least 95%, at least 98%, or at least 99% sequence identity to any one or more of the aforementioned constructs;
(xiii) when administered to a subject elicits one or more of the following properties at sites of neurodegeneration comprising the antigen bound by the CAR: (i) inhibits macrophage activity or activation (wherein over-activation includes microglia which possess at least one activity or increase in an activity characteristic of activated microglia such as (1) a change in morphology, (2) migration to inflammatory sites, (3) production of neurotoxic or inflammatory cytokines such as IL-1, (4) interaction with neural plaques or 13 amyloid deposits, (5) synthesis of neurotoxic proteins, (6) secretion of proteases and/or reactive oxygen species, (7) induction of amyloid production by neighboring cells, (8) destruction of myelin), (ii) inhibits macrophages, (iii) inhibits secretion of inflammatory cytokines, proteases, and oxidants such as IL-1β, TNFα, NO and other neurotoxins by macrophages or microglia, (iv) promotes secretion of secretes more of one or more of IL-10, IL-4, and TGF-beta, and (v) inhibits inflammation or neurodegeneration;
(xiv) when administered to a subject with a neurodegenerative disease inhibits inflammation and/or produces anti-inflammatory mediators at sites of inflammation in the CNS;
(xv) when administered to a subject with a neurodegenerative disease inhibits or prevents at least one of at a site of neurodegeneration:
(1) microglia cell over-activation (as above-defined),
(2) neurotoxic macrophage activity and/or secretion of neurotoxic moieties thereby;
(3) loss of neuronal function;
(4) increased microglia cell number or macrophage number;
(5) the production of inflammatory proteins or inflammatory activities at sites of neurodegeneration, and
(6) neuronal death;
(xvi) when administered to a subject at risk of developing a neurodegenerative disease slows the onset of the neurodegenerative disease;
(xvii) when administered to a subject with a neurodegenerative disease slows the progression of the neurodegenerative disease;
(xviii) when administered to a subject with a neurodegenerative disease repairs or increases neural cell function or slows the loss of neural cell function;
(xix) when administered to a subject with a neurodegenerative disease increases cognition or inhibits or slows the loss of cognition in the treated subject;
(xx) when administered to a subject with a neurodegenerative disease reduces or slows the loss of neuromotor function and/or paralysis in the treated subject;
(xxi) when administered to a subject with a neurodegenerative disease reduces the number or size of neural lesions or plaques in the treated subject;
(xxii) when administered to a subject with a neurodegenerative disease reduces the number of or severity of seizures in the treated subject;
(xxiii) when administered to a subject expresses increased levels of IL-10 in response to mSOD1 antigen in the subject;
(xxiv) when administered to a subject expresses increased levels of the cell surface markers of any one or more of GITR, PD-1 and/or CTLA-4 in response to mSOD1 antigen in the subject;
(xxv) when administered to a subject inhibits superoxide generation in response to mSOD1 antigen and/or anti-CD3 in the subject;
(xxvi) when administered to a subject inhibits TNF-α production in response to mSOD1 antigen in the subject;
(xxvii) said cell is engineered to express a construct comprising DG05 (SEQ ID NO: 5);
(xxviii) said cell is engineered to express one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs;
(xxix) when administered to a subject expresses increased levels of IL-10 and/or IL-4 in response to amyloid beta antigen in the subject;
(xxx) when administered to a subject inhibits superoxide generation in response to amyloid beta antigen and/or anti-CD3 in the subject;
(xxxi) when administered to a subject inhibits IL-6 production in response to amyloid beta antigen and/or anti-CD3 in the subject;
(xxxii) when administered to a subject protects cells of the subject from hydrogen peroxide toxicity;
(xxxiii) said cell is engineered to express a construct comprising DG03 (SEQ ID NO: 3);
(xxxiv) said cell is engineered to express one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs;
(xxxv) when administered to a subject protects cells of the subject from hydrogen peroxide toxicity;
(xxxvi) said cell is engineered to express one or more of the following constructs: NDMM human catalase construct (SEQ ID NO: 52), NDMM Nrf2 (Keap1 inhibitor peptide) construct (SEQ ID NO: 51), NDMM BDNF construct (SEQ ID NO: 53), NDMM IGF-1 construct (SEQ ID NO: 54), and/or a construct comprising at least 90%, at least 95%, at least 98%, or at least 99% sequence identity to any one or more of the aforementioned constructs; or any combination of the foregoing.

84-118. (canceled)

119. A composition suitable for therapy comprising recombinant or engineered cells according to claim 82, and a pharmaceutically acceptable carrier, which optionally further comprises at least one stabilizer and/or an additive that promotes the ability of the cells contained therein to cross the BBB or the cells are attached or complexed with a moiety additive that promotes the ability of the cells to cross the BBB.

120-121. (canceled)

122. A method of treating a subject comprising a neurodegenerative disease or condition, exhibiting one or more risk factors associated with the development of a neurodegenerative disease or condition, and/or exhibiting one or more signs or symptoms associated with the diagnosis of a neurodegenerative disease or condition, comprising:
(i) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (e.g. overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease;
(ii) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease, and a CAR of said CAR-expressing cells includes DG01 (SEQ ID NO: 1), DG02 (SEQ ID NO: 2), DG03 (SEQ ID NO: 3), DG04 (SEQ ID NO: 4), and/or a construct comprising at least 90%, at least 95%, at least 98%, or at least 99% sequence identity to any one or more of the aforementioned constructs;
(iii) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease, and said cells are engineered to express any one or more of the following: DG03 (SEQ ID NO: 3); DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3ζ) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3ζ) (SEQ ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs;
(iv) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Alzheimer's disease, and a CAR of said CAR-expressing cells includes DG01 (SEQ ID NO: 1), DG02 (SEQ ID NO: 2), DG03 (SEQ ID NO: 3), DG04 (SEQ ID NO: 4), and/or a construct comprising at least 90%, at least 95%, at least 98%, or at least 99% sequence identity to any one or more of the aforementioned constructs, and further wherein said cells are engineered to express one or more neurodegenerative disease modifying molecules (NDMMs), optionally wherein said cells are engineered to express one or more of the following constructs: NDMM human catalase construct (SEQ ID NO: 52), NDMM Nrf2 (Keap1 inhibitor peptide) construct (SEQ ID NO: 51), NDMM BDNF construct (SEQ ID NO: 53), and/or NDMM IGF-1 construct (SEQ ID NO: 54), and/or a construct comprising at least 90%, at least 95%, at least 98%, or at least 99% sequence identity to any one or more of the aforementioned constructs;
(v) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS;
(vi) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS, and a CAR of said CAR-expressing cells includes DG05 (SEQ ID NO: 5), DG06 (SEQ ID NO: 6), DG07 (SEQ ID NO: 7), and/or a construct comprising at least 90%, at least 95%, at least 98%, or at least 99% sequence identity to any one or more of the aforementioned constructs;
(vii) administering an effective amount of cells which are engineered to express a chimeric antigen receptor (“CAR”) or an NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS, and said cells are engineered to express any one or more of the following: DG05 (SEQ ID NO: 5); DG05-CD28-CD3ζ (also referred to as DG05-28-3ζ) (SEQ ID NO: 24); DG05-CD28tm-DAP10-CD3ζ (also referred to as DG05-28tm-10-3ζ) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3ζ) (SEQ ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs;
(viii) administering an effective amount of cells which are engineered to express a chimeric antigen receptor (“CAR”) or a NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with ALS, and a CAR of said CAR-expressing cells includes DG05 (SEQ ID NO: 5), DG06 (SEQ ID NO: 6), DG07 (SEQ ID NO: 7), and/or a construct comprising at least 90%, at least 95%, at least 98%, or at least 99% sequence identity to any one or more of the aforementioned constructs, and further wherein said cells are engineered to express one or more neurodegenerative disease modifying molecules (NDMMs), optionally wherein said cells are engineered to express one or more of the following constructs: NDMM human catalase construct (SEQ ID NO: 52), NDMM Nrf2 (Keap1 inhibitor peptide) construct (SEQ ID NO: 51), NDMM BDNF construct (SEQ ID NO: 53), NDMM IGF-1 construct (SEQ ID NO: 54), and/or a construct comprising at least 90%, at least 95%, at least 98%, or at least 99% sequence identity to any one or more of the aforementioned constructs;
(ix) administering an effective amount of cells which are engineered to express a chimeric antigen receptor (“CAR”) or a NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Parkinson's disease;
(x) administering an effective amount of cells which are engineered to express a chimeric antigen receptor (“CAR”) or a NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said cells are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Parkinson's disease, and a CAR of said CAR-expressing cells includes DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs;
(xi) administering an effective amount of cells which are engineered to express a chimeric antigen receptor (“CAR”) or a NDMM, wherein the CAR and the NDMM may be expressed by the same or different cells, which targets at least one (i) aberrant protein which is expressed in the central nervous system at site(s) of neurodegeneration associated with a specific neurodegenerative disease and is associated with the pathology of said specific neurodegenerative disease or condition and/or (ii) a protein which is aberrantly expressed (overexpressed) in the central nervous system at site(s) of neurodegeneration associated with a specific neurodegenerative disease and is associated with the pathology of said specific neurodegenerative disease or condition, wherein said are administered under conditions whereby they are in contact with said site(s) of neurodegeneration comprising said targeted protein and thereby prevent, inhibit or treat the neurodegenerative disease or condition and/or one or more symptoms associated with the neurodegenerative disease or condition which is characterized by the expression of said aberrant or aberrantly expressed protein, wherein said targeted protein is a protein associated with Parkinson's disease, and a CAR of said CAR-expressing cells includes DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs, and further wherein said cells are engineered to express one or more neurodegenerative disease modifying molecules (NDMMs), optionally wherein said cells are engineered to express one or more of the following constructs: NDMM human catalase construct (SEQ ID NO: 52), NDMM Nrf2 (Keap1 inhibitor peptide) construct (SEQ ID NO: 51), NDMM BDNF construct (SEQ ID NO: 53), NDMM IGF-1 construct (SEQ ID NO: 54), and/or a construct comprising at least 90%, at least 95%, at least 98%, or at least 99% sequence identity to any one or more of the aforementioned constructs.

123-135. (canceled)

136. A nucleic acid which encodes a chimeric antigen receptor (CAR) and optionally a NDMM selected from:
(1) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said protein is a protein associated with Alzheimer's disease,
(2) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said protein is a form of amyloid beta associated with Alzheimer's disease;
(3) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said protein is a protein associated with ALS disease;
(4) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said protein is mutated or aberrantly expressed SOD1;
(5) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said protein is a protein associated with Parkinson's disease;
(6) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said protein is a form of alpha-synuclein associated with Parkinson's disease;
(7) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes DG01 (SEQ ID NO: 1), DG02 (SEQ ID NO: 2), DG03 (SEQ ID NO: 3), DG04 (SEQ ID NO: 4), and/or a construct comprising at least 90%, at least 95%, at least 98%, or at least 99% sequence identity to any one or more of the aforementioned constructs;
(8) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes any one or more of the following: DG03-CD28-CD3ζ (also referred to as DG03-28-3ζ) (SEQ ID NO: 22); DG03-CD28tm-DAP10-CD3ζ (also referred to as DG03-28tm-10-3ζ) (SEQ ID NO: 45); DG03-CD28tm-CD44-CD3ζ (also referred to as DG03-28tm-44-3) (SEQ ID NO: 46); DG03-CD28tm-4-1-BB-CD3ζ (also referred to as DG03-28tm-BB-3ζ) (SEQ ID NO: 47); DG03-CD28tm-CD3ζ (also referred to as DG03-28tm-3) ID NO: 48); DG03-CD28 (also referred to as DG03-28) (SEQ ID NO: 49); DG03-CD28tm (SEQ ID NO: 50), and/or a construct comprising at least 90%, at least 95%, at least 98%, or at least 99% sequence identity to any one or more of the aforementioned constructs;
(9) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes DG05 (SEQ ID NO: 5), DG06 (SEQ ID NO: 6), DG07 (SEQ ID NO: 7), and/or a construct comprising at least 90%, at least 95%, at least 98%, or at least 99% sequence identity to any one or more of the aforementioned constructs;
(10) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes any one or more of the following constructs: DG05-CD28-CD3ζ (also referred to as DG05-28-3ζ) (SEQ ID NO: 24); DG05-CD28tm-DAP10-CD3ζ (also referred to as DG05-28tm-10-3) (SEQ ID NO: 40); DG05-CD28tm-CD44-CD3ζ (also referred to as DG05-28tm-44-3ζ) (SEQ ID NO: 41); DG05-CD28tm-CD3ζ (also referred to as DG05-28tm-3) ID NO: 42); DG05-CD28 (also referred to as DG05-28) (SEQ ID NO: 43); DG05-CD28tm (also referred to as DG05-28tm) (SEQ ID NO: 44), and/or a construct comprising at least 90%, at least 95%, at least 98%, or at least 99% sequence identity to any one or more of the aforementioned constructs; or
(11) one which encodes a chimeric antigen receptor (CAR) comprising (i) at least one ligand binding moiety which binds to an aberrant protein associated with the pathology of a neurodegenerative disease or a protein which is aberrantly (overexpressed) in the central nervous system at site(s) of neurodegeneration which protein is associated with the pathology of a specific neurodegenerative disease or condition and (ii) optionally at least one signaling domain, e.g., a costimulatory signaling domain, and (iii) further optionally an NDMM, the expression of which are optionally controlled by the same or different inducible or constitutive promoters, wherein said nucleic acid encodes DG08 (SEQ ID NO: 8), DG09 (SEQ ID NO: 9), DG10 (SEQ ID NO: 10), DG11 (SEQ ID NO: 11), and/or a construct comprising at least 90%, at least 95%, at least 98%, or at least 99% sequence identity to any one or more of the aforementioned constructs.

137-146. (canceled)

147. A modified Treg which comprises a nucleic acid which encodes a chimeric antigen receptor (CAR) according to claim 136.

148-157. (canceled)

158. A modified Treg according to claim 147 which is further engineered to express or overexpress IL-10, optionally viral IL-10, and/or IL-4.
